(19) United States
(12) Patent Application Publication
(10) Pub. No.: US 2026/0209320 A1
(43) Pub. Date: Jul. 23, 2026

(54) ANTI-HUNTINGTIN ANTIBODIES

(71) Applicant: Alchemab Therapeutics Ltd., London (GB)

(72) Inventors: Jane Katharine Osbourn, London (GB); Ralph Raymond Minter, London (GB); Donna Finch, London (GB); Jacob Daniel Galson, London (GB); Laura Sophie Mitchell, London (GB); Jinwoo Leem, London (GB); Jorge Norman Dias Do Nascimeinto, London (GB); James McCarthy, London (GB); April Rose Woulfe, London (GB); Paulina Maria Kolasinska-Zwierz, London (GB); Sofia Pereira Constantino Romano, London (GB)

(73) Assignee: Alchemab Therapeutics Ltd., London (GB)

(21) Appl. No.: 19/136,443

(22) PCT Filed: Dec. 7, 2023
(86) PCT No.: PCT/EP2023/084806

§ 371 Date: Jun. 6, 2025

(30) Foreign Application Priority Data
Dec. 7, 2022 (EP) 22212053.7
Apr. 21, 2023 (GB) 2305916.5

(51) Int. Cl. C07K 16/18; A61K 39/00; A61P 25/28

(52) U.S. Cl. CPC C07K 16/18; A61P 25/28; A61K 2039/505; C07K 2317/33; C07K 2317/34; C07K 2317/565; C07K 2317/567; C07K 2317/73; C07K 2317/92; C07K 2317/94

(57) ABSTRACT

Antibodies that bind the HTT proteins and fragments thereof are described. Compositions comprising these antibodies, as well as methods including therapeutic methods are also described.

This application claims priority from EP 22212053.7 filed 7 Dec. 2022 and GB 2305916.5 filed 21 Apr. 2023, the contents and elements of each of which are herein incorporated by reference for all purposes.

### FIELD OF THE INVENTION

The present invention relates to antibodies and fragments thereof capable of binding to Huntingtin (HTT), and particularly, although not exclusively, to improved therapeutic antibodies. Methods for using anti-HTT antibodies in the treatment of neurological disorders are also described.

### BACKGROUND

Huntington's Disease (HD) is a neurodegenerative monogenic autosomal-dominant disorder resulting from CAG expansion in Exon 1 of the coding gene for Huntingtin protein (HTT). Despite its well-defined genetic origin, molecular/cellular mechanisms underlying HD are complex. Current therapies focus on clinical manifestations (e.g. monoamine depleting agents, tiapride/dopamine D2 receptor antagonists, and anti-depressants) (Dash D and Mestre T A (2020) Therapeutic Update on Huntington's Disease: Symptomatic treatments and emerging disease modifying therapies Neurotherapeutics. 2020 October; 17(4): 1645-1659). These therapies reduce some symptoms caused by HD but are not disease modifying. Although oligonucleotide, gene and cell therapies are advancing through clinical development, there have been significant setbacks which may reflect delivery issues, complexity of new modalities, lack of discrimination between pathological/physiological functions of HTT, and issues with clinical trial design acceptance by patients. There remains a need for improved therapies to Huntington's disease.

Alzheimer's disease (AD) is a neurodegenerative disease that results in progressive loss of brain cells. According to the World Alzheimer Report 2016 (Comas-Herrera et al (2016) World Alzheimer Report 2016 www.alzint.org/resource/world-alzheimer-report-2016), there were 46.8 million people worldwide living with dementia in 2015, and this number will reach 131.5 million in 2050. New therapies to Alzheimer's disease are being actively sought to modify the course of the disease. Current candidates targeting beta-amyloid, Tau, and innate immunity in the brain have in some cases shown pharmacodynamic effects on pathological mechanisms in clinical trials but have yet to demonstrate convincing disease modification in late-stage clinical trials to date. There remains a need for improved disease modifying therapies in Alzheimer's Disease (Golde T E (2022) Neurotherapeutics 19, 209-227).

The present invention has been devised in light of the above considerations.

### SUMMARY OF THE INVENTION

The present invention concerns novel and improved antibodies to HTT. By studying the immune responses of individuals showing resilience to neurodegeneration, despite increased risk of disease, and comparing that to progressive illness, the present inventors identified a cluster of related antibody heavy chains (VH) that was convergent across resilient individuals. Target deconvolution showed that the convergent VHs may bind HTT. The present inventors further identified candidate antibodies derived from these identified VHs, resulting in antibodies that are expected to be able to slow or reverse neurodegeneration, identified using an unbiased approach to both antibody discovery and target identification. In particular, a representative heavy chain was paired with appropriate light chains and expressed in IgG1 format as antibodies referred to herein as ATL5331, ATL5334, and ATL5335. These antibodies were developed so as to further improve properties not limited to improved binding potency, improved pharmacokinetic properties, reduced toxicity, and improved stability. These steps go beyond routine optimisation and required analysis of cohort diversity, extensive testing, investigation of multiple beneficial and mechanistic properties simultaneously, and guided engineering to produce antibodies not found in naturally occurring populations. These novel antibodies with improved properties include antibodies referred to herein as ATL5895, ATL5901, and ATL5667, and affinity matured versions thereof. The antibodies described herein are expected to slow or reverse neurodegeneration by binding to mutant HTT (mHTT) and/or aggregated HTT, in particular extracellular forms thereof.

In a first aspect, the disclosure provides an isolated antibody or antibody fragment thereof which specifically binds to Huntingtin (HTT) protein or a fragment thereof, the antibody comprising a heavy chain variable (VH) domain comprising CDRs HCDR1, HCDR2 and HCDR3, and a light chain variable (VL) domain comprising CDRs LCDR1, LCDR2 and LCDR3, wherein: i. HCDR1 has amino acid sequence KAWMS (SEQ ID NO:1); ii. HCDR2 has amino acid sequence RIKSGIDAGTTDYAAPVKG (SEQ ID NO:2); iii. HCDR3 has amino acid sequence PPYYYYYGLDV (SEQ ID NO: 3), or a sequence comprising one or two substitutions compared with PPYYYYYGLDV (SEQ ID NO: 3), wherein the substitutions are at positions selected from: 95 and 97, wherein the substitutions are selected from: Y97F and P95S, wherein the position numbering is Kabat; iv. LCDR1 has an amino acid sequence TGTSSDVGSYNLVS (SEQ ID NO:4); v. LCDR2 has an amino acid sequence EVNKRPS (SEQ ID NO: 5); and vi. LCDR3 has an amino acid sequence GSYAGTANV (SEQ ID NO:6), or an amino acid sequence comprising one, two, three or four amino acid substitutions compared with GSYAGTANV (SEQ ID NO: 6), wherein the substitutions are at positions selected from: 92, 95, 89, and 91. The substitutions may be selected from: A92G, A95E, and G89V, and Y91F, wherein the position numbering is Kabat.

Thus, the antibody may have the HCDR1, HCDR2 and HCDR3 of ATL 5895, ATL_6194, ATL_6195, ATL_6374, ATL_6375, ATL_6376, ATL_6377, ATL_6378, ATL_6199, ATL_6200, ATL_6202, ATL_6203, ATL_6204 and/or ATL_6205, and the LCDR1, LCDR2 and LCDR3 of ATL 5895, ATL_6194, ATL_6195, ATL_6374, ATL_6375, ATL_6376, ATL_6377, ATL_6378, ATL_6199, ATL_6200, ATL_6202, ATL_6203, ATL_6204 and/or ATL_6205.

Embodiments of any aspect may have any one or more of the following optional features.

The isolated antibody or fragment thereof may have improved binding to mutated and/or aggregated HTT protein compared to non-mutated and/or non-aggregated HTT protein, wherein relative binding to mutated and/or aggregated and non-mutated and/or non-aggregated HTT is measured by determining the ratio of EC50 values for a HTT protein or fragment thereof comprising 25Q repeats in Exon 1 and a HTT protein or fragment thereof comprising 48Q repeats in Exon 1. The isolated antibody or fragment thereof may have a ratio of EC50 for binding to a HTT protein or fragment thereof comprising 25Q repeats in Exon 1 and a HTT protein or fragment thereof comprising 48Q repeats in Exon 1 of at least 1.5, at least 1.6, at least 1.7, at least 1.8, at least 1.9 or at least 2, as measured by sandwich ELISA. The HTT or HTT fragment comprising 48Q repeats may comprise the sequence of SEQ ID NOs: 44 or 46, and/or the HTT or HTT fragment comprising 25Q repeats may comprise the sequence of SEQ ID NOs: 43 or 45. The sandwich ELISA may be performed as described herein (Examples, Materials and Methods).

In embodiments the antibody of fragment comprises a heavy chain variable (VH) domain comprising CDRs HCDR1, HCD2 and HCDR3, wherein: i. HCDR1 has amino acid sequence KAWMS (SEQ ID NO: 1); ii. HCDR2 has amino acid sequence RIKSGIDAGTTDYAAPVKG (SEQ ID NO:2); iii. HCDR3 has amino acid sequence PPYYYYYGLDV (SEQ ID NO:3). In some such embodiments, the antibody or fragment comprises a light chain variable (VL) domain comprising CDRs LCDR1, LCDR2 and LCDR3, wherein: i. LCDR1 has amino acid sequence TGTSSDVGSYNLVS (SEQ ID NO:4); ii. LCDR2 has amino acid sequence EVNKRPS (SEQ ID NO:5); iii. LCDR3 has amino acid sequence GSYAGTANV (SEQ ID NO:6). In other such embodiments, the antibody or fragment comprises a light chain variable (VL) domain comprising CDRs LCDR1, LCDR2 and LCDR3, wherein: i. LCDR1 has amino acid sequence TGTSSDVGSYNLVS (SEQ ID NO:4); ii. LCDR2 has amino acid sequence EVNKRPS (SEQ ID NO:5); and iii. LCDR3 has amino acid sequence VSYGGTENV (SEQ ID NO:162). In other such embodiments, the antibody comprises a light chain variable (VL) domain comprising CDRs LCDR1, LCDR2 and LCDR3, wherein: i. LCDR1 has amino acid sequence TGTSSDVGSYNLVS (SEQ ID NO:4); ii. LCDR2 has amino acid sequence EVNKRPS (SEQ ID NO:5); and iii. LCDR3 has amino acid sequence VSFAGTANV (SEQ ID NO: 160). In other such embodiments, the antibody or fragment comprises a light chain variable (VL) domain comprising CDRs LCDR1, LCDR2 and LCDR3, wherein: i. LCDR1 has amino acid sequence TGTSSDVGSYNLVS (SEQ ID NO: 4); ii. LCDR2 has amino acid sequence EVNKRPS (SEQ ID NO:5); and iii. LCDR3 has amino acid sequence VSYAGTANV (SEQ ID NO: 161). Examples of antibodies according to these embodiments include ATL_5895, ATL_6194, ATL_6195, ATL_6374, ATL_6375, ATL_6199, ATL_6200, ATL_6204, and ATL6205.

In embodiments the antibody or fragment comprises: a heavy chain variable (VH) domain comprising CDRs HCDR1, HCD2 and HCDR3, wherein: i. HCDR1 has amino acid sequence KAWMS (SEQ ID NO: 1); ii. HCDR2 has amino acid sequence RIKSGIDAGTTDYAAPVKG (SEQ ID NO:2); iii. HCDR3 has amino acid sequence PPFYYYYGLDV (SEQ ID NO: 158); and a light chain variable (VL) domain comprising CDRs LCDR1, LCDR2 and LCDR3, wherein: i. LCDR1 has amino acid sequence TGTSSDVGSYNLVS (SEQ ID NO:4); ii. LCDR2 has amino acid sequence EVNKRPS (SEQ ID NO:5); and iii. LCDR3 comprising amino acid sequence VSYGGTENV (SEQ ID NO:162). Examples of antibodies according to these embodiments include ATL_6376 and ATL_6202.

In embodiments, the antibody or fragment comprises a heavy chain variable (VH) domain comprising CDRs HCDR1, HCD2 and HCDR3, wherein: i. HCDR1 has amino acid sequence KAWMS (SEQ ID NO: 1); ii. HCDR2 has amino acid sequence RIKSGIDAGTTDYAAPVKG (SEQ ID NO:2); iii. HCDR3 has amino acid sequence SPYYYYYGLDV (SEQ ID NO:157). In some such embodiments, the antibody or fragment has a light chain variable (VL) domain comprising CDRs LCDR1, LCDR2 and LCDR3, wherein: i. LCDR1 has amino acid sequence TGTSSDVGSYNLVS (SEQ ID NO:4); ii. LCDR2 has amino acid sequence EVNKRPS (SEQ ID NO:5); iii. LCDR3 has amino acid sequence VSYAGTANV (SEQ ID NO:161). Examples of antibodies according to these embodiments include ATL_6377 and ATL_6203. In other such embodiments, the antibody or fragment comprises a light chain variable (VL) domain comprising CDRs LCDR1, LCDR2 and LCDR3, wherein: i. LCDR1 has amino acid sequence TGTSSDVGSYNLVS (SEQ ID NO:4); ii. LCDR2 has amino acid sequence EVNKRPS (SEQ ID NO:5); iii. LCDR3 has amino acid sequence VSYGGTENV (SEQ ID NO: 162). Examples of antibodies according to these embodiments include ATL_6378.

The VH domain may be a human VH domain. The antibody or fragment thereof may have the framework sequence of ATL_0006199 VH:
EVOLVESGGGLVKPGGSLRLSCAASGFTFN (SEQ ID NO: 98)-[CDRH1]-WVRQAPGKGLEWVG (SEQ ID NO: 99)-[CDRH2]-RFTISRDDSKNTLYLQMNSLKTEDTAVYWCSP (SEQ ID NO: 169)-[CDRH3]-WGQGTTVTVSS (SEQ ID NO: 101). The antibody or fragment thereof may have the framework sequence of ATL_0006200 VH (and ATL6374 VH and ATL_6194 VH):
EVOLVESGGGLVKPGGSLRLSCAASGFTFN (SEQ ID NO: 98)-[CDRH1]-WVRQAPGKGLEWVG (SEQ ID NO: 99)-[CDRH2]-RFTISRDDSKNTLYLQMNSLKTEDTAVYYCVP (SEQ ID NO: 170)-[CDRH3]-WGQGTTVTVSS (SEQ ID NO: 101). The antibody or fragment thereof may have the framework sequence of ATL_0006202 VH:
EVQLVESGGGLVKPGGSLRLSCAASGFTFN (SEQ ID NO: 98)-[CDRH1]-WVRQAPGKGLEWVG (SEQ ID NO: 99)-[CDRH2]-RFTISRDDSKNTLYLQMNSLKTEDTAVYYCSP (SEQ ID NO: 171)-[CDRH3]-WGQGTTVTVSS (SEQ ID NO: 101). The antibody or fragment thereof may have the framework sequence of ATL_0006203 VH (and ATL_5895 VH and ATL_6204 VH):
EVQLVESGGGLVKPGGSLRLSCAASGFTFN (SEQ ID NO: 98)-[CDRH1]-WVRQAPGKGLEWVG (SEQ ID NO: 99)-[CDRH2]-RFTISRDDSKNTLYLQMNSLKTEDTAVYYCIP (SEQ ID NO: 172)-[CDRH3]-WGQGTTVTVSS (SEQ ID NO: 101). The antibody or fragment thereof may have the framework sequence of ATL_0006205 VH (and ATL_6375 VH, ATL_6376 VH, ATL6377VH and ATL_6378 VH):
EVOLVESGGGLVKPGGSLRLSCAASGFTFN (SEQ ID NO: 98)-[CDRH1]-WVRQAPGKGLEWVG (SEQ ID NO: 99)-[CDRH2]-RFTISRDDSKNTLYLQMNSLKTEDTAVYWCVP (SEQ ID NO: 173)-[CDRH3]-WGQGTTVTVSS (SEQ ID NO: 101). The antibody or fragment thereof may have the framework sequence of ATL_006195 VH:

(SEQ ID NO: 98)
EVQLVESGGGLVKPGGSLRLSCAASGFTFN
-[CDRH1]-
(SEQ ID NO: 99)
WVRQAPGKGLEWVG
-[CDRH2]-
(SEQ ID NO: 180)
RFTISRDDSKNTLYLQMNSLKTEDTAVYYCTP
-[CDRH3]-
(SEQ ID NO: 101)
WGQGTTVTVSS.

In embodiments, the VL domain is a human VL domain. In embodiments, the antibody or fragment thereof has a VL domain framework sequence of ATL_0005895 VL:

(SEQ ID NO: 131)
QSALTQPRSVSGSPGQSVTISC
-[CDRL1]-
(SEQ ID NO: 133)
WYQQHPGKAPKLMIY
-[CDRL2]-
(SEQ ID NO: 138)
GVPDRFSGSKSGATASLTISGLQAEDEADYYC
-[CDRL3]-
(SEQ ID NO: 139)
FGTGTKLTVL.

In embodiments, HCDR1, HCDR2 and HCDR3 of the VH domain are within a germline framework. In embodiments, LCDR1, LCDR2, LCDR3 of the VL domain are within a germline framework.

In embodiments, the heavy chain variable domain comprises the amino acid sequence of any of ATL_5895 VH (SEQ ID NO: 7), ATL_6204 VH (SEQ ID NO: 7), ATL_6199 VH (SEQ ID NO:144), ATL_6374 VH (SEQ ID NO:148), ATL_6194 VH (SEQ ID NO:145), ATL_6375 VH (SEQ ID NO: 145), ATL_6200 VH (SEQ ID NO: 145), ATL_6202 VH (SEQ ID NO: 146), ATL_6203 VH (SEQ ID NO: 147), ATL_6205 VH (SEQ ID NO: 148), ATL_6376 VH (SEQ ID NO:175), ATL_6377 VH (SEQ ID NO:176), ATL_6195 VH (SEQ ID NO: 179), ATL_6378 VH (SEQ ID NO: 176). In some such embodiments, the light chain variable domain comprises the amino acid sequence of any of ATL_5895 VL (SEQ ID NO: 8), ATL_6199 VL (SEQ ID NO: 8), ATL_6195 VL (SEQ ID NO: 8), ATL_6002 VL (SEQ ID NO: 8), ATL_6374 VL (SEQ ID NO:153), ATL_6375 (SEQ ID NO:153), ATL_6376 VL (SEQ ID NO:153), ATL_6378 VL (SEQ ID NO:153), ATL_6194 VL (SEQ ID NO: 154), ATL_6377 VL (SEQ ID NO: 154), ATL_6203 VL (SEQ ID NO: 154), ATL_6204 VL (SEQ ID NO: 155), ATL_6205 VL (SEQ ID NO: 156), ATL_6202 VL (SEQ ID NO: 159).

In embodiments, the heavy chain variable domain comprises the amino acid sequence of any of ATL_5895 VH (SEQ ID NO: 7), ATL_6376 VH (SEQ ID NO: 175), ATL_6377 VH (SEQ ID NO: 176), or a sequence comprising at most 1, 2 or 3 mutations compared to any of these sequences. In some such embodiments the light chain variable domain comprises the amino acid sequence of any of ATL_5895 VL (SEQ ID NO:8), ATL_6376 VL (SEQ ID NO:153), ATL_6377 VL (SEQ ID NO: 154), or a sequence comprising at most 1, 2 or 3 mutations compared to any of these sequences.

In embodiments, the heavy chain variable domain comprises amino acid sequence

(ATL_5895 VH)
(SEQ ID NO: 7)
EVQLVESGGGLVKPGGSLRLSCAASGFTFNKAWMSWVRQAPGKGLEWVGRIKSGIDAGTTDYAAP
VKGRFTISRDDSKNTLYLQMNSLKTEDTAVYYCIPPPYYYYYGLDVWGQGTTVTVSS.

In some such embodiments, the light chain variable domain comprises amino acid sequence

(ATL_5895 VL)
(SEQ ID NO: 8)
QSALTQPRSVSGSPGQSVTISCTGTSSDVGSYNLVSWYQQHPGKAPKLMIYEVNKRPSGVPDRFS
GSKSGATASLTISGLQAEDEADYYCGSYAGTANVFGTGTKVTVL.

In embodiments, the heavy chain variable domain comprises amino acid sequence

(ATL_6374 VH)
(SEQ ID NO: 148)
EVQLVESGGGLVKPGGSLRLSCAASGFTFNKAWMSWVRQAPGKGLEWVGRIKSGIDAGTTDYAAP
VKGRFTISRDDSKNTLYLQMNSLKTEDTAVYWCVPPPYYYYYGLDVWGQGTTVTVSS.

In some such embodiments the light chain variable domain comprises amino acid sequence

(ATL_6374 VL)
(SEQ ID NO: 153)
QSALTQPRSVSGSPGQSVTISCTGTSSDVGSYNLVSWYQQHPGKAPKLMIYEVNKRPSGVPDRFS
GSKSGATASLTISGLQAEDEADYYCVSYGGTENVFGTGTKVTVL.

In embodiments, the heavy chain variable domain comprises amino acid sequence

(ATL_6375 VH)
(SEQ ID NO: 145)
EVQLVESGGGLVKPGGSLRLSCAASGFTFNKAWMSWVRQAPGKGLEWVGRIKSGIDAGTTDYAAP
VKGRFTISRDDSKNTLYLQMNSLKTEDTAVYYCVPPPYYYYYGLDVWGQGTTVTVSS.

In some such embodiments the light chain variable domain comprises amino acid sequence

(ATL_6375 VL)
(SEQ ID NO: 153)
QSALTQPRSVSGSPGQSVTISCTGTSSDVGSYNLVSWYQQHPGKAPKLMIYEVNKRPSGVPDRFS
GSKSGATASLTISGLQAEDEADYYCVSYGGTENVFGTGTKVTVL.

In embodiments, the heavy chain variable domain comprises amino acid sequence

(ATL_6376 VH)
(SEQ ID NO: 175)
EVQLVESGGGLVKPGGSLRLSCAASGFTFNKAWMSWVRQAPGKGLEWVGRIKSGIDAGTTDYAAP
VKGRFTISRDDSKNTLYLQMNSLKTEDTAVYYCVPPPFYYYYGLDVWGQGTTVTVSS.

In some such embodiments, the light chain variable domain comprises amino acid sequence

(ATL_6376 VL)
(SEQ ID NO: 153)
QSALTQPRSVSGSPGQSVTISCTGTSSDVGSYNLVSWYQQHPGKAPKLMIYEVNKRPSGVPDRFS
GSKSGATASLTISGLQAEDEADYYCVSYGGTENVFGTGTKVTVL.

In embodiments the heavy chain variable domain comprises amino acid sequence

(ATL_6377 VH)
(SEQ ID NO: 176)
EVQLVESGGGLVKPGGSLRLSCAASGFTFNKAWMSWVRQAPGKGLEWVGRIKSGIDAGTTDYAAP
VKGRFTISRDDSKNTLYLQMNSLKTEDTAVYYCVPSPYYYYYGLDVWGQGTTVTVSS.

In some such embodiments the light chain variable domain sequence comprises amino acid sequence

(SEQ ID NO: 154)
QSALTQPRSVSGSPGQSVTISCTGTSSDVGSYNLVSWYQQHPGKA
PKLMIYEVNKRPSGVPDRFSGSKSGATASLTISGLQAEDEADYYC
VSYAGTANVFGTGTKVTVL
(ATL_6377 VL).

In embodiments, the heavy chain variable domain comprises amino acid sequence

(SEQ ID NO: 176)
EVQLVESGGGLVKPGGSLRLSCAASGFTFNKAWMSWVRQAPGKGL
EWVGRIKSGIDAGTTDYAAPVKGRFTISRDDSKNTLYLQMNSLKT
EDTAVYYCVPSPYYYYYGLDVWGQGTTVTVSS
(ATL_6378 VH).

In some such embodiments the light chain variable domain comprises amino acid sequence

(SEQ ID NO: 153)
QSALTQPRSVSGSPGQSVTISCTGTSSDVGSYNLVSWYQQHPGKA
PKLMIYEVNKRPSGVPDRFSGSKSGATASLTISGLQAEDEADYYC
VSYGGTENVFGTGTKVTVL
(ATL_6378 VL).

In embodiments, the heavy chain variable domain comprises amino acid sequence

(SEQ ID NO: 144)
EVQLVESGGGLVKPGGSLRLSCAASGFTFNKAWMSWVRQAPGKGL
EWVGRIKSGIDAGTTDYAAPVKGRFTISRDDSKNTLYLQMNSLKT
EDTAVYWCSPPPYYYYYGLDVWGQGTTVTVSS
(ATL_6199 VH).

In some such embodiments the light chain variable domain comprises amino acid sequence

(SEQ ID NO: 8)
QSALTQPRSVSGSPGQSVTISCTGTSSDVGSYNLVSWYQQHPGKA
PKLMIYEVNKRPSGVPDRFSGSKSGATASLTISGLQAEDEADYYC
GSYAGTANVFGTGTKVTVL
(ATL_6199 VL).

In embodiments, the heavy chain variable domain comprises amino acid sequence

(SEQ ID NO: 145)
EVQLVESGGGLVKPGGSLRLSCAASGFTFNKAWMSWVRQAPGKGL
EWVGRIKSGIDAGTTDYAAPVKGRFTISRDDSKNTLYLQMNSLKT
EDTAVYYCVPPPYYYYYGLDVWGQGTTVTVSS
(ATL_6200 VH).

In some such embodiments, the light chain variable domain comprises amino acid sequence

(SEQ ID NO: 8)
QSALTQPRSVSGSPGQSVTISCTGTSSDVGSYNLVSWYQQHPGKA
PKLMIYEVNKRPSGVPDRFSGSKSGATASLTISGLQAEDEADYYC
GSYAGTANVFGTGTKVTVL
(ATL_6002 VL).

In embodiments, the heavy chain variable domain comprises amino acid sequence

(SEQ ID NO: 146)
EVQLVESGGGLVKPGGSLRLSCAASGFTFNKAWMSWVRQAPGKGL
EWVGRIKSGIDAGTTDYAAPVKGRFTISRDDSKNTLYLQMNSLKT
EDTAVYYCSPPPFYYYYGLDVWGQGTTVTVSS
(ATL_6202 VH).

In some such embodiments, the light chain variable domain comprises amino acid sequence

(SEQ ID NO: 159)
QSALTQPRSVSGSPGQSVTISCTGTSSDVGSYNLVSWYQQHPGKA
PKLMIYEVNKRPSGVPDRFSGSKSGATASLTISGLQAEDEADYYC
VSYGGTENVFGTGTKVTVL
(ATL_6202 VL).

In embodiments, the heavy chain variable domain comprises amino acid sequence

(SEQ ID NO: 147)
EVQLVESGGGLVKPGGSLRLSCAASGFTFNKAWMSWVRQAPGKGL
EWVGRIKSGIDAGTTDYAAPVKGRFTISRDDSKNTLYLQMNSLKT
EDTAVYYCIPSPYYYYYGLDVWGQGTTVTVSS
(ATL_6203 VH).

In some such embodiments, the light chain variable domain comprises amino acid sequence

(SEQ ID NO: 154)
QSALTQPRSVSGSPGQSVTISCTGTSSDVGSYNLVSWYQQHPGKA
PKLMIYEVNKRPSGVPDRFSGSKSGATASLTISGLQAEDEADYYC
VSYAGTANVFGTGTKVTVL
(ATL_6203 VL).

In embodiments, the heavy chain variable domain comprises amino acid sequence

(SEQ ID NO: 7)
EVQLVESGGGLVKPGGSLRLSCAASGFTFNKAWMSWVRQAPGKGL
EWVGRIKSGIDAGTTDYAAPVKGRFTISRDDSKNTLYLQMNSLKT
EDTAVYYCIPPPYYYYYGLDVWGQGTTVTVSS
(ATL_6204 VH).

In some such embodiments the light chain variable domain comprises amino acid sequence

(SEQ ID NO: 155)
QSALTQPRSVSGSPGQSVTISCTGTSSDVGSYNLVSWYQQHPGKA
PKLMIYEVNKRPSGVPDRFSGSKSGATASLTISGLQAEDEADYYC
VSFAGTANVFGTGTKVTVL
(ATL_6204 VL).

In embodiments, the heavy chain variable domain comprises amino acid sequence

(SEQ ID NO: 148)
EVQLVESGGGLVKPGGSLRLSCAASGFTFNKAWMSWVRQAPGKGL
EWVGRIKSGIDAGTTDYAAPVKGRFTISRDDSKNTLYLQMNSLKT
EDTAVYWCVPPPYYYYYGLDVWGQGTTVTVSS
(ATL_6205 VH).

In some such embodiments the light chain variable domain comprises amino acid sequence

(SEQ ID NO: 156)
QSALTQPRSVSGSPGQSVTISCTGTSSDVGSYNLVSWYQQHPGKA
PKLMIYEVNKRPSGVPDRFSGSKSGATASLTISGLQAEDEADYYC
VSYAGTANVFGTGTKVTVL
(ATL_6205 VL).

In embodiments, the heavy chain variable domain comprises amino acid sequence

(ATL_6194 VH)
(SEQ ID NO: 145)
EVQLVESGGGLVKPGGSLRLSCAASGFTFNKAWMSWVRQAPGKGLEWVGR
IKSGIDAGTTDYAAPVKGRFTISRDDSKNTLYLQMNSLKTEDTAVYYCVP
PPYYYYYGLDVWGQGTTVTVSS.

In some such embodiments, the light chain variable domain comprises amino acid sequence

(ATL_6194 VL)
(SEQ ID NO: 154)
QSALTQPRSVSGSPGQSVTISCTGTSSDVGSYNLVSWYQQHPGKAPKLMI
YEVNKRPSGVPDRFSGSKSGATASLTISGLQAEDEADYYCVSYAGTANVF
GTGTKVTVL.

In embodiments, the heavy chain variable domain comprises amino acid sequence

(ATL_6195 VH)
(SEQ ID NO: 179)
EVQLVESGGGLVKPGGSLRLSCAASGFTFNKAWMSWVRQAPGKGLEWVGR
IKSGIDAGTTDYAAPVKGRFTISRDDSKNTLYLQMNSLKTEDTAVYYCTP
PPYYYYYGLDVWGQGTTVTVSS.

In some such embodiments, the light chain variable domain comprises amino acid sequence

(ATL_6195 VL)
(SEQ ID NO: 8)
QSALTQPRSVSGSPGQSVTISCTGTSSDVGSYNLVSWYQQHPGKAPKLMI
YEVNKRPSGVPDRFSGSKSGATASLTISGLQAEDEADYYCGSYAGTANVF
GTGTKVTVL.

In embodiments the heavy chain variable domain comprises a variable domain comprising an amino acid sequence that has at least 95% sequence identity or that comprises at most 1, 2 or 3 substitutions compared to any one of the heavy chain variable domains above (SEQ ID Nos: 7, 148, 145, 175, 176, 144, 146, 147, 179) and/or the light chain variable domain comprises an amino acid sequence that has at least 90%, at least 95% sequence identity, or that comprises at most 1, 2, 3, 4 or 5 substitutions compared to any one of the light chain variable domains above (SEQ ID Nos: 8, 153, 154, 159, 155, 156).

In embodiments, the HTT protein is human or mouse HTT. In embodiments, the isolated antibody or fragment thereof binds to a region located within Exon 1 of HTT. In embodiments, the isolated antibody or fragment thereof binds to HTT or a fragment thereof comprising at least a portion of Exon 1, with a lower EC50 value compared with a reference antibody as measured by sandwich ELISA. In embodiments, the HTT has 25Q repeats or 48 repeats in Exon 1. In embodiments, the isolated antibody or fragment thereof has improved binding to mutated and/or aggregated HTT protein compared to non-mutated and/or non-aggregated HTT protein. In embodiments, relative binding to mutated and/or aggregated and non-mutated and/or non-aggregated HTT is measured by determining the ratio of EC50 values for a HTT protein or fragment thereof comprising 25Q repeats in Exon 1 and a HTT protein or fragment thereof comprising 48Q repeats in Exon 1. In embodiments, the HTT protein or fragment thereof is a fragment corresponding to Exon 1. In embodiments, the EC50 is as measured by sandwich ELISA. In embodiments, the isolated antibody or fragment thereof has a higher relative binding to mutated and/or aggregated and non-mutated and/or non-aggregated HTT compared to a reference antibody (such as e.g. ATL_0005059). In embodiments, the isolated antibody or fragment thereof has a ratio of EC50 for binding to a HTT protein or fragment thereof comprising 25Q repeats in Exon 1 and a HTT protein or fragment thereof comprising 48Q repeats in Exon 1 of at least 1.15, at least 1.18, at least 1.2, at least 1.3, at least 1.4, at least 1.5, at least 1.6, at least 1.7, at least 1.8, at least 1.9 or at least 2 as measured by sandwich ELISA. The HTT or HTT fragment comprising 48Q repeats may comprise the sequence of SEQ ID NOs: 44 or 46. The HTT or HTT fragment comprising 25Q repeats may comprise the sequence of SEQ ID NOs: 43 or 45. The sandwich ELISA may be performed as described herein (Examples, Materials and Methods). Thus, the antibodies described herein may be able to preferentially bind mutated HTT, thereby decreasing the seeding propensity of mutated HTT (i.e. reducing mutated HTT aggregation).

The isolated antibody or fragment thereof may increase phagocytosis of a HTT protein or fragment thereof comprising exon 1 comprising 48Q repeats by a cell. The cell may be a microglia cell, optionally an iPSC derived human microglia cell. The isolated antibody or fragment thereof may increase phagocytosis of a HTT protein or fragment thereof comprising exon 1 comprising 48Q repeats by a cell in a dose dependent manner. The increased phagocytosis may be measured by detecting phagocytosis of beads coated with the HTT protein or fragment coated with a pH-sensitive fluorescent dye. Increased phagocytosis may be measured as described herein (Materials and Methods).

The isolated antibody or fragment thereof may reduce the rate of aggregation of a HTT protein or fragment thereof comprising exon 1 comprising 48Q repeats in a cell free assay. The reduced rate of aggregation may be measured using a FRASE assay. The reduced rate of aggregation may be measured as described herein (Materials and Methods). Immunodepletion of a solution comprising the HTT protein or fragment thereof with the isolated antibody or fragment thereof may result in a Delta t50 for aggregation of the HTT protein or fragment thereof of at most 0.1, at most 0.2, or at most 0.3. The aggregation of the HTT protein or fragment thereof is measured in the present of HTT fibrils (e.g. from recombinant HTT) and/or brain homogenate from one or more R6/2 mice.

In embodiments, the reference antibody comprises: (a) a heavy chain variable (VH) domain with the following CDRs: i. HCDR1 with amino acid sequence NAWMN (SEQ ID NO: 35); ii. HCDR2 with amino acid sequence HIRTQAEGGTSDYAAPVKG (SEQ ID NO: 36); iii. HCDR3 with amino acid sequence PPYYYYYGLDV (SEQ ID NO: 3); and (b) a light chain variable (VL) domain with the following CDRs: i. LCDR1 with amino acid sequence TGASSDVGTYDLVS (SEQ ID NO: 37); ii. LCDR2 with amino acid sequence EVNKRPS (SEQ ID NO:5); and iii. LCDR3 with amino acid sequence CSYAGYSTV (SEQ ID NO: 38). In embodiments, the reference antibody is NI-302.8F1 described in U.S. Pat. No. 11,401,325.

In embodiments, the isolated antibody or fragment thereof binds mutated and/or aggregated HTT protein as determined by measuring immunoprecipitation of mutant HTT with the isolated antibody or fragment thereof. In embodiments, mutant HTT is a HTT protein or fragment thereof. In embodiments, mutant HTT is a HTT fragment comprising exon 1. In embodiments, mutant HTT is a HTT protein or fragment that comprises more than 35 glutamine residues in its polyQ tract. Preferential binding to the more aggregation-prone, likely more pathological form of HTT may advantageously inhibit the templating potential of the aggregated/mutant HTT, thus inhibiting the progression of a disease associated with protein aggregation. For example, an isolated antibody or fragment described herein may immunoprecipitated a mutated and/or aggregated HTT protein comprising 110 CAG repeats (HTT₁₁₀). As another example, the mutated and/or aggregated HTT protein may immunoprecipitated a mutated and/or aggregated HTT protein comprising about 120 CAG repeats (e.g. extracted from a cell or tissue sample such as a sample from a R6/2 transgenic mouse). R6/2 transgenic mice express the 5′ end of the human HTT gene, including exon 1 with approximately 120 CAG repeats and display a neurological phenotype similar to the features of HD in humans. As a further example, an isolated antibody or fragment as described herein may immunoprecipitate a mutated and/or aggregated HTT protein comprising about 115 to 150 CAG repeats (e.g. extracted from a cell or tissue sample such as a sample from a R6/1 transgenic mouse). R6/1 transgenic mice ubiquitously express a transgene comprising the 5′ end of the mutated human HTT gene comprising approximately 1 kb of 5′ UTR sequences, exon 1 (carrying expanded CAG repeats with 115 to 150 CAG repeats) and the first 262 bp of intron 1. R6/1 mice exhibit a progressive neurological phenotype that mimics many of the features of Huntington's Disease (Mangiarini et al; Cell; 1996) including an accumulation of aggregates over time (Hansson et al; EJN; 2001).

In embodiments, the isolated antibody or fragment thereof reduces aggregation of mutant HTT, wherein the aggregation of the mutant HTT is measured as the presence and/or concentration of HTT aggregates in the brain of a transgenic mouse model of Huntington's disease. The mouse model may be a R6/1 mouse. Treatment of said mice with the isolated antibody or fragment thereof for 12 weeks or more may result in a statistically significant decrease in the concentration of HTT aggregates in the striatum and/or cortex. In embodiments, the antibody or fragment binds to mutant HTT in vivo. The mutant HTT may be a HTT protein or fragment thereof, optionally a fragment comprising exon 1, that comprises more than 35 glutamine residues, or between 115 and 150 glutamine residues in its polyQ tract. In embodiments, the isolated antibody or fragment thereof does not reduce the level of non-mutated and/or non-aggregated HTT in the brain of a transgenic mouse model of Huntington's disease treated with the isolated antibody or fragment thereof, wherein the level of the non-mutated and/or non-aggregated HTT is measured as the concentration of soluble and/or non-mutated HTT said mouse, optionally wherein the mouse is a R6/1 mouse.

In embodiments, the antibody or fragment thereof maintains a percentage monomer above 95% or above 97% after incubation at −80° C., 4° C., 21° C., 40° C. for 4 weeks and/or after 10× freeze-thaw cycles. In embodiments, the antibody or fragment thereof binds to a HTT protein comprising Exon 1 of HTT and 48 glutamine residues in its polyQ tract following incubation at −80° C., 4° C., 21° C. and 40° C. for 4 weeks and/or 10× freeze-thaw cycles, as assessed by ELISA. In some such embodiments, said binding is not significantly different from the binding of said antibody to said HTT protein prior to incubation and/or 10× freeze-thaw cycles.

In embodiments, the isolated antibody or fragment thereof binds to HTT or a fragment thereof comprising at least a portion of Exon 1, with an EC50 value or at most 15 nM, at most 12 nM, at most 10 nM or at most 5 nM as measured using by sandwich ELISA. The HTT may have 25Q repeats or 48 repeats in Exon 1. The HTT or HTT fragment may comprise the sequence of SEQ ID NOs: 43, 44, 45 or 46, The sandwich ELISA may be performed as described herein (Examples, Materials and Methods).

In embodiments, the isolated antibody or fragment thereof recognises an epitope in a region corresponding to Exon 1 of the HTT gene. In embodiments, the isolated antibody or fragment thereof recognises an epitope located in the polyP region of HTT. In embodiments, the isolated antibody or fragment thereof recognises an epitope comprising the amino acid sequence QQQQPPPPPPPPPPP (SEQ ID NO: 47) or PQPQPPPPPPPPPPP (SEQ ID NO: 48). In embodiments, the isolated antibody or fragment thereof is capable of crossing the blood-brain barrier. In embodiments, the isolated antibody or fragment thereof is a bispecific antibody further comprising a region that binds to the transferrin receptor. A bispecific antibody comprising an isolated antibody or antibody fragment according to any preceding embodiment, such as a single-chain variable fragment (scFv) according to the first aspect, and a binding moiety (such as a scFV, nanobody or aptamer) that binds to a brain receptor, such as the transferrin receptor.

In embodiments, the isolated antibody or antibody fragment comprises a single-chain variable fragment (scFv) or fragment antigen-binding region (Fab). In embodiments, the isolated antibody or antibody fragment comprises an antibody constant region. In embodiments, the isolated antibody comprises a whole antibody. Optionally the whole antibody may be an IgG1 antibody.

In a further aspect, the disclosure provides an isolated antibody VH domain of an isolated antibody or antibody fragment according to the preceding aspects.

In a further aspect, the disclosure provides an isolated antibody VL domain of an isolated antibody or antibody fragment according to the first or second aspects.

In a further aspect, the disclosure provides a composition comprising an isolated antibody, antibody fragment, antibody VH domain or antibody VL domain according to the first or second aspects.

In a further aspect, the disclosure a host cell in vitro transformed with a nucleic acid molecule encoding an antibody or antibody fragment thereof according to the first or second aspects.

In a further aspect, the disclosure provides a method of producing an antibody or antibody fragment (including e.g. an antibody VH or VL domain) according to any embodiment of the first or second aspect, the method comprising culturing host cells in vitro transformed with a nucleic acid molecule encoding the antibody or antibody fragment under conditions suitable for the production of said antibody or antibody fragment. The method may further comprise isolating and/or purifying said antibody or antibody fragment. The method may further comprise formulating the antibody or antibody fragment into a composition including at least one additional component.

In a further aspect, the disclosure provides a DNA molecule or set of DNA molecules encoding an antibody or antibody fragment thereof according to the first or second aspects.

In a further aspect, the disclosure provides a vector or set of vectors encoding the DNA molecule or molecules according to the first or second aspects.

In a further aspect, the disclosure provides a host cell comprising the vector or set of vectors according to the first or second aspects.

In a further aspect, the disclosure provides a method of treating a disease or disorder in a subject, comprising administering to the subject a therapeutically effective amount of an isolated antibody or antibody fragment thereof according to the first or second aspects. The treatment may prevent and/or reduces seeding and/or aggregation of mutant HTT in the subject. The disease or disorder may be Huntington's disease, Alzheimer's disease, or frontotemporal dementia. In embodiments, the treatment comprises administration of a further therapeutic agent, simultaneously or sequentially with the isolated antibody or antibody fragment. In embodiments, the treatment prevents and/or reduces aggregation of mutant HTT in the subject (including e.g. in the subject's brain) without reducing the level of non-mutated and/or non aggregated HTT in the subject (including e.g. in the subject's brain).

In a further aspect, the disclosure provides the use of the isolated antibody or antibody fragment thereof according to the first or second aspects in the manufacture of a medicament for the treatment of a disorder or disease. The disorder or disease may be Huntington's disease, Alzheimer's disease, or frontotemporal dementia.

In a further aspect, the disclosure provides a composition comprising an isolated antibody or antibody fragment thereof according to the first or second aspects. The composition may comprise a pharmaceutically acceptable excipient, vehicle or carrier. The composition may be for use in the treatment of a disease or disorder. The disease or disorder may be Huntington's disease, Alzheimer's disease, or frontotemporal dementia.

In a further aspect, the disclosure relates to a method of diagnosing or monitoring the progression of a disease or disorder characterised by the presence of mutated and/or aggregated HTT protein in a patient, the method comprising exposing a sample obtained from the patient to an antibody or fragment thereof as described herein.

In a further aspect, the disclosure provides a method of determining the effect of a therapy such as e.g. a drug on the presence of aggregated HTT protein in a patient, the method comprising exposing a sample obtained from the patient to an antibody or fragment thereof as described herein. The methods of the preceding aspect may comprise determining a level of HTT protein in the sample from the patient by detecting the antibody or fragment thereof, or the binding between the HTT protein and the antibody or fragment thereof. The methods may comprise comparing the determined levels with a predetermined threshold, or levels determined for one or more control samples. The sample may be a blood sample or a sample of cerebrospinal fluid.

In a further aspect, the disclosure provides a method of preventing or reducing seeding and/or aggregation of mutant HTT in a subject in need thereof, wherein the method comprised administering to the subject a therapeutic amount of an isolated antibody or antibody fragment thereof.

The disclosure also explicitly includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows schematically a process for unbiased antibody discovery and target identification. A. Workflow of antibody identification. B. Alignment of seven antibodies discovered in an Alzheimer patient cohort displaying CDR3 sequence homology to a known HTT binder (NI-302.8F1 described in U.S. Pat. No. 11,401,325 B2).

FIG. 2 illustrates schematically a phage display selection process. A. Generation of a functional scFv library. Suitable phagemids containing a VL sub-library were designed and generated from a healthy donor panel. A VH derived from an AD resilient individual was cloned upstream of the VL sub-library to create a functional (fused VH and VL) scFv library. B. Phages displaying the AD resilient library of scFV were used for selections. Two to three rounds of selection were performed on immobilised HTT exon 1 antigen (Human HTT exon 1 with 48Q repeats, see Table 1 for sequence) with every selection round narrowing down the pool of scFv to those with antigen binding properties. Phage ELISA was used to confirm target antigen binding and unique scFv which bind were converted to IgG1 for confirmation of antigen binding.

FIG. 3 shows ELISA results for antibodies of the disclosure. A. Phage ELISA for binding of phage displaying antibody fragments corresponding to the antibody sequences selected to human mutated HTT (mHTT) with 48Q repeats (in each subsection of the plot, bars of different colours indicate different concentrations of mHTT: from left to right: 10 μg/ml, 2 μg/ml and 0.4 μg/ml). B. IgG1 antibody ELISA on human HTT Exon1 (48Q) protein, showing strong dose-dependent binding for 5 out of the 7 IgG1-converted antibodies tested.

FIG. 4 illustrates a VH and phage display VL pairing. A. VH sequence identified as convergent in AD-resilient individuals is indicated as ATL_0005042 (ATL_5042). The homologous HTT-binding antibody is CA_0000274 (also referred to as ATL_0005059 or NI-302.8F1). B. Following phage display selections on HTT Exon 1 and sequence analysis, the functionally-paired VLs (ATL_0005331-5335) were aligned to the CA_0000274 VL.

FIG. 5 shows the representation of peptides on a peptide array used for HTT epitope mapping. The PEPperMAP® Epitope Mapping and peptide screening of human IgG1 antibodies ATL_0005331, ATL_0005335 and ATL_0005566 was performed against the sequences of human, cyno and mouse huntingtin exon 1. The huntingtin sequences were converted into linear 15 amino acid peptides with a peptide-peptide overlap of 14 amino acids for high resolution epitope data.

FIG. 6 shows results of epitope mapping and peptide screening for ATL_5331. Upper panel: intensity plots showing the corrected intensity values to the human, cyno_1, cyno_2 (two different sequences that are the likely cynomolgus monkey Huntingtin protein sequence) and mouse huntingtin sequences, sorted from the N-terminus of human huntingtin protein to the C-terminus of mouse huntingtin protein. IgG was tested at 1 μg/ml fluorescence intensity values plotted in green, IgG tested at 10 μg/ml fluorescence intensity values plotted in red with the addition of 1000 to each intensity value to aid visualisation. The X-axis indicates species of HTT. Antibody ATL_0005331 shows to two distinct signals across HTT exon1. A number of antibody responses are observed against epitope-like sequence patterns formed by adjacent peptides with a consensus motif at each site. Lower panel: epitope mapping based on the human, cyno_1, cyno_2 and mouse huntingtin sequences for ATL_0005331. The amino acids from HTT exon 1 derived peptides for which ATL_0005331 demonstrated binding are shown in bold and the peptides showing the highest relative binding at each site of the different species HTT exon 1 are underlined.

FIG. 7 shows results of epitope mapping and peptide screening for ATL_5335. Upper panel: intensity plots showing the corrected intensity values to the human, cyno_1, cyno_2 and mouse huntingtin sequences, sorted from the N-terminus of human huntingtin protein to the C-terminus of mouse huntingtin protein. IgG tested at 1 μg/ml fluorescence intensity values plotted in green, IgG tested at 10 μg/ml fluorescence intensity values plotted in red with the addition of 2000 to each intensity value to aid visualisation. X-axis indicates species of HTT. Antibody ATL_0005335 shows two distinct signals across HTT exon1. A number of antibody responses are observed against epitope-like sequence patterns formed by adjacent peptides with a consensus motif at each site. Lower panel: epitope mapping based on the human, cyno_1, cyno_2 and mouse huntingtin sequences for ATL_0005335. The amino acids from HTT exon 1 derived peptides for which ATL_0005335 demonstrated binding are shown in bold and the peptides showing the highest relative binding at each site of the different species HTT exon 1 are underlined.

FIG. 8 shows the results of epitope and peptide screening for ATL_5566 Upper panel: Intensity plots showing the corrected intensity values to the human, cyno_1, cyno_2 and mouse huntingtin sequences, sorted from the N-terminus of human huntingtin protein to the C-terminus of mouse huntingtin protein. IgG tested at 1 μg/ml fluorescence intensity values plotted in green, IgG tested at 10 μg/ml fluorescence intensity values plotted in red with the addition of 1000 to each intensity value to aid visualisation. X-axis indicates species of HTT. Antibody ATL_0005566 shows two distinct signals across HTT exon1. A number of antibody responses are observed against epitope-like sequence patterns formed by adjacent peptides with a consensus motif at each site. Lower panel: epitope mapping based on the human, cyno_1, cyno_2 and mouse huntingtin sequences for ATL_0005566. The amino acids from HTT exon 1 derived peptides for which ATL_0005566 demonstrated binding are shown in bold and the peptides showing the highest relative binding at each site of the different species HTT exon 1 are underlined.

FIG. 9 illustrates a workflow for a 3-week stability study, a low pH hold, and freeze thaw cycles tests. The six indicated antibodies were normalised to 5 mg/ml and then used in the 3-week stability study, freeze thaw study, or low pH hold studies. Quality control analysis was performed involving SEC-HPLC (size exclusion high performance liquid chromatography), SDS-PAGE (sodium dodecyl sulfate-polyacrylamide gel electrophoresis), cIEF (capillary isoelectric focusing), and thermal shift analysis.

FIG. 10 shows results of a 3-week stability study or freeze thaw cycles: SEC-HPLC. SEC-HPLC chromatograms for the 6 antibodies indicated (A: ATL_5331, ATL_5334, ATL_5335; B: ATL_5555, ATL_5556, ATL5557), after three weeks incubation at the indicated temperatures (−80 C, +4 C, +21 C, +40 C) or 5 freeze-thaw cycles.

FIG. 11 shows results of a Low pH hold study: SEC-HPLC. Antibodies were prepared at a concentration of 5 mg/ml of PBS. Acetic acid was added dropwise to pH 3.5 and Tris base (pH 11) was added to neutralise the solution to pH 7.2-7.4. Chromatograms for ATL_5331 and ATL_5335 at 0, 15, 30, 60, or 120 minutes after pH neutralisation.

FIG. 12 shows results of an assessment of charge heterogeneity by cIEF. A. pI (isoelectric point) of each of the six antibodies before the 3-week stability study. B-1 to B-3. pI of each of the six antibodies after the 3-week stability study at the indicated temperatures. C-1 to C-3. pI of each of the six antibodies after the 3-week stability study at the at −80° C. or 5 freeze-thaw cycles (5×FT).

FIG. 13 shows results of a HTT pharmacokinetic (PK) study. ATL_0005335 serum levels in mice detected via ELISA, 0, 1, 4, 8, 24, 72 and 144 hours post treatment with 10 or 20 mg/Kg.

FIG. 14 illustrates schematically a workflow for antibody variant panel triage to identify lead antibodies.

FIG. 15 shows results of an analysis of recombinant HTT Exon 1 ELISA (48Q GST—i.e. HTT Exon-1 48Q with a glutathione S-transferase tag used to purify the antigen by affinity chromatography), bars and Thermostability (dots) of variant antibodies. Melting temperatures were measured using SYPRO Orange.

FIG. 16 shows results of HTT Sandwich ELISA for ATL_5895, ATL_5901, ATL5567; and ATL_5059 (As indicated within each panel, top row of table). A-C. HTT Exon 1 25Q ELISA repeat 1. D-G. HTT Exon 1 25Q ELISA repeat 2. H-J. HTT Exon 1 25Q ELISA repeat 3. K-N. HTT Exon 1 48Q ELISA repeat 1. O-R. HTT Exon 1 48Q ELISA repeat 2. S-V. HTT Exon 1 48Q ELISA repeat 3.

FIG. 17 shows results of HTT immunoprecipitation. A. Immunoprecipitation of HTT by ATL_5335 was tested on a U-2 OS cell line expressing 110 CAG repeats resulting in high molecular weight HTT species (indicated as “HTT₁₁₀”), and a parental (control) line (indicated as “HTT_{WT}”). The anti-HTT 1C2 antibody (Sigma Aldrich MAB1574) was used for detection. B. Immunoprecipitation of HTT by ATL_0005895, ATL_0005901, ATL_0005567 was tested with a homogenate derived from R6/2 mouse brain tissue (indicated as “R62 Brain homog”) or a homogenate derived from non-transgenic brain tissue (indicated as “non-Tg brain homog”). The anti-HTT MW8 antibodies (MABN2529 Sigma) and 1C2 (MAB1574 Sigma-Aldrich) were used for detection. C. Immunoprecipitation of HTT by ATL_0005895 was tested with a homogenate derived from human Huntington Disease brain tissue. The anti-HTT antibodies HD1 and MW1 (MABN2427 Millipore) were used for detection. D. Densitometry analysis of blot in FIG. 17C performed using Image J analysis software (NIH).

FIG. 18 shows the results of a FRET-based mHTT (FRASE) assay to assess the ability of antibodies to bind to seed-competent HTT (48Q) species and affect HTT aggregation. (A) shows the rate of aggregation of seed-competent HTT (mHTT) (A t50 values) in the presence of fibrils produced from recombinant HTT after immunodepletion of seed was carried out using the indicated antibodies (ATL5895; ATL5901; MW8 and MW1). Data shown is a representative example from three biological repeats. Error bars represent+/−SD, from n=3, technical replicates in a single biological replicate. (B) shows the rate of aggregation of amount of R6/2 brain added as seed competent HTT (A t50 values), referred to in the figure as “seed”, in the presence of brain homogenates of R6/2 mouse brains after immunodepletion of seed was carried out using the indicated antibodies. Immunodepletion of seed was carried out using the indicated antibodies (ATL5895; ATL5901; MW8 and MW1) on protein G beads (incubation of R6/2 “seed” for 1 hour at 4 degrees with antibody on protein G beads, followed by removal of antibody bound beads and seed if antibody has bound it, remaining solution used in the FRASe aggregation assay). Data shown are representative of 3 biological replicates, each using brain from a different R6/2 mouse. Error bars represent+/−SD, from n=3, technical replicates in a single biological replicate. These data show that immunodepletion of seed with antibodies ATL_0005895 and ATL_0005901 reduced the ability of HTT seeds from both aforementioned sources (Fibrils from recombinant HT and brain homogenates from R6/2 mice) to increase the rate of in vitro aggregation. MW8 (Millipore; MABN2529) binds to aggregated HTT and also achieves this effect to some extent. MW1 (Millipore; MABN2427) binds to PolyQ region of HTT, and cannot achieve this.

FIG. 19 shows a dose-dependent increase in phagocytosis of 48QHTT-coated beads by iPSC-derived microglia in the presence of ATL5895. The uptake of Q48 HTT Exon 1-coated pHrodo™ red beads by induced pluripotent stem cell (iPSC) microglia was measured in the presence of ATL_5895 or a human IgG1 isotype control antibody, ATL5338 which binds to fluorescein. ATL_5895 increases the total red area signal over time, compared with the isotype control antibody. The graph shows the area under the curve (AUC), calculated from the total red fluorescent signal per well over 4 hours of reaction, versus the concentration of antibody in log nM.

FIG. 20 shows the results of an in vivo pharmacokinetic (PK) study of antibodies of the disclosure. (A) shows ATL_0005567 and 5901 serum levels in mice detected via ELISA, 0, 1, 4, 8, 24, 72 and 144 hours post treatment with 10 mg/Kg antibody via intraperitoneal (IP) injection. (B) shows ATL_0005895 serum levels in mice detected via ELISA, 0, 1, 4, 8, 24, 72 and 144 hours post treatment with 1, 10 or 60 mg/Kg antibody via IP injection. (C) shows ATL_0005895 cerebrospinal fluid (CSF) levels in mice detected via ELISA, 4 and 144 hours post treatment with 1, 10 and 60 mg/kg antibody via IP injection. ATL_0005895 1 mg/kg and 144 hour 10 mg/Kg CSF levels were below the limit of quantification of the assay.

FIG. 21 shows the results of an indirect ELISA for binding of the indicated antibodies to HTT exon 1 48Q or lysozyme control. (A) ATL_6199, (B) ATL_6200, (C) ATL_6202, (D) ATL_6203, (E ATL_6204, (F) ATL_6205, (G) ATL_6194, (H) ATL_6195. Absorbance was measured at 450 nm.

FIG. 22 shows the results of a sandwich ELISA for binding of the indicated antibodies to HTT Exon 1 Q48. Repeat 1 was carried out using an antibody starting concentration of 133 nM and results are shown in (A)-(F), (M)-(N). (A) ATL_6199, (B) ATL_6200, (C) ATL_6202, (D) ATL_6203, (E) ATL_6204, (F) ATL_6205, (M) ATL_6194, (N) ATL_6195. Repeat 2 was carried out using an antibody concentration of 400 nM and results are shown in (G)-(L). (G) ATL_6199, (H) ATL_6200, (I) ATL_6202, (J) ATL_6203, (K) ATL_6204, (L) ATL_6205, (O) ATL_6194, (P) ATL_6195. Absorbance was measured at 450 nm.

FIG. 23 shows the results of a sandwich ELISA for binding of the indicated antibodies to HTT Exon 1 Q48. (A) ATL_6183, (B) ATL_6184, (C) ATL_6185, (D) ATL_6186, and (E) control antibody ATL_5338. Absorbance was measured at 450 nm.

FIG. 24 shows the sequences of the framework (FW) regions and complementarity determining regions (CDRs) of antibodies of the present disclosure defined by Kabat. (A) Sequences of HFW1; HCDR1; HFW2; HCDR2; HFW3; HCDR3; HFW4 for the indicated antibodies defined by Kabat. (B) Sequence of LFW1; LCDR1; LFW2; LCDR2; LFW3; LCDR3; LFW4 for the indicated antibodies defined by Kabat. A-1, A-2 show heavy chain sequences for antibodies described in Examples 1-10, 13-16 and 18. A-3, A-4 show heavy chain sequences for antibodies described in Examples 11-12. A-5 shows heavy chain sequences for antibodies described in Examples 17-18. B-1, B-2 show light chain sequences for antibodies described in Examples 1-10, 13-16 and 18. B-3, B-4 show light chain sequences for antibodies described in Examples 11-12. B-5 shows light chain sequences for antibodies described in Examples 17-18.

FIG. 25 shows the results of live animal PET/CT scans using radiolabelled antibody and results of a gamma counting assay. (A and B) Percentage of injected dose (% ID) per gram of blood detected via gamma counting, 0-168 hours post dosing for the C57BL/6J and R6/1 mice (A) in the 11-12 week-old cohort and (B) in the 14-15 week-old cohort. (C and D) Ex vivo biodistribution of ⁸⁹Zr-Df-ATL5895 at 168 hours post dosing in C57BL/6J (left) and R6/1 (right) mice (C) at 11-12 weeks of age and (D) at 14-15 weeks of age, as assessed by gamma counting analysis. (E and F) Brain biodistribution determined by PET/CT imaging of 89Zr-Df-ATL5895 over 168 hours in C57BL/6J and R6/1 mice in the 14-15-week-old cohort. (E) Representative coronal, sagittal and transverse PET/CT images of WT and R6-1 mice.

FIG. 26 shows the results of an immunoassay (meso scale discovery (MSD) assay) to assess the effect of ATL_5895 (ATLX_1095) on HTT aggregate load in the striatum and cortex of R6/1 mice. (A) Aggregated HTT increases in Striatum and Cortex over time. (B) ATL_5895 (ATLX-1095) treatment of R6/1 mice for 12 weeks resulted in a statistically significant decrease in HTT aggregates (MW8/4C9+) in the striatum and cortex. (C) ATL_5895 (ATLX-1095) treatment does not affect the level of mutant Soluble HTT over time. (D) ATL_5895 (ATLX-1095) does not affect the level of endogenous mouse HTT over time.

FIG. 27 shows results of a manufacturability study of ATL_5895 (ATLX_1095). A. SEC-HPLC Chromatograms of ATL_5895-002 4-week thermal stability study samples. Samples run on a Zorbax GF-250 SEC-HPLC column (Agilent). B. SEC-HPLC Chromatograms of ATL_5895-002 10× freeze-thaw samples. Samples run on a TSKgel G3000SWxl column (TOSOH Bioscience). C. cIEF electropherograms of ATL_5895-002 4-week thermal stability study samples. D. cIEF electropherograms of ATL_5895-002 10× freeze-thaw samples vs unstressed control samples. E. Reduced CE-SDS Traces for ATL_5895-002 thermal stability study samples and 10× freeze-thaw cycles. F. HTT Exon-1 Sandwich ELISA of ATL_5895-002 4-week thermal stability study samples and 10× freeze-thaw cycle samples. G. Melting temperatures (Tm1/Tm2) and aggregation temperatures (Tagg) measured for ATL_0005895-002 at 5 mg/mL in 20 mM Histidine-acetate, 150 mM NaCl pH 5.5. H. SEC-HPLC chromatograms of ATL_5895 initial (TO) solubility study samples at 11.90 mg/mL, 23.91 mg/mL, 44.95 mg/mL and 89.96 mg/mL. I. SEC-HPLC chromatograms of ATL_5895 solubility study samples after 1-week incubation at 21° C. at 11.90 mg/mL, 23.91 mg/mL, 44.95 mg/mL and 89.96 mg/mL.

FIG. 28 shows results of a study of binding of ATL5895, ATL6376 and ATL6377 to murine HTT protein, using direct ELISA. Human lysozyme was used as a control antigen. ATL5338 was used as a negative isotype control.

FIG. 29 shows results of a study of binding of affinity optimised antibodies to HTT exon1 or Mutant HTT exon1 48Q. A. HTT exon 1 epitope peptide binding response measured by Octet for mAbs shown tested at 15 nM each. Binding responses confirm that mAbs bind to HTT exon 1 and show increased propensity to bind in comparison to the parent ATL_5895. B. HTT exon 1 epitope peptide binding response measured by Octet for mAbs shown tested at 15 nM each. Binding responses confirm that mAbs bind to HTT exon 1 and show increased propensity to bind in comparison to the parent ATL_5895. C. Mutant HTT exon 1 binding response measured by Octet for mAbs shown tested at 25 nM each. Binding responses confirm that mAbs bind to HTT exon 1 and show increased propensity to bind in comparison to the parent ATL_5895.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Disclosed herein are antibodies and fragments thereof that are capable of specifically binding to Huntingtin (HTT) protein or a fragment thereof. The present disclosure refers to antibodies described herein using references specified as “ATL_000xxxx”, “ATL_xxxx” or “xxxx”, where “xxxx” is a four digits reference number specific to an antibody described herein. All of the above notations are used interchangeably to refer to the same antibody or a portion thereof (e.g. a VH, VL or part thereof, of the antibody). For example, antibody ATL_0005895 is interchangeably referred to herein as ATL 5895 and 5895.

As used herein, an antibody capable of “specific binding” or “specifically binding” a target is one able to bind through the association of the epitope recognition site with an epitope within the target. It is distinct from non-specific binding, for example Fc-mediated binding, ionic and/or hydrophobic interactions. In other words, an antibody which specifically binds a target recognises and binds to a specific protein structure within it rather than to proteins generally.

and is widely distributed throughout the CNS.

HTT is a soluble 3144 amino acid (384Kda) protein (in its non-expanded form), widely distributed throughout the central nervous system (CNS) and linked to the neurodegenerative disorder Huntington's disease (HD). HD is caused by an expansion of the trinucleotide repeat CAG in exon 1 of HTT encoding a polyglutamine (polyQ) tract near the N-terminus of toxic mutant huntingtin (mHTT). In healthy individuals the length of the CAG repeats typically ranges from 9 to 35 CAG repeats, whereas repeat numbers in excess of 40 result in disease expression. CAG repeats of 36-39 are associated with reduced penetrance whereby some develop HD and others do not.

The elongated polyQ tract in mutant huntingtin results in protein misfolding and leads to reduced solubility. These insoluble aggregates of mHTT are highly toxic to neurons, ultimately leading to neuronal cell death. Toxic accumulation of mHTT occurs in different parts of the brain and aggregation may occur in the nucleus, cytoplasm, and extracellularly. In addition, mutant aggregates demonstrate “seeding” potential, i.e. these proteins spontaneously aggregate and aggregates accelerate the rate of aggregation of mHTT when assessed e.g. in cell-free assays, causing spread of pathological HTT in the CNS. Advantageously, antibodies of the present disclosure may bind the toxic extracellular mHTT and prevent propagation of mHTT via removal of aggregated mHTT and/or inhibition of mHTT seeding potential. The human gene (Gene ID: 3064) encoding HTT is located at 4p16.3, and is large, spanning 180 kb and consisting of 67 exons. Reference non-human HTT amino acid and coding sequences are available in public databases.

A reference human HTT exon 1 amino acid sequence is provided as “Wild type Huntingtin (HTT) exon 1” or “Mutant Huntingtin (HTT) exon 1” below (see below and Table 1). However, as used herein, the terms “HTT” and “HTT exon 1” encompass truncations, derivatives, and variants of the sequences of HTT exon 1 provided herein, and may refer to any protein with at least 80%, at least 90%, or at least 95% sequence identity to “Wild type Huntingtin (HTT) exon 1” or “Mutant Huntingtin (HTT) exon 1” below. In some embodiments, the antibodies described herein are capable of specifically binding a peptide or protein having or comprising the amino acid sequence of “Wild type Huntingtin (HTT) exon 1” or “Mutant Huntingtin (HTT) exon 1” (including a full length HTT protein comprising said sequence, or a fragment of said protein comprising said sequence), or a fragment thereof.

In some embodiments, the antibodies described herein are capable of specifically binding a HTT protein or protein fragment comprising or consisting of a HTT variant amino acid sequence. In some embodiments, the HTT variant protein or fragment comprises an amino acid sequence that has at least 70%, at least 80%, at least 85%, at least 90%, at least 95%, at least 95%, or at least 99% identity with “Wild type Huntingtin (HTT) exon 1” or “Mutant Huntingtin (HTT) exon 1” In some embodiments, the antibodies disclosed herein are capable of specifically binding a HTT fragment comprising at least 20%, 30%, 40%, 50%, 60%, 70%, 80%, 90% 95% or more of the HTT amino acid sequence, or a HTT variant sequence.

In some embodiments, antibodies and fragments of the disclosure bind to regions in exon 1 of HTT. Exon 1 of HTT may comprise an elongated polyQ repeat caused by an expanded CAG repeat (e.g., A CAG repeat in excess of 35-40). Any HTT protein or fragment thereof comprising a polyQ tract having 36 or more glutamines may be considered as pathological, meaning HD disease expression may occur. Thus, the term “mutant HTT” (mHTT) as used herein refers to a HTT protein or fragment thereof that has at least 36 glutamines in its polyQ repeat region. Exon 1 HTT with 35 or fewer glutamines may be considered as non-pathological. HTT proteins or fragments thereof comprising 35 or fewer glutamines in the polyQ repeat regions will be referred to as wild-type (WT) HTT. The terms “mutant HTT” and “aggregated HTT” are used herein interchangeably to refer to HTT with an expanded polyQ tract (also referred to as “high molecular weight HTT”), such as e.g. HTT with 36 or more glutamines.

In some embodiments, HTT exon 1 has a polyQ tract containing 9 to 35 glutamines. In some embodiments, the polyQ tract contains 25 glutamines. For example, human HTT exon 1 may have a polyQ tract containing 25 glutamines. A human HTT exon 1 having a polyQ tract containing 25 glutamines may have the following sequence (referred to as “Wild type Huntingtin (HTT) exon 1”):

>Human_HTT_exon_1_25Q
(SEQ ID NO: 43)
MKHHHHHHNTSSNSMSPILGYWKIKGLVQPTRLLLEYLEEKYEEHLYERDEGDKWRNKKF
ELGLEFPNLPYYIDGDVKLTQSMAIIRYIADKHNMLGGCPKERAEISMLEGAVLDIRYGVSRIA
YSKDFETLKVDFLSKLPEMLKMFEDRLCHKTYLNGDHVTHPDFMLYDALDVVLYMDPMCLD
AFPKLVCFKKRIEAIPQIDKYLKSSKYIAWPLQGWQATFGGGDHPPTSGSGGGGGWMSEN
LYFQGAMATLEKLMKAFESLKSFQQQQQQQQQQQQQQQQQQQQQQQQQPPPPPPPPP
PPQLPQPPPQAQPLLPQPQPPPPPPPPPPGPAVAEEPLHRP.

In some embodiments, HTT exon 1 has a polyQ tract containing more than 40 glutamines. In some embodiments, the polyQ tract contains 48 glutamines. For example, human HTT exon 1 may have a polyQ tract containing 48 glutamines. A human HTT exon 1 having a polyQ tract containing 48 glutamines may have the following sequence (referred to as “Mutant Huntingtin (HTT) exon 1”):

>Human HTT_exon_1_48Q
(SEQ ID NO: 44)
MKHHHHHHNTSSNSMSPILGYWKIKGLVQPTRLLLEYLEEKYEEHLYERDEGDKWRNKKF
ELGLEFPNLPYYIDGDVKLTQSMAIIRYIADKHNMLGGCPKERAEISMLEGAVLDIRYGVSRIA
YSKDFETLKVDFLSKLPEMLKMFEDRLCHKTYLNGDHVTHPDFMLYDALDVVLYMDPMCLD
AFPKLVCFKKRIEAIPQIDKYLKSSKYIAWPLQGWQATFGGGDHPPTSGSGGGGGWMSEN
LYFQGAMATLEKLMKAFESLKSFQQQQQQQQQQQQQQQQQQQQQQQQQQQQQQQQQ
QQQQQQQQQQQQQQQPPPPPPPPPPPQLPQPPPQAQPLLPQPQPPPPPPPPPPGPAVA
EEPLHRP.

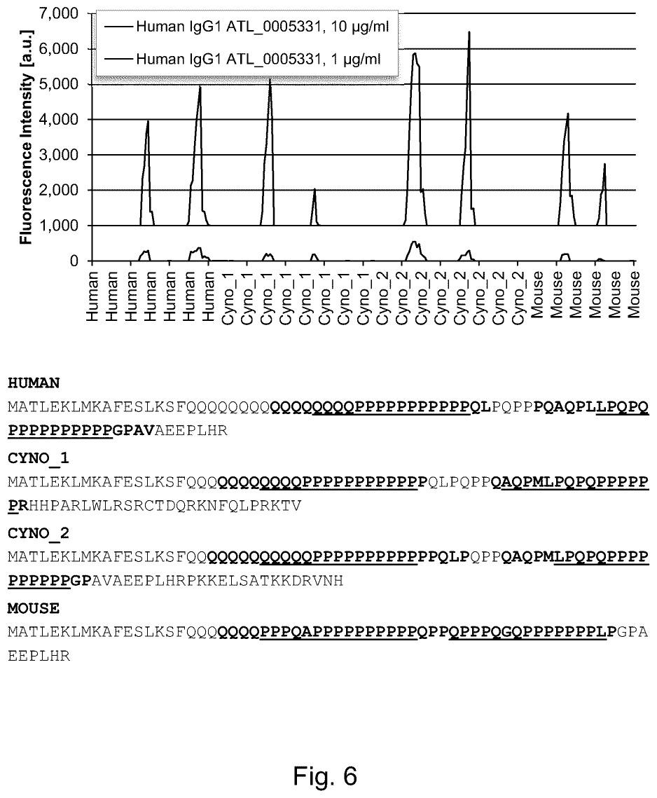

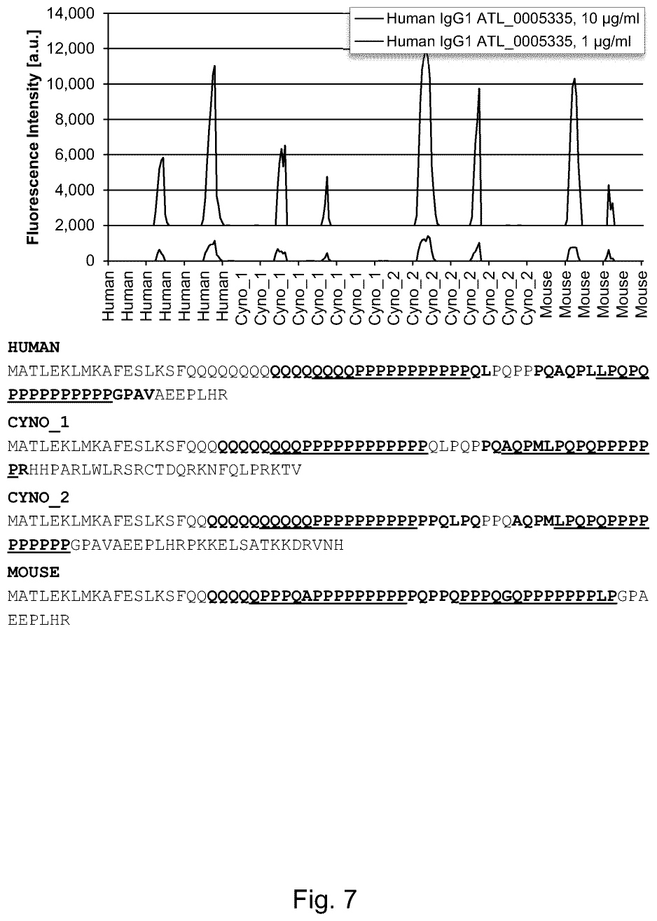

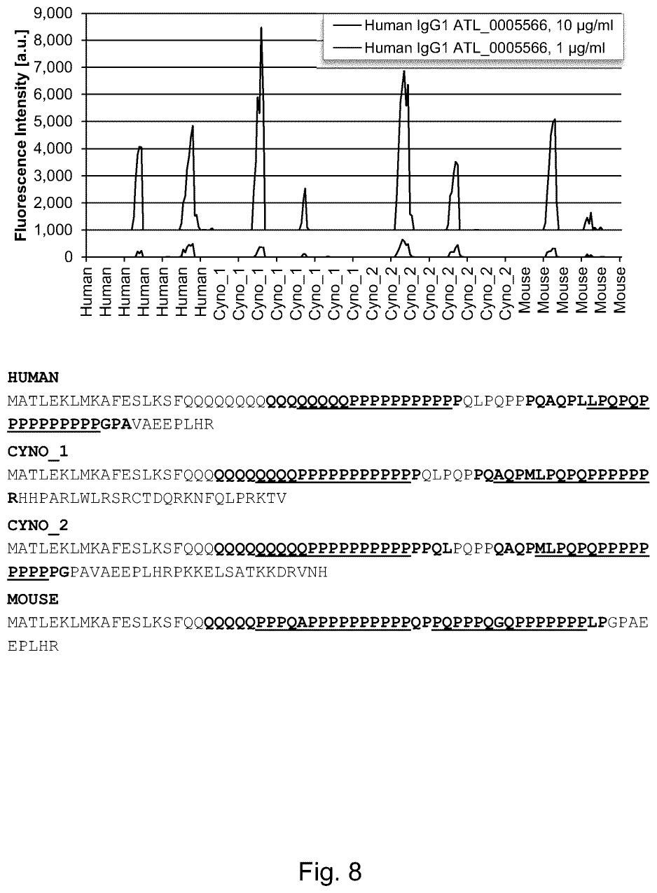

The binding region(s) of antibodies described herein may be located in the polyP region, the polyQ/PolyP region, the P rich region, the C-terminal region, and/or the N-terminal region of HTT. In some embodiments, the antibodies bind an epitope within the polyP and/or polyQ/polyP tract of HTT. In some embodiments, the epitope comprises or is comprised within amino acid sequence QQQQPPPPPPPPPPP (SEQ ID NO: 47) or PQPQPPPPPPPPPPP (SEQ ID NO: 48) of human HTT, or corresponding regions in homolog proteins. Binding regions for exemplary antibodies of the disclosure to various HTT proteins are shown on FIGS. 6 to 8 (bold regions). In embodiments, the binding region(s) of antibodies described herein are located in any of the regions in bold on FIG. 6, 7 or 8. The regions in exon 1 of HTT are discussed by Angelopoulou, E., et al. (Exploring the role of high-mobility group box 1 (HMGB1) protein in the pathogenesis of Huntington's disease. J Mol Med 98, 325-334 (2020).) In embodiments, the antibodies bind to an epitope within exon 1 of HTT. In embodiments, the antibodies bind to an epitope within exon 1 of HTT that does not comprise the polyQ tract. In embodiments, the antibodies may not bind to proteins derived from other CAG repeat-containing genes other than HTT. In other words, the antibodies of the present disclosure may advantageously not show off target binding to other proteins expressed from CAG repeat genes (such as e.g. ataxins). In embodiments, the antibodies of the disclosure may bind to high molecular weight/aggregated HTT and soluble forms of HTT/wild type HTT.

HTT may be human HTT or mouse HTT. Suitably, HTT may be human HTT. HTT may refer to human HTT unless context indicates otherwise. In other embodiments, for example when the individual to be treated is a non-human mammal, HTT may non-human HTT.

An antibody or fragment thereof may bind human HTT, and also bind mouse (murine) HTT antigen. For example, an antibody or fragment thereof may also bind mouse HTT antigen with the amino acid sequence set forth in SEQ ID NO: 177. Cross-reactivity with mouse HTT antigen may be determined by direct ELISA, for example direct ELISA performed as described herein (Example 18, Materials and Methods).

The present disclosure relates primarily to antibody molecules, whether whole antibody (e.g. IgG, such as IgG4) or antibody fragments (e.g. scFv, Fab, (single-domain) dAb). Antibody antigen binding regions (also referred to as “antigen binding portions”) are provided, as are antibody VH and VL domains. Within VH and VL domains are provided complementarity determining regions, CDR's, which may be provided within different framework regions, FR's, to form VH or VL domains as the case may be. An antigen binding site may consist of an antibody VH domain and/or a VL domain.

Antibodies according to the present disclosure may be provided in isolated form. The term “antibody” encompasses a fragment or derivative thereof, or a synthetic antibody or synthetic antibody fragment.

The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 25 792-799).

An antibody or fragment thereof may be a monoclonal antibody. Monoclonal antibodies (mAbs) are homogenous populations of antibodies specifically targeting a single epitope on an antigen.

Fragments of antibodies, such as Fab and Fab2 fragments may also be provided as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parent antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the VH and VL partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

The term “ScFv molecules” refers to molecules wherein the VH and VL partner domains are covalently linked, e.g. by a flexible oligopeptide. Fab, Fv, ScFv and dAb antibody fragments can all be expressed in and secreted from E. coli, thus allowing the facile production of large amounts of the said fragments. Whole antibodies, and F(ab′) 2 fragments are “bivalent”. The term “bivalent” means that the said antibodies and F(ab′) 2 fragments have two antigen combining sites. In contrast, Fab, Fv, ScFv and dAb fragments are monovalent, having only one antigen combining site.

Antibodies according to the present disclosure may be detectably labelled or, at least, capable of detection. For example, the antibody may be labelled with a radioactive atom or a coloured molecule or a fluorescent molecule or a molecule which can be readily detected in any other way. Suitable detectable molecules include fluorescent proteins, luciferase, enzyme substrates, and radiolabels. The binding moiety (antibody or fragment thereof) may be directly labelled with a detectable label or it may be indirectly labelled. For example, the binding moiety may be an unlabelled antibody which can be detected by another antibody which is itself labelled. Alternatively, the second antibody may have bound to it biotin and binding of labelled streptavidin to the biotin is used to indirectly label the first antibody.

A “fragment” of an antibody may comprise any number of residues of a “parental” antibody, whilst retaining target binding ability. A fragment may lack effector function, for example may be entirely unable to bind or show diminished binding to the Fc receptor, relative to the parent. A fragment is typically smaller than the parental antibody. A fragment may comprise 50%, 60%, 70%, 80%, 90%, 95% or more of the contiguous or non-contiguous amino acids of the parental antibody. A fragment may comprise 50, 100, 150, 200, 250, 300 or more contiguous or non-contiguous amino acids of the parental antibody. A fragment may comprise deletions in the Fc region, or of the Fc region. A fragment may retain the CDRs and/or the variable domains of the parental antibody, unaltered. In some embodiments, a fragment is an Fab fragment or an F(ab′)2 fragment.

CDR sequences are described herein using the Kabat definition (Kabat, E. A. et al., Sequences of Proteins of Immunological Interest.).

Antibodies according to the present disclosure may have the CDR's of antibody ATL_5895, in which:
i. HCDR1 has amino acid sequence KAWMS (SEQ ID NO:1);
ii. HCDR2 has amino acid sequence RIKSGIDAGTTDYAAPVKG (SEQ ID NO:2);
iii. HCDR3 has amino acid sequence PPYYYYYGLDV (SEQ ID NO:3);
iv. LCDR1 has amino acid sequence TGTSSDVGSYNLVS (SEQ ID NO:4);
v. LCDR2 has amino acid sequence EVNKRPS (SEQ ID NO:5);
vi. LCDR3 has amino acid sequence GSYAGTANV (SEQ ID NO:6).

Antibodies according to the present disclosure may have the V_{H} and/or V_{L} sequence of antibody ATL_5895, in which: (a) the V_{H} of ATL_5895 has the sequence:
EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCIPPPYYYYYGLDVWGQGTTVTVSS (SEQ ID NO: 7); and (b) the V_{L} of ATL_5895 has the sequence:
QSALTQPRSVSGSPGQSVTISCTGTSSDVGSYNLVSWYQQHPGKAPKLMIYEVNKRPSGVPDRFS GSKSGATASLTISGLQAEDEADYYCGSYAGTANVFGTGTKVTVL (SEQ ID NO:8).

Antibodies according to the present disclosure may have the CDRs of antibody ATL_5901, in which:
i. HCDR1 has amino acid sequence KAWMS (SEQ ID NO:1);
ii. HCDR2 has amino acid sequence RIKSGIDAGTTDYAAPVKG (SEQ ID NO:2);
iii. HCDR3 has amino acid sequence PPYYYYYGLDV (SEQ ID NO:3);
iv. LCDR1 has amino acid sequence TGTSSDVGGYKLVS (SEQ ID NO:9);
v. LCDR2 has amino acid sequence EVSKRPS (SEQ ID NO: 10);
vi. LCDR3 has amino acid sequence SSYAGSSVV (SEQ ID NO:11).

Antibodies according to the present disclosure may have the V_{H} and/or V_{L} sequence of antibody ATL_5901 in which: (a) the V_{H} of ATL_5901 has the sequence:
EVOLVESGGGLVKPGGSLRLSCAASGFTFNKAWMSWVRQAPGKGLEWVGRIKSGIDAGTTDYAAP VKGRFTISRDDSKATLYLQMNSLKTEDTAVYYCIPPPYYYYYGLDVWGQGTTVTVSS (SEQ ID NO: 12); and (b) the V_{L} of ATL_5901 has the sequence:
QSALTQPASVSGSPGQSITISCTGTSSDVGGYKLVSWYQQHPGRAPKLMIYEVSKRPSGVSSRFSG SKSGSTASLTISGLQAEDEADYYCSSYAGSSVVFGGGTKLTVL (SEQ ID NO:13).

Antibodies according to the present disclosure may have the CDRs of antibody ATL_5567, in which:
i. HCDR1 has amino acid sequence KAWMN (SEQ ID NO:14);
ii. HCDR2 has amino acid sequence RIKSGIDGGTTDYAAPVQG (SEQ ID NO: 15);
iii. HCDR3 has amino acid sequence PPYYYYYGLDV (SEQ ID NO:3);
iv. LCDR1 has amino acid sequence TGTSSDIGSYNLVS (SEQ ID NO:16);
v. LCDR2 has amino acid sequence EGSKRPS (SEQ ID NO: 17);
vi. LCDR3 has amino acid sequence SSYAGFSTLV (SEQ ID NO:18).

Antibodies according to the present disclosure may have the V_{H} and/or V_{L} sequence of antibody ATL_5567 in which: (a) the V_{H} of ATL_5567 has the sequence: (a)
EVOLVESGGGLVKPGGSLRLSCAASGFTFNKAWMNWVRQAPGKGLEWVGRIKSGIDGGTTDYAA PVQGRFTISRDDSKNTVYLQMNSLNTEDTAVYYCIPPPYYYYYGLDVWGQGTTVTVSS (SEQ ID NO: 19); and (b) the V_{L} of ATL 5567 has the sequence:
QSALTQPASVSGSPGQSITISCTGTSSDIGSYNLVSWYQQHPGNAPKPLIYEGSKRPSGVSARFSGS KSGNTASLTISGLQPEDEADYYCSSYAGFSTLVFGGGTKVTVL (SEQ ID NO:20).

Antibodies of the present disclosure may have the CDRs of antibody ATL_5331, in which:
i. HCDR1 has amino acid sequence KAWMN (SEQ ID NO:14);
ii. HCDR2 has amino acid sequence RIKSGIDGGTTDYAAPVQG (SEQ ID NO:15);
iii. HCDR3 has amino acid sequence PPYYYYYGLDV (SEQ ID NO:3);
iv. LCDR1 has amino acid sequence TGTSSDVGSYNLVS (SEQ ID NO:4);
v. LCDR2 has amino acid sequence EVNKRPS (SEQ ID NO:5);
vi. LCDR3 has amino acid sequence GSYAGTNNV (SEQ ID NO:21).

Antibodies according to the present disclosure may have the V_{H} and/or V_{L} sequence of antibody ATL_5331 in which: (a) the V_{H} of ATL_5331 (AC_0737) has the sequence:
EVOLVESGGGLVKPGGSLRLSCAASGFTFNKAWMNWVRQAPGKGLEWVGRIKSGIDGGTTDYAA PVQGRFTISRDDSKNTVYLQMNSLNTEDTAVYYCIPPPYYYYYGLDVWGQGTTVTVSS (SEQ ID NO: 19); and (b) the V_{L} of ATL 5331 has the sequence:
QSALTQPRSVSGSPGQSITISCTGTSSDVGSYNLVSWFQQHPGKAPKLIIYEVNKRPSGVPDRFSGS KSGNTASLTVSGLQAEDEADYYCGSYAGTNNVFGTGTKLTVL (SEQ ID NO:22)

Antibodies of the present disclosure may have CDRs of ATL_5334:
i. HCDR1 has amino acid sequence KAWMN (SEQ ID NO:14);

ii. HCDR2 has amino acid sequence RIKSGIDGGTTDYAAPVQG (SEQ ID NO:15);
iii. HCDR3 has amino acid sequence PPYYYYYGLDV (SEQ ID NO:3);
iv. LCDR1 has amino acid sequence TGTSSDVGGYKLVS (SEQ ID NO:9);
v. LCDR2 has amino acid sequence EVSKRPS (SEQ ID NO:10);
vi. LCDR3 has amino acid sequence CSYAGSSVV (SEQ ID NO: 23).

Antibodies according to the present disclosure may have the V_{H} and/or V_{L} sequence of antibody ATL_5334, in which: (a) the V_{H} of ATL_5334 (AC_0737) has the sequence:
EVOLVESGGGLVKPGGSLRLSCAASGFTFNKAWMNWVRQAPGKGLEWVGRIKSGIDGGTTDYAA PVQGRFTISRDDSKNTVYLQMNSLNTEDTAVYYCIPPPYYYYYGLDVWGQGTTVTVSS (SEQ ID NO: 19); and (b) the V_{L} of ATL 5334 has the sequence:
QSALTQPASVSGSPGQSITISCTGTSSDVGGYKLVSWYQQHPGRAPKLMIYEVSKRPSGVSSRFSG SKSGSTASLTISGLQAEDEADYYCCSYAGSSVVFGGGTKLTVL (SEQ ID NO: 24).

Antibodies of the present disclosure may have CDRs of ATL_5335:
i. HCDR1 has amino acid sequence KAWMN (SEQ ID NO:14);
ii. HCDR2 has amino acid sequence RIKSGIDGGTTDYAAPVQG (SEQ ID NO:15);
iii. HCDR3 has amino acid sequence PPYYYYYGLDV (SEQ ID NO:3);
iv. LCDR1 has amino acid sequence TGTSSDIGSYNLVS (SEQ ID NO:16);
v. LCDR2 has amino acid sequence EGSKRPS (SEQ ID NO: 17);
vi. LCDR3 has amino acid sequence SSYAGFNTLV (SEQ ID NO: 25).

Antibodies according to the present disclosure may have the V_{H} and/or V_{L} sequence of antibody

ATL_5335, in which: (a) the V_{H} ATL_5335 (AC_0737) has the sequence:
EVOLVESGGGLVKPGGSLRLSCAASGFTFNKAWMNWVRQAPGKGLEWVGRIKSGIDGGTTDYAA PVQGRFTISRDDSKNTVYLQMNSLNTEDTAVYYCIPPPYYYYYGLDVWGQGTTVTVSS (SEQ ID NO: 19); and (b) the V_{L} of ATL_5335 has the sequence:
QSALTQPASVSGSPGQSITISCTGTSSDIGSYNLVSWYQQHPGNAPKPLIYEGSKRPSGVSARFSGS KSGNTASLTISGLQPEDEADYYCSSYAGFNTLVFGGGTKVTVL (SEQ ID NO: 26).

Antibodies of the present disclosure may have CDRs of ATL_5555, in which:
i. HCDR1 has amino acid sequence KAWMS (SEQ ID NO:1);
ii. HCDR2 has amino acid sequence RIKSGIDGGTTDYAAPVKG (SEQ ID NO: 27);
iii. HCDR3 has amino acid sequence PPYYYYYGLDV (SEQ ID NO:3);
iv. LCDR1 has amino acid sequence TGTSSDVGSYNLVS (SEQ ID NO:4);
v. LCDR2 has amino acid sequence EVNKRPS (SEQ ID NO:5);
vi. LCDR3 has amino acid sequence GSYAGTNNV (SEQ ID NO:21).

Antibodies according to the present disclosure may have the V_{H} and/or V_{L} sequence of antibody ATL_5555, in which: (a) the V_{H} ATL_5555 (AC_1269) has the sequence:
EVOLVESGGGLVKPGGSLRLSCAASGFTFNKAWMSWVRQAPGKGLEWVGRIKSGIDGGTTDYAA PVKGRFTISRDDSKNTLYLQMNSLKTEDTAVYYCIPPPYYYYYGLDVWGQGTTVTVSS (SEQ ID NO: 28); and (b) the V_{L} of ATL_5555 has the sequence:
QSALTQPRSVSGSPGQSVTISCTGTSSDVGSYNLVSWYQQHPGKAPKLMIYEVNKRPSGVPDRFS GSKSGNTASLTISGLQAEDEADYYCGSYAGTNNVFGTGTKVTVL (SEQ ID NO: 29)

Antibodies of the present disclosure may have CDRs of ATL_5556, in which:
i. HCDR1 has amino acid sequence KAWMS (SEQ ID NO:1);
ii. HCDR2 has amino acid sequence RIKSGIDGGTTDYAAPVKG (SEQ ID NO: 27);
iii. HCDR3 has amino acid sequence PPYYYYYGLDV (SEQ ID NO:3);
iv. LCDR1 has amino acid sequence TGTSSDVGGYKLVS (SEQ ID NO:9);
v. LCDR2 has amino acid sequence EVSKRPS (SEQ ID NO: 10);
vi. LCDR3 has amino acid sequence CSYAGSSVV (SEQ ID NO: 23)

Antibodies according to the present disclosure may have the V_{H} and/or V_{L} sequence of antibody ATL_5556, in which: (a) the V_{H} of ATL_5556 (AC_1269) has the sequence:
EVOLVESGGGLVKPGGSLRLSCAASGFTFNKAWMSWVRQAPGKGLEWVGRIKSGIDGGTTDYAA PVKGRFTISRDDSKNTLYLQMNSLKTEDTAVYYCIPPPYYYYYGLDVWGQGTTVTVSS (SEQ ID NO: 28); and (b) the V_{L} of ATL_5556 has the sequence:
QSALTQPASVSGSPGQSITISCTGTSSDVGGYKLVSWYQQHPGRAPKLMIYEVSKRPSGVSSRFSG SKSGSTASLTISGLQAEDEADYYCCSYAGSSVVFGGGTKLTVL (SEQ ID NO: 24).

Antibodies of the present disclosure may have CDRs of ATL_5557, in which:
i. HCDR1 has amino acid sequence KAWMS (SEQ ID NO:1);
ii. HCDR2 has amino acid sequence RIKSGIDGGTTDYAAPVKG (SEQ ID NO: 27);
iii. HCDR3 has amino acid sequence PPYYYYYGLDV (SEQ ID NO:3);
iv. LCDR1 has amino acid sequence TGTSSDIGSYNLVS (SEQ ID NO:16);
V. LCDR2 has amino acid sequence EGSKRPS (SEQ ID NO:17);
vi. LCDR3 has amino acid sequence SSYAGENTLV (SEQ ID NO: 25).

Antibodies according to the present disclosure may have the V_{H} and/or V_{L} sequence of antibody ATL_5557, in which: (a) the V_{H} ATL_5557 (AC_1269) has the sequence:
EVOLVESGGGLVKPGGSLRLSCAASGFTFNKAWMSWVRQAPGKGLEWVGRIKSGIDGGTTDYAA PVKGRFTISRDDSKNTLYLQMNSLKTEDTAVYYCIPPPYYYYYGLDVWGQGTTVTVSS (SEQ ID NO: 28); and (b) the V_{L} of ATL_5557 has the sequence:
QSALTQPASVSGSPGQSITISCTGTSSDIGSYNLVSWYQQHPGKAPKLMIYEGSKRPSGVSNRFSG SKSGNTASLTISGLQAEDEADYYCSSYAGFNTLVFGGGTKLTVL (SEQ ID NO: 30).

Antibodies of the present disclosure may have CDRs of any of antibodies ATL_6199, ATL_6200, ATL_6202, ATL_6203, ATL_6204, ATL_6205, ATL_6194, ATL_6195, ATL_6374, ATL_6375, ATL_6376, ATL_6377, ATL_6378, as provided in Table 1. Antibodies according to the present disclosure may have the V_{H} and/or V_{L} sequence of any of antibodies ATL_6199, ATL_6200, ATL_6202, ATL_6203, ATL_6204, ATL_6205, ATL_6194, ATL_6195, ATL_6374, ATL_6375, ATL_6376, ATL_6377, ATL_6378, as provided below:

-the VH of ATL_6199:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYWCSPPPYYYYYGLDVWGQGTTVT
VSS,
and
-the VL of ATL_6199:
QSALTQPRSVSGSPGQSVTISCTGTSSDVGSYNLVSWYQQHPGKAPKLMIYEVNKRPSGV
PDRFSGSKSGATASLTISGLQAEDEADYYCGSYAGTANVFGTGTKVTVL;
the VH of ATL_6200:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCVPPPYYYYYGLDVWGQGTTVTV
SS;
the VL of ATL_6200:
QSALTQPRSVSGSPGQSVTISCTGTSSDVGSYNLVSWYQQHPGKAPKLMIYEVNKRPSGV
PDRFSGSKSGATASLTISGLQAEDEADYYCGSYAGTANVFGTGTKVTVL;
-the VH of ATL_6202:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCSPPPFYYYYGLDVWGQGTTVTV
SS,
the VL of ATL_6202:
QSALTQPRSVSGSPGQSVTISCTGTSSDVGSYNLVSWYQQHPGKAPKLMIYEVNKRPSGV
PDRFSGSKSGATASLTISGLQAEDEADYYCVSYGGTENVFGTGTKVTVL
the VH of ATL_6203:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCIPSPYYYYYGLDVWGQGTTVTV
SS,
-the VL of ATL_6203:
QSALTQPRSVSGSPGQSVTISCTGTSSDVGSYNLVSWYQQHPGKAPKLMIYEVNKRPSGV
PDRFSGSKSGATASLTISGLQAEDEADYYCVSYAGTANVFGTGTKVTVL
the VH of ATL_6204:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCIPPPYYYYYGLDVWGQGTTVTV
SS,
-the VL of ATL_6204:
QSALTQPRSVSGSPGQSVTISCTGTSSDVGSYNLVSWYQQHPGKAPKLMIYEVNKRPSGV
PDRFSGSKSGATASLTISGLQAEDEADYYCVSFAGTANVFGTGTKVTVL
the VH of ATL_6205:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYWCVPPPYYYYYGLDVWGQGTTVT
VSS;
-the VL of ATL_6205:
QSALTQPRSVSGSPGQSVTISCTGTSSDVGSYNLVSWYQQHPGKAPKLMIYEVNKRPSGV
PDRFSGSKSGATASLTISGLQAEDEADYYCVSYAGTANVFGTGTKVTVL
the VH of ATL_6194:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCVPPPYYYYYGLDVWGQGTTVTV
SS;
-the VL of ATL_6194:
QSALTQPRSVSGSPGQSVTISCTGTSSDVGSYNLVSWYQQHPGKAPKLMIYEVNKRPSGV
PDRFSGSKSGATASLTISGLQAEDEADYYCVSYAGTANVFGTGTKVTVL
-the VH of ATL_6195:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCTPPPYYYYYGLDVWGQGTTVTV
SS;
the VL of ATL_6195:
QSALTQPRSVSGSPGQSVTISCTGTSSDVGSYNLVSWYQQHPGKAPKLMIYEVNKRPSGV
PDRFSGSKSGATASLTISGLQAEDEADYYCGSYAGTANVFGTGTKVTVL
-the VH of ATL_6374:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYWCVPPPYYYYYGLDVWGQGTTVT
VSS;
the VL of ATL_6374:
QSALTQPRSVSGSPGQSVTISCTGTSSDVGSYNLVSWYQQHPGKAPKLMIYEVNKRPSGV
PDRFSGSKSGATASLTISGLQAEDEADYYCVSYGGTENVFGTGTKVTVL
-the VH of ATL_6375:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCVPPPYYYYYGLDVWGQGTTVTV
SS;
the VL of ATL_6375:
QSALTQPRSVSGSPGQSVTISCTGTSSDVGSYNLVSWYQQHPGKAPKLMIYEVNKRPSGV
PDRFSGSKSGATASLTISGLQAEDEADYYCVSYGGTENVFGTGTKVTVL
-the VH of ATL_6376:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCVPPPFYYYYGLDVWGQGTTVTV
SS;
the VL of ATL_6376:
QSALTQPRSVSGSPGQSVTISCTGTSSDVGSYNLVSWYQQHPGKAPKLMIYEVNKRPSGV
PDRFSGSKSGATASLTISGLQAEDEADYYCVSYGGTENVFGTGTKVTVL
-the VH of ATL_6377:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCVPSPYYYYYGLDVWGQGTTVTV
SS;
the VL of ATL_6377:
QSALTQPRSVSGSPGQSVTISCTGTSSDVGSYNLVSWYQQHPGKAPKLMIYEVNKRPSGV
PDRFSGSKSGATASLTISGLQAEDEADYYCVSYAGTANVFGTGTKVTVL
-the VH of ATL_6378:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCVPSPYYYYYGLDVWGQGTTVTV
SS;
the VL of ATL_6378:
QSALTQPRSVSGSPGQSVTISCTGTSSDVGSYNLVSWYQQHPGKAPKLMIYEVNKRPSGV
PDRFSGSKSGATASLTISGLQAEDEADYYCVSYGGTENVFGTGTKVTVL.

Antibodies of the present disclosure may have CDRs of any of antibodies ATL_6183, ATL_6184, ATL_6185, ATL_6186, as provided in Table 1. Antibodies according to the present disclosure may have the V_{H} and/or V_{L} sequence of any of antibodies ATL_6183, ATL_6184, ATL_6185, ATL_6186, as provided below:

-the VH of ATL6183:
EVOLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCTPPPYYYYGLDVWGQGTTVTVS
S;
the VL of ATL6183:
QSALTQPRSVSGSPGQSVTISCTGTSSDVGSYNLVSWYQQHPGKAPKLMIYEVNKRPSGV
PDRFSGSKSGATASLTISGLQAEDEADYYCGSYAGTANVFGTGTKVTVL
-the VH of ATL6184:
EVQLVESGGGLVKPGGSLRLSCAASGFTFNKAWMSWVRQAPGKGLEWVGRIKSGIDAGT
TDYAAPVKGRFTISRDDSKATLYLQMNSLKTEDTAVYYCTPPPYYYYGLDVWGQGTTVTVS
S;
the VL of ATL6184:
QSALTQPASVSGSPGQSITISCTGTSSDVGGYKLVSWYQQHPGRAPKLMIYEVSKRPSGVS
SRFSGSKSGSTASLTISGLQAEDEADYYCSSYAGSSVVFGGGTKLTVL
the VH of ATL6185:
EVQLVESGGGLVKPGGSLRLSCAASGFTFNKAWMSWVRQAPGKGLEWVGRIKSGIDAGT
TDYAAPVKGRFTISRDDSKNTLYLQMNSLKTEDTAVYYCTPPPYYYYGLNVWGQGTTVTVS
S;
the VL of ATL6185:
QSALTQPRSVSGSPGQSVTISCTGTSSDVGSYNLVSWYQQHPGKAPKLMIYEVNKRPSGV
PDRFSGSKSGATASLTISGLQAEDEADYYCGSYAGTANVFGTGTKVTVL
-the VH of ATL6186:
EVQLVESGGGLVKPGGSLRLSCAASGFTFNKAWMSWVRQAPGKGLEWVGRIKSGIDAGT
TDYAAPVKGRFTISRDDSKATLYLQMNSLKTEDTAVYYCTPPPYYYYGLNVWGQGTTVTVS
S;
the VL of ATL6186:
QSALTQPASVSGSPGQSITISCTGTSSDVGGYKLVSWYQQHPGRAPKLMIYEVSKRPSGVS
SRFSGSKSGSTASLTISGLQAEDEADYYCSSYAGSSVVFGGGTKLTVL.

In an antibody according to the present disclosure at least one of the sequences (i) to (vi) may vary. A variant may have one, two, three, four, five or more (such as e.g. up to 10) amino acid substitutions in one or more of the sequences (i) to (vi). In embodiments, an antibody according to the disclosure comprises CDRs with sequences that have 1, 2 or 3 substitutions compared to the sequences (i) to (vi) of any antibody described above. For example, an antibody according to the disclosure may comprise CDRs with the sequences of any antibody above, except that 1, 2 or 3 of the CDRs comprise a substitution, where the total number of substitutions across the CDRs does not exceed 3.

The VH and VL chain CDRs 1-3 of any of the antibodies described above may also be particularly useful in conjunction with a number of different framework regions. Accordingly, light and/or heavy chains having CDRs 1-3 as described above may possess an alternative framework region. Suitable framework regions are known in the art and are described for example in M. Lefranc & G. Le Franc (2001) “The Immunoglobulin Facts Book”, Academic Press.

In this specification, antibodies may have VH and/or VL regions comprising an amino acid sequence that has a high percentage sequence identity to the VH and/or VL amino acid sequences described above.

For example, antibodies according to the present invention include antibodies that bind HTT and have a VH region that comprises an amino acid sequence having at least 70%, more preferably one of at least 75%, 80%, 85%, 90%, 95%, 96%, 97%, 98%, 99% or 100%, sequence identity to the VH region amino acid sequence of any antibody described above, such as e.g. ATL5895, or ATL5901, or 5567.

Alternatively or additionally, antibodies of the disclosure may have a VL region that comprises an amino acid sequence having at least 70%, more preferably one of at least 75%, 80%, 85%, 90%, 95%, 96%, 97%, 98%, 99% or 100%, sequence identity to the VL region amino acid sequence of any antibody described above, such as e.g. ATL5895, or ATL5901, or 5567.

Overall percentage identity of a variable region or full length heavy/light chain sequence may be combined with specified CDR sequences from the same antibody.

The antibodies of the present disclosure may comprise one or more substitutions within the framework of the VH and/or VL region. As used herein, a “substitution” refers to the exchange of one amino acid for another at a specific position, relative to the same position in a baseline molecule. In some embodiments, the baseline molecules are exemplified antibodies herein, for example ATL_5331; ATL 5334, or ATL 5335.

In some embodiments, antibodies of the disclosure comprise one or more VH framework substitutions at positions selected from the group: 72, 73, 76, 77, 78, 82A, 82B 83, 86 and 87 of the VH domain, according to Kabat numbering. In some embodiments, the substitutions are selected from the following VH domain substitutions: D72E, D73E, N76A, T77A, V78L, N82AA, S82BT, S87BA, N83K, D86E, and T87A. In embodiments, the VH framework substitutions are selected from: 78L, 83K, and 76A. In embodiments, the VH framework substitutions are: (i) 78L and 83K, or (ii) 76A, 78L, and 83K.

In some embodiments, the VL substitutions are at positions selected from the following positions: 8, 19, 36, 42, 46, 47, 60, 69, 75, 80, 104. In some embodiments, the substitutions are selected from R8A, I19V, F36Y, N42K, P46L, I47M, A60N, N69A, V751, P80A and V104L.

Sequences and properties of antibodies of the disclosure may be compared with a “reference antibody”. As used herein, a reference antibody is an antibody which binds the same target as the antibodies of the invention but differs in one or more physical property. For example, a reference antibody may differ in at least one amino acid residue in CDRH1, CDRH2, CDRH3, CDRL1, CDRL2, CDRL3, the VH framework, the VL framework, the heavy chain backbone, the light chain backbone, the Fc region and/or the hinge region, so long as they bind to the same target, preferably the same epitope, as the antibodies of the disclosure. Reference antibodies may be isotype matched to the antibodies of the disclosure. Reference antibodies may bind the same epitope, or block, sterically hinder or otherwise compete for the same epitope as the antibodies of the disclosure. Reference antibodies may be known in the art, or may possess the CDRs and/or variable domains of an antibody in the art whilst being otherwise identical to the antibodies of the disclosure. For example, ATL_5059 is a reference antibody (which does not have identical CDRs or framework regions to the antibodies of the disclosure), and is also disclosed as “NI-302.8F1” in U.S. Pat. No. 11,401,325 B2.

Preferred antibodies possess one or more residues that differs from a reference antibody capable of binding the same target, and have one or more improved property relative to said reference antibody. Differences may be in CDRs and/or framework residues of the variable domains. In some embodiments, the antibodies differ from a reference antibody in their CDRs, and can bind the same target, optionally at the same or a similar epitope. For example, antibodies according to the present invention may exhibit improved binding potency to exon 1 HTT, such as improved binding potency to WT HTT and/or improved binding potency to mHTT. It is non-trivial to identify what, if any, residues within an antibody may improve one or more properties, without the exercise of hindsight. This may be achieved by taking a “parent” reference antibody and painstakingly altering individual, or combinations of, residues and analysing the results. Alternatively, guided design such as analysis of variants within naturally arising antibody families so as to identify candidate substitutions, or phage display analysis of antibodies, may be performed and collated to produce improved antibodies. Candidate substitutions may be identified from multiple sources and combined for further testing to produce even more advantageous antibodies. The antibodies of the present disclosure were identified independently of any prior art antibody, through analysis of convergence of B cell repertoires of resilient individuals, or derived from such antibodies. Thus, the antibodies of the present disclosure advantageously are derived from naturally occurring protective antibodies, the sequence of which could not have been arrived based on any disclosure in the prior art. For example, it was only through the significant investigation and guided design described herein that the present inventors were able to produce antibodies ATL_5895, ATL_5901, and ATL_5667, derived from ATL_5331, ATL_5334, and ATL_5335, respectively.

In embodiments, the isolated antibody or antibody fragment thereof which specifically binds to Huntingtin (HTT) protein or a fragment thereof, comprises a heavy chain variable (VH) domain comprising CDRs HCDR1, HCDR2 and HCDR3, wherein: i. HCDR1 has amino acid sequence KAWMN (SEQ ID NO: 14), or an amino acid sequence comprising an amino acid substitution compared with KAWMN (SEQ ID NO:14), optionally wherein the substitution is at position 35, optionally wherein the substitution is N35S, wherein the position numbering is Kabat; ii. HCDR2 has amino acid sequence RIKSGIDGGTTDYAAPVQG (SEQ ID NO:15), or an amino acid sequence comprising one or two amino acid substitutions compared with RIKSGIDGGTTDYAAPVQG (SEQ ID NO: 15); optionally wherein the substitutions are at positions selected from: 64, 54 or 53, optionally wherein the substitutions are selected from: Q64K, D53E, and G54A, wherein the position numbering is Kabat; and iii. HCDR3 has amino acid sequence PPYYYYYGLDV (SEQ ID NO: 3), or a sequence comprising one, two, three or four substitutions compared with PPYYYYYGLDV (SEQ ID NO: 3), optionally wherein the substitution(s) are at positions selected from: 95, 97, 100A, 100B, 100C, optionally wherein the substitutions are selected from: Y97F, P95S, Y100AG, G100BL and L100C wherein the position numbering is Kabat.

The isolated antibody or antibody fragment thereof may further comprise a light chain variable (VL) domain comprising CDRs LCDR1, LCDR2 and LCDR3, wherein: iv. LCDR1 has an amino acid sequence selected from: TGTSSDVGSYNLVS (SEQ ID NO:4), TGTSSDVGGYKLVS (SEQ ID NO:9), and TGTSSDIGSYNLVS (SEQ ID NO:16); v. LCDR2 has an amino acid sequence selected from: EVNKRPS (SEQ ID NO:5), EVSKRPS (SEQ ID NO:10), and EGSKRPS (SEQ ID NO:17); vi. LCDR3 has amino acid sequence selected from: GSYAGTNNV (SEQ ID NO:21); an amino acid sequence comprising one, two or three amino acid substitutions compared with GSYAGTNNV (SEQ ID NO:21), optionally wherein the substitutions are selected from: 92, 95, 89, and 91, optionally wherein the substitution is selected from: A92G, N95A, G89V and Y91F; CSYAGSSVV (SEQ ID NO: 23); an amino acid sequence comprising one or two amino acid substitution compared with CSYAGSSVV (SEQ ID NO: 23), optionally wherein the amino acid substitution(s) are selected at positions selected from: 89, 95, optionally wherein the substitutions are selected from: C89S, N95A; SSYAGENTLV (SEQ ID NO: 25); and an amino acid sequence comprising one or two amino acid substitutions compared with SSYAGFNTLV (SEQ ID NO: 25), optionally wherein the amino acid substitution(s) are at positions N95 and/or T95, optionally wherein the substitutions are selected from N95S and/or T95A, wherein the position numbering is Kabat.

In a related embodiment, the antibody comprises: a heavy chain variable (VH) domain comprising CDRs HCDR1, HCDR2 and HCDR3, and optionally a light chain variable (VL) domain comprising CDRs LCDR1, LCDR2 and LCDR3, wherein the CDRs HCDR1, HCDR2, HCDR3, LCDR1, LCDR2 and LCDR3 are CDR's comprising between one and ten substitutions compared with the following CDRs:
i. HCDR1 with amino acid sequence KAWMN (SEQ ID NO: 14) or KAWMS (SEQ ID NO:1), or an amino acid sequence comprising one amino acid substitution compared with KAWMN, optionally wherein the sequence of HCDR1 comprises one substitution compared to KAWMN (SEQ ID NO:14) or KAWMS (SEQ ID NO:1);
ii. HCDR2 with amino acid sequence RIKSGIDGGTTDYAAPVQG (SEQ ID NO: 15), RIKSGIDAGTTDYAAPVKG (SEQ ID NO:2), RIKSGIDGGTTDYAAPVKG (SEQ ID NO: 27), or, optionally wherein the sequence of HCDR2 comprises one, two, three, four, five, or six, substitutions compared with RIKSGIDGGTTDYAAPVQG (SEQ ID NO:15), RIKSGIDAGTTDYAAPVKG (SEQ ID NO: 2), or RIKSGIDGGTTDYAAPVKG (SEQ ID NO: 27);
iii. HCDR3 with amino acid sequence PPYYYYYGLDV (SEQ ID NO: 3), optionally wherein the sequence of HCDR3 comprises one, two, three or four substitutions compared with PPYYYYYGLDV (SEQ ID NO: 3);
iv. LCDR1 with amino acid sequence TGTSSDVGSYNLVS (SEQ ID NO:4), TGTSSDVGGYKLVS (SEQ ID NO:9), or TGTSSDIGSYNLVS (SEQ ID NO:16), optionally wherein the sequence of LCDR1 comprises one or two substitutions compared with TGTSSDVGSYNLVS (SEQ ID NO:4), TGTSSDVGGYKLVS (SEQ ID NO:9), or TGTSSDIGSYNLVS (SEQ ID NO:16);
v. LCDR2 with amino acid sequence EVNKRPS (SEQ ID NO:5), EVSKRPS (SEQ ID NO: 10), or EGSKRPS (SEQ ID NO:17);
vi. LCDR3 with amino acid sequence: GSYAGTNNV (SEQ ID NO:21), GSYAGTANV (SEQ ID NO:6), CSYAGSSVV (SEQ ID NO: 23), SSYAGSSVV (SEQ ID NO:11), SSYAGFSTLV (SEQ ID NO:18), or SSYAGFNTLV (SEQ ID NO: 25), optionally wherein the sequence of LCDR3 comprises one, two or three substitutions compared to GSYAGTNNV (SEQ ID NO:21), GSYAGTANV (SEQ ID NO:6), CSYAGSSVV (SEQ ID NO: 23), SSYAGSSVV (SEQ ID NO:11), SSYAGFSTLV (SEQ ID NO:18), or SSYAGFNTLV (SEQ ID NO: 25).

In embodiments, the antibody comprises: a heavy chain variable (VH) domain comprising CDRs HCDR1, HCD2 and HCDR3, wherein: i. HCDR1 has amino acid sequence KAWMS (SEQ ID NO:1); ii. HCDR2 has amino acid sequence RIKSGIDAGTTDYAAPVKG (SEQ ID NO:2); and iii. HCDR3 has amino acid sequence PPYYYYYGLDV (SEQ ID NO:3). In some such embodiments, the antibody comprises a light chain variable (VL) domain comprising CDRs LCDR1, LCDR2 and LCDR3, wherein: i. LCDR1 has amino acid sequence TGTSSDVGSYNLVS (SEQ ID NO:4); ii. LCDR2 has amino acid sequence EVNKRPS (SEQ ID NO:5); iii. LCDR3 has amino acid sequence GSYAGTANV (SEQ ID NO: 6). In other embodiments, the antibody comprises a light chain variable (VL) domain comprising CDRs LCDR1, LCDR2 and LCDR3, wherein: i. LCDR1 has amino acid sequence TGTSSDVGGYKLVS (SEQ ID NO:9); ii. LCDR2 has amino acid sequence EVSKRPS (SEQ ID NO: 10); and iii. LCDR3 has amino acid sequence SSYAGSSVV (SEQ ID NO:11).

In embodiments, the antibody comprises: a heavy chain variable (VH) comprising CDRs HCDR1, HCD2 and HCDR3, wherein: i. HCDR1 has amino acid sequence KAWMN (SEQ ID NO:14); ii. HCDR2 has amino acid sequence RIKSGIDGGTTDYAAPVQG (SEQ ID NO: 15); iii. HCDR3 has amino acid sequence PPYYYYYGLDV (SEQ ID NO:3). In some such embodiments, the antibody comprises a light chain variable (VL) domain comprising CDRs LCDR1, LCDR2 and LCDR3, wherein: i. LCDR1 has amino acid sequence TGTSSDVGSYNLVS (SEQ ID NO:4); ii. LCDR2 has amino acid sequence EVNKRPS (SEQ ID NO:5); and iii. LCDR3 comprising amino acid sequence GSYAGTNNV (SEQ ID NO: 21). In other embodiments, the antibody comprises a light chain variable (VL) domain comprising CDRs LCDR1, LCDR2 and LCDR3, wherein: i. LCDR1 has amino acid sequence TGTSSDVGGYKLVS (SEQ ID NO:9); ii. LCDR2 has amino acid sequence EVSKRPS (SEQ ID NO: 10); and iii. LCDR3 has amino acid sequence CSYAGSSVV (SEQ ID NO: 23). In other embodiments, the antibody comprises a light chain variable (VL) domain comprising CDRs LCDR1, LCDR2 and LCDR3, wherein: i. LCDR1 has amino acid sequence TGTSSDIGSYNLVS (SEQ ID NO: 16); ii. LCDR2 has amino acid sequence EGSKRPS (SEQ ID NO: 17); and iii. LCDR3 has amino acid sequence SSYAGFNTLV (SEQ ID NO: 25). In other embodiments, the antibody comprises a light chain variable (VL) domain comprising CDRs LCDR1, LCDR2 and LCDR3, wherein: i. LCDR1 has amino acid sequence TGTSSDIGSYNLVS (SEQ ID NO:16); ii. LCDR2 has amino acid sequence EGSKRPS (SEQ ID NO:17); and iii. LCDR3 has amino acid sequence SSYAGFSTLV (SEQ ID NO:18).

In embodiments, the antibody comprises: a heavy chain variable (VH) domain comprising CDRs HCDR1, HCD2 and HCDR3, wherein: i. HCDR1 has amino acid sequence KAWMS (SEQ ID NO:1); ii. HCDR2 has amino acid sequence RIKSGIDGGTTDYAAPVKG (SEQ ID NO: 27); iii. HCDR3 has amino acid sequence PPYYYYYGLDV (SEQ ID NO:3). In some such embodiments, the antibody comprises a light chain variable (VL) domain comprising CDRs LCDR1, LCDR2 and LCDR3, wherein: i. LCDR1 has amino acid sequence TGTSSDVGSYNLVS (SEQ ID NO:4); ii. LCDR2 has amino acid sequence EVNKRPS (SEQ ID NO:5); iii. LCDR3 has amino acid sequence GSYAGTNNV (SEQ ID NO: 21). In other embodiments, the antibody comprises a light chain variable (VL) domain comprising CDRs LCDR1, LCDR2 and LCDR3, wherein: i. LCDR1 has amino acid sequence TGTSSDVGGYKLVS (SEQ ID NO:9); ii. LCDR2 has amino acid sequence EVSKRPS (SEQ ID NO: 10); iii. LCDR3 has amino acid sequence CSYAGSSVV (SEQ ID NO: 23). In other embodiments, the antibody comprises a light chain variable (VL) domain comprising CDRs LCDR1, LCDR2 and LCDR3, wherein: i. LCDR1 has amino acid sequence TGTSSDIGSYNLVS (SEQ ID NO: 16); ii. LCDR2 has amino acid sequence EGSKRPS (SEQ ID NO:17); iii. LCDR3 has amino acid sequence SSYAGFNTLV (SEQ ID NO: 25).

In embodiments, the antibody comprises: a heavy chain variable (VH) domain comprising CDRs HCDR1, HCD2 and HCDR3, wherein: i. HCDR1 has amino acid sequence KAWMS (SEQ ID NO:1); ii. HCDR2 has amino acid sequence RIKSGIDGGTTDYAAPVKG (SEQ ID NO: 27); iii. HCDR3 has amino acid sequence PPYYYYYGLDV (SEQ ID NO:3), or SPYYYYYGLDV (SEQ ID NO: 157), or PPFYYYYGLDV (SEQ ID NO: 158), or PPYYYYGLNV (SEQ ID NO: 159). In some such embodiments, the antibody comprises a light chain variable (VL) domain comprising CDRs LCDR1, LCDR2 and LCDR3, wherein: i. LCDR1 has amino acid sequence TGTSSDVGSYNLVS (SEQ ID NO:4), or TGTSSDVGGYKLVS (SEQ ID NO:9); ii. LCDR2 has amino acid sequence EVNKRPS (SEQ ID NO:5); or EVSKRPS (SEQ ID NO: 10); iii. LCDR3 has amino acid sequence SSYAGSSVV (SEQ ID NO: 11), or VSFAGTANV (SEQ ID NO: 160), or VSYAGTANV (SEQ ID NO: 161), or VSYGGTENV (SEQ ID NO: 162). In other such embodiments, the antibody comprises a light chain variable (VL) domain comprising CDRs LCDR1, LCDR2 and LCDR3, wherein: i. LCDR1 has amino acid sequence TGTSSDVGGYKLVS (SEQ ID NO:9); ii. LCDR2 has amino acid sequence EVSKRPS (SEQ ID NO: 10); iii. LCDR3 has amino acid sequence CSYAGSSVV (SEQ ID NO: 23). In other such embodiments, the antibody comprises a light chain variable (VL) domain comprising CDRs LCDR1, LCDR2 and LCDR3, wherein: i. LCDR1 has amino acid sequence TGTSSDIGSYNLVS (SEQ ID NO: 16); ii. LCDR2 has amino acid sequence EGSKRPS (SEQ ID NO: 17); iii. LCDR3 has amino acid sequence SSYAGFNTLV (SEQ ID NO: 25).

The VH domain may be a human VH domain. The antibody or fragment thereof may have a VH domain framework sequence selected from: (a) the framework sequence of [ATL_0005331 VH]: EVOLVESGGGLVKPGGSLRLSCAASGFTFN (SEQ ID NO: 98)-CDRH1-WVRQAPGKGLEWVG (SEQ ID NO: 99)-CDRH2-RFTISRDDSKNTVYLQMNSLNTEDTAVYYCIP (SEQ ID NO: 100)-CDRH3-WGQGTTVTVSS (SEQ ID NO: 101), (b) the framework sequence of [ATL_0005334 VH]: EVQLVESGGGLVKPGGSLRLSCAASGFTFN (SEQ ID NO: 98)-CDRH1-WVRQAPGKGLEWVG (SEQ ID NO: 99)-CDRH2-RFTISRDDSKNTVYLQMNSLNTEDTAVYYCIP (SEQ ID NO: 100)-CDRH3-WGQGTTVTVSS (SEQ ID NO: 101), and (c) the framework sequence of [ATL_0005335 VH]: EVOLVESGGGLVKPGGSLRLSCAASGFTFN (SEQ ID NO: 98)-CDRH1-WVRQAPGKGLEWVG (SEQ ID NO: 99)-CDRH2-RFTISRDDSKNTVYLQMNSLNTEDTAVYYCIP (SEQ ID NO: 100)-CDRH3-WGQGTTVTVSS (SEQ ID NO: 101). The antibody or fragment thereof may have a VH domain framework sequence selected from: (a) the framework sequence of ATL_0006199 VH: EVOLVESGGGLVKPGGSLRLSCAASGFTFN (SEQ ID NO: 98)-[CDRH1]-WVRQAPGKGLEWVG (SEQ ID NO: 99)-[CDRH2]-RFTISRDDSKNTLYLQMNSLKTEDTAVYWCSP (SEQ ID NO: 169)-[CDRH3]-WGQGTTVTVSS (SEQ ID NO: 101); (b) the framework sequence of ATL_0006200 VH (and ATL_0006374): EVOLVESGGGLVKPGGSLRLSCAASGFTFN (SEQ ID NO: 98)-[CDRH1]-WVRQAPGKGLEWVG (SEQ ID NO: 99)-[CDRH2]-RFTISRDDSKNTLYLQMNSLKTEDTAVYYCVP (SEQ ID NO: 170)-[CDRH3]-WGQGTTVTVSS (SEQ ID NO: 101); (c) the framework sequence of ATL_0006202 VH: EVOLVESGGGLVKPGGSLRLSCAASGFTFN (SEQ ID NO: 98)-[CDRH1]-WVRQAPGKGLEWVG (SEQ ID NO: 99)-[CDRH2]-RFTISRDDSKNTLYLQMNSLKTEDTAVYYCSP (SEQ ID NO: 171)-[CDRH3]-WGQGTTVTVSS (SEQ ID NO: 101); (d) the framework sequence of ATL_0006203 VH: EVOLVESGGGLVKPGGSLRLSCAASGFTFN (SEQ ID NO: 98)-[CDRH1]-WVRQAPGKGLEWVG (SEQ ID NO: 99)-[CDRH2]-RFTISRDDSKNTLYLQMNSLKTEDTAVYYCIP (SEQ ID NO: 172)-[CDRH3]-WGQGTTVTVSS (SEQ ID NO: 101); (e) the framework sequence of ATL_0006205 VH (and ATL_0006375, ATL_0006376, ATL_0006377, and ATL_0006378): EVOLVESGGGLVKPGGSLRLSCAASGFTFN (SEQ ID NO: 98)-[CDRH1]-WVRQAPGKGLEWVG (SEQ ID NO: 99)-[CDRH2]-RFTISRDDSKNTLYLQMNSLKTEDTAVYWCVP (SEQ ID NO: 173)-[CDRH3]-WGQGTTVTVSS (SEQ ID NO: 101). The isolated antibody or fragment thereof may comprise one or more framework substitutions in the VH domain (e.g. compared to the above framework sequences). The one or more framework substitutions may be at positions selected from: 72, 73, 76, 77, 78, 82A, 82B, 83, 86 and 87. The one or more framework substitutions may be at positions selected from: 72, 73, 76, 77, 78, 82A, 82B, 83, 86, 87, 91, and 93. The one or more framework substitutions in the VH domain may be selected from the group consisting of: at position 72: E (e.g. D72E), at position 73: E (e.g. D73E), at position 76: A (e.g.N76A), at position 77: A (e.g. T77A), at position 78: L (e.g. V78L), at position 82A: A (e.g. N82AA), at position 82B: T (e.g. S82BT), at position 82B: A (e.g. S87BA), at position 83: K (e.g. N83K), at position 86: E (e.g. D86E), at position 87: A (e.g. T87A), at position 91: W (Y91W), and at position 93: M, S or V (I93M, I93S, I93V); wherein position numbering is Kabat. Such mutations in the framework regions may advantageously remove liabilities, improving the stability and reducing the immunogenicity of the resulting antibody. For example, mutations D72E and/or D73E may reduce the risk of isomerisation, N76A and/or T77A may reduce the risk of deamination, D86E and/or T87A may reduce the risk of isomerisation, mutations N82AA, S82BT, N83K and/or S87BA may reduce the risk of deamination. In embodiments, the framework substitutions are selected from: 78L, 83K, and 76A, optionally wherein the framework substitutions are: (i) 78L and 83K, or (ii) 76A, 78L, and 83K.

In embodiments, the VL domain is a human VL domain. In embodiments, the antibody or fragment thereof has a VL domain framework sequence selected from: (a) the framework sequence of [ATL_0005331 VL]: QSALTQPRSVSGSPGQSITISC (SEQ ID NO: 102)-CDRL1-WFQQHPGKAPKLIIY (SEQ ID NO: 103)-CDRL2-GVPDRFSGSKSGNTASLTVSGLQAEDEADYYC (SEQ ID NO: 104)-CDRL3-FGTGTKLTVL (SEQ ID NO: 105); (b) the framework sequence of [ATL_0005334 VL]: QSALTQPASVSGSPGQSITISC ID (SEQ NO: 106)-CDRL1-WYQQHPGRAPKLMIY (SEQ ID NO: 107)-CDRL2-GVSSRFSGSKSGSTASLTISGLQAEDEADYYC (SEQ ID NO: 108)-CDRL3-FGGGTKLTVL (SEQ ID NO: 109); and (c) the framework sequence of [ATL_0005335 VL]: QSALTQPASVSGSPGQSITISC (SEQ ID NO: 106)-CDRL1-WYQQHPGNAPKPLIY (SEQ ID NO: 110)-CDRL2-GVSARFSGSKSGNTASLTISGLQPEDEADYYC (SEQ ID NO: 111)-CDRL3-FGGGTKVTVL (SEQ ID NO: 112). In embodiments, the isolated antibody or fragment thereof comprises one or more framework substitutions in the VL domain (e.g. compared to the above framework sequences). The one or more framework mutations may be at positions selected from: 8, 19, 36, 42, 46, 47, 60, 69, 75, 80, 104. The one or more framework substitutions in the VL domain may be selected from the group consisting of: at position 8: R (e.g. R8A), at position 19: V (e.g. I19V), at position 36: Y (e.g. F36Y), at position 42: K (e.g. N42K), at position 46: L (e.g. P46L), at position 47: M (e.g. I47M or L47M), at position 60: N (e.g. A60N), at position 69: A (e.g. N69A), at position 75: I (e.g. V751), at position 80: A (e.g. P80A), and at position 104: V or L (e.g. L104V or V104L), wherein the position numbering is Kabat. The one or more framework mutations may be at positions selected from: (i) in antibody ATL_0005334, an antibody derived therefrom such as ATL_0005586, ATL_0005900, ATL_0005556, ATL_0005901, or an antibody that comprises the VL sequence of any of ATL_0005586, ATL_0005900, ATL_0005901, ATL_0005556 (e.g. SED ID Nos: 13, 24): 8 (optionally wherein the substituted amino acid is R); (ii) in antibody ATL_0005331 or an antibody derived therefrom such as ATL_0005577, ATL_0005891, ATL_0005890, ATL_0005559, ATL_0005563, ATL_0005896, ATL_0005894, ATL_0005895, ATL_0005555 or an antibody that comprises the VL sequence of any of ATL_0005577, ATL_0005891, ATL_0005890, ATL_0005559, ATL_0005563, ATL_0005896, ATL_0005894, ATL_0005895, ATL_0005555 (e.g. SEQ ID Nos: 22, 32, 29, 40, 41, 8): 19 (optionally wherein the substituted amino acid is V), 36 (optionally wherein the substituted amino acid is Y), 47 (optionally wherein the substituted amino acid is M), 69 (optionally wherein the substituted amino acid is A), 75 (optionally wherein the substituted amino acid is I), 104 (optionally wherein the substituted amino acid is V); (iii) in antibody ATL_0005335 or an antibody derived therefrom such as ATL_0005572, ATL_0005567, ATL_0005557 or an antibody that comprises the VL sequence of any of ATL_0005572, ATL_0005567, ATL_0005557 (e.g. SEQ ID Nos: 30, 26, 20, 39): 42 (optionally wherein the substituted amino acid is R), 46 (optionally wherein the substituted amino acid is K), 47 (optionally wherein the substituted amino acid is M), 60 (optionally wherein the substituted amino acid is N), 69 (optionally wherein the substituted amino acid is A), 80 (optionally wherein the substituted amino acid is A), 104 (optionally wherein the substituted amino acid is L). Such mutations in the framework regions may advantageously remove liabilities, improving the stability and reducing the immunogenicity of the resulting antibody. For example, substitution N69A (or 70A) may reduce the risk of deamination.

In embodiments, HCDR1, HCDR2 and HCDR3 of the VH domain are within a germline framework. In embodiments, LCDR1, LCDR2, LCDR3 of the VL domain are within a germline framework.

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCIPPPYYYYYGLDVWGQGTTVTVSS (SEQ ID NO: 7) (ATL_5895 VH). In some such embodiments, the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCGSYAGTANVFGTGTKVTVL (SEQ ID NO:8) (ATL_5895 VL).

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKATLYLQMNSLKTEDTAVYYCIPPPYYYYYGLDVWGQGTTVTVSS (SEQ ID NO: 12) (ATL_5901 VH). In some such embodiments, the light chain variable domain comprises amino acid sequence QSALTQPASVSGSPGQSITISCTGTSSDVGGYKLVSWYQQHPGRAPKLMIYEVSKRPSGVSSRFSG SKSGSTASLTISGLQAEDEADYYCSSYAGSSVVFGGGTKLTVL (SEQ ID NO:13) (ATL_5901 VL). In embodiments, the heavy chain variable domain comprises amino acid sequence EVOLVESGGGLVKPGGSLRLSCAASGFTFNKAWMNWVRQAPGKGLEWVGRIKSGIDGGTTDYAA PVQGRFTISRDDSKNTVYLQMNSLNTEDTAVYYCIPPPYYYYYGLDVWGQGTTVTVSS (SEQ ID NO: 19) (ATL_5567 VH). In some such embodiments, the light chain variable domain comprises amino acid sequence QSALTQPASVSGSPGQSITISCTGTSSDIGSYNLVSWYQQHPGNAPKPLIYEGSKRPSGVSARFSGS KSGNTASLTISGLQPEDEADYYCSSYAGFSTLVFGGGTKVTVL (SEQ ID NO:20) (ATL_5567 VL). EVOLVESGGGLVKPGGSLRLSCAASGFTFNKAWMNWVRQAPGKGLEWVGRIKSGIDGGTTDYAA PVQGRFTISRDDSKNTVYLQMNSLNTEDTAVYYCIPPPYYYYYGLDVWGQGTTVTVSS (SEQ ID NO: 19) (ATL_5331 VH). In some such embodiments, the light chain variable domain comprises amino acid sequence

(ATL_5331 VL)
(SEQ ID NO: 22)
QSALTQPRSVSGSPGQSITISCTGTSSDVGSYNLVSWFQQHPGKAPKLII
YEVNKRPSGVPDRFSGSKSGNTASLTVSGLQAEDEADYYCGSYAGTNNVF
GTGTKLTVL.

In embodiments, the heavy chain variable domain comprises amino acid sequence EVOLVESGGGLVKPGGSLRLSCAASGFTFNKAWMNWVRQAPGKGLEWVGRIKSGIDGGTTDYAA PVQGRFTISRDDSKNTVYLQMNSLNTEDTAVYYCIPPPYYYYYGLDVWGQGTTVTVSS (SEQ ID NO: 19) (ATL_5334 VH). In some such embodiments, the light chain variable domain sequence comprises amino acid sequence

(ATL_5334 VL)
(SEQ ID NO: 24)
QSALTQPASVSGSPGQSITISCTGTSSDVGGYKLVSWYQQHPGRAPKLMI
YEVSKRPSGVSSRFSGSKSGSTASLTISGLQAEDEADYYCCSYAGSSVVF
GGGTKLTVL.

In embodiments, the heavy chain variable domain comprises amino acid sequence EVOLVESGGGLVKPGGSLRLSCAASGFTFNKAWMNWVRQAPGKGLEWVGRIKSGIDGGTTDYAA PVQGRFTISRDDSKNTVYLQMNSLNTEDTAVYYCIPPPYYYYYGLDVWGQGTTVTVSS (SEQ ID NO: 19) (ATL_5335 VH). In some such embodiments, the light chain variable domain sequence

(ATL_5335 VL)
(SEQ ID NO: 26)
QSALTQPASVSGSPGQSITISCTGTSSDIGSYNLVSWYQQHPGNAPKPLI
YEGSKRPSGVSARFSGSKSGNTASLTISGLQPEDEADYYCSSYAGFNTLV
FGGGTKVTVL.

In embodiments, the heavy chain variable domain comprises amino acid sequence EVOLVESGGGLVKPGGSLRLSCAASGFTFNKAWMSWVRQAPGKGLEWVGRIKSGIDGGTTDYAA PVKGRFTISRDDSKNTLYLQMNSLKTEDTAVYYCIPPPYYYYYGLDVWGQGTTVTVSS (SEQ ID NO: 28) (ATL_5555 VH). In some such embodiments, the light chain variable domain sequence

(ATL_5555 VL)
(SEQ ID NO: 29)
QSALTQPRSVSGSPGQSVTISCTGTSSDVGSYNLVSWYQQHPGKAPKLMI
YEVNKRPSGVPDRFSGSKSGNTASLTISGLQAEDEADYYCGSYAGTNNVF
GTGTKVTVL.

In embodiments, the heavy chain variable domain comprises amino acid sequence EVOLVESGGGLVKPGGSLRLSCAASGFTFNKAWMSWVRQAPGKGLEWVGRIKSGIDGGTTDYAA PVKGRFTISRDDSKNTLYLQMNSLKTEDTAVYYCIPPPYYYYYGLDVWGQGTTVTVSS (SEQ ID NO: 28) (ATL_5556 VH). In some such embodiments, the light chain variable domain comprises amino acid sequence

(ATL_5556 VL)
(SEQ ID NO: 24)
QSALTQPASVSGSPGQSITISCTGTSSDVGGYKLVSWYQQHPGRAPKLMI
YEVSKRPSGVSSRFSGSKSGSTASLTISGLQAEDEADYYCCSYAGSSVVF
GGGTKLTVL.

In embodiments, the heavy chain variable domain comprises amino acid sequence EVOLVESGGGLVKPGGSLRLSCAASGFTFNKAWMSWVRQAPGKGLEWVGRIKSGIDGGTTDYAA PVKGRFTISRDDSKNTLYLQMNSLKTEDTAVYYCIPPPYYYYYGLDVWGQGTTVTVSS (SEQ ID NO: 28) (ATL_5557 VH). In some such embodiments, the light chain variable domain comprises amino acid sequence

(ATL_5557 VL)
(SEQ ID NO: 30)
QSALTQPASVSGSPGQSITISCTGTSSDIGSYNLVSWYQQHPGKAPKLMI
YEGSKRPSGVSNRFSGSKSGNTASLTISGLQAEDEADYYCSSYAGFNTLV
FGGGTKLTVL.

In embodiments, the heavy chain variable domain comprises amino acid sequence EVOLVESGGGLVKPGGSLRLSCAASGFTFNKAWMNWVRQAPGKGLEWVGRIKSGIDAGTTDYAAP VQGRFTISRDDSKNTVYLQMNSLNTEDTAVYYCIPPPYYYYYGLDVWGQGTTVTVSS (SEQ ID NO: 31) (ATL_5577 VH). In some such embodiments, the light chain variable domain comprises amino acid sequence QSALTQPRSVSGSPGQSITISCTGTSSDVGSYNLVSWFQQHPGKAPKLIIYEVNKRPSGVPDRFSGS KSGNTASLTVSGLQAEDEADYYCGSYAGTNNVFGTGTKLTVL (SEQ ID NO: 22) (ATL_5577 VL). In embodiments, the heavy chain variable domain comprises amino acid sequence EVOLVESGGGLVKPGGSLRLSCAASGFTFNKAWMNWVRQAPGKGLEWVGRIKSGIDAGTTDYAAP VQGRFTISRDDSKNTVYLQMNSLNTEDTAVYYCIPPPYYYYYGLDVWGQGTTVTVSS (SEQ ID NO: 31) (ATL_5891 VH). In some such embodiments, the light chain variable domain comprises amino acid sequence

(ATL_5891 VL)
(SEQ ID NO: 32)
QSALTQPRSVSGSPGQSVTISCTGTSSDVGSYNLVSWYQQHPGKAPKLII
YEVNKRPSGVPDRFSGSKSGNTASLTISGLQAEDEADYYCGSYAGTNNVF
GTGTKVTVL.

In embodiments, the heavy chain variable domain comprises amino acid sequence EVOLVESGGGLVKPGGSLRLSCAASGFTFNKAWMNWVRQAPGKGLEWVGRIKSGIDAGTTDYAAP VQGRFTISRDDSKNTVYLQMNSLNTEDTAVYYCIPPPYYYYYGLDVWGQGTTVTVSS (SEQ ID NO: 31) (ATL_5586 VH). In some such embodiments, the light chain variable domain comprises amino acid sequence

(ATL_5586 VL)
(SEQ ID NO: 13)
QSALTQPASVSGSPGQSITISCTGTSSDVGGYKLVSWYQQHPGRAPKLMI
YEVSKRPSGVSSRFSGSKSGSTASLTISGLQAEDEADYYCSSYAGSSVVF
GGGTKLTVL.

In embodiments, the heavy chain variable domain comprises amino acid sequence EVOLVESGGGLVKPGGSLRLSCAASGFTFNKAWMNWVRQAPGKGLEWVGRIKSGIDAGTTDYAAP VQGRFTISRDDSKNTVYLQMNSLNTEDTAVYYCIPPPYYYYYGLDVWGQGTTVTVSS (SEQ ID NO: 31) (ATL_5890 VH). In some such embodiments, the light chain variable domain comprises amino acid sequence

(ATL_5890 VL)
(SEQ ID NO: 29)
QSALTQPRSVSGSPGQSVTISCTGTSSDVGSYNLVSWYQQHPGKAPKLMI
YEVNKRPSGVPDRFSGSKSGNTASLTISGLQAEDEADYYCGSYAGTNNVF
GTGTKVTVL.

In embodiments, the heavy chain variable domain comprises amino acid sequence EVOLVESGGGLVKPGGSLRLSCAASGFTFNKAWMNWVRQAPGKGLEWVGRIKSGIDGGTTDYAA PVQGRFTISRDDSKNTVYLQMNSLNTEDTAVYYCIPPPYYYYYGLDVWGQGTTVTVSS (SEQ ID NO: 19) (ATL_5559 VH). In some such embodiments, light chain variable domain comprises amino acid sequence QSALTQPRSVSGSPGQSITISCTGTSSDVGSYNLVSWFQQHPGKAPKLIIYEVNKRPSGVPDRFSGS KSGATASLTVSGLQAEDEADYYCGSYAGTNNVFGTGTKLTVL (SEQ ID NO: 42) (ATL_5559 VL), In embodiments, the heavy chain variable domain comprises amino acid sequence EVOLVESGGGLVKPGGSLRLSCAASGFTFNKAWMNWVRQAPGKGLEWVGRIKSGIDGGTTDYAA PVQGRFTISRDDSKNTVYLQMNSLNTEDTAVYYCIPPPYYYYYGLDVWGQGTTVTVSS (SEQ ID NO: 19) (ATL_5572 VH). In some such embodiments, the light chain variable domain comprises amino acid sequence QSALTQPASVSGSPGQSITISCTGTSSDIGSYNLVSWYQQHPGKAPKPLIYEGSKRPSGVSNRFSGS KSGNTASLTISGLQAEDEADYYCSSYAGFNTLVFGGGTKLTVL (SEQ ID NO: 39) (ATL_5572VL), or

(ATL_5563 VH)
(SEQ ID NO: 19)
EVQLVESGGGLVKPGGSLRLSCAASGFTFNKAWMNWVRQAPGKGLEWVGR
IKSGIDGGTTDYAAPVQGRFTISRDDSKNTVYLQMNSLNTEDTAVYYCIP
PPYYYYYGLDVWGQGTTVTVSS.

In embodiments, the light chain variable domain comprises amino acid sequence

(ATL_5563 VL)
(SEQ ID NO: 32)
QSALTQPRSVSGSPGQSVTISCTGTSSDVGSYNLVSWYQQHPGKAPKLII
YEVNKRPSGVPDRFSGSKSGNTASLTISGLQAEDEADYYCGSYAGTNNVF
GTGTKVTVL.

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCIPPPYYYYYGLDVWGQGTTVTVSS (SEQ ID NO: 7) (ATL_5896 VH). In some such embodiments, the light chain variable domain comprises amino acid sequence

(SEQ ID NO: 40)
QSALTQPRSVSGSPGQSVTISCTGTSSDVGSYNLVSWYQQHPGKA
PKLMIYEVNKRPSGVPDRFSGSKSGATASLTISGLQAEDEADYYC
GSYAGTNNVFGTGTKVTVL
(ATL_5896 VL).

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCIPPPYYYYYGLDVWGQGTTVTVSS (SEQ ID NO: 7) (ATL_5894 VH). In some such embodiments, the light chain variable domain comprises amino acid sequence

(SEQ ID NO: 41)
QSALTQPRSVSGSPGQSVTISCTGTSSDVGSYNLVSWYQQHPGKA
PKLIIYEVNKRPSGVPDRFSGSKSGATASLTISGLQAEDEADYYC
GSYAGTANVFGTGTKVTVL
(ATL_5894 VL).

In embodiments, the heavy chain variable domain comprises amino acid sequence EVOLVESGGGLVKPGGSLRLSCAASGFTFNKAWMSWVRQAPGKGLEWVGRIKSGIDGGTTDYAA PVKGRFTISRDDSKNTLYLQMNSLKTEDTAVYYCIPPPYYYYYGLDVWGQGTTVTVSS (SEQ ID NO: 28) (ATL_5900 VH). In some such embodiments, the light chain variable domain comprises amino acid sequence

(SEQ ID NO: 13)
QSALTQPASVSGSPGQSITISCTGTSSDVGGYKLVSWYQQHPGRA
PKLMIYEVSKRPSGVSSRFSGSKSGSTASLTISGLQAEDEADYYC
SSYAGSSVVFGGGTKLTVL
(ATL_5900 VL).

In embodiments, the heavy chain variable domain comprises amino acid sequence EVOLVESGGGLVKPGGSLRLSCAASGFTFNKAWMNWVRQAPGKGLEWVGRIKSGIDAGTTDYAAP VQGRFTISRDDSKNTVYLQMNSLNTEDTAVYYCIPPPYYYYYGLDVWGQGTTVTVSS (SEQ ID NO: 31) (ATL_5891 VH). In some such embodiments, the light chain variable domain comprises amino acid sequence

(SEQ ID NO: 32)
QSALTQPRSVSGSPGQSVTISCTGTSSDVGSYNLVSWYQQHPGKA
PKLIIYEVNKRPSGVPDRFSGSKSGNTASLTISGLQAEDEADYYC
GSYAGTNNVFGTGTKVTVL
(ATL_5891 VL).

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYWCSPPPYYYYYGLDVWGQGTTVTVSS (SEQ ID NO: 144) (ATL_6199 VH). In some such embodiments, the light chain variable domain comprises amino acid sequence

(SEQ ID NO: 8)
QSALTQPRSVSGSPGQSVTISCTGTSSDVGSYNLVSWYQQHPGKA
PKLMIYEVNKRPSGVPDRFSGSKSGATASLTISGLQAEDEADYYC
GSYAGTANVFGTGTKVTVL
(ATL_6199 VL).

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCVPPPYYYYYGLDVWGQGTTVTVSS (SEQ ID NO: 145) (ATL_6200 VH). In some such embodiments, the light chain variable domain comprises amino acid sequence

(SEQ ID NO: 8)
QSALTQPRSVSGSPGQSVTISCTGTSSDVGSYNLVSWYQQHPGKA
PKLMIYEVNKRPSGVPDRFSGSKSGATASLTISGLQAEDEADYYC
GSYAGTANVFGTGTKVTVL
(ATL_6002 VL).

In embodiments, the heavy chain variable domain comprises amino acid sequence EVQLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCSPPPFYYYYGLDVWGQGTTVTVSS (SEQ ID NO: 146) (ATL_6202 VH). In some such embodiments, the light chain variable domain comprises amino acid sequence

(SEQ ID NO: 153)
QSALTQPRSVSGSPGQSVTISCTGTSSDVGSYNLVSWYQQHPGKA
PKLMIYEVNKRPSGVPDRFSGSKSGATASLTISGLQAEDEADYYC
VSYGGTENVFGTGTKVTVL
(ATL_6202 VL).

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCIPSPYYYYYGLDVWGQGTTVTVSS (SEQ ID NO: 147) (ATL_6203 VH). In some such embodiments, the light chain variable domain comprises amino acid sequence

(SEQ ID NO: 154)
QSALTQPRSVSGSPGQSVTISCTGTSSDVGSYNLVSWYQQHPGKA
PKLMIYEVNKRPSGVPDRFSGSKSGATASLTISGLQAEDEADYYC
VSYAGTANVFGTGTKVTVL
(ATL_6203 VL).

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCIPPPYYYYYGLDVWGQGTTVTVSS (SEQ ID NO: 7) (ATL_6204 VH). In some such embodiments, the light chain variable domain comprises amino acid sequence

(SEQ ID NO: 155)
QSALTQPRSVSGSPGQSVTISCTGTSSDVGSYNLVSWYQQHPGKA
PKLMIYEVNKRPSGVPDRFSGSKSGATASLTISGLQAEDEADYYC
VSFAGTANVFGTGTKVTVL
(ATL_6204 VL).

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYWCVPPPYYYYYGLDVWGQGTTVTVSS (SEQ ID NO: 148) (ATL_6205 VH). In some such embodiments, the light chain variable domain comprises amino acid sequence

(SEQ ID NO: 156)
QSALTQPRSVSGSPGQSVTISCTGTSSDVGSYNLVSWYQQHPGKA
PKLMIYEVNKRPSGVPDRFSGSKSGATASLTISGLQAEDEADYYC
VSYAGTANVFGTGTKVTVL
(ATL_6205 VL).

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCTPPPYYYYGLDVWGQGTTVTVSS (SEQ ID NO: 149) (ATL_6183 VH). In some such embodiments, the light chain variable domain comprises amino acid sequence

(SEQ ID NO: 8)
QSALTQPRSVSGSPGQSVTISCTGTSSDVGSYNLVSWYQQHPGKA
PKLMIYEVNKRPSGVPDRFSGSKSGATASLTISGLQAEDEADYYC
GSYAGTANVFGTGTKVTVL
(ATL_6183 VL).

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKATLYLQMNSLKTEDTAVYYCTPPPYYYYGLDVWGQGTTVTVSS (SEQ ID NO: 150) (ATL_6184 VH). In some such embodiments, the light chain variable domain comprises amino acid sequence

(SEQ ID NO: 13)
QSALTQPASVSGSPGQSITISCTGTSSDVGGYKLVSWYQQHPGRA
PKLMIYEVSKRPSGVSSRFSGSKSGSTASLTISGLQAEDEADYYC
SSYAGSSVVFGGGTKLTVL
(ATL_6184 VL).

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCTPPPYYYYGLNVWGQGTTVTVSS (SEQ ID NO: 151) (ATL_6185 VH). In some such embodiments, the light chain variable domain comprises amino acid sequence

(SEQ ID NO: 8)
QSALTQPRSVSGSPGQSVTISCTGTSSDVGSYNLVSWYQQHPGKA
PKLMIYEVNKRPSGVPDRFSGSKSGATASLTISGLQAEDEADYYC
GSYAGTANVFGTGTKVTVL
(ATL_6185 VL).

In embodiments, the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKATLYLQMNSLKTEDTAVYYCTPPPYYYYGLNVWGQGTTVTVSS (SEQ ID NO: 152) (ATL_6186 VH). In some such embodiments, the light chain variable domain comprises amino acid sequence

(SEQ ID NO: 13)
QSALTQPASVSGSPGQSITISCTGTSSDVGGYKLVSWYQQHPGRA
PKLMIYEVSKRPSGVSSRFSGSKSGSTASLTISGLQAEDEADYYC
SSYAGSSVVFGGGTKLTVL
(ATL_6186 VL).

In embodiments, heavy chain variable domain comprises a variable domain comprising an amino acid sequence that has at least 91% sequence identity to any one of the heavy chain variable domains above (e.g. SEQ ID Nos: 7, 12, 19, 28, 31). In embodiments, the light chain variable domain comprises an amino acid sequence that has at least 90% sequence identity to any one of the light chain variable domains above (e.g. SEQ IDs Nos: 8, 13, 20, 22, 24, 26, 29, 30, 32, 39, 40, 41, 42). Any of the above heavy chains above (e.g. SEQ ID Nos: 7, 12, 19, 28, 31) may be combined with any of the above light chains (e.g. SEQ IDs Nos: 8, 13, 20, 22, 24, 26, 29, 30, 32, 39, 40, 41, 42).

In some embodiments, the antibodies of the invention have improved binding potency compared to a reference antibody. Improved binding potency may be the result of one or more residues in the CDRs, or of framework residues within the V_{H} or V_{L} regions. Binding potency relates to the half maximal effective concentration (EC50) value, or the concentration required to obtain 50% binding. Binding potency may be measured using ELISA-based assays as known in the art, such as HTT sandwich ELISA.

In some embodiments, the isolated antibodies or fragments thereof bind to mHTT and/or aggregated HTT protein (or a fragment thereof, preferably comprising exon 1) as determined by immunoprecipitation (e.g. immunoprecipitation of mHTT and/or aggregated HTT using an antibody of the disclosure or a fragment thereof).

In some embodiments, antibodies or fragments thereof according to the present disclosure are able to cross the blood-brain barrier. In some embodiments, the antibodies are bispecific antibodies. For example, antibodies according to the present disclosure may have one scFV chain that binds a receptor in the brain, such as the transferrin receptor (Yu et al., Sci Transl Med. 2014 Nov. 5; 6 (261): 261ra154.), as well as a scFv chain that binds HTT as described herein. In some embodiments, antibodies according to the disclosure comprise an antibody or fragment thereof that binds HTT as described herein (such as e.g. an antibody, scFV, sdAb, etc.), and a further binding moiety that binds to another target. The other target may be a receptor in the brain, such as the transferring receptor. The further binding moiety may be an antibody, a scFv, a nanobody, or an aptamer. The two binding moieties of such a bispecific molecule may form a fusion protein.

Also described herein as single domain antibodies (sdAbs), otherwise known as nanobodies, comprising the heavy chain CDRs and/or the VH sequence of any antibody described herein. Thus, also described herein are antibodies or fusion molecules comprising a nanobody that binds HTT as described herein, and a nanobody that binds a receptor in the brain. Also described herein are antibodies of fusion molecules comprising a scFV chain or nanobody that binds HTT as described herein, and an aptamer that binds a receptor in the brain.

Isolated nucleic acids encoding an antibody, antigen binding fragment, or polypeptide as described herein are provided. Also provided is a vector comprising a nucleic acid described herein, and a host cell comprising the vector. For example, the host cell may be a eukaryotic, or mammalian, e.g. Chinese Hamster Ovary (CHO), cell or may be a prokaryotic cell, e.g. E. coli. In some embodiments, the vector is a viral vector, for example a bacteriophage.

Further provided are methods for making an antibody, or antigen binding fragment or polypeptide as described herein is provided, the method comprising culturing a host cell as described herein under conditions suitable for the expression of a vector encoding the antibody, or antigen binding fragment or polypeptide, and isolating and/or purifying the antibody, or antigen binding fragment or polypeptide. The method further comprises formulating the antibody or antibody fragment into a composition including at least one additional component.

The antibodies and fragments thereof described herein may find use in therapy.

A subject to be treated or diagnosed may be any animal or human. The subject is preferably mammalian, more preferably human. The subject may be male or female. The subject may be a patient. Therapeutic uses may be in human or animals (veterinary use).

Medicaments and pharmaceutical compositions according to aspects of the present invention may be formulated for administration by a number of routes, including but not limited to, parenteral, intravenous, intra-arterial, intramuscular, oral and nasal. The medicaments and compositions may be formulated for injection.

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 5 204 (s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans. The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients, i.e. the anti-CFH antibodies used in the composition. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Conditions treatable in accordance with the present disclosure include any in which HTT plays a role, including neurodegenerative disorders, and in particular those characterised by pathological accumulation in the brain of abnormal protein aggregates, for example aggregation of mHTT. Conditions treatable in accordance with the present disclosure include any polyQ (polyglutamine) related diseases (see e.g. Cell Transplant. 2014; 23 (4-5): 441-58). PolyQ diseases are neurodegenerative disorders caused by expanded CAG repeats in a particular protein. They include: six spinocerebellar ataxias (SCA) types 1, 2, 6, 7, 17; Machado-Joseph disease (MJD/SCA3); Huntington's disease (HD); dentatorubral pallidoluysian atrophy (DRPLA); and spinal and bulbar muscular atrophy, X-linked 1 (SMAX1/SBMA). The antibodies of the present disclosure were originally identified through analysis of resilient Alzheimer's disease patients, indicating relevance beyond HD to other aggregation-related neurodegenerative diseases. A neurodegenerative disease or disorder can comprise one or more of the following: Huntington's disease; Alzheimer's disease (AD); frontotemporal dementia; Parkinson's disease (PD); amyotrophic lateral sclerosis (ALS); prion diseases; Lewy body disease; Spinal muscular atrophy (SMA); Motor Neuron Disease (MND); progressive supranuclear palsy (PSP); spinocerebellar ataxias (SCA) types 1, 2, 6, 7 and 17; Machado-Joseph disease (MJD/SCA3); dentatorubral pallidoluysian atrophy (DRPLA); spinal bulbar muscular atrophy X-linked type 1 (SMAX1/SBMA); Anderson-Fabry (X-linked Fabry Disease); and DNAJB6 Myopathies.

The antibodies of the disclosure may be used in therapy with further therapeutic agents. As used herein, a “further therapeutic agent” is an additional compound, protein, vector, antibody, cell or entity with a therapeutic effect. The antibodies may be co-administered with a further therapeutic agent. The antibodies may be co-formulated with a further therapeutic agent. The antibodies may be sequentially administered before or after a further therapeutic agent.

In some embodiments, the antibodies and fragments thereof described herein may find use in a method of diagnosis or monitoring the progression of a disease or disorder characterised by the presence of mutated or aggregated HTT protein in a patient. The presence of mutated and/or aggregated HTT is indicative for progression of the disease. Levels of HTT may be quantified in cerebrospinal fluid or blood and samples derived from a patient. The levels of mutated and/or aggregated protein may be quantified using any technique known in the art. A variety of assays are available including ELISA, flow cytometry, Western blot. Thus, also described herein are methods of detecting the presence and/or amount of mutated or aggregated HTT protein in a sample (e.g. a sample obtained from a patient diagnosed as having or suspected to have a disease or disorder characterised by the presence of mutated or aggregated HTT protein), the method comprising using an antibody or fragment thereof as described herein (e.g. to label, isolate, etc. mutant or aggregated HTT present in the sample).

The antibodies described herein may be used as biomarkers indicating that a subject is likely to have or to develop a disease or disorder characterised by the presence of mutated or aggregated HTT protein. For example, a method of diagnosis of a disease or disorder characterised by the presence of mutated or aggregated HTT protein in a patient may comprise obtaining BCR sequence data from the subject and determining, using said sequence data, whether the subject's BCR repertoire comprises one or more antibodies that are likely to bind to HTT (e.g. antibodies as described herein, such as e.g. antibodies having at least 70%, at least 80%, at least 85%, at least 90%, at least 95% or at least 99% homology to any specific antibody or antibody fragment described herein), wherein a subject whose BCR repertoire comprises one or more antibodies that are likely to bind to HTT is likely have or be at risk of developing a disease or disorder characterised by the presence of mutated or aggregated HTT.

The antibodies described herein may be used as biomarkers indicating that a subject is likely to response to therapy using an antibody or antibody fragment as described herein. A method of determining whether a subject is likely to respond to treatment with an antibody or fragment thereof as described herein, the method comprising obtaining BCR sequence data from the subject and determining, using said sequence data, whether the subject's BCR repertoire comprises one or more antibodies that are likely to bind to HTT (e.g. antibodies as described herein, such as e.g. antibodies having at least 70%, at least 80%, at least 85%, at least 90%, at least 95% or at least 99% homology to any specific antibody or antibody fragment described herein), wherein a subject whose BCR repertoire does not comprise one or more antibodies that are likely to bind to HTT is likely to respond to treatment with an antibody or antibody fragment thereof as described herein. Thus, also described herein is a method of treating a subject who has been diagnosed as having or likely to have a disease or disorder associated with HTT and/or polyQ aggregation (e.g. a neurodegenerative disorder), the method comprising: obtaining BCR sequence data from the subject; determining, using said sequence data, whether the subject's BCR repertoire comprises one or more antibodies that are likely to bind to HTT; and administering to a subject whose BCR repertoire does not comprise one or more antibodies that are likely to bind to HTT a therapeutically effective amount of an antibody or antibody fragment thereof as described herein.

Some methods of the present disclosure involve a sample containing cells. The sample may be a culture of cells grown in vitro. For example, the culture may comprise a suspension of cells or cells cultured in a culture plate or dish. Methods according to the present disclosure may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism.

According to some aspects of the present disclosure a kit of parts is provided comprising an antibody according to the present invention. In some embodiments, the kit comprises an antibody according to the present invention and one or more of: reagents for use in immunochemistry; the antibodies immobilised to a solid support; means for labelling the antibodies; means for linking the antibodies to a cytotoxic moiety; a further therapeutic agent.

Percentage (%) sequence identity is defined as the percentage of amino acid residues in a candidate sequence that are identical with residues in a comparative sequence after aligning the sequences and introducing gaps if necessary, to achieve the maximum sequence identity, and not considering any conservative substitutions as part of the sequence identity. Sequence identity is preferably calculated over the entire length of the respective sequences. Where the aligned sequences are of different length, sequence identity of the shorter comparison sequence may be determined over the entire length of the longer given sequence or, where the comparison sequence is longer than the given sequence, sequence identity of the comparison sequence may be determined over the entire length of the shorter given sequence. Sequence identity may be defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters may be used, with a gap creation penalty=12 and gap extension penalty=4. Use of GAP may be preferred but other algorithms may be used, e.g. BLAST (which uses the method of Altschul et al. (1990) J. Mol. Biol. 215:405-410), FASTA (which uses the method of Pearson and Lipman (1988) PNAS USA 85:2444-2448), SSEARCH (Smith and Waterman (1981) J. Mol Biol. 147:195-197;), HMMER3 (Johnson L S et al BMC Bioinformatics. 2010 Aug. 18; 11( ):431) or the TBLASTN program, of Altschul et al. (1990) supra, generally employing default parameters (see for example Pearson Curr Prot Bioinformatics (2013) Chapt 3 Uniy 3.1 doi:10.1002/0471250953.bi0301s42). In particular, the psi-Blast algorithm may be used (Altschul et al. Nucl. Acids Res. (1997) 25 3389-3402). Sequence identity and similarity may also be determined using Genomequest™ software (Gene-IT, Worcester MA USA). Sequence comparisons are preferably made over the full-length of the relevant sequences to be compared. The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations. Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps. It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

All references cited herein are incorporated herein by reference in their entirety. For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

### Sequences

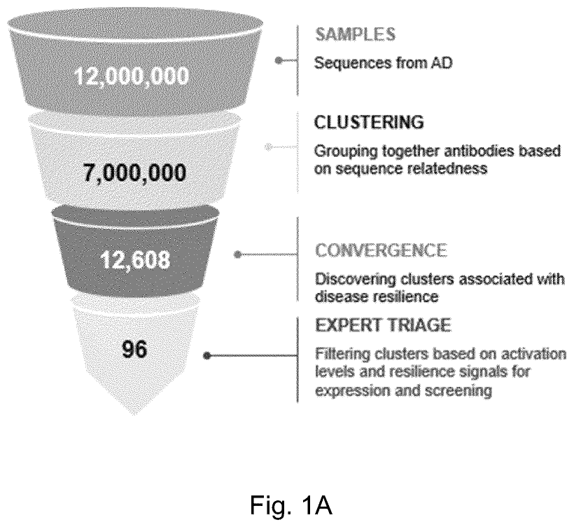

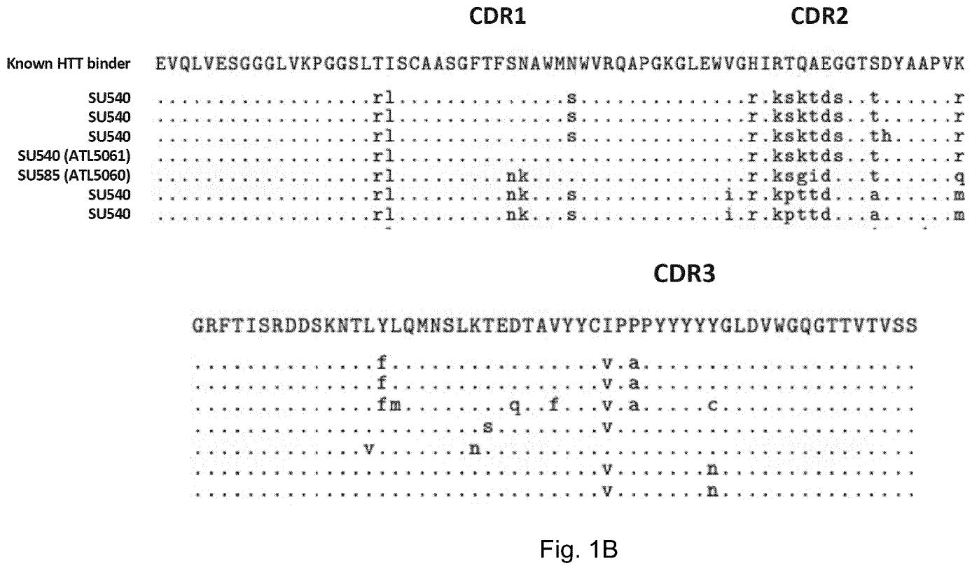

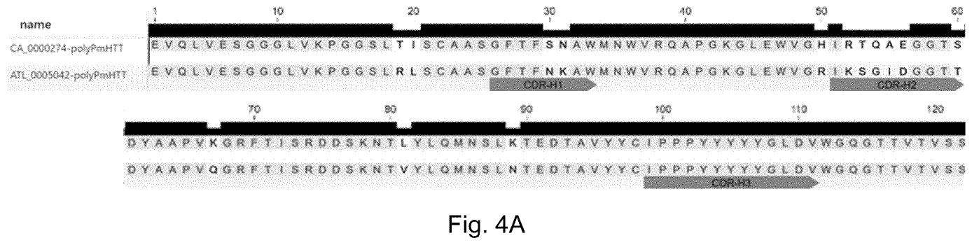

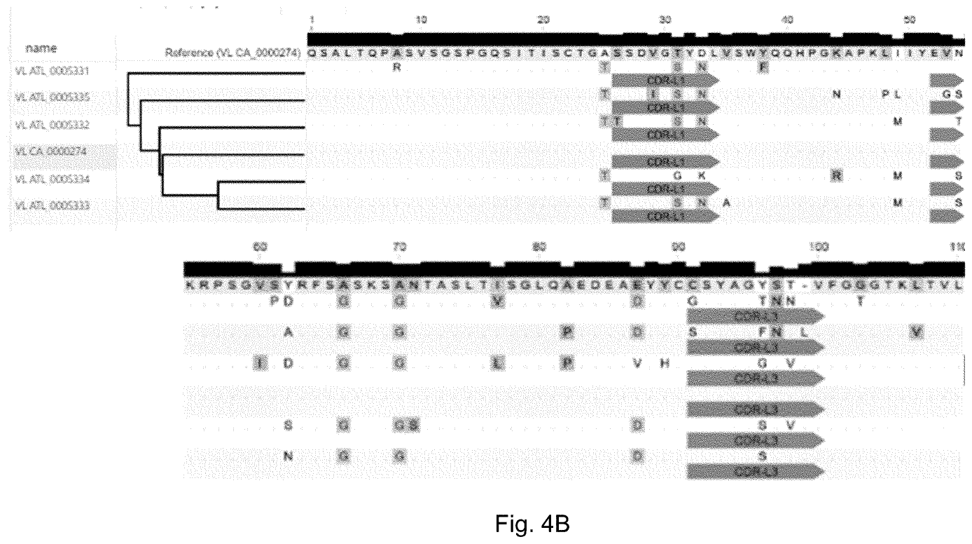

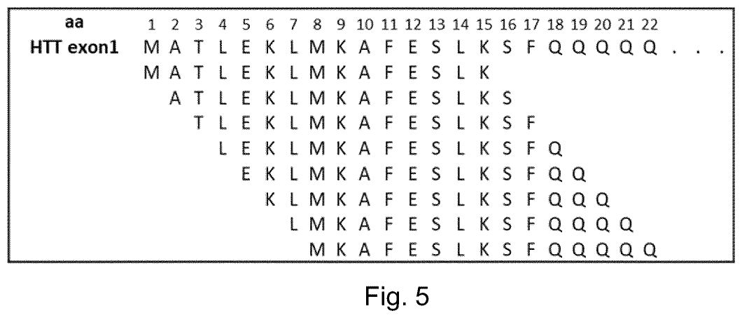

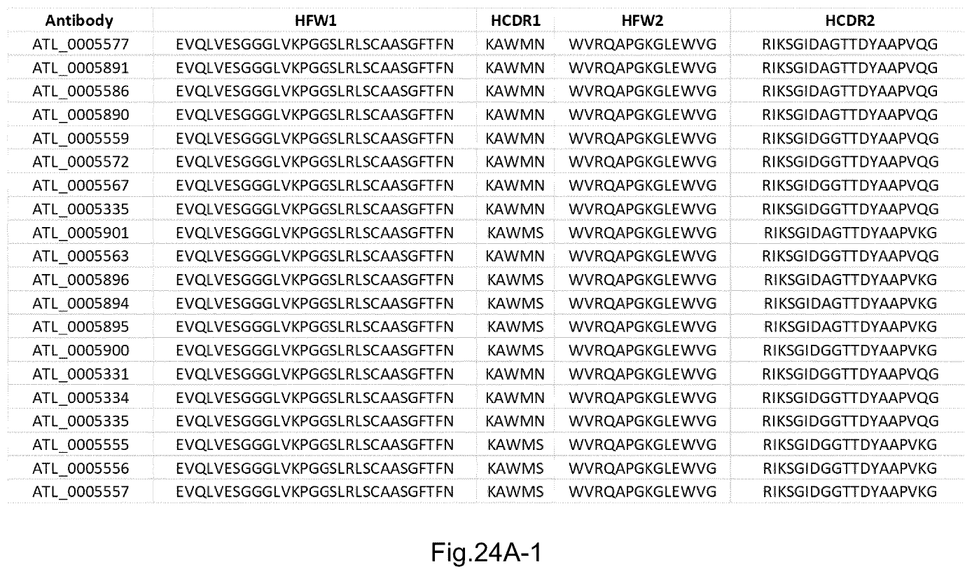

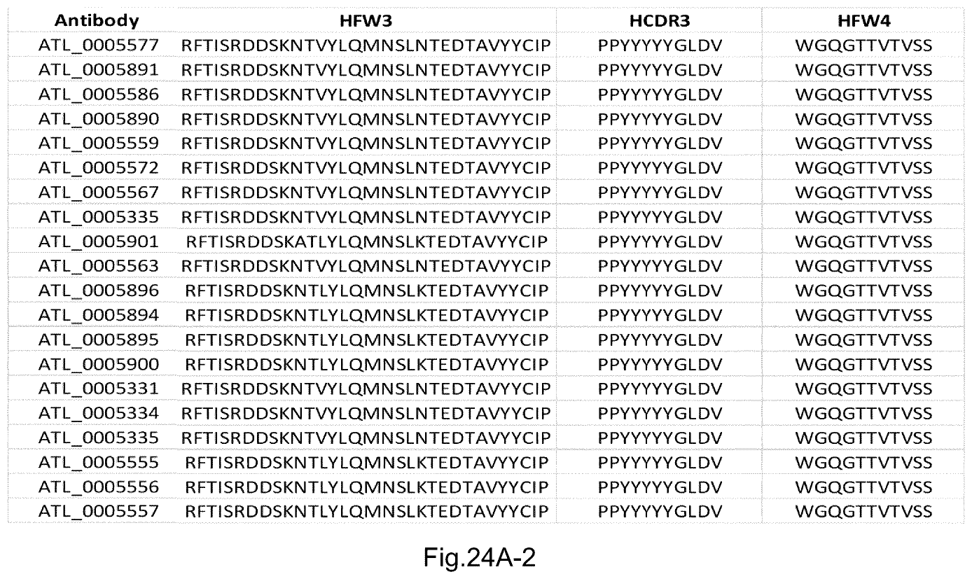

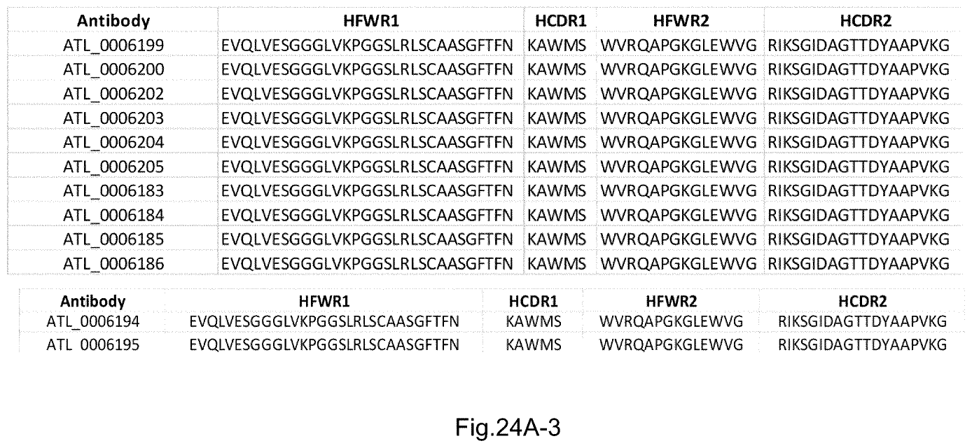

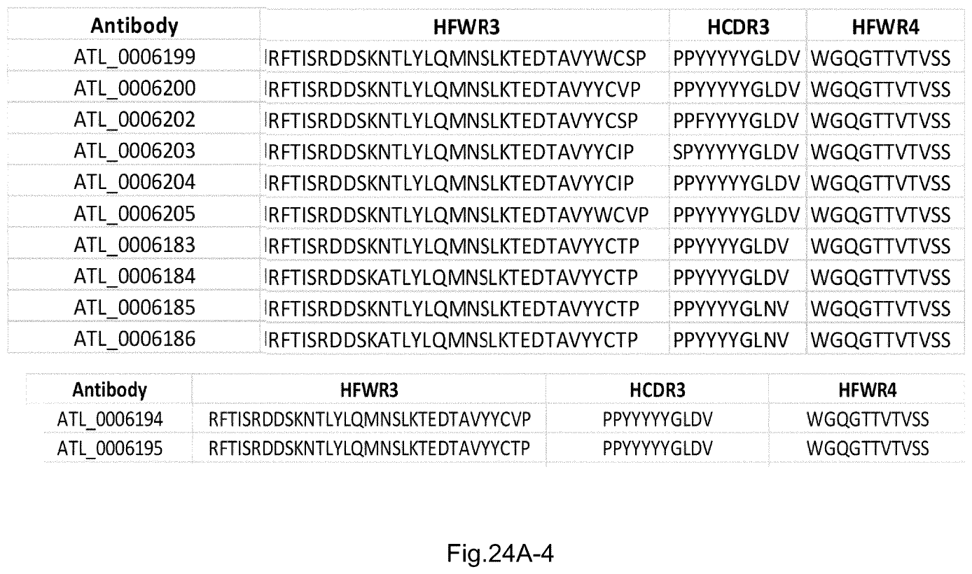

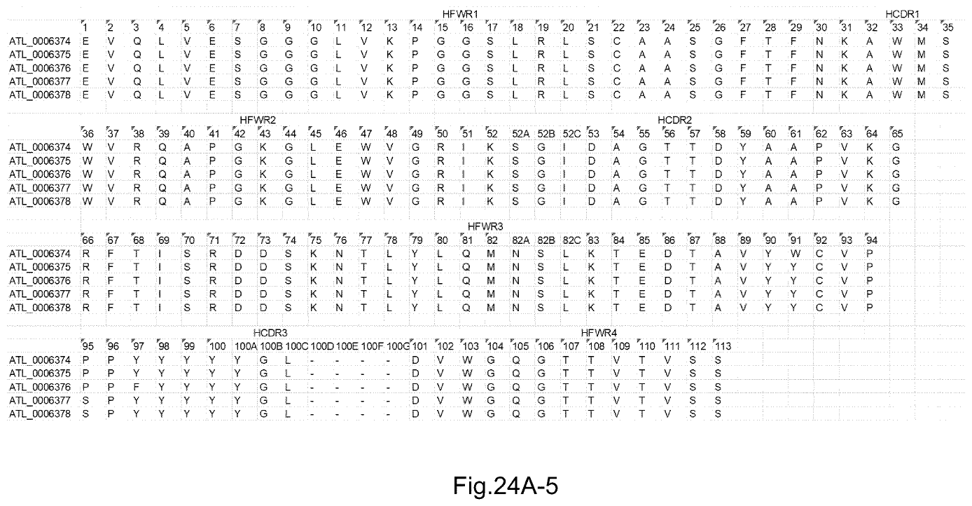

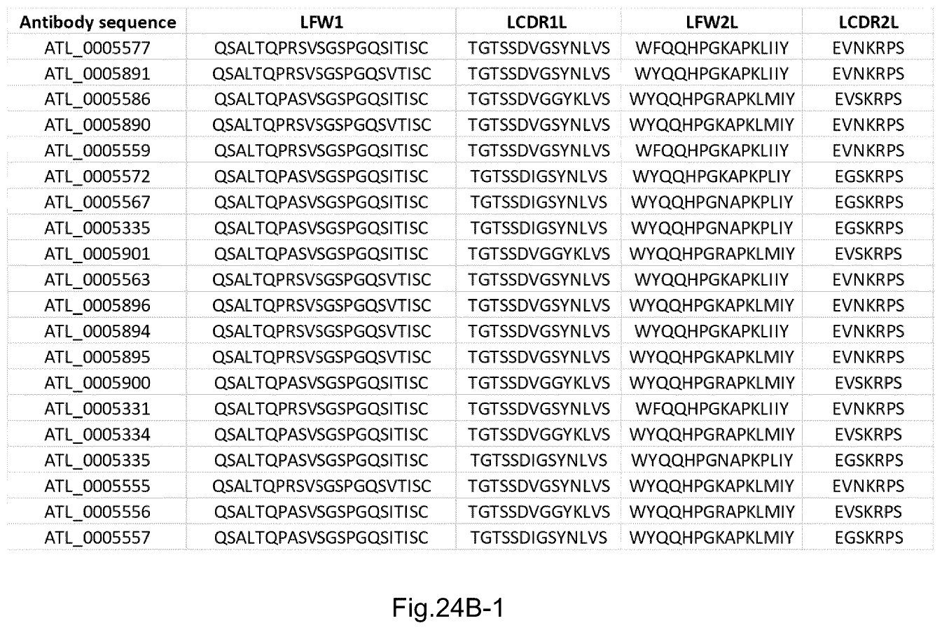

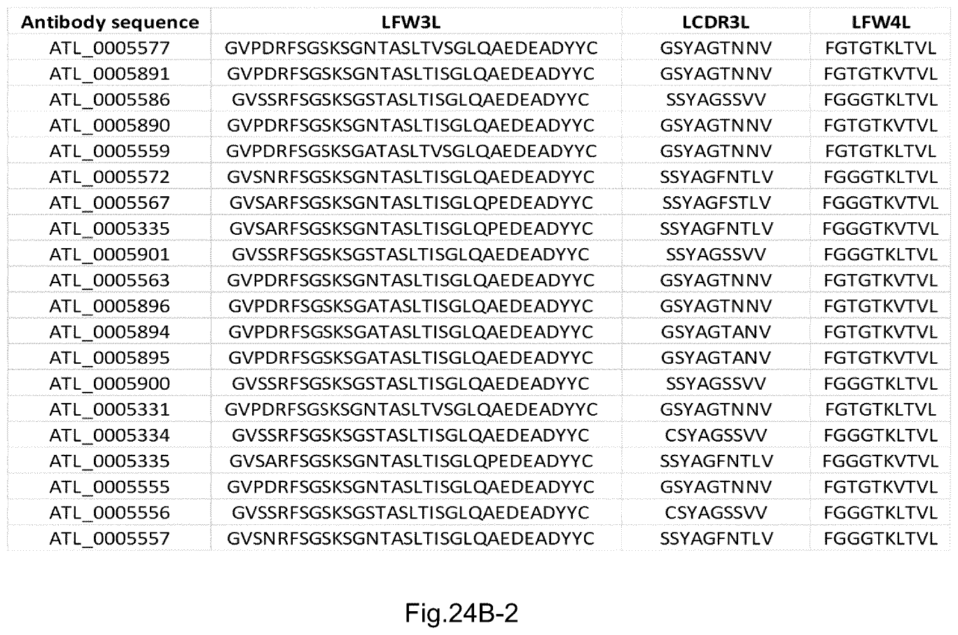

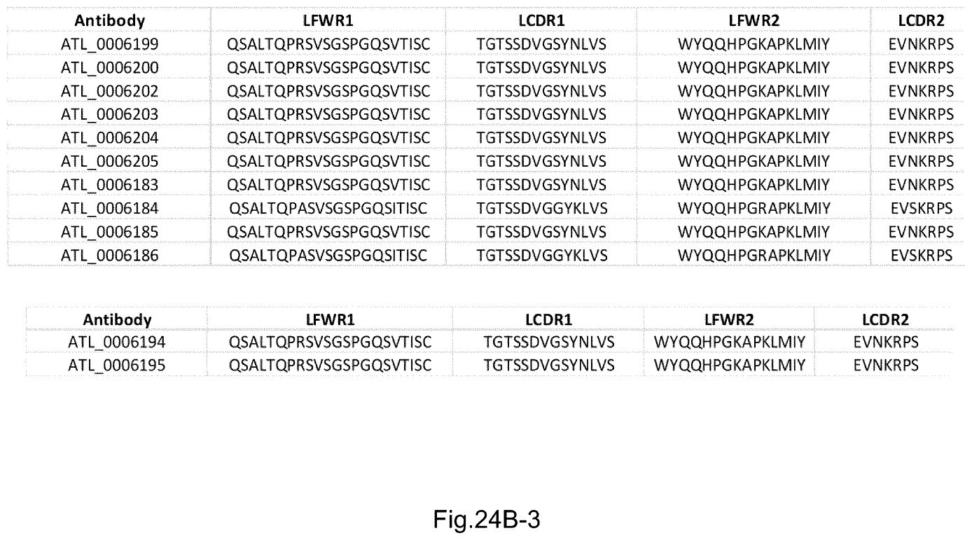

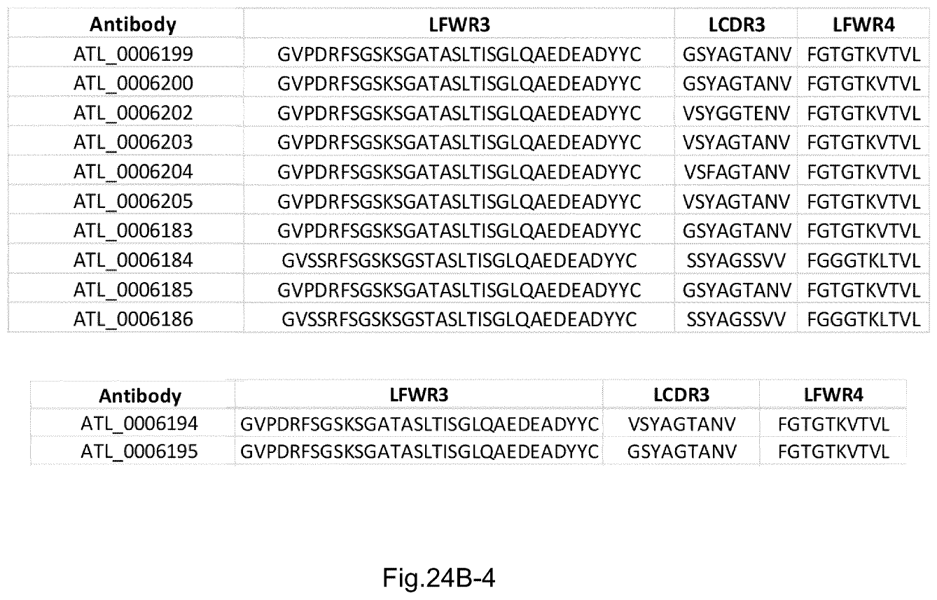

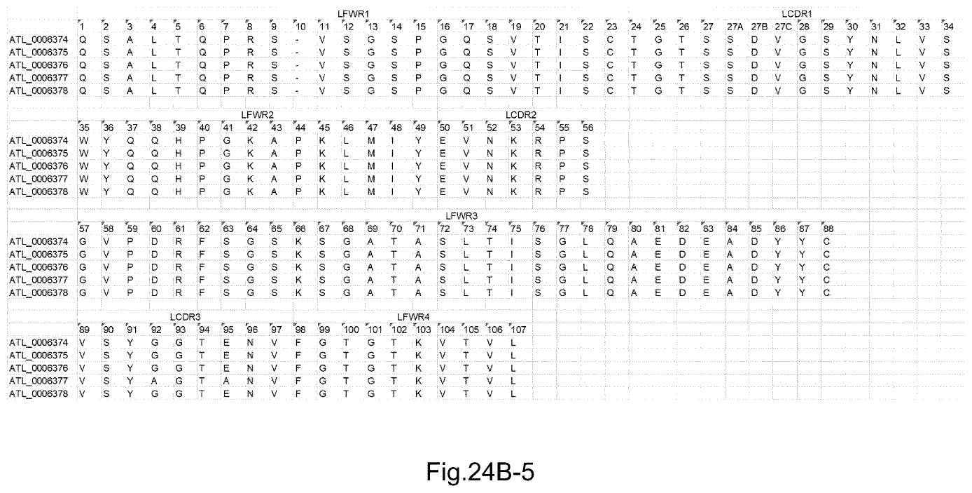

Sequences referred to in the present disclosure. Note that “ATL_” identifiers are
antibody identifiers and “AC_” identifiers refer to unique individual chains,
some of which are shared between different antibodies.

| SEQ ID | | |
|---|---|---|
| NO | Description | Sequence |
| 1 | ATL_5895 HCDR1 | KAWMS |
| | HCDR1_ATL_0006194; | |
| | HCDR1_ATL_0006195; | |
| | HCDR1_ATL_0006199; | |
| | HCDR1_ATL_0006200; | |
| | HCDR1_ATL_0006202; | |
| | HCDR1_ATL_0006203; | |
| | HCDR1_ATL_0006204; | |
| | HCDR1_ATL_0006205; | |
| | HCDR1_ATL_0006183; | |
| | HCDR1_ATL_0006184; | |
| | HCDR1_ATL_0006185; | |
| | HCDR1_ATL_0006186; | |
| | HCDR1_ATL_0006374; | |
| | HCDR1_ATL_0006375; | |
| | HCDR1_ATL_0006376; | |
| | HCDR1_ATL_0006377; | |
| | HCDR1_ATL_0006378 | |
| 2 | ATL_5895 HCDR2 | RIKSGIDAGTTDYAAPVKG |
| | HCDR2_ATL_0006194; | |
| | HCDR2_ATL_0006195; | |
| | HCDR2_ATL_0006200; | |
| | HCDR2_ATL_0006202; | |
| | HCDR2_ATL_0006203; | |
| | HCDR2_ATL_0006204; | |
| | HCDR2_ATL_0006205; | |
| | HCDR2_ATL_0006183; | |
| | HCDR2_ATL_0006184; | |
| | HCDR2_ATL_0006185; | |
| | HCDR2_ATL_0006186; | |
| | HCDR2_ATL_0006374; | |
| | HCDR2_ATL_0006375; | |
| | HCDR2_ATL_0006376; | |
| | HCDR2_ATL_0006377; | |
| | HCDR2_ATL_0006378 | |
| 3 | ATL_5895 HCDR3 | PPYYYYYGLDV |
| | HCDR3_ATL_0006204; | |
| | HCDR3_ATL_0006199; | |
| | HCDR3_ATL_0006183; | |
| | HCDR3_ATL_0006184; | |
| | HCDR3_ATL_0006200; | |
| | HCDR3_ATL_0006205; | |
| | HCDR3_ATL_0006374; | |
| | HCDR3_ATL_0006375; | |
| | HCDR3_ATL_0006194; | |
| | HCDR3_ATL_0006195; | |
| 4 | ATL_5895 LCDR1; | TGTSSDVGSYNLVS |
| | LCDR1_ATL_0006194; | |
| | LCDR1_ATL_0006195; | |
| | LCDR1_ATL_0006374; | |
| | LCDR1_ATL_0006375; | |
| | LCDR1_ATL_0006376; | |
| | LCDR1_ATL_0006377; | |
| | LCDR1_ATL_0006378 | |
| 5 | ATL_5895 LCDR2; | EVNKRPS |
| | LCDR2_ATL_0006194; | |
| | LCDR2_ATL_0006195; | |
| | LCDR2_ATL_0006374; | |
| | LCDR2_ATL_0006375; | |
| | LCDR2_ATL_0006376; | |
| | LCDR2_ATL_0006377; | |
| | LCDR2_ATL_0006378 | |
| 6 | ATL_5895 LCDR3; | GSYAGTANV |
| | LCDR3_ATL_0006195; | |
| 7 | ATL_5895 VH | EVQLVESGGGLVKPGGSLRLSCAASGFTFNKAWMSWVR |
| | (AC_1754) | QAPGKGLEWVGRIKSGIDAGTTDYAAPVKGRFTISRDDSK |
| | | NTLYLQMNSLKTEDTAVYYCIPPPYYYYYGLDVWGQGTT |
| | | VTVSS |
| 8 | ATL_5895 VL | QSALTQPRSVSGSPGQSVTISCTGTSSDVGSYNLVSWYQ |
| | (AC_1756) | QHPGKAPKLMIYEVNKRPSGVPDRFSGSKSGATASLTISG |
| | | LQAEDEADYYCGSYAGTANVFGTGTKVTVL |
| 1 | ATL_5901 HCDR1 | KAWMS |
| 2 | ATL_5901 HCDR2 | RIKSGIDAGTTDYAAPVKG |
| 3 | ATL_5901 HCDR3 | PPYYYYYGLDV |
| 9 | ATL_5901 LCDR1 | TGTSSDVGGYKLVS |
| 10 | ATL_5901 LCDR2 | EVSKRPS |
| 11 | ATL_5901 LCDR3 | SSYAGSSVV |
| 12 | ATL_5901 VH | EVQLVESGGGLVKPGGSLRLSCAASGFTFNKAWMSWVR |
| | (AC_1760) | QAPGKGLEWVGRIKSGIDAGTTDYAAPVKGRFTISRDDSK |
| | | ATLYLQMNSLKTEDTAVYYCIPPPYYYYYGLDVWGQGTT |
| | | VTVSS |
| 13 | ATL_5901 VL | QSALTQPASVSGSPGQSITISCTGTSSDVGGYKLVSWYQ |
| | (AC-1280) | QHPGRAPKLMIYEVSKRPSGVSSRFSGSKSGSTASLTISG |
| | | LQAEDEADYYCSSYAGSSVVFGGGTKLTVL |
| 14 | ATL_5567 HCDR1 | KAWMN |
| 15 | ATL_5567 HCDR2 | RIKSGIDGGTTDYAAPVQG |
| 3 | ATL_5567 HCDR3 | PPYYYYYGLDV |
| 16 | ATL_5567 LCDR1 | TGTSSDIGSYNLVS |
| 17 | ATL_5567 LCDR2 | EGSKRPS |
| 18 | ATL_5567 LCDR3 | SSYAGFSTLV |
| 19 | ATL_5567 VH | EVQLVESGGGLVKPGGSLRLSCAASGFTFNKAWMNWVR |
| | (AC_0737) | QAPGKGLEWVGRIKSGIDGGTTDYAAPVQGRFTISRDDS |
| | | KNTVYLQMNSLNTEDTAVYYCIPPPYYYYYGLDVWGQGT |
| | | TVTVSS |
| 20 | ATL_5567 VL | QSALTQPASVSGSPGQSITISCTGTSSDIGSYNLVSWYQQ |
| | (AC_0001281) | HPGNAPKPLIYEGSKRPSGVSARFSGSKSGNTASLTISGL |
| | | QPEDEADYYCSSYAGFSTLVFGGGTKVTVL |
| 14 | ATL_5331 HCDR1 | KAWMN |
| 15 | ATL_5331 HCDR2 | RIKSGIDGGTTDYAAPVQG |
| 3 | ATL_5331 HCDR3 | PPYYYYYGLDV |
| 4 | ATL_5331 LCDR1 | TGTSSDVGSYNLVS |
| 5 | ATL_5331 LCDR2 | EVNKRPS |
| 21 | ATL_5331 LCDR3 | GSYAGTNNV |
| 19 | ATL_5331 VH | EVQLVESGGGLVKPGGSLRLSCAASGFTFNKAWMNWVR |
| | (AC_0737) | QAPGKGLEWVGRIKSGIDGGTTDYAAPVQGRFTISRDDS |
| | | KNTVYLQMNSLNTEDTAVYYCIPPPYYYYYGLDVWGQGT |
| | | TVTVSS |
| 22 | ATL_5331 VL | QSALTQPRSVSGSPGQSITISCTGTSSDVGSYNLVSWFQ |
| | (AC_1036) | QHPGKAPKLIIYEVNKRPSGVPDRFSGSKSGNTASLTVSG |
| | | LQAEDEADYYCGSYAGTNNVFGTGTKLTVL |
| 14 | ATL_5334 HCDR1 | KAWMN |
| 15 | ATL_5334 HCDR2 | RIKSGIDGGTTDYAAPVQG |
| 3 | ATL_5334 HCDR3 | PPYYYYYGLDV |
| 9 | ATL_5334 LCDR1 | TGTSSDVGGYKLVS |
| 10 | ATL_5334 LCDR2 | EVSKRPS |
| 23 | ATL_5334 LCDR3 | CSYAGSSVV |
| 19 | VH ATL_5334 | EVQLVESGGGLVKPGGSLRLSCAASGFTFNKAWMNWVR |
| | (AC_0737): | QAPGKGLEWVGRIKSGIDGGTTDYAAPVQGRFTISRDDS |
| | | KNTVYLQMNSLNTEDTAVYYCIPPPYYYYYGLDVWGQGT |
| | | TVTVSS |
| 24 | VL ATL_5334 | QSALTQPASVSGSPGQSITISCTGTSSDVGGYKLVSWYQ |
| | (AC_1039) | QHPGRAPKLMIYEVSKRPSGVSSRFSGSKSGSTASLTISG |
| | | LQAEDEADYYCCSYAGSSVVFGGGTKLTVL |
| 14 | ATL_5335 HCDR1 | KAWMN |
| 15 | ATL_5335 HCDR2 | RIKSGIDGGTTDYAAPVQG |
| 3 | ATL_5335 HCDR3 | PPYYYYYGLDV |
| 16 | ATL_5335 LCDR1 | TGTSSDIGSYNLVS |
| 17 | ATL_5335 LCDR2 | EGSKRPS |
| 25 | ATL_5335 LCDR3 | SSYAGFNTLV |
| 19 | VH ATL_5335 | EVQLVESGGGLVKPGGSLRLSCAASGFTFNKAWMNWVR |
| | (AC_0737) | QAPGKGLEWVGRIKSGIDGGTTDYAAPVQGRFTISRDDS |
| | | KNTVYLQMNSLNTEDTAVYYCIPPPYYYYYGLDVWGQGT |
| | | TVTVSS |
| 26 | VL ATL_5335 | QSALTQPASVSGSPGQSITISCTGTSSDIGSYNLVSWYQQ |
| | (AC_1040) | HPGNAPKPLIYEGSKRPSGVSARFSGSKSGNTASLTISGL |
| | | QPEDEADYYCSSYAGFNTLVFGGGTKVTVL |
| 1 | ATL_5555 HCDR1 | KAWMS |
| 27 | ATL_5555 HCDR2 | RIKSGIDGGTTDYAAPVKG |
| 3 | ATL_5555 HCDR3 | PPYYYYYGLDV |
| 4 | ATL_5555 LCDR1 | TGTSSDVGSYNLVS |
| 5 | ATL_5555 LCDR2 | EVNKRPS |
| 21 | ATL_5555 LCDR3 | GSYAGTNNV |
| 28 | VH ATL_5555 | EVQLVESGGGLVKPGGSLRLSCAASGFTFNKAWMSWVR |
| | (AC_1269): | QAPGKGLEWVGRIKSGIDGGTTDYAAPVKGRFTISRDDS |
| | | KNTLYLQMNSLKTEDTAVYYCIPPPYYYYYGLDVWGQGT |
| | | TVTVSS |
| 29 | VL ATL_5555 | QSALTQPRSVSGSPGQSVTISCTGTSSDVGSYNLVSWYQ |
| | (AC_1270) | QHPGKAPKLMIYEVNKRPSGVPDRFSGSKSGNTASLTISG |
| | | LQAEDEADYYCGSYAGTNNVFGTGTKVTVL |
| 1 | ATL_5556 HCDR1 | KAWMS |
| 27 | ATL_5556 HCDR2 | RIKSGIDGGTTDYAAPVKG |
| 3 | ATL_5556 HCDR3 | PPYYYYYGLDV |
| 9 | ATL_5556 LCDR1 | TGTSSDVGGYKLVS |
| 10 | ATL_5556 LCDR2 | EVSKRPS |
| 23 | ATL_5556 LCDR3 | CSYAGSSVV |
| 28 | VH ATL_5556 | EVQLVESGGGLVKPGGSLRLSCAASGFTFNKAWMSWVR |
| | (AC_1269): | QAPGKGLEWVGRIKSGIDGGTTDYAAPVKGRFTISRDDS |
| | | KNTLYLQMNSLKTEDTAVYYCIPPPYYYYYGLDVWGQGT |
| | | TVTVSS |
| 24 | VL ATL_5556 | QSALTQPASVSGSPGQSITISCTGTSSDVGGYKLVSWYQ |
| | (AC_1039) | QHPGRAPKLMIYEVSKRPSGVSSRFSGSKSGSTASLTISG |
| | | LQAEDEADYYCCSYAGSSVVFGGGTKLTVL |
| 1 | ATL_5557 HCDR1 | KAWMS |
| 27 | ATL_5557 HCDR2 | RIKSGIDGGTTDYAAPVKG |
| 3 | ATL_5557 HCDR3 | PPYYYYYGLDV |
| 16 | ATL_5557 LCDR1 | TGTSSDIGSYNLVS |
| 17 | ATL_5557 LCDR2 | EGSKRPS |
| 25 | ATL_5557 LCDR3 | SSYAGFNTLV |
| 28 | VH ATL_5557 | EVQLVESGGGLVKPGGSLRLSCAASGFTFNKAWMSWVR |
| | (AC_1269): | QAPGKGLEWVGRIKSGIDGGTTDYAAPVKGRFTISRDDS |
| | | KNTLYLQMNSLKTEDTAVYYCIPPPYYYYYGLDVWGQGT |
| | | TVTVSS |
| 30 | VL ATL_5557 | QSALTQPASVSGSPGQSITISCTGTSSDIGSYNLVSWYQQ |
| | (AC_1271) | HPGKAPKLMIYEGSKRPSGVSNRFSGSKSGNTASLTISGL |
| | | QAEDEADYYCSSYAGFNTLVFGGGTKLTVL |
| 31 | VH ATL_5577 | EVQLVESGGGLVKPGGSLRLSCAASGFTFNKAWMNWVR |
| | (AC_1291) | QAPGKGLEWVGRIKSGIDAGTTDYAAPVQGRFTISRDDS |
| | | KNTVYLQMNSLNTEDTAVYYCIPPPYYYYYGLDVWGQGT |
| | | TVTVSS |
| 22 | VL ATL_5577 | QSALTQPRSVSGSPGQSITISCTGTSSDVGSYNLVSWFQ |
| | (AC_0001036) | QHPGKAPKLIIYEVNKRPSGVPDRFSGSKSGNTASLTVSG |
| | | LQAEDEADYYCGSYAGTNNVFGTGTKLTVL |
| 31 | VH ATL_5891 | EVQLVESGGGLVKPGGSLRLSCAASGFTFNKAWMNWVR |
| | (AC_1291) | QAPGKGLEWVGRIKSGIDAGTTDYAAPVQGRFTISRDDS |
| | | KNTVYLQMNSLNTEDTAVYYCIPPPYYYYYGLDVWGQGT |
| | | TVTVSS |
| 32 | VL ATL_5891 | QSALTQPRSVSGSPGQSVTISCTGTSSDVGSYNLVSWYQ |
| | (AC_1277) | QHPGKAPKLIIYEVNKRPSGVPDRFSGSKSGNTASLTISGL |
| | | QAEDEADYYCGSYAGTNNVFGTGTKVTVL |
| 31 | VH ATL_5586 | EVQLVESGGGLVKPGGSLRLSCAASGFTFNKAWMNWVR |
| | (AC_1291) | QAPGKGLEWVGRIKSGIDAGTTDYAAPVQGRFTISRDDS |
| | | KNTVYLQMNSLNTEDTAVYYCIPPPYYYYYGLDVWGQGT |
| | | TVTVSS |
| 13 | VL ATL_5586 | QSALTQPASVSGSPGQSITISCTGTSSDVGGYKLVSWYQ |
| | (AC_1280) | QHPGRAPKLMIYEVSKRPSGVSSRFSGSKSGSTASLTISG |
| | | LQAEDEADYYCSSYAGSSVVFGGGTKLTVL |
| 33 | VH ATL_5059 | EVQLVESGGGLVKPGGSLTISCAASGFTFSNAWMNWVR |
| | (AC_0000754) | QAPGKGLEWVGHIRTQAEGGTSDYAAPVKGRFTISRDDS |
| | | KNTLYLQMNSLKTEDTAVYYCIPPPYYYYYGLDVWGQGT |
| | | TVTVSS |
| 34 | VL ATL_5059 | QSALTQPASVSGSPGQSITISCTGASSDVGTYDLVSWYQ |
| | (AC_0000755) | QHPGKAPKLIIYEVNKRPSGVSYRFSASKSANTASLTISGL |
| | | QAEDEAEYYCCSYAGYSTVFGGGTKLTVL |
| 35 | ATL_5059 HCDR1 | NAWMN |
| 36 | ATL_5059 HCDR2 | HIRTQAEGGTSDYAAPVKG |
| 3 | ATL_5059 HCDR3 | PPYYYYYGLDV |
| 37 | ATL_5059 LCDR1 | TGASSDVGTYDLVS |
| 5 | ATL_5059 LCDR2 | EVNKRPS |
| 38 | ATL_5059 LCDR3 | CSYAGYSTV |
| 19 | VH ATL_5572 | EVQLVESGGGLVKPGGSLRLSCAASGFTFNKAWMNWVR |
| | (AC_0737) | QAPGKGLEWVGRIKSGIDGGTTDYAAPVQGRFTISRDDS |
| | | KNTVYLQMNSLNTEDTAVYYCIPPPYYYYYGLDVWGQGT |
| | | TVTVSS |
| 39 | VL ATL_5572 | QSALTQPASVSGSPGQSITISCTGTSSDIGSYNLVSWYQQ |
| | (AC_0001286) | HPGKAPKPLIYEGSKRPSGVSNRFSGSKSGNTASLTISGL |
| | | QAEDEADYYCSSYAGFNTLVFGGGTKLTVL |
| 19 | VH ATL_5563 | EVQLVESGGGLVKPGGSLRLSCAASGFTFNKAWMNWVR |
| | (AC_0737) | QAPGKGLEWVGRIKSGIDGGTTDYAAPVQGRFTISRDDS |
| | | KNTVYLQMNSLNTEDTAVYYCIPPPYYYYYGLDVWGQGT |
| | | TVTVSS |
| 32 | VL ATL_5563 | QSALTQPRSVSGSPGQSVTISCTGTSSDVGSYNLVSWYQ |
| | (AC_1277) | QHPGKAPKLIIYEVNKRPSGVPDRFSGSKSGNTASLTISGL |
| | | QAEDEADYYCGSYAGTNNVFGTGTKVTVL |
| 7 | VH ATL_5896 | EVQLVESGGGLVKPGGSLRLSCAASGFTFNKAWMSWVR |
| | (AC_1754) | QAPGKGLEWVGRIKSGIDAGTTDYAAPVKGRFTISRDDSK |
| | | NTLYLQMNSLKTEDTAVYYCIPPPYYYYYGLDVWGQGTT |
| | | VTVSS |
| 40 | VL ATL_5896 | QSALTQPRSVSGSPGQSVTISCTGTSSDVGSYNLVSWYQ |
| | (AC_1752) | QHPGKAPKLMIYEVNKRPSGVPDRFSGSKSGATASLTISG |
| | | LQAEDEADYYCGSYAGTNNVFGTGTKVTVL |
| 7 | VH ATL_5894 | EVQLVESGGGLVKPGGSLRLSCAASGFTFNKAWMSWVR |
| | (AC_1754) | QAPGKGLEWVGRIKSGIDAGTTDYAAPVKGRFTISRDDSK |
| | | NTLYLQMNSLKTEDTAVYYCIPPPYYYYYGLDVWGQGTT |
| | | VTVSS |
| 41 | VL ATL_5894 | QSALTQPRSVSGSPGQSVTISCTGTSSDVGSYNLVSWYQ |
| | (AC_0001755) | QHPGKAPKLIIYEVNKRPSGVPDRFSGSKSGATASLTISGL |
| | | QAEDEADYYCGSYAGTANVFGTGTKVTVL |
| 28 | VH ATL_5900 | EVQLVESGGGLVKPGGSLRLSCAASGFTFNKAWMSWVR |
| | (AC_1269) | QAPGKGLEWVGRIKSGIDGGTTDYAAPVKGRFTISRDDS |
| | | KNTLYLQMNSLKTEDTAVYYCIPPPYYYYYGLDVWGQGT |
| | | TVTVSS |
| 13 | VL ATL_5900 | QSALTQPASVSGSPGQSITISCTGTSSDVGGYKLVSWYQ |
| | (AC_1280) | QHPGRAPKLMIYEVSKRPSGVSSRFSGSKSGSTASLTISG |
| | | LQAEDEADYYCSSYAGSSVVFGGGTKLTVL |
| 19 | ATL_5559 VH | EVQLVESGGGLVKPGGSLRLSCAASGFTFNKAWMNWVR |
| | (AC_0000737) | QAPGKGLEWVGRIKSGIDGGTTDYAAPVQGRFTISRDDS |
| | | KNTVYLQMNSLNTEDTAVYYCIPPPYYYYYGLDVWGQGT |
| | | TVTVSS |
| 42 | ATL_5559 VL | QSALTQPRSVSGSPGQSITISCTGTSSDVGSYNLVSWFQ |
| | (AC_0001273) | QHPGKAPKLIIYEVNKRPSGVPDRFSGSKSGATASLTVSG |
| | | LQAEDEADYYCGSYAGTNNVFGTGTKLTVL |
| 31 | ATL_5890 VH | EVQLVESGGGLVKPGGSLRLSCAASGFTFNKAWMNWVR |
| | (AC_0001291) | QAPGKGLEWVGRIKSGIDAGTTDYAAPVQGRFTISRDDS |
| | | KNTVYLQMNSLNTEDTAVYYCIPPPYYYYYGLDVWGQGT |
| | | TVTVSS |
| 29 | ATL_5890 VL | QSALTQPRSVSGSPGQSVTISCTGTSSDVGSYNLVSWYQ |
| | (AC_0001270) | QHPGKAPKLMIYEVNKRPSGVPDRFSGSKSGNTASLTISG |
| | | LQAEDEADYYCGSYAGTNNVFGTGTKVTVL |
| 19 | ATL_5060 VH | EVQLVESGGGLVKPGGSLRLSCAASGFTFNKAWMNWVR |
| | (same VH as ATL5042- | QAPGKGLEWVGRIKSGIDGGTTDYAAPVQGRFTISRDDS |
| | VH only, used for phage | KNTVYLQMNSLNTEDTAVYYCIPPPYYYYYGLDVWGQGT |
| | display) | TVTVSS |
| 34 | ATL5060 VL | QSALTQPASVSGSPGQSITISCTGASSDVGTYDLVSWYQ |
| | (AC_0000755) | QHPGKAPKLIIYEVNKRPSGVSYRFSASKSANTASLTISGL |
| | | QAEDEAEYYCCSYAGYSTVFGGGTKLTVL (SEQ ID NO: |
| | | 34) |
| 114 | ATL5061 VH | EVQLVESGGGLVKPGGSLRLSCAASGFTFSNAWMNWVR |
| | (AC_0000756) | QAPGKGLEWVGRIKSKTDSGTTDYAAPVRGRFTISRDDS |
| | | KNTLYLQMNSLKSEDTAVYYCVPPPYYYYYGLDVWGQGT |
| | | TVTVSS |
| 34 | ATL5061 (VL) | QSALTQPASVSGSPGQSITISCTGASSDVGTYDLVSWYQ |
| | (AC_0000755) | QHPGKAPKLIIYEVNKRPSGVSYRFSASKSANTASLTISGL |
| | | QAEDEAEYYCCSYAGYSTVFGGGTKLTVL (SEQ ID NO: |
| | | 34) |
| 43 | WT Human HTT Exon 1 | MKHHHHHHNTSSNSMSPILGYWKIKGLVQPTRLLLEYLEE |
| | 25Q | KYEEHLYERDEGDKWRNKKFELGLEFPNLPYYIDGDVKLT |
| | | QSMAIIRYIADKHNMLGGCPKERAEISMLEGAVLDIRYGVS |
| | | RIAYSKDFETLKVDFLSKLPEMLKMFEDRLCHKTYLNGDH |
| | | VTHPDFMLYDALDVVLYMDPMCLDAFPKLVCFKKRIEAIP |
| | | QIDKYLKSSKYIAWPLQGWQATFGGGDHPPTSGSGGGG |
| | | GWMSENLYFQGAMATLEKLMKAFESLKSFQQQQQQQQ |
| | | QQQQQQQQQQQQQQQQQPPPPPPPPPPPQLPQPPPQ |
| | | AQPLLPQPQPPPPPPPPPPGPAVAEEPLHRP |
| 44 | Mutant Human HTT Exon | MKHHHHHHNTSSNSMSPILGYWKIKGLVQPTRLLLEYLEE |
| | 1 48Q | KYEEHLYERDEGDKWRNKKFELGLEFPNLPYYIDGDVKLT |
| | | QSMAIIRYIADKHNMLGGCPKERAEISMLEGAVLDIRYGVS |
| | | RIAYSKDFETLKVDFLSKLPEMLKMFEDRLCHKTYLNGDH |
| | | VTHPDFMLYDALDVVLYMDPMCLDAFPKLVCFKKRIEAIP |
| | | QIDKYLKSSKYIAWPLQGWQATFGGGDHPPTSGSGGGG |
| | | GWMSENLYFQGAMATLEKLMKAFESLKSFQQQQQQQQ |
| | | QQQQQQQQQQQQQQQQQQQQQQQQQQQQQQQQQQ |
| | | QQQQQQPPPPPPPPPPPQLPQPPPQAQPLLPQPQPPPP |
| | | PPPPPPGPAVAEEPLHRP. |
| 45 | WT Human HTT Exon 1 | MGSSHHHHHHSQDPNSGGGGGWMSENLYFQGAMATLE |
| | 25Q His | KLMKAFESLKSFQQQQQQQQQQQQQQQQQQQQQQQQ |
| | | QPPPPPPPPPPPQLPQPPPQAQPLLPQPQPPPPPPPPPP |
| | | GPAVAEEPLHRP |
| 46 | Mutant Human HTT Exon | MGSSHHHHHHSQDPNSGGGGGWMSENLYFQGAMATLE |
| | 1 48Q His | KLMKAFESLKSFQQQQQQQQQQQQQQQQQQQQQQQQ |
| | | QQQQQQQQQQQQQQQQQQQQQQQQPPPPPPPPPPP |
| | | QLPQPPPQAQPLLPQPQPPPPPPPPPPGPAVAEEPLHRP |
| 47 | Epitope 1 | QQQQPPPPPPPPPPP |
| 48 | Epitope 2 | LPQPQPPPPPPPPPPP |
| 49 | Human HTT exon 1 | MATLEKLMKAFESLKSFQQQQQQQQQQQQQQQQPPPP |
| | | PPPPPPPQLPQPPPQAQPLLPQPQPPPPPPPPPPGPAVA |
| | | EEPLHR |
| 50 | Cyno HTT exon 1 (Cyno | MATLEKLMKAFESLKSFQQQQQQQQQQQPPPPPPPPPP |
| | 1) | PPQLPQPPQAQPMLPQPQPPPPPPRHHPARLWLRSRCT |
| | | DQRKNFQLPRKTV |
| 51 | Cyno HTT exon 1 (Cyno | MATLEKLMKAFESLKSFQQQQQQQQQQQQPPPPPPPPP |
| | 2) | PPPQLPQPPQAQPMLPQPQPPPPPPPPPPGPAVAEEPLH |
| | | RPKKELSATKKDRVNH |
| 52 | Mouse HTT exon 1 | MATLEKLMKAFESLKSFQQQQQQQPPPQAPPPPPPPPP |
| | | PQPPQPPPQGQPPPPPPPLPGPAEEPLHR |
| 54 | Peptide 1 | QQQQQQQQPPPPPPP |
| 55 | Peptide 2 | QQQQQQQPPPPPPPP |
| 56 | Peptide 3 | QQQQQQPPPPPPPPP |
| 57 | Peptide 4 | QQQQQPPPPPPPPPP |
| 58 | Peptide 5 | QQQPPPPPPPPPPPQ |
| 59 | Peptide 6 | QQPPPPPPPPPPPQL |
| 60 | Peptide 7 | PQAQPLLPQPQPPPP |
| 61 | Peptide 8 | QAQPLLPQPQPPPPP |
| 62 | Peptide 9 | AQPLLPQPQPPPPPP |
| 63 | Peptide 10 | QPLLPQPQPPPPPPP |
| 64 | Peptide 11 | PLLPQPQPPPPPPPP |
| 65 | Peptide 12 | LLPQPQPPPPPPPPP |
| 66 | Peptide 13 | LPQPQPPPPPPPPPP |
| 67 | Peptide 14 | PQPQPPPPPPPPPPG |
| 68 | Peptide 15 | QPQPPPPPPPPPPGP |
| 69 | Peptide 16 | PQPPPPPPPPPPGPA |
| 70 | Peptide 17 | QPPPPPPPPPPGPAV |
| 71 | Peptide 18 | QQQPPPPPPPPPPPP |
| 72 | Peptide 19 | PQAQPMLPQPQPPPP |
| 73 | Peptide 20 | QAQPMLPQPQPPPPP |
| 74 | Peptide 21 | AQPMLPQPQPPPPPP |
| 75 | Peptide 22 | QPMLPQPQPPPPPPR |
| 76 | Peptide 23 | QQQQQQQQQQPPPPP |
| 77 | Peptide 24 | QQQQQQQQQPPPPPP |
| 78 | Peptide 25 | QQPPPPPPPPPPPP |
| 79 | Peptide 26 | QPPPPPPPPPPPPP |
| 80 | Peptide 27 | PPPPPPPPPPPPPP |
| 81 | Peptide 28 | QPMLPQPQPPPPPPP |
| 82 | Peptide 29 | PMLPQPQPPPPPPPP |
| 83 | Peptide 30 | MLPQPQPPPPPPPPP |
| 84 | Peptide 31 | LPQPQPPPPPPPPPP |
| 85 | Peptide 32 | QQQQQPPPQAPPPPP |
| 86 | Peptide 33 | QQQQPPPQAPPPPPP |
| 87 | Peptide 34 | QQQPPPQAPPPPPPP |
| 88 | Peptide 35 | QQPPPQAPPPPPPPP |
| 89 | Peptide 36 | QPPPQAPPPPPPPPP |
| 90 | Peptide 37 | PPPQAPPPPPPPPPP |
| 91 | Peptide 38 | PPQAPPPPPPPPPPQ |
| 92 | Peptide 39 | PQAPPPPPPPPPPQP |
| 93 | Peptide 40 | QAPPPPPPPPPPQPP |
| 94 | Peptide 41 | PPQPPPQGQPPPPPP |
| 95 | Peptide 42 | PQPPPQGQPPPPPPP |
| 96 | Peptide 43 | QPPPQGQPPPPPPPL |
| 97 | Peptide 44 | PPPQGQPPPPPPPLP |
| 98 | HFW1 5331, 5334, 5335, | EVQLVESGGGLVKPGGSLRLSCAASGFTFN |
| | 6374, 6375, 6376, 6377, | |
| | 6378, 6194, 6195 | |
| 99 | HFW2 5331, 5334, 5335, | WVRQAPGKGLEWVG |
| | 6374, 6375, 6376, 6377, | |
| | 6378, 6194, 6195 | |
| 100 | HFW3 5331, 5334, 5335 | RFTISRDDSKNTVYLQMNSLNTEDTAVYYCIP |
| 101 | HFW4 5331, 5334, 5335, | WGQGTTVTVSS |
| | 6374, 6375, 6376, 6377, | |
| | 6378, 6194, 6195 | |
| 102 | LFW1 5331 | QSALTQPRSVSGSPGQSITISC |
| 103 | LFW2 5331 | WFQQHPGKAPKLIIY |
| 104 | LFW3 5331 | GVPDRFSGSKSGNTASLTVSGLQAEDEADYYC |
| 105 | LFW4 5331 | FGTGTKLTVL |
| 106 | LFW1 5334, 5335 | QSALTQPASVSGSPGQSITISC |
| 107 | LFW2 5334 | WYQQHPGRAPKLMIY |
| 108 | LFW3 5334 | GVSSRFSGSKSGSTASLTISGLQAEDEADYYC |
| 109 | LFW4 5334 | FGGGTKLTVL |
| 110 | LFW2 5335 | WYQQHPGNAPKPLIY |
| 111 | LFW3 5335 | GVSARFSGSKSGNTASLTISGLQPEDEADYYC |
| 112 | LFW4 5335 | FGGGTKVTVL |
| 113 | Consensus binding motif | [P/Q]Q[P/Q]QPPPPPPPPPPP |
| 53 | Known HTT binder | EVQLVESGGGLVKPGGSLRLSCAASGFTFSNAWMSWVR |
| | (FIG. 1B) | QAPGKGLEWVGRIKSKTDSGTTDYAAPVRGRFTISRDDS |
| | | KNTLFLQMNSLKTEDTAVYYCVPAPYYYYYGLDVWGQGT |
| | | TVTVSS |
| 114 | FIG. 1.B (SU540, 1^{st} | EVQLVESGGGLVKPGGSLRLSCAASGFTFSNAWMSWVR |
| | sequence) | QAPGKGLEWVGRIKSKTDSGTTDYAAPVRGRFTISRDDSK |
| | | NTLFLQMNSLKTEDTAVYYCVPAPYYYYYGLDVWGQGTT |
| | | VTVSS |
| 115 | FIG. 1.B (SU540, 3^{rd} | EVQLVESGGGLVKPGGSLRLSCAASGFTFSNAWMSWVR |
| | sequence) | QAPGKGLEWVGRIKSKTDSGTTHYAAPVRGRFTISRDDSK |
| | | NTLFMQMNSLKTEQTAFYYCVPAPYYYYCGLDVWGQGTT |
| | | VTVSS |
| 116 | FIG. 1.B | EVQLVESGGGLVKPGGSLRLSCAASGFTFNKAWMNWVR |
| | (SU585(ATL5060)) | QAPGKGLEWVGRIKSGIDGGTTDYAAPVQGRFTISRDDSK |
| | | NTVYLQMNSLNTEDTAVYYCIPPPYYYYYGLDVWGQGTTV |
| | | TVSS |
| 117 | FIG. 1.B (SU540, 6^{th} | EVQLVESGGGLVKPGGSLRLSCAASGFTFNKAWMSWVR |
| | sequence) | QAPGKGLEWIGRIKPTTDGGTADYAAPVMGRFTISRDDSK |
| | | NTLYLQMNSLKTEDTAVYYCVPPPYYYYNGLDVWGQGTT |
| | | VTVSS |
| 118 | FIG. 4A (1^{st} SEQ- | EVQLVESGGGLVKPGGSLTISCAASGFTFSNAWMNWVRQ |
| | CA_0000274, also | APGKGLEWVGHIRTQAEGGTSDYAAPVKGRFTISRDDSKN |
| | referred to as | TLYLQMNSLKTEDTAVYYCIPPPYYYYYGLDVWGQGTTVT |
| | ATL_0005059 or NI- | VSS |
| | 302.8F1), SEQ ID NO: 33 | |
| | [duplicate sequence] | |
| 119 | Peptide of a peptide array | MATLEKLMKAFESLKSFQQQQQ |
| | for HTT epitope mapping | |
| | (FIG. 5) | |
| 120 | Peptide of a peptide array | MATLEKLMKAFESLK |
| | for HTT epitope mapping | |
| | (FIG. 5) | |
| 121 | Peptide of a peptide array | ATLEKLMKAFESLKS |
| | for HTT epitope mapping | |
| | (FIG. 5) | |
| 122 | Peptide of a peptide array | TLEKLMKAFESLKSF |
| | for HTT epitope mapping | |
| | (FIG. 5) | |
| 123 | Peptide of a peptide array | LEKLMKAFESLKSFQ |
| | for HTT epitope mapping | |
| | (FIG. 5) | |
| 124 | Peptide of a peptide array | EKLMKAFESLKSFQQ |
| | for HTT epitope mapping | |
| | (FIG. 5) | |
| 125 | Peptide of a peptide array | KLMKAFESLKSFQQQ |
| | for HTT epitope mapping | |
| | (FIG. 5) | |
| 126 | Peptide of a peptide array | LMKAFESLKSFQQQQ |
| | for HTT epitope mapping | |
| | (FIG. 5) | |
| 127 | Peptide of a peptide array | MKAFESLKSFQQQQQ |
| | for HTT epitope mapping | |
| | (FIG. 5) | |
| 128 | FIG. 24A (HCDR2 of | RIKSGIDAGTTDYAAPVQG |
| | ATL_0005577, 5891, | |
| | 5586, 5890) | |
| 129 | FIG. 24A (HFW3 of | RFTISRDDSKATLYLQMNSLKTEDTAVYYCIP |
| | ATL_0005901) | |
| 130 | FIG. 24A (HFW3 of | RFTISRDDSKNTLYLQMNSLKTEDTAVYYCIP |
| | ATL_0005894, 5895, | |
| | 5896, 5900) | |
| 131 | FIG. 24B (LFW1 of | QSALTQPRSVSGSPGQSVTISC |
| | ATL_0005891, 5890, | |
| | 5563, 5896, 5894, 5895, | |
| | 6374, 6202, 6375, 6376, | |
| | 6377, 6194, 6195) | |
| 132 | FIG. 24B (LFW2L of | WYQQHPGKAPKLIIY |
| | ATL_0005563, 5894, | |
| | 5891) | |
| 133 | FIG. 24B (LFW2L of | WYQQHPGKAPKLMIY |
| | ATL_0005896, 5895, | |
| | 5890, 6374, 6375, 6376, | |
| | 6377, 6378, 6194, 6195) | |
| 134 | FIG. 24B (LFW2L of | WYQQHPGKAPKPLIY |
| | ATL_0005572) | |
| 135 | FIG. 24B (LFW3L of | GVPDRFSGSKSGNTASLTISGLQAEDEADYYC |
| | ATL_0005891, 5563, | |
| | 5890) | |
| 136 | FIG. 24B (LFW3L of | GVPDRFSGSKSGATASLTVSGLQAEDEADYYC |
| | ATL_0005559) | |
| 137 | FIG. 24B (LFW3L of | GVSNRFSGSKSGNTASLTISGLQAEDEADYYC |
| | ATL_0005572) | |
| 138 | FIG. 24B (LFW3L of | GVPDRFSGSKSGATASLTISGLQAEDEADYYC |
| | ATL_0005896, 5894, | |
| | 5895, 6374, 6375, 6376, | |
| | 6377, 6378, 6194, 6195) | |
| 139 | FIG. 24B (LFW4L of | FGTGTKVTVL |
| | ATL_0005891, 5563, | |
| | 5896, 5894, 5895, 5891, | |
| | 5890, 6374, 6375, 6376, | |
| | 6377, 6378, 6194, 6195) | |
| 140 | FIG. 4B(4th SEQ-VL | QSALTQPASVSGSPGQSITISCTGTTSDVGSYNLVSWYQQ |
| | CA_0000274) | HPGKAPKLMIYEVTKRPSGISDRFSGSKSGNTASLTLSGLQ |
| | | PEDEAVYHCCSYAGGSVVFGGGTKLTVL |
| 141 | FIG. 4B (6th SEQ-VL | QSALTQPASVSGSPGQSITISCTGTSSDVGSYNLASWYQQ |
| | ATL_0005333) | HPGKAPKLMIYEVSKRPSGVSNRFSGSKSGNTASLTISGL |
| | | QAEDEADYYCCSYAGSSTVFGGGTKLTVL |
| 142 | HA control peptide | YPYDVPDYAG |
| 143 | Polio control peptide | KEVPALTAVETGAT |
| 144 | ATL_0006199 VH | EVQLVESGGGLVKPGGSLRLSCAASGFTFNKAWMSWVR |
| | | QAPGKGLEWVGRIKSGIDAGTTDYAAPVKGRFTISRDDSK |
| | | NTLYLQMNSLKTEDTAVYWCSPPPYYYYYGLDVWGQGTT |
| | | VTVSS |
| 145 | ATL_0006200 VH; | EVQLVESGGGLVKPGGSLRLSCAASGFTFNKAWMSWVR |
| | ATL_0006375 VH; | QAPGKGLEWVGRIKSGIDAGTTDYAAPVKGRFTISRDDSK |
| | ATL_0006194 | NTLYLQMNSLKTEDTAVYYCVPPPYYYYYGLDVWGQGTT |
| | | VTVSS |
| 146 | ATL_0006202 VH | EVQLVESGGGLVKPGGSLRLSCAASGFTFNKAWMSWVR |
| | | QAPGKGLEWVGRIKSGIDAGTTDYAAPVKGRFTISRDDSK |
| | | NTLYLQMNSLKTEDTAVYYCSPPPFYYYYGLDVWGQGTT |
| | | VTVSS |
| 147 | ATL_0006203 VH | EVQLVESGGGLVKPGGSLRLSCAASGFTFNKAWMSWVR |
| | | QAPGKGLEWVGRIKSGIDAGTTDYAAPVKGRFTISRDDSK |
| | | NTLYLQMNSLKTEDTAVYYCIPSPYYYYYGLDVWGQGTTV |
| | | TVSS |
| 7 | ATL_0006204 VH | EVQLVESGGGLVKPGGSLRLSCAASGFTFNKAWMSWVR |
| | | QAPGKGLEWVGRIKSGIDAGTTDYAAPVKGRFTISRDDSK |
| | | NTLYLQMNSLKTEDTAVYYCIPPPYYYYYGLDVWGQGTTV |
| | | TVSS |
| 148 | ATL_0006205 VH; | EVQLVESGGGLVKPGGSLRLSCAASGFTFNKAWMSWVR |
| | ATL_0006374 VH | QAPGKGLEWVGRIKSGIDAGTTDYAAPVKGRFTISRDDSK |
| | | NTLYLQMNSLKTEDTAVYWCVPPPYYYYYGLDVWGQGTT |
| | | VTVSS |
| 149 | ATL_0006183 VH | EVQLVESGGGLVKPGGSLRLSCAASGFTFNKAWMSWVR |
| | | QAPGKGLEWVGRIKSGIDAGTTDYAAPVKGRFTISRDDSK |
| | | NTLYLQMNSLKTEDTAVYYCTPPPYYYYGLDVWGQGTTV |
| | | TVSS |
| 150 | ATL_0006184 VH | EVQLVESGGGLVKPGGSLRLSCAASGFTFNKAWMSWVR |
| | | QAPGKGLEWVGRIKSGIDAGTTDYAAPVKGRFTISRDDSK |
| | | ATLYLQMNSLKTEDTAVYYCTPPPYYYYGLDVWGQGTTVT |
| | | VSS |
| 151 | ATL_0006185 VH | EVQLVESGGGLVKPGGSLRLSCAASGFTFNKAWMSWVR |
| | | QAPGKGLEWVGRIKSGIDAGTTDYAAPVKGRFTISRDDSK |
| | | NTLYLQMNSLKTEDTAVYYCTPPPYYYYGLNVWGQGTTV |
| | | TVSS |
| 152 | ATL_0006186 VH | EVQLVESGGGLVKPGGSLRLSCAASGFTFNKAWMSWVR |
| | | QAPGKGLEWVGRIKSGIDAGTTDYAAPVKGRFTISRDDSK |
| | | ATLYLQMNSLKTEDTAVYYCTPPPYYYYGLNVWGQGTTVT |
| | | VSS |
| 8 | ATL_0006199 VL | QSALTQPRSVSGSPGQSVTISCTGTSSDVGSYNLVSWYQ |
| | | QHPGKAPKLMIYEVNKRPSGVPDRFSGSKSGATASLTISG |
| | | LQAEDEADYYCGSYAGTANVFGTGTKVTVL |
| 8 | ATL_0006200 VL | QSALTQPRSVSGSPGQSVTISCTGTSSDVGSYNLVSWYQ |
| | | QHPGKAPKLMIYEVNKRPSGVPDRFSGSKSGATASLTISG |
| | | LQAEDEADYYCGSYAGTANVFGTGTKVTVL |
| 153 | ATL_0006202 VL; | QSALTQPRSVSGSPGQSVTISCTGTSSDVGSYNLVSWYQ |
| | ATL_0006374 VL; | QHPGKAPKLMIYEVNKRPSGVPDRFSGSKSGATASLTISG |
| | ATL_0006375 VL; | LQAEDEADYYCVSYGGTENVFGTGTKVTVL |
| | ATL_0006376 VL; | |
| | ATL_0006378 VL | |
| 154 | ATL_0006203 VL; | QSALTQPRSVSGSPGQSVTISCTGTSSDVGSYNLVSWYQ |
| | ATL_0006377 VL; | QHPGKAPKLMIYEVNKRPSGVPDRFSGSKSGATASLTISG |
| | ATL_0006205; | LQAEDEADYYCVSYAGTANVFGTGTKVTVL |
| | ATL_0006194 VL | |
| 155 | ATL_0006204 VL | QSALTQPRSVSGSPGQSVTISCTGTSSDVGSYNLVSWYQ |
| | | QHPGKAPKLMIYEVNKRPSGVPDRFSGSKSGATASLTISG |
| | | LQAEDEADYYCVSFAGTANVFGTGTKVTVL |
| 156 | ATL_0006205 VL (SEQ | QSALTQPRSVSGSPGQSVTISCTGTSSDVGSYNLVSWYQ |
| | ID NO: 154-duplicate | QHPGKAPKLMIYEVNKRPSGVPDRFSGSKSGATASLTISG |
| | sequence) | LQAEDEADYYCVSYAGTANVFGTGTKVTVL |
| 8 | ATL_0006183 VL; | QSALTQPRSVSGSPGQSVTISCTGTSSDVGSYNLVSWYQ |
| | ATL_0006185 VL | QHPGKAPKLMIYEVNKRPSGVPDRFSGSKSGATASLTISG |
| | | LQAEDEADYYCGSYAGTANVFGTGTKVTVL |
| 13 | ATL_0006184 VL | QSALTQPASVSGSPGQSITISCTGTSSDVGGYKLVSWYQQ |
| | ATL_0006186 VL | HPGRAPKLMIYEVSKRPSGVSSRFSGSKSGSTASLTISGL |
| | | QAEDEADYYCSSYAGSSVVFGGGTKLTVL |
| 157 | HCDR3_ATL_0006203; | SPYYYYYGLDV |
| | HCDR3_ATL_0006377; | |
| | HCDR3_ATL_0006378 | |
| 158 | HCDR3_ATL_0006202; | PPFYYYYGLDV |
| | HCDR3_ATL_0006376 | |
| 159 | HCDR3_ATL_0006185; | PPYYYYGLNV |
| | HCDR3_ATL_0006186 | |
| 9 | LCDR1_ATL_0006184; | TGTSSDVGGYKLVS |
| | LCDR1_ATL_0006186 | |
| 4 | ;LCDR1_ATL_0006199; | TGTSSDVGSYNLVS |
| | LCDR1_ATL_0006200; | |
| | LCDR1_ATL_0006202; | |
| | LCDR1_ATL_0006203; | |
| | LCDR1_ATL_0006204; | |
| | LCDR1_ATL_0006183; | |
| | LCDR1_ATL_0006185 | |
| 5 | LCDR2_ATL_0006199; | EVNKRPS |
| | LCDR2_ATL_0006200; | |
| | LCDR2_ATL_0006202; | |
| | LCDR2_ATL_0006203; | |
| | LCDR2_ATL_0006204; | |
| | LCDR2_ATL_0006205; | |
| | LCDR2_ATL_0006183; | |
| | LCDR2_ATL_0006185 | |
| 10 | LCDR2_ATL_0006184; | EVSKRPS |
| | LCDR2_ATL_0006186 | |
| 6 | LCDR3_ATL_0006199; | GSYAGTANV |
| | LCDR3_ATL_0006200; | |
| | LCDR3_ATL_0006179; | |
| | LCDR3_ATL_0006181; | |
| | LCDR3_ATL_0006183; | |
| | LCDR3_ATL_0006185 | |
| 11 | LCDR3_ATL_0006184; | SSYAGSSVV |
| | LCDR3_ATL_0006186 | |
| 160 | LCDR3_ATL_0006204 | VSFAGTANV |
| 161 | LCDR3_ATL_0006203; | VSYAGTANV |
| | LCDR3_ATL_0006205; | |
| | LCDR3_ATL_0006377; | |
| | LCDR3_ATL_0006194 | |
| 162 | LCDR3_ATL_0006202; | VSYGGTENV |
| | LCDR3_ATL_0006374; | |
| | LCDR3_ATL_0006375; | |
| | LCDR3_ATL_0006376; | |
| | LCDR3_ATL_0006378 | |
| 163 | ATL_5895 and ATL_6204 | YCIPPPYYYYYGLDV |
| | Extended CDR3H | |
| 164 | ATL_6199 Extended | WCSPPPYYYYYGLDV |
| | CDR3H | |
| 165 | ATL_6200 Extended | YCVPPPYYYYYGLDV |
| | CDR3H | |
| 166 | ATL_6202 Extended | YCSPPPFYYYYGLDV |
| | CDR3H | |
| 167 | ATL_6203 Extended | YCIPSPYYYYYGLDV |
| | CDR3H | |
| 168 | ATL_6205 Extended | WCVPPPYYYYYGLDV |
| | CDR3H | |
| 169 | ATL_6199 HFWR3 | RFTISRDDSKNTLYLQMNSLKTEDTAVYWCSP |
| 170 | ATL_6200 HFWR3 | RFTISRDDSKNTLYLQMNSLKTEDTAVYYCVP |
| | ATL6194 HFWR3 | |
| 171 | ATL_6202 HFWR3 | RFTISRDDSKNTLYLQMNSLKTEDTAVYYCSP |
| 172 | ATL_6202; ATL_6024 | RFTISRDDSKNTLYLQMNSLKTEDTAVYYCIP |
| | HFWR3 | |
| 173 | ATL_6205; 6374 HFWR3 | RFTISRDDSKNTLYLQMNSLKTEDTAVYWCVP |
| 174 | Synthetic peptide used in | GYSLPQPQPPPPPPPPPP |
| | phage display | |
| | experiments | |
| 175 | ATL_0006376 VH | EVQLVESGGGLVKPGGSLRLSCAASGFTFNKAWMSWVR |
| | | QAPGKGLEWVGRIKSGIDAGTTDYAAPVKGRFTISRDDSK |
| | | NTLYLQMNSLKTEDTAVYYCVPPPFYYYYGLDVWGQGTT |
| | | VTVSS |
| 176 | ATL_0006377 VH; | EVQLVESGGGLVKPGGSLRLSCAASGFTFNKAWMSWVR |
| | ATL_0006378 VH | QAPGKGLEWVGRIKSGIDAGTTDYAAPVKGRFTISRDDSK |
| | | NTLYLQMNSLKTEDTAVYYCVPSPYYYYYGLDVWGQGTT |
| | | VTVSS |
| 177 | Murine HTT antigen used | MGSSHHHHHHSQDPNSGGGGGWMSENLYFQGAMATLE |
| | in Murine HTT ELISA | KLMKAFESLKSFQQQQQQQPPPQAPPPPPPPPPQPPQP |
| | experiment | PPQGQPPPPPPPLPGPAEEPLHRP |
| 178 | ATL_0006374 HFWR3 | RFTISRDDSKNTLYLQMNSLKTEDTAVYWCVP |
| 179 | ATL_0006195 VH | EVQLVESGGGLVKPGGSLRLSCAASGFTFNKAWMSWVR |
| | | QAPGKGLEWVGRIKSGIDAGTTDYAAPVKGRFTISRDDSK |
| | | NTLYLQMNSLKTEDTAVYYCTPPPYYYYYGLDVWGQGTT |
| | | VTVSS |
| 8 | ATL_0006195 VL | QSALTQPRSVSGSPGQSVTISCTGTSSDVGSYNLVSWYQ |
| | | QHPGKAPKLMIYEVNKRPSGVPDRFSGSKSGATASLTISG |
| | | LQAEDEADYYCGSYAGTANVFGTGTKVTVL |
| 180 | ATL_0006195 HFWR3 | RFTISRDDSKNTLYLQMNSLKTEDTAVYYCTP |

### EXAMPLES

The examples below demonstrate the process of identification and characterisation of exemplary HTT antibodies of the disclosure.

### Materials and Methods

### Capillary Isoelectric Focusing

Charge variant analysis was performed on lead antibodies by preparing a master mix to dilute antibody samples to run on a cIEF cartridge on a Maurice instrument (Protein Simple). The master mix had a final concentration of methyl cellulose 0.35%, pharmalyte 3-10 4%, 10 mM arginine, pI markers 4.09 and 9.99 0.01%. Samples were diluted at 0.15-0.25 mg/ml in the master mix and were run for 1 minute at 1500 volts followed by 4.5 minutes at 3000 volts. A system suitability standard (Protein Simples) was also run at the beginning and end of the run. Data generated for the same samples through stress conditions were overlaid to compare the charge species profile.

### 5×FT Cycles

mAb samples at 5 mg/ml were freeze thawed from −80 C storage to room temperature (RT) over 5 cycles within an 8 hour period. A final sample following 5 freeze thaw cycles was diluted to 0.7 mg/ml for analysing purity on SEC-HPLC.

### SEC-HPLC

Antibody samples were diluted to 0.7 mg/ml in 20 mM histidine acetate, 150 mM NaCl pH 5.5 to run on a Zorbax GF-250 SEC-HPLC column (Agilent) on a Vanquish Flex (Thermo). Samples were separated by size in mobile phase 20 mM Sodium phosphate, 300 mM Sodium sulfate and 100 mM Arginine at a flow rate of 0.75 ml/minute at 25 C for 25 minutes per sample. Chromeleon software (Thermo) was used to integrate the chromatograms,

### Recombinant HTT Exon-1 Binding ELISA

Indirect ELISAs were performed using 2 different constructs of recombinant HTT Exon-1 with His- and GST-tags (WT Human HTT Exon 1 25Q GST and WT Human HTT Exon 1 48Q GST) and 2 different constructs of recombinant HTT Exon-1 with His-tag only (WT Human HTT Exon 1 25Q His and WT Human HTT Exon 1 48Q His). Each construct or the irrelevant protein lysozyme as a control was coated onto a Nunc Maxsorp plate at 5 μg/ml in PBS and incubated overnight at 4° C. Plates were blocked with PBS+2% non-fat milk powder for 2 hours at room temperature. Buffer was discarded and plates washed three times with PBS+0.1% Tween-20. Test or control antibodies were added to the wells at 100 μg/ml in PBS+2% non-fat milk powder and incubated for 1 hour at room temperature. Buffer was discarded and plates washed three times with PBS+0.1% Tween-20. HRP-conjugated secondary antibody in PBS+2% non-fat milk powder was added to the plate and incubated for 30 minutes at room temperature. Buffer was discarded and plates washed three times with PBS+0.1% Tween-20. TMB substrate was added to each well and allowed to develop over 5-10 minutes at room temperature before being stopped with 0.2M NaOH. Plates were read measuring absorbance at 450 nm.

### Thermal Shift Assay

Protein thermal shift measurements were performed on a QuantiStudio 5 real time qPCR (ThermoFisher). Antibodies were diluted to 0.1-0.25 with the addition of 10× Sypro orange protein gel stain (Thermo #S6651). Samples were loaded into the qPCR in a 384 well Microamp plates. Samples were run through a temperature range of 25-95 C with 2-minute intervals. Protein melt curves were analysed using Protein Thermal Shift software to determine Tm values, which relate to the stability of the antibody.

### HTT Sandwich ELISA

To assess binding potency of antibodies to HTT protein, antibodies were assessed in a sandwich ELISA format for binding to HTT exon 1 with 25Q repeats or 48Q repeats (see “Sequences”) for sequences) and EC50 calculated. The anti-HTT capture antibody (MerckMillipore; #MABN2427) was diluted to 4.17 μg/ml in 1×ELISA coating buffer (Biolegend; #421701). 50 μl was added per well of 96 well plate, and left overnight at 4° C. The plate was washed with PBS/0.1% Tween. The plate was blocked with 50 μl/well of blocking buffer (1% BSA/PBS). The plate was washed with PBS/0.1% Tween. 50 μl of diluted antigen of interest (HTT exon1 48Q GST or HTT exon1 25Q GST) and lysozyme (negative antigen control) were added to the plate at 0.04 μg/ml. The plate was incubated for 1 hr at RT on a plate shaker (300 rpm). The plate was washed with PBS/0.1% Tween. An 8-point 3-fold serial dilution of test antibody (generally staring at 400 nM) was added to the plate. Another set of wells received 50 μl per well buffer only (blank control). The plate was incubated for 1 hr at RT on a plate shaker (300 rpm). The plate was washed with PBS/0.1% Tween. 50 μl of anti-Human IgG HRP was added per well (80 ng/ml; Jackson ImmunoResearch; #109-035-097; Lot: 160716). The plate was washed with PBS/0.1% Tween and 50 μl of TMB solution (Lifetechnology; #002023) added. Incubated for 6 minutes at RT in dark. 50 μl of stopping solution added to plate (Fither chemical, #12933634). Absorbance was read on CLARIO Star at 450 nm. Background (average from wells with no test antibody) subtracted from all values. EC50 (the half maximal concentration) values were calculated using a non-linear, four parameter curve fitting, with no constraints.

### In Vivo PK Study

To determine serum PK of the lead antibodies, in vivo PK experiment was performed with ATL_0005335 (parent antibody of ATL_0005567). Female, 6-8-week-old, C57BL6J mice received ATL_0005335 at 10 and 20 mg/Kg by IP injection and serum was collected 1, 4, 8, 24, 72 and 144 hours post administration. Antibody levels were assessed via ELISA of serum samples.

### HTT Immunoprecipitation U-2 OS Cell Lysate

Immunoprecipitation was performed using Protein G coated Dynabeads™ and Magnetic rack (Thermosfiher; 10014D). After incubation with antibody, beads were washed using magnetic rack and incubated with U-2 OS cell lysate (from parental or HTT 110CAG expressing version). Beads were again washed and then the captured protein eluted and analysed via automated western blot (Bio-techne; Jess). Detection was with the anti-HTT antibody 1C2 (Merck/Millipore MAB1574).

HTT Immunoprecipitation with R6/2 Mouse Brains

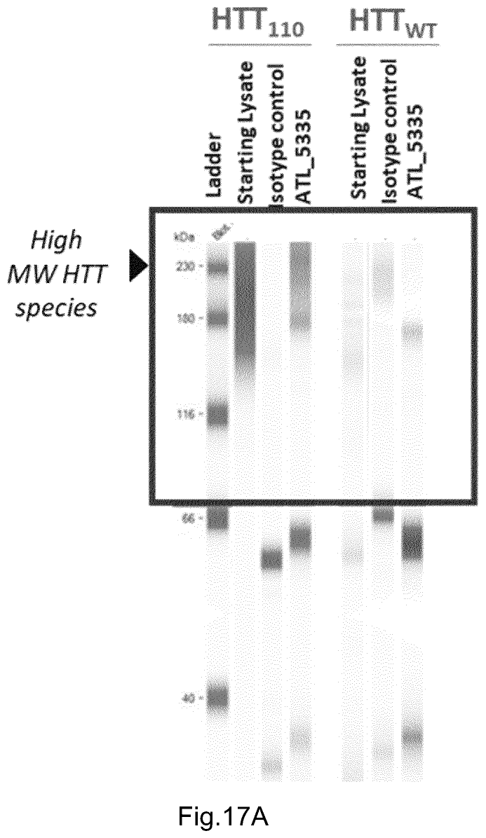

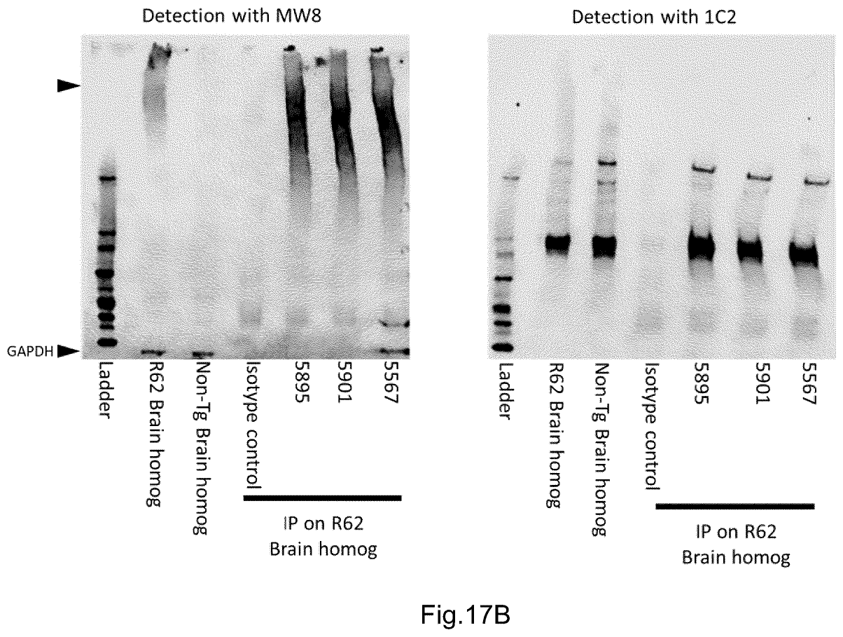

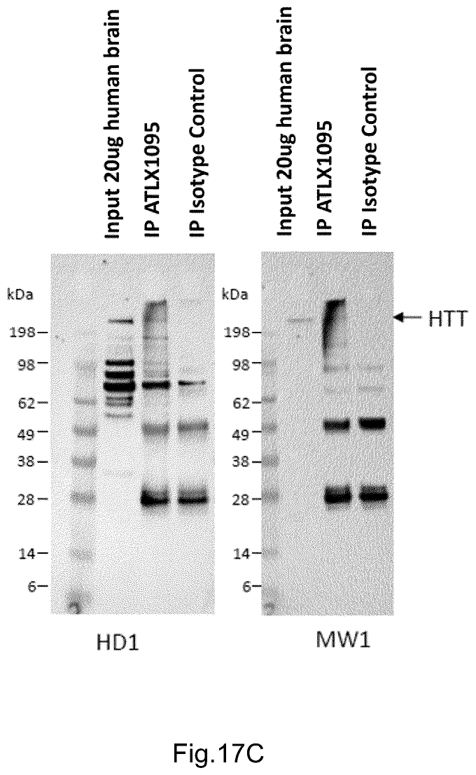

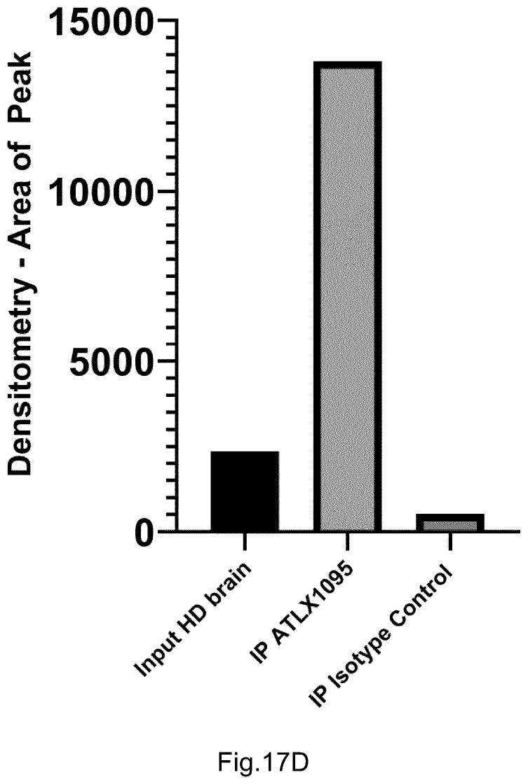

Immunoprecipitation was performed using Protein G coated Dynabeads™ and Magnetic rack (Thermofisher; 10014D). After incubation with antibody, beads were washed using magnetic rack and incubated with brain homogenate from R6/2 mice or non-transgenic (non-Tg) litter mates. Beads were again washed and then the captured protein eluted and analysed via western blot. Detection was with the anti-HTT antibodies MW8 (Merck/Millipore MABN2529) and 1C2 (MAB1574 Sigma-Aldrich). High Molecular weight HTT material was successfully immunoprecipitated by ATL_0005895, ATL_0005901 and ATL_0005567 (FIG. 17B) and ATL_0005335 (data not shown).

HTT Immunoprecipitation with Human Huntington Disease Brain

Brain homogenate was prepared from superior temporal gyrus tissue from postmortem brain of a Huntington's Disease patient. Homogenate was prepared in BLB (Brain Lysis Buffer: 10 mM Tris-HCl pH 7.4, 0.8 M NaCl, 1 mM EDTA, 10% sucrose) containing benzonase and protease inhibitors. Homogenate was centrifuged at 2,700×g and the supernatant was used for IP. Protein G coated dynabeads were coupled to isotype control antibody or ATL_0005895 and then IP carried out on brain homogenate in brain lysis buffer overnight on a rotating wheel at 4° C. On next day beads were washed in BLB then heat denatured in western blot sample buffer and analysed via western blot. Detection was with the anti-HTT antibodies HD1 and MW1 (MABN2427 Millipore).

### Phagocytosis Assay

To monitor phagocytic activity in culture, latex beads (Invitrogen, 11564067) coated with Q48HTT were labelled with pH-sensitive pHrodo™ Red Succinimidyl Ester (Thermo Fisher, P36600). Induced pluripotent stem cell (iPSC)-derived microglia (Fujifilm, C1110) were seeded at 23000 cells/well in a 96-well plate and rested for 3 days with 50% media change on day 3. On day 4, the 0.5% latex beads solution was incubated with the antibodies at 1, 2.1, 4.2, 8.3, 16.7, 33.3, 133.3, 266.7 and 333.3 (nM) and was added to microglia at 1/80 dilution. Phagocytosis was monitored by Incucyte (Sartorius) over 4 hours.

Phagocytosis of the beads induces red fluorescent signal in response to the intracellular environment with low pH. Changes in the total area of the red fluorescent signal over time are indicative of the rate of phagocytosis and the amount of bait taken up. To assess the impact of ATL_0005895 (also referred to herein as ATLX_1095) on phagocytosis, the beads-48QHtt-pHrodo™ Red complex was incubated with either ATL_0005895 or human IgG1 Isotype control antibody for 1 hour prior to exposing the cells to the antibody-treated beads-48QHtt-pHrodo™ Red complex.

### In Vivo PK Assay

To determine the serum PK of a selection of HTT antibodies, an in vivo PK experiment was performed with ATL_5567 and ATL_5901 at 10 mg/kg. Male, 6-8-week-old, C57BL6J mice received 10 mg/kg of relevant antibody by intraperitoneal injection (IP) and serum was collected 1, 4, 8, 24, 72 and 144 hours post administration. Antibody levels were assessed via an ELISA of serum samples. ATL_5895 was tested at 1, 10 and 60 mg/kg to determine serum and CSF PK. Male, 6-8-week-old, C57BL6J mice received 10 mg/kg of relevant antibody by IP injection. Serum was collected 1, 4, 8, 24, 72 and 144 hours post administration. CSF was collected 4 and 144 hours post administration.

### In Vivo PD Assay

To assess the effect ATL_5895 on HTT aggregate load in R6/1 mice, an in vivo PD experiment was performed with 60 mg/Kg ATL_5895. Mice used were mixed gender R6/1, (Jackson Laboratory Stock No: 006471). They were 5 weeks old at study start and were treated for up to 12 weeks (17 weeks old). Non-Tg littermates were used as a wild-type control. Mice were dosed once weekly via IP with 60 mg/Kg ATL_5895 or vehicle control (Histidine acetate buffer). Tissue samples were collected after 0, 4, 8 and 12-week treatments. Samples for HTT analysis were snap frozen in liquid nitrogen and stored at −80 degrees Celsius until analysis. For analysis, samples were prepared as lysates in MSD lysis buffer supplemented with NaF, PMSF, Protease Inhibitor Cocktail 1 (Mini, EDTA-free, Cat #04693159001, Roche) 2 (Sigma, Cat. #P5726) & 3 (Sigma, Cat. #P0044). Samples from striatum and cortex were then analysed for aggregated HTT via meso scale discovery (MSD) assay using the 4C9/MW8 antibody pair; Soluble mutant HTT levels were determined via MSD assay using the 2B7/MW1 antibody pair; mouse endogenous HTT levels were determined via MSD assay using the 2B7/D7F7 antibody pair. MW1 (MABN2427 Sigma), MW8 (MABN2529 Sigma), 4C9 (Coriell CH03157) and 2B7 (Coriell CH03023) are anti-HTT mouse monoclonal antibodies. D7F7 is an anti-HTT rabbit monoclonal antibody. Statistical significance was assessed using Mann Whitney test.

### Phage Display

Phage libraries of ATL_5895-derived scFv sequences were generated. These libraries comprised: (1) VH sequences that were either soft randomised in CDR3H (1105 to V117-IMGT numbering, corresponds to 93 to 102 Kabat) or hard randomised in Y103 (Y91), 1105 (I93), P106 (P94 Kabat), G114 (G100B) and L115 (L100C) (IMGT numbering, Kabat in brackets), and (2) the VL of ATL_5895, or CDR3L soft randomised variants (randomisation in G105 to V117-IMGT numbering, corresponds to 89 to 97 Kabat). Soft randomization was performed with degenerate oligonucleotides synthesized with 70-10-10-10 mixtures of nucleotide bases, with the original (ATL_5895's) nucleotide in excess. Hard randomization was performed with degenerate oligonucleotides with NNS codons. For each library, 3 rounds of phage display were performed. Phage display was performed against immobilized human HTT exon 1 48Q GST protein or biotinylated GYSLPQPQPPPPPPPPPP peptide in solution. Phage libraries were incubated with the antigen, followed by washes to removed unbound phages. Bound phages were then eluted using trypsin (selections) or IgG Elution Buffer (biotinylated peptide selections). TG1 cells were infected with the eluted phages and then plated on selective media. Colonies from rounds 2 and 3 were sequenced and phage ELISA against at least one of the antigens was performed to evaluate binding of the phage clones. Sequences with improved binding in phage ELISA relative to ATL_5895 were expressed in a IgG1 format.

### Indirect ELISA

Indirect ELISA was performed using HTT Exon-1 48Q GST. Irrelevant protein lysozyme was used as a control. Each construct was coated onto a Nunc Maxisorp plate at 5 μg/ml in PBS and incubated overnight at 4° C. Plates were blocked with PBS+3% non-fat milk powder for 1 hour at room temperature. Buffer was discarded and plates washed three times with PBS+0.1% Tween-20. An 8-point 3-fold serial dilution of test or control antibodies (starting at 60 μg/ml) were added to the wells in PBS+3% non-fat milk powder and incubated for 1 hour at room temperature. For each antigen, a well received 50 μl buffer only (blank control). The plate was incubated for 1 hour at room temperature. Buffer was discarded and plates washed three times with PBS+0.1% Tween-20. HRP-conjugated secondary antibody in PBS+2% non-fat milk powder was added to the plate and incubated for 1 hour at room temperature. Buffer was discarded and plates washed three times with PBS+0.1% Tween-20. TMB substrate was added to each well and allowed to develop for 2 minutes at room temperature. Reaction was stopped with 0.5 sulphuric acid. Absorbance at 450 nm was read for each well.

### Seeding Assay

To determine the ability of antibodies to bind to seed competent HTT species and affect HTT aggregation, the aggregation rate of FRET tagged recombinant HTT was assessed via the FRASE assay as described in Ast et al. (mHTT Seeding Activity: A Marker of Disease Progression and Neurotoxicity in Models of Huntington's Disease, Molecular Cell, Vol. 71, Issue 5, P675-688.E6, Sep. 6, 2018—incorporated herein by reference). Briefly, soluble glutathione S-transferase (GST) HTT exon-1 (HTTex1) fusion proteins with 48 glutamines C-terminally fused to CyPet or YPet (GST-Ex1Q48-CyPet or GST-Ex1Q48-YPet) were produced in E. coli BL21-CodonPlus-RPBL21-CodonPlus-RP and affinity-purified on glutathione-Sepharose beads. Purified proteins were dialyzed over night at 4° C. against 50 mM Tris-HCl PH 7.4, 150 mM NaCl, 1 mM EDTA and 5% glycerol, snap-frozen in liquid N2 and stored at −80° C.

R6/2 frozen brain tissue was cut on dry ice, weighed and homogenized in a 10-fold excess (w/v) of ice-cold 10 mM Tris-HCl PH 7.4, 0.8 M NaCl, 1 mM EDTA, 10% sucrose, 0.25 U/μl benzonase and complete protease inhibitor cocktail with a dounce homogenizer. The homogenate was incubated for 1 hour at 4° C. on a rotating wheel and centrifuged for 20 min at 2,700×g (4° C.) to remove cell debris.

The two recombinant Ex1Q48-CyPet and -Ex1Q48-YPet proteins were cleaved with PreScission protease (PSP) to release GST and to initiate the spontaneous aggregation of the fusion proteins Ex1Q48-CyPet and Ex1Q48-Ypet. This aggregation leads to a time- and concentration-dependent increase of FRET. Aggregation was tested in the presence of 10 nM fibrils produced from recombinant HTT, and 2.5 μg brain homogenates from R6/2 mice. Immunodepletion of seed with antibodies ATL_0005895 and ATL_0005901 of the disclosure, as well as MW8 (Millipore; MABN2529), and MW1 (Millipore; MABN2427) was tested. Immunodepletion used 25 μl of Protein G beads (life technologies) and 6 μg antibody.

### In Vitro Selectivity Assay

To assess selectivity of antibodies, ATL_5895, ATL_5901 and ATL_5567 were run in a screen for binding against fixed HEK293 cells expressing 6105 individual full-length human plasma membrane proteins, secreted and cell surface-tethered human secreted proteins, as well as a further 400 human heterodimers, followed by a series of confirmatory screens, all performed on the Retrogenix cell microarray platform (Charles River). HTT was not a protein on this screening panel, so was spotted as an antigen in gelatin on to the screening slide as a positive control for the fixed version of the assay.

### Live Animal PET/CT Scans to Assess Pharmacokinetics and Brain Penetration

To assess pharmacokinetics and brain penetration of ATL_5895, a PET labelled version of the antibody was prepared and live animal PET as well as gamma counting of post-mortem tissues was performed.

ATL-5895 was radiolabelled with zirconium-89 (89Zr) in a two-step procedure:
1. ATL-5895 was first conjugated to the metal chelating agent deferoxamine (DfO) using the bifunctional chelator p-SCN-Bn-DfO. Following this DfO-ATL-5895 was purified by size exclusion chromatography (SEC).
2. DfO-ATL-5895 was subsequently radiolabelled with 89Zr at room temperature and the final product, 89Zr-DfO-ATL-5895, was purified by SEC. Following this a 20 μL aliquot of 89Zr-DfO-ATL-5895 was injected onto a size exclusion HPLC system to allow for assessment of radiochemical purity and DfO-ATL5895 concentration.

Transgenic female R6/1 mice (Jackson Laboratory Stock No: 006471) and age-matched C57BL/6J controls at ages 11-12 weeks and 14-15 weeks were used. Mice were dosed with 89Zr-Df-ATL5895 100 μl, 1.5±0.3 MBq 1.15 mg/kg. PET/CT scans were performed under anaesthesia (1.5-2.5% isoflurane), static PET images were acquired at 1, 24, 48, 72, 168 hours post dosing using a Molecubes β-CUBE. Each PET scan was followed by CT scan using the Molecubes X-Cube. Image analysis was performed using PMOD software.

Blood samples were collected via capillary tail method (20 μl) at 0, 1, 6, 12, 24, 48, 72, 168 hours post dosing and assessed for 89Zr-Df-ATL5895 levels via gamma counting. Ex vivo tissue gamma counting was performed 168 hours after dosing. The Perkin Elmer Wallac Wizard Gamma counter was used for the ex-vivo organ biodistribution data.

### Manufacturability—ATL5895

Thermal stability study. ATLX_1095 (ATL_5895) expressed from CHO cells and purified in one stage at 5 mg/mL in 20 mM Histidine-acetate, 150 mM NaCl, pH 5.5 was subjected to temperatures of −80° C., +4° C., +21° C. and +40° C., for 4 weeks.

10× Freeze-thaw cycle study. ATLX_1095 (ATL_5895) at 5 mg/mL in 20 mM Histidine-acetate, 150 mM NaCl, pH 5.5 were frozen at −80° C. and thawed at room temperature (21° C.) for a minimum of 30 minutes over 10 cycles within a 24-hour period.

Protein Thermal Shift and Light Scattering. Protein thermal shift measurements were performed on an Uncle (Unchained labs) on unstressed ATLX_1095 (ATL_5895) at 5 mg/mL in 20 mM Histidine-acetate, 150 mM NaCl, pH 5.5 in triplicate. 8.8 μL of sample was loaded into three wells of a Uni (Unchained labs-propriety strip of sixteen 9 μL quartz cuvettes placed inside a blue metal frame with silicone seals). Laser settings were set to achieve an initial fluorescence in the 300-350 nm range of 10,000-50,000 counts. Antibodies were ramped from 25° C.-95° C. at a rate of 0.5° C./min and excited at 266 nm while simultaneously monitoring fluorescence emission and SLS. Melting temperature (Tm1/Tm2) and aggregation temperature (Tagg/Tonset) were analysed using Uncle Analysis software v6 (Unchained Labs). Tm measurement was calculated from the barycentric mean (BCM) of the fluorescence intensity curves from 300-430 nm while Tagg and Tonset were calculated from the intensity of light scattered at 266 nm.

CE-SDS. CE-SDS analysis was performed on the Maurice (ProteinSimple, Bio-Techne). Samples were diluted with Protein Simple 1× Sample Buffer to ~1 mg/mL and 50 μL volume. For reduced samples, 2.5 μL of 14.2M 2-Mercaptoethanol was added. The sample solutions were then transferred to a 96-well plate and centrifuged at 1000×g for 10 minutes before placing in the Maurice. The injection was performed at 4600V for 20 seconds, the separation was performed at 5750V for 25 minutes for reduced samples. Results were analysed on Compass for iCE software (Bio-Techne) and Chromeleon software (Thermo).

cIEF. cIEF analysis was performed on the Maurice (ProteinSimple, Bio-Techne). ATLX_1095 (ATL_5895) samples at 5 mg/mL in 20 mM Histidine-acetate, 150 mM NaCl, pH 5.5 were diluted to ~1 mg/mL with ultrapure water. Antibody sample was added to a master mix containing 0.35% methyl cellulose, 4% pharmalyte 3-10, 10 mM arginine, 0.01% pH 4.09 μl marker and 0.01% 9.99 μl marker to a final antibody concentration of 0.15-0.25 mg/mL. Samples separation was performed at 1500V for 1 minute, followed by 3000V for x minutes. Results were analysed on Compass for iCE software (Bio-Techne) and Chromeleon software (Thermo).

SEC-HPLC. Antibody samples were diluted to ~1 mg/ml in 20 mM histidine acetate, 150 mM NaCl pH 5.5 and filtered through a 0.22 μm filter. Approximately 25 μg of sample was loaded onto either a Zorbax GF-250 SEC-HPLC column (Agilent) or a TSKgel G3000SWxl column (TOSOH Bioscience) on a Vanquish Flex (Thermo) by injecting 25 μL of 1 mg/ml sample. Samples were isocratically eluted with 20 mM sodium phosphate, 300 mM sodium sulfate and 100 mM arginine at a flow rate of 0.75 mL/min at 25° C. for 25 minutes (Zorbax column) or 40 minutes (TSKgel column) per sample. Chromeleon software (Thermo) was used to integrate the chromatograms to determine monomeric purity.

HTT Sandwich ELISA. To assess binding potency of stressed antibody samples to HTT protein, antibodies were assessed in a sandwich ELISA format for binding to HTT exon 1 containing 48Q repeats (see Table 1 for antigen sequences). The anti-HTT capture antibody (MerckMillipore; #MABN2427) was diluted to 4.17 μg/mL in 1×ELISA coating buffer (Biolegend; #421701). 50 μL was added per well of 96 well plate, and left overnight at 4° C. The following day, the plate was washed with PBS/0.1% Tween. The plate was subsequently blocked with 50 μL/well of blocking buffer (1% BSA/PBS). The plate was washed with PBS/0.1% Tween. 50 μl of diluted antigen (HTT exon1 48Q GST) and lysozyme (negative antigen control) were added to the plate at 0.04 μg/ml. The plate was incubated for 1 hr at RT on a plate shaker (300-400 rpm). The plate was washed with PBS/0.1% Tween. An 8-point 3-fold serial dilution of test antibody and isotype control antibody (starting at 60 μg/mL) was added to the plate. The plate was incubated for 1 hr at RT on a plate shaker (300-400 rpm). The plate was washed with PBS/0.1% Tween. 50 μl of anti-Human IgG HRP was 10 added per well (80 ng/ml; Jackson ImmunoResearch, #109-035-097). The plate was washed with PBS/0.1% Tween, 50 μL of TMB solution (Lifetechnology, #002023) was added and the plate was incubated for 6-9 minutes at room temperature in dark. 50 μL of stopping solution (0.5 M sulphuric acid) added to plate (Fisher chemical, #12933634). Absorbance was read on CLARIO Star at 450 nm. Data analysis performed using GraphPad Prism 10 Software (10.1.0.316) EC50 (the half maximal concentration) values were calculated using a non-linear, four parameter curve fitting, with no constraints.

Production of ATL_5895 for Solubility Assessment. Recombinant antibody ATL_5895 was expressed transiently from ExpiCHO-S cells according to the manufacturers protocol using Expifectamine reagent (Thermo). Transfections were cultured for 13 days at 32° C. with addition of feeds, followed by harvesting by removing cells and mixing supernatant with diatomaceous earth (Sartorius) and filtration through a 0.22 μm PES membrane. Harvested supernatant was purified using protein A chromatography, eluted with 50 mM sodium acetate pH 3.6. Elution fractions were pooled and buffer exchanged into 20 mM Histidine acetate, 150 mM sodium chloride pH 5.5 and stored at 4° C., followed by −80° C. long term storage.

Solubility Assessment. Approximately 100 mL of ATL_5895 at 11.90 mg/mL was concentrated to a maximum concentration of 89.96 mg/mL by tangential flow filtration (TFF) using a Minimate EVO Tangential Flow Filtration System (Pall/Cytiva, PCode: OAPMPUNV) and a Minimate TFF Capsule 30K Omega membrane (Pall/Cytiva, PCode: OA030C12). 100 μL samples were retrieved from the sample reservoir at 3 time points. Sample concentration was determined by absorbance at 280 nm by UV/Vis spectrophotometry on the Lunatic (Unchained labs).

Samples were assessed for aggregation by SEC-HPLC. Antibody samples were diluted to ~1 mg/ml in 20 mM histidine acetate, 150 mM NaCl pH 5.5 and filtered through a 0.22 μm filter. Approximately 25 μg of sample was loaded onto a TSKgel G3000SWxl column (TOSOH Bioscience) on a Vanquish Flex (Thermo) by injecting 25 μL of 1 mg/ml sample. Samples were isocratically eluted with 20 mM sodium phosphate, 300 mM sodium sulfate and 100 mM arginine at a flow rate of 0.75 mL/min at 25° C. for 25 minutes (Zorbax column) or 40 minutes (TSKgel column) per sample. Chromeleon software (Thermo) was used to integrate the chromatograms to determine monomeric purity.

### Murine HTT ELISA

To assess binding potency of antibodies to murine HTT protein, ATL5895, ATL6376 and ATL6377 were assessed in a direct ELISA format for binding to murine HTT and human lysozyme (as negative control antigen). Murine HTT antigen (SEQ ID NO: 177) or lysozyme were directly absorbed to ELISA plate at 3 μg/ml (50 μl per well) and incubated overnight at 4 C. The plate was washed with PBS. Plates were blocked with 200 μl/well of blocking solution (1% BSA w/v in PBS) for 1 hour at room temperature. Following this, the blocking solution was removed and antibodies to be assessed were diluted in dilution series (1 μM to 0.05 nM) in blocking solution (1% BSA w/v in PBS) and applied to the plate. Plates were incubated at room temperature for 1 hour. The plate was washed with PBS/0.1% Tween. Anti-Human IgG HRP (Jackson ImmunoResearch; #109-035-097) was added to plate and incubated for 1 hour at room temperature to detect antibody binding. The plate was washed with PBS/0.1% Tween and TMB solution (Life Technology; #002023) added. Plates were incubated for 5 minutes at room temperature prior to the addition of stopping solution (0.5M sulphuric acid). Absorbance read on Molecular Devices FilterMaxF5 plate reader at 450 nm. Analysis was performed using a non-linear fit curve fitting algorithm on GraphPad prism and EC50 calculated.

### Example 1-Convergence Analysis of AD Cohort to Identify VH Sequences Associated with Resilience

Convergent sequence clusters derived from the antibody repertoire of resilient groups of individuals can be used to identify disease-specific antibody sequences. In the case of neurodegeneration, resilience can be defined as a long-term absence of symptoms despite having a strong disease predisposition.

Candidate protective antibodies were identified from a resilient sub-group of at-risk patients from a cohort of patients at risk of dementia using cognitive scores and biomarkers (in collaboration with European Prevention of Alzheimer's Dementia (EPAD)). FIG. 1A shows the workflow used in the present example for identifying convergent VH sequences from the AD dataset. Resilience was defined as those patients at risk of Alzheimer's Disease (AD) with significantly reduced β-amyloid in their cerebrospinal fluid (CSF) compared with healthy controls, which correlates strongly with increased B-amyloid deposition in the brain. This subgroup of patients also had low levels of pTau in CSF, which indicates significantly less neuronal damage compared with AD progressors, and demonstrated continued normal cognitive function compared with age-matched progressing individuals with adverse B-amyloid and p-Tau CSF biomarkers.

The present inventors identified one cluster of related antibody heavy chains that was convergent across resilient individuals, but not present in control individuals (FIG. 1B). On FIG. 1B, the sequence labelled as “Known HTT Binder” is the sequence labelled herein as ATL_0005059, also referred to as NI-302.8F1 and described in U.S. Pat. No. 11,401,325 B2. It is important to note that the antibodies described herein were identified through a process that is independent of this prior antibody. Indeed, the present antibodies were identified as related antibody heavy chains that are convergent across individuals that are resilient to neurodegenerative diseases. These antibodies were identified to bind HTT and were therefore aligned to the known antibody for context. In other words, the presently described antibodies were not developed by adaptation of an existing antibody. Rather, the presently described antibodies were discovered by analysis of naturally occurring protective (and therefore likely therapeutically effective) antibodies and optimisation thereof, wherein identification was completely target and sequence agnostic, and optimisation was completely independent of the prior art antibodies and instead further improved on the already superior antibodies derived from the naturally occurring sequences. These antibodies showed signatures of immune activation, supporting their role in resilience. To deconvolute the target of this cluster of VHs, they were compared with a database of antibodies with known binding specificity curated from the literature. One VH in the cluster had an identical CDR3 amino acid sequence to a known binder of Huntingtin, a protein encoded by the Htt gene which is strongly linked to Huntington's Disease. This finding indicated that the convergent VHs in the cluster may also bind HTT protein.

Two of these VHs (ATL5060 and ATL5061-see sequences in Table 1 and FIG. 1B) were expressed as antibodies using the light chain (VL) from the known binder. Both antibodies were confirmed for binding to HTT by ELISA.

The discovery of antibodies to HTT in resilient AD ‘at risk’ patients could be considered unexpected. Aggregated and mutated HTT (mHTT) protein is reported as the main cause of Huntington's Disease, however, presence of mutated or aggregated protein has been associated with other neurodegenerative diseases. HTT aggregates accumulate in the cytoplasm of dystrophic neurons and in microglia in AD brains (Singhrao, S et al. Huntingtin Protein Colocalizes with Lesions of Neurodegenerative Diseases: An Investigation in Huntington's, Alzheimer's, and Pick's Diseases. Exp Neurol 150, 213 (1998)) as well as neurons of the hippocampus and pre-frontal cortex in a separate AD study (Axenhus, M, et al. Huntingtin Levels are Elevated in Hippocampal Post-Mortem Samples of Alzheimer's Disease Brain. Curr Alzheimer Res 17, 858 (2020)). Accumulation of HTT is associated with the formation of tau fibrils and tangles in both HD and AD (Masnata, M, et al. Targeting Tau to Treat Clinical Features of Huntington's Disease. Front Neurol 11, 580732 (2020)). Moreover, a small percentage of Frontotemporal dementia (FTD) and amyotrophic lateral sclerosis (ALS) patients was also reported to have a CAG codon expansion in Htt (CAG>40) (at a rate 4.4 times higher than in healthy individuals) (Dewan, R. et al. Pathogenic Huntingtin Repeat Expansions in Patients with Frontotemporal Dementia and Amyotrophic Lateral Sclerosis. Neuron 109, 448 (2021)). Additionally, it has been shown that mHTT polyglutamine expression increases the seeding properties of aggregated TDP-43 in a cellular model (Coudert, L. et al. Phosphorylated and aggregated TDP-43 with seeding properties are induced upon mutant Huntingtin (mHtt) polyglutamine expression in human cellular models. Cell Mol Life Sci 76:2615 (2019)). HTT dysfunction may therefore play an aetiological role in a broader set of neurodegenerative diseases than previously thought and at earlier stages of pathology than Tau, beta amyloid or TDP-43 mediated changes.

In addition to the two individuals from the EPAD cohort, homologs were found in another individual with AD from a separate cohort, and in two individuals who were genetically predisposed to, but expressing resilience towards FTD (data not shown). This further supports the role of mHTT in various neurodegenerative conditions.

### Example 2—Phage Display to Derive Optimal VJ/VL Pairing

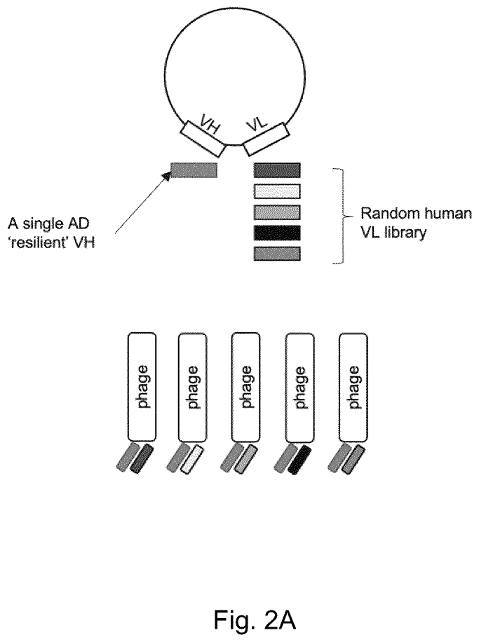

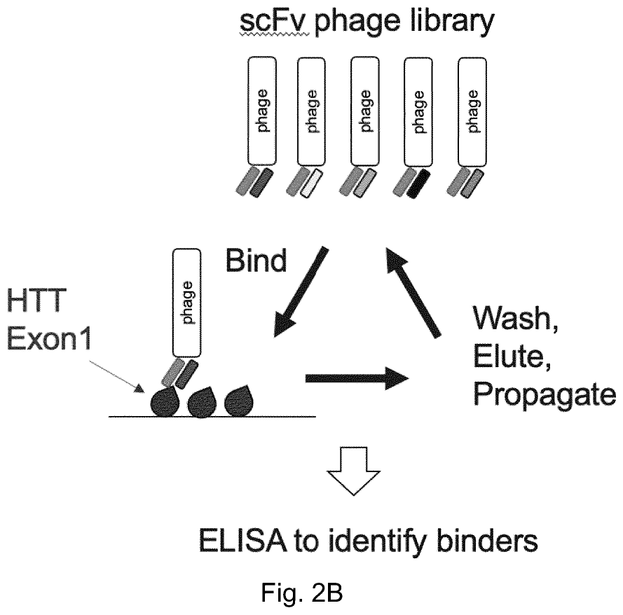

Building on the discovery of the antibody ATL_5060, combinatorial phage display was used to identify the optimal VL partners for the VHs. The VH sequence from ATL_5060 was combined with >1 million potential VL partners in a phage display library before selecting on the HTT protein to enrich for functional binders. This process is illustrated in FIGS. 2A and B.

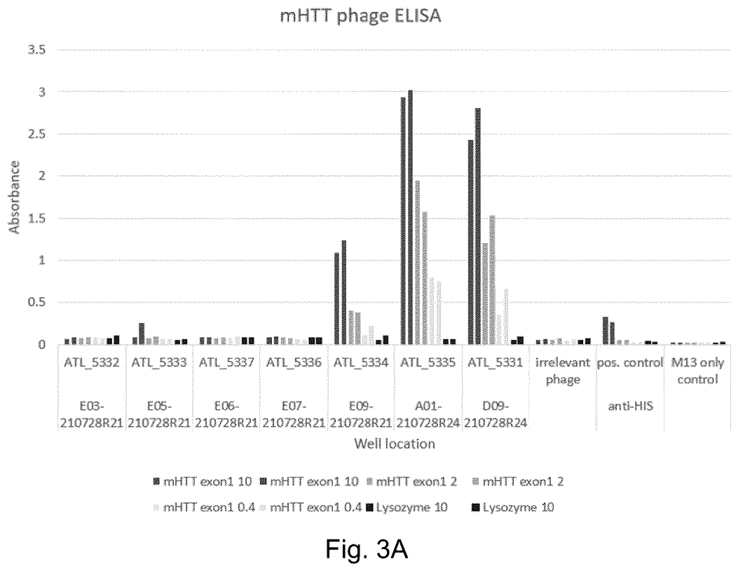

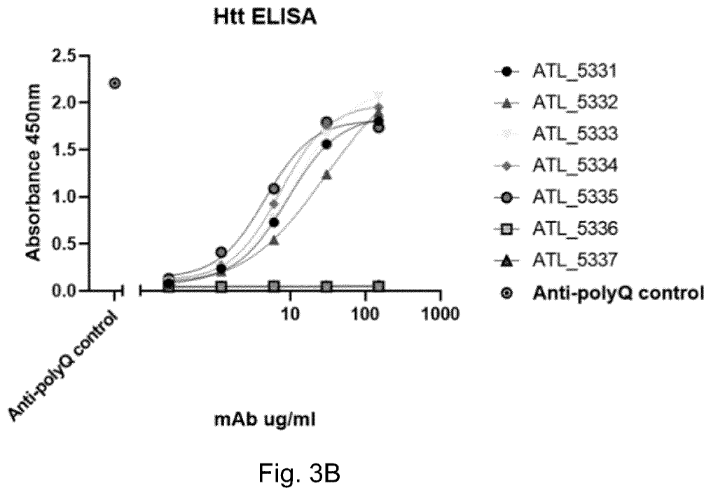

Phage selections resulted in seven unique scFv sequences (labelled ATL_0005331 to 5337) and mHTT phage ELISA suggested two strong binders (ATL_5331; ATL_5335) and one moderate binder (ATL_5334) (FIG. 3A). Following phage display enrichment, the resulting human IgG1 antibodies containing selected VL sequences were prepared and tested for binding to HTT protein by ELISA (FIG. 3B). The two assays shown on FIGS. 3A and 3B are both indirect ELISAs to measure binding to mHTT coated on a plate. However, the test sample is different between A and B. In A the test samples are phage displaying antibody fragments corresponding to the antibody sequences selected. In B the test samples are the antibody sequences formatted as human IgG. The experiment shown in A is a screen where higher concentrations of mHTT are used to ensure detection of levels of binding. The experiment shown in B is the determination of the binding equilibrium where the concentration of mHTT used gives increased sensitivity. The minimal differences in absorbance observed in A can be due to the variability of the phage in each test sample, including different display levels of the antibody fragments and different concentrations of phage in the test samples applied to the experiment, which results in variable concentrations of antibody fragments in the test samples. By contrast, in B, there is little variability in the amount of antibody used in the test sample, as this can be easily measured based on protein concentration.

ATL_5331, ATL_5334, and ATL_5335 were selected for further optimisation.

FIG. 4A shows the convergent VH sequence as identified in AD-resilient individuals (ATL_0005042). FIG. 4B shows the functionally paired VL (ATL_0005331-5335), aligned to CA_0000274 VL (also referred to herein as ATL_0005059 or NI-302.8F1 and described in U.S. Pat. No. 11,401,325 B2) from the phage display selections on HTT Exon 1 and sequence analysis.

### Example 3—Epitope Mapping

To assess the epitopes of the antibodies to HTT protein (ATL_5331; ATL_5566 (=ATL_5334 with free cysteines removed), and ATL_5335), antibodies were assessed against a peptide array constructed from peptide fragments corresponding to the sequences of human, cynomolgus monkey (cyno, 2 different referent sequences that are the likely cynomolgus monkey HTT protein sequence) and mouse HTT exon 1.

The peptide array consisted of 15mer linear peptides with 14 amino acid overlap across HTT exon 1 (see FIG. 5) allowing for high resolution epitope mapping. Species cross-reactivity was tested in parallel using two cynomolgus isoforms and one mouse isoform for HTT exon 1.

Table 2 shows the sequences used to generate peptides for the array.

TABLE 2
Reference sequences used to generate peptides.

| | Sequence |
|---|---|
| >Human HTT exon 1 | MATLEKLMKAFESLKSFQQQQQQQQQQQQQQQQPPPPPPPP |
| | PPPQLPQPPPQAQPLLPQPQPPPPPPPPPPGPAVAEEPLHR |
| | (SEQ ID NO: 49) |
| >Cyno HTT exon 1 (Cyno 1) | MATLEKLMKAFESLKSFQQQQQQQQQQQPPPPPPPPPPPP |
| | PQPPQAQPMLPQPQPPPPPPRHHPARLWLRSRCTDQRKNFQL |
| | PRKTV (SEQ ID NO: 50) |
| >Cyno HTT exon 1 (Cyno 2) | MATLEKLMKAFESLKSFQQQQQQQQQQQQPPPPPPPPPPPP |
| | LPQPPQAQPMLPQPQPPPPPPPPPPGPAVAEEPLHRPKKELSA |
| | TKKDRVNH (SEQ ID NO: 51) |
| >Mouse HTT exon 1 | MATLEKLMKAFESLKSFQQQQQQQPPPQAPPPPPPPPPPQPP |
| | QPPPQGQPPPPPPPLPGPAEEPLHR (SEQ ID NO: 52) |

The resulting huntingtin peptide microarrays contained HTT peptides printed in duplicate and were framed by additional HA (YPYDVPDYAG, 48 spots) and polio (KEVPALTAVETGAT, 48 spots) control peptides. The huntingtin peptide microarrays were incubated with the antibody samples at concentrations of 1 μg/ml and 10 μg/ml followed by staining with secondary (Goat anti-human IgG (H+L) DyLight680 at 0.2 μg/ml) and control antibodies (Mouse monoclonal anti-HA (12CA5) DyLight800 at 0.2 μg/ml) as well as read-out with an Innopsys InnoScan 710-IR Microarray Scanner. Quantification of spot intensities and peptide annotation were done with PepSlide® Analyzer. Briefly, quantification of spot intensities and peptide annotation were based on the 16-bit gray scale tiff files. Microarray image analysis was done with PepSlide® Analyzer. Fluorescence intensities of each spot were broken down into raw, foreground and background signal, and median foreground intensities (referred to as “corrected intensity”) and spot-to-spot deviations of spot duplicates calculated. Maximum spot-to-spot deviation of 40% was permitted otherwise the corresponding intensity value was zeroed. Plots of averaged spot intensities for the assays with the human antibody samples against the antigen sequences from the N-terminus of human huntingtin protein to the C-terminus of mouse huntingtin protein were used to visualize overall spot intensities. The intensity plots were correlated with peptide and intensity maps as well as with visual inspection of the microarray scans to identify the epitopes of the antibody samples.

FIGS. 6 to 8 show the binding intensity of the three selected monoclonal antibodies (mAbs) and to regions of HTT exon 1. All mAbs showed similar binding profiles across different species, and binding was primarily observed at two distinct sites of HTT exon 1. Alignment revealed the consensus motif of the main antibody responses (indicated in bold letters in FIGS. 6 to 8 (lower panels)). The consensus motif for binding shared between binding sites on exon 1 was identified as [P/Q]Q[P/Q]QPPPPPPPPPPP (SEQ ID NO: 113).

Table 3 is a summary table of peptides with binding detected for at least one of the concentrations of antibodies.

TABLE 3
Summary table of peptides with binding detected to HTT Exon 1 peptides.

| | | Human | Human | Human | Human | Human | Human |
|---|---|---|---|---|---|---|---|
| | | IgG1 | IgG1 | IgG1 | IgG1 | IgG1 | IgG1 |
| | | ATL_ | ATL_ | ATL_ | ATL_ | ATL_ | ATL_ |
| | | 0005331, | 0005331, | 0005335, | 0005335, | 0005566, | 0005566, |
| Peptide | Sp | 1 μg/ml | 10 μg/ml | 1 μg/ml | 10 μg/ml | 1 μg/ml | 10 μg/ml |
| QQQQQQQQPPPPPPP | H | 131 | 0 | 0 | 729 | 0 | 470.75 |
| (SEQ ID NO: 54) | | | | | | | |
| QQQQQQQPPPPPPPP | H | 197 | 1344 | 416.75 | 2099.75 | 0 | 1787.5 |
| (SEQ ID NO: 55) | | | | | | | |
| QQQQQQPPPPPPPPP | H | 275.5 | 1671.25 | 625.5 | 3191.75 | 200.75 | 2785.25 |
| (SEQ ID NO: 56) | | | | | | | |
| QQQQQPPPPPPPPPP | H | 237.75 | 2616 | 409.25 | 3677.25 | 119.5 | 3074 |
| (SEQ ID NO: 57) | | | | | | | |
| QQQQPPPPPPPPPPP | H | 291.25 | 2949.75 | 292.75 | 3836.5 | 227.75 | 3040 |
| (SEQ ID NO: 47) | | | | | | | |
| QQQPPPPPPPPPPPQ | H | 0 | 382.5 | 0 | 592.75 | 0 | 0 |
| (SEQ ID NO: 58) | | | | | | | |
| QQPPPPPPPPPPPQL | H | 0 | 397.25 | 0 | 176 | 0 | 0 |
| (SEQ ID NO: 59) | | | | | | | |
| PQAQPLLPQPQPPPP | H | 0 | 119.25 | 0 | 326 | 18.5 | 231.75 |
| (SEQ ID NO: 60) | | | | | | | |
| QAQPLLPQPQPPPPP | H | 259.75 | 1137.5 | 434 | 1511 | 269.75 | 996.75 |
| (SEQ ID NO: 61) | | | | | | | |
| AQPLLPQPQPPPPPP | H | 229.25 | 1264.25 | 621.5 | 3471.25 | 157.25 | 1243 |
| (SEQ ID NO: 62) | | | | | | | |
| QPLLPQPQPPPPPPP | H | 273.5 | 2248.25 | 844.75 | 5414.5 | 351.75 | 2238.5 |
| (SEQ ID NO: 63) | | | | | | | |
| PLLPQPQPPPPPPPP | H | 279.5 | 2947.5 | 936.5 | 6684 | 453.75 | 2709.5 |
| (SEQ ID NO: 64) | | | | | | | |
| LLPQPQPPPPPPPPP | H | 373.5 | 3491.75 | 932 | 8523.5 | 398.25 | 3381 |
| (SEQ ID NO: 65) | | | | | | | |
| LPQPQPPPPPPPPPP | H | 372.75 | 3963.5 | 1129.5 | 9012.75 | 475.5 | 3836.75 |
| (SEQ ID NO: 66) | | | | | | | |
| PQPQPPPPPPPPPPG | H | 84 | 411.5 | 335.25 | 1629 | 0 | 512 |
| (SEQ ID NO: 67) | | | | | | | |
| QPQPPPPPPPPPPGP | H | 134.5 | 381.5 | 233.5 | 1126 | 0 | 554 |
| (SEQ ID NO: 68) | | | | | | | |
| PQPPPPPPPPPPGPA | H | 90.25 | 145.25 | 77.5 | 415.25 | 0 | 116 |
| (SEQ ID NO: 69) | | | | | | | |
| QPPPPPPPPPPGPAV | H | 85.5 | 16.25 | 0 | 166.25 | 0 | 0 |
| (SEQ ID NO: 70) | | | | | | | |
| QQQQQQQQPPPPPPP | C1 | 0 | 378 | 381 | 0 | 0 | 1489 |
| (SEQ ID NO: 54) | | | | | | | |
| QQQQQQQPPPPPPPP | C1 | 118.5 | 1762 | 666.75 | 2230.25 | 61.5 | 2481.5 |
| (SEQ ID NO: 55) | | | | | | | |
| QQQQQQPPPPPPPPP | C1 | 206.25 | 2289.5 | 520.5 | 3541.75 | 207 | 4895.75 |
| (SEQ ID NO: 56) | | | | | | | |
| QQQQQPPPPPPPPPP | C1 | 136 | 3154.75 | 535.5 | 4317.25 | 358 | 4306.5 |
| (SEQ ID NO: 57) | | | | | | | |
| QQQQPPPPPPPPPPP | C1 | 195 | 4157.25 | 390.75 | 3341.5 | 354 | 7481.25 |
| (SEQ ID NO: 47) | | | | | | | |
| QQQPPPPPPPPPPPP | C1 | 137.25 | 3100 | 474.5 | 4507.5 | 347.5 | 4783 |
| (SEQ ID NO: 71) | | | | | | | |
| PQAQPMLPQPQPPPP | C1 | | | 79 | 452.75 | 0 | 250 |
| (SEQ ID NO: 72) | | | | | | | |
| QAQPMLPQPQPPPPP | C1 | 178.25 | 503.5 | 186.75 | 1192.5 | 108 | 1109 |
| (SEQ ID NO: 73) | | | | | | | |
| AQPMLPQPQPPPPPP | C1 | 182 | 1033.5 | 443 | 2745 | 103 | 1527.75 |
| (SEQ ID NO: 74) | | | | | | | |
| QPMLPQPQPPPPPPR | C1 | 91 | 112 | 82 | 374 | 3.5 | 86 |
| (SEQ ID NO: 75) | | | | | | | |
| QQQQQQQQQQPPPPP | C2 | 0 | 148.5 | 0 | 524.25 | 0 | 0 |
| (SEQ ID NO: 76) | | | | | | | |
| QQQQQQQQQPPPPPP | C2 | 145.5 | 994.25 | 562.5 | 3260 | 127.5 | 1255.5 |
| (SEQ ID NO: 77) | | | | | | | |
| QQQQQQQQPPPPPPP | C2 | 259.25 | 2698 | 990.5 | 7008 | 274 | 3189.75 |
| (SEQ ID NO: 54) | | | | | | | |
| QQQQQQQPPPPPPPP | C2 | 461.5 | 4074.5 | 1158.25 | 8866.75 | 436.5 | 4731.5 |
| (SEQ ID NO: 55) | | | | | | | |
| QQQQQQPPPPPPPPP | C2 | 548.5 | 4825.5 | 1220.25 | 9511.5 | 649 | 5352 |
| (SEQ ID NO: 56) | | | | | | | |
| QQQQQPPPPPPPPPP | C2 | 545.75 | 4866 | 1108.5 | 10012.25 | 583.75 | 5895.5 |
| (SEQ ID NO: 57) | | | | | | | |
| QQQQPPPPPPPPPPP | C2 | 379 | 4579 | 1400.75 | 9591 | 432.75 | 4588.5 |
| (SEQ ID NO: 47) | | | | | | | |
| QQQPPPPPPPPPPPP | C2 | 474 | 4498.25 | 1274 | 8368.75 | 478.75 | 5358 |
| (SEQ ID NO: 71) | | | | | | | |
| QQPPPPPPPPPPPP | C2 | 206.25 | 946.75 | 506.75 | 3295.25 | 96 | 577 |
| (SEQ ID NO: 78) | | | | | | | |
| QPPPPPPPPPPPP | C2 | 141.25 | 1035.5 | 127.5 | 1907.5 | 10.75 | 528.75 |
| (SEQ ID NO: 79) | | | | | | | |
| PPPPPPPPPPPPPP | C2 | 99.25 | 321 | 0 | 604.5 | 0 | 0 |
| (SEQ ID NO: 80) | | | | | | | |
| QAQPMLPQPQPPPPP | C2 | 0 | 0 | 0 | 87 | 0 | 0 |
| (SEQ ID NO: 73) | | | | | | | |
| QAQPMLPQPQPPPPP | C2 | 51.5 | 0 | 0 | 0 | 0 | 187.25 |
| (SEQ ID NO: 73) | | | | | | | |
| AQPMLPQPQPPPPPP | C2 | 153.5 | 887.75 | 168 | 447.5 | 155.5 | 1253.5 |
| (SEQ ID NO: 74) | | | | | | | |
| QPMLPQPQPPPPPPP | C2 | 152.25 | 1705 | 421.25 | 2674 | 183.5 | 1405 |
| (SEQ ID NO: 81) | | | | | | | |
| PMLPQPQPPPPPPPP | C2 | 155.75 | 2174 | 522.75 | 4677.25 | 180.5 | 2122 |
| (SEQ ID NO: 82) | | | | | | | |
| MLPQPQPPPPPPPPP | C2 | 255.5 | 4248.25 | 772 | 6015.5 | 337 | 2510.5 |
| (SEQ ID NO: 83) | | | | | | | |
| LPQPQPPPPPPPPPP | C2 | 294 | 5483 | 1000.5 | 7744.5 | 446 | 2404.5 |
| (SEQ IS NO: 84) | | | | | | | |
| PQPQPPPPPPPPPPG | C2 | 45.5 | 463.5 | 0 | 0 | 83 | 0 |
| (SEQ ID NO: 67) | | | | | | | |
| QPQPPPPPPPPPPGP | C2 | 50 | 478.5 | 0 | 0 | | |
| (SEQ ID NO: 68) | | | | | | | |
| QQQQQPPPQAPPPPP | M | 0 | 0 | 0 | 286 | 0 | 246.75 |
| (SEQ ID NO: 85) | | | | | | | |
| QQQQPPPQAPPPPPP | M | 0 | 633.25 | 599.75 | 3088.5 | 163 | 1296.25 |
| (SEQ ID NO: 86) | | | | | | | |
| QQQPPPQAPPPPPPP | M | 161.5 | 1543 | 753.5 | 5804.75 | 197 | 2393.25 |
| (SEQ ID NO: 87) | | | | | | | |
| QQPPPQAPPPPPPPP | M | 180.75 | 2409.25 | 757.5 | 7816 | 214.5 | 3498.25 |
| (SEQ ID NO: 88) | | | | | | | |
| QPPPQAPPPPPPPPP | M | 190.5 | 2742.25 | 778.25 | 8300.25 | 314.25 | 3956.5 |
| (SEQ ID NO: 89) | | | | | | | |
| PPPQAPPPPPPPPPP | M | 179.5 | 3165 | 741.5 | 7372 | 316 | 4083 |
| (SEQ ID NO: 90) | | | | | | | |
| PPQAPPPPPPPPPPQ | M | 0 | 825.25 | 194.5 | 3521.25 | 3.75 | 1015.5 |
| (SEQ ID NO: 91) | | | | | | | |
| PQAPPPPPPPPPPQP | M | 0 | 841.5 | 0 | 1883.5 | 0 | 0 |
| (SEQ ID NO: 92) | | | | | | | |
| QAPPPPPPPPPPQPP | M | 0 | 233.75 | 0 | 0 | 0 | 0 |
| (SEQ ID NO: 93) | | | | | | | |
| PPQPPPQGQPPPPPP | M | 55 | 197 | 255.5 | 0 | 0 | 287.25 |
| (SEQ ID NO: 94) | | | | | | | |
| PQPPPQGQPPPPPPP | M | 41.5 | 884.5 | 612.5 | 2275 | 97.75 | 449 |
| (SEQ ID NO: 95) | | | | | | | |
| QPPPQGQPPPPPPPL | M | 14.5 | 997.25 | 120 | 899.25 | 0 | 219 |
| (SEQ ID NO: 96) | | | | | | | |
| PPPQGQPPPPPPPLP | M | 0 | 1745 | 143 | 1250.5 | 0 | 0 |
| (SEQ ID NO: 97) | | | | | | | |

Sp = species, H = human, M = Mouse, C1 = cyno_1, C2 = cyno_2. Data = corrected fluorescence intensities.

In conclusion, epitope mapping suggests that the three selected antibodies have a complex binding site in HTT exon 1 and bind preferentially to peptides with multiple C-terminal prolines.

### Example 4—Stability Study

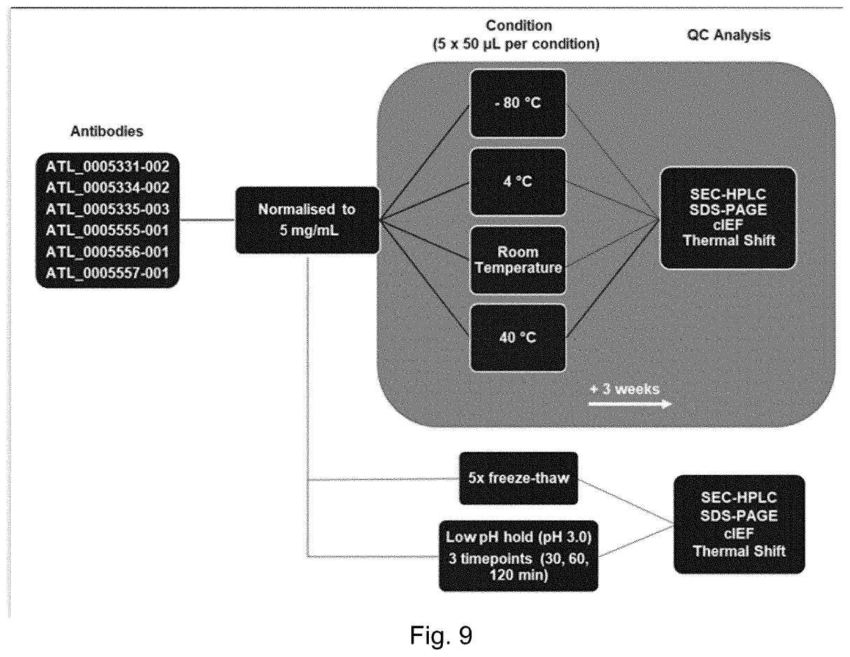

To assess the stability profile of the HTT exon-1 mAbs, a 3-week accelerated stability study was carried out on the three selected mAbs described above and their fully germlined equivalents: ATL_5331; ATL_5334; ATL_5335; ATL_5555 (ATL_5331 fully germlined); ATL_5556 (ATL_5334 fully germlined); and ATL_5557 (ATL_5335 fully germlined). The workflow for the stability study is illustrated in FIG. 9.

The antibodies were stored at −80° C., 4° C., room temperature, or 40° C. for three weeks and a sample was subjected to five repetitive freeze thaws from −80 C to room temperature. A quality control analysis followed including the assessment of purity, aggregation, degradation, charge variants and thermostability.

Protein aggregation during antibody storage must be kept to a minimum as it can cause immunogenic reactions. Size exclusion chromatography (SEC)-HPLC was used to evaluate the purity and aggregation of the antibodies in the 3-week stability study as well as those subjected to a 5× freeze thaw cycle.

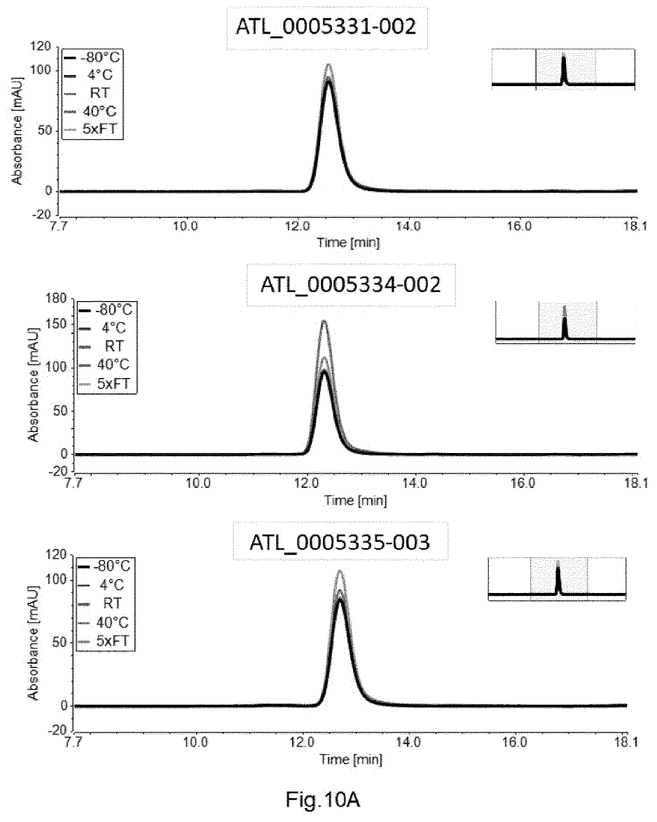

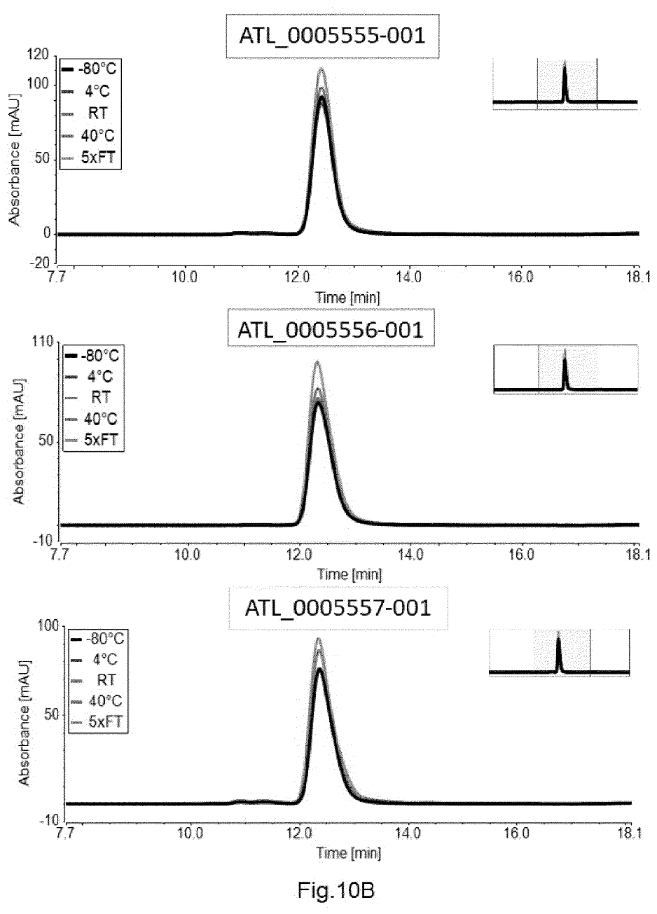

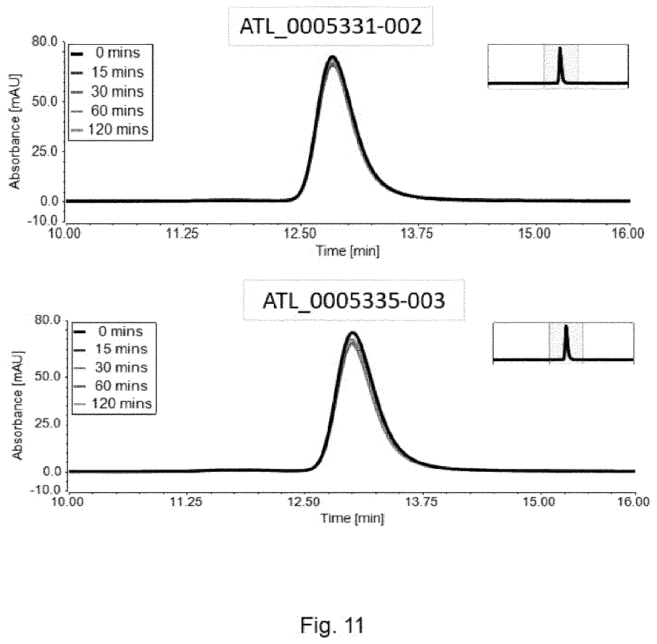

FIG. 10 shows that no soluble aggregates form following incubation at −80° C., 4° C., room temperature (21° C.) and 40° C. for 3 weeks (SEC-HPLC). Table 4 further supports the excellent stability of the 6 tested antibodies and shows low levels of high molecular weight species (HMWS) and low levels of low molecular weight species (LMWS) indicating that there is low aggregation and degradation propensity of the antibodies up to 40° C., which is further supported by the high percentage of monomers in each of the samples >95% throughout the study.

TABLE 4
Results from SEC-HPLC.

| Antibody | Condition | | % HMWS | % Monomer | % LMWS |
|---|---|---|---|---|---|
| ATL_0005331 | −80° | C. | 0.5 | 99.35 | 0.15 |
| | 4° | C. | 0.5 | 99.31 | 0.19 |
| | 21° | C. | 0.48 | 99.26 | 0.27 |
| | 40° | C. | 0.49 | 99.76 | 0.75 |
| | 5XFT | | 0.5 | 99.33 | 0.17 |
| ATL_0005334 | −80° | C. | 0.62 | 98.91 | 0.47 |
| | 4° | C. | 0.61 | 98.92 | 0.47 |
| | 21° | C. | 0.60 | 98.89 | 0.51 |
| | 40° | C. | 0.69 | 98.21 | 1.10 |
| | 5XFT | | 0.63 | 98.91 | 0.46 |
| ATL_0005335 | −80° | C. | 1.15 | 98.85 | N/A |
| | 4° | C. | 1.17 | 98.83 | N/A |
| | 21° | C. | 1.25 | 98.54 | 0.21 |
| | 40° | C. | 1.17 | 97.91 | 0.91 |
| | 5XFT | | 1.86 | 98.14 | N/A |
| ATL_0005555 | −80° | C. | 1.77 | 98.23 | N/A |
| | 4° | C. | 1.79 | 98.21 | N/A |
| | 21° | C. | 1.74 | 98.06 | 0.20 |
| | 40° | C. | 1.66 | 97.32 | 1.02 |
| | 5XFT | | 1.85 | 98.15 | N/A |
| ATL_0005556 | −80° | C. | 0.35 | 99.65 | N/A |
| | 4° | C. | 0.37 | 99.63 | N/A |
| | 21° | C. | 0.48 | 99.05 | 0.47 |
| | 40° | C. | 0.48 | 98.39 | 1.13 |
| | 5XFT | | 0.46 | 99.54 | N/A |
| ATL_0005557 | −80° | C. | 2.43 | 97.57 | N/A |
| | 4° | C. | 2.49 | 97.51 | N/A |
| | 21° | C. | 2.65 | 97.09 | 0.25 |
| | 40° | C. | 2.19 | 96.59 | 1.10 |

RT = Room temperature 21° C.; FT = freeze-thaw; HMWS = high molecular weight species; LWMS = low molecular weight species.

No detectable soluble aggregates were formed for antibodies ATL_5331 or ATL_5335 following a low pH hold step (at pH 3.5) for up to 120 minutes (SEC-HPLC) (FIG. 11 and table 5).

TABLE 5
Low pH hold SEC-HPLC results

| Sample | Low pH hold timepoint (min) | % Monomer | % HMWS |
|---|---|---|---|
| ATL_0005331 | 0 | 98.93 | 0.68 |
| | 15 | 98.58 | 0.92 |
| | 30 | 98.97 | 0.8 |
| | 60 | 98.51 | 0.91 |
| | 120 | 98.77 | 0.69 |
| ATL_0005335 | 0 | 98.19 | 1.56 |
| | 15 | 98.12 | 1.7 |
| | 30 | 97.87 | 1.84 |
| | 60 | 98.42 | 1.38 |
| | 120 | 98.10 | 1.66 |

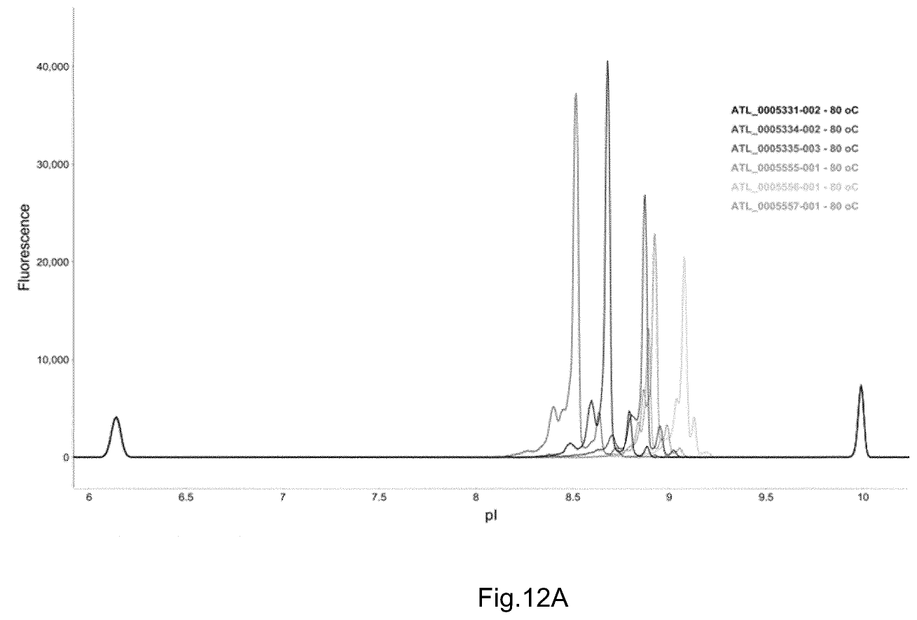

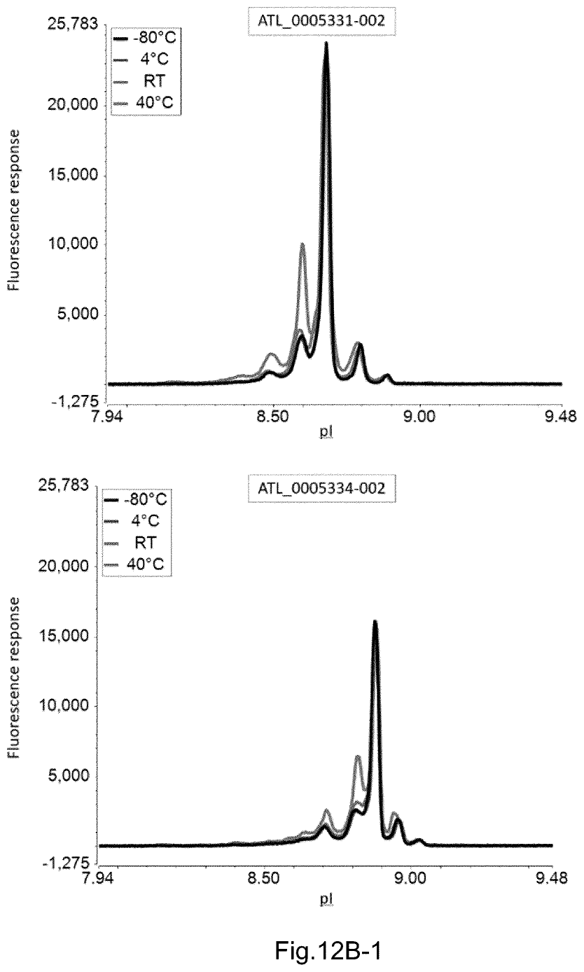

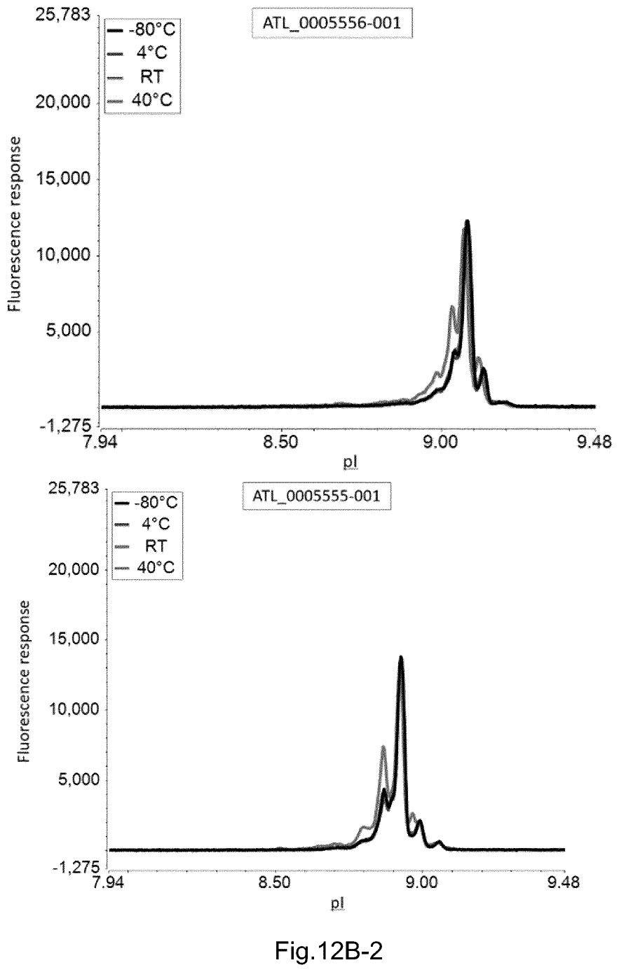

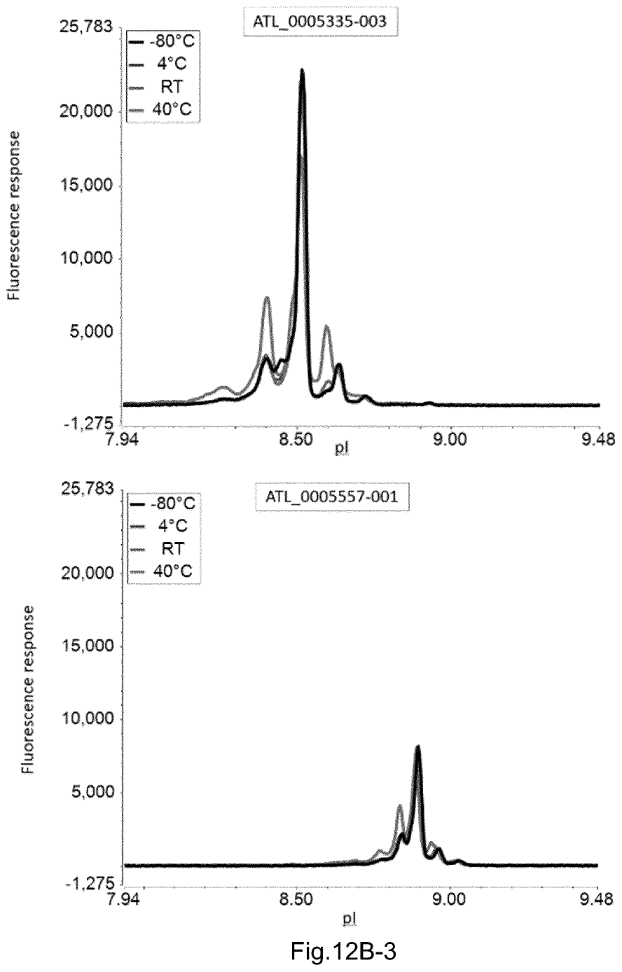

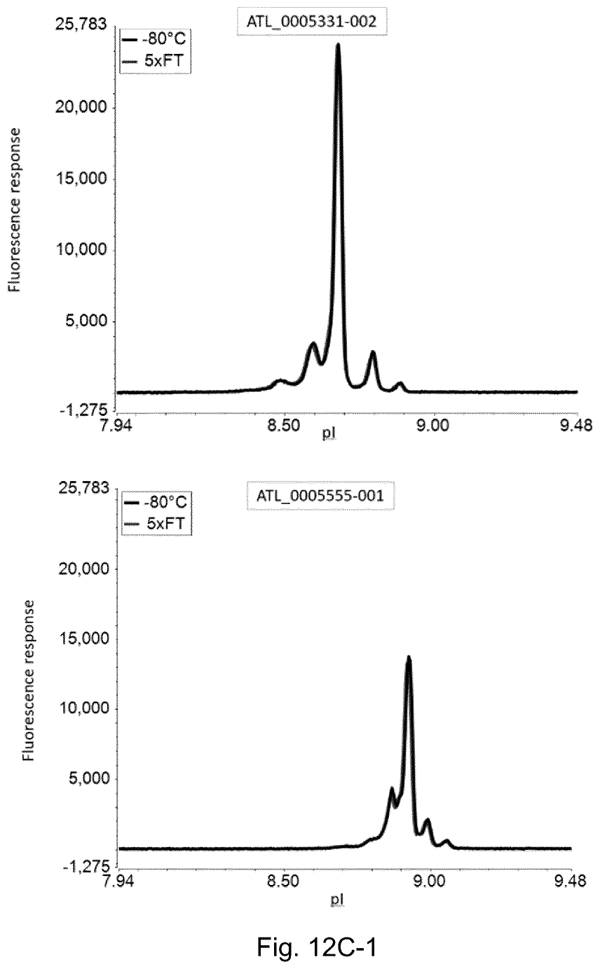

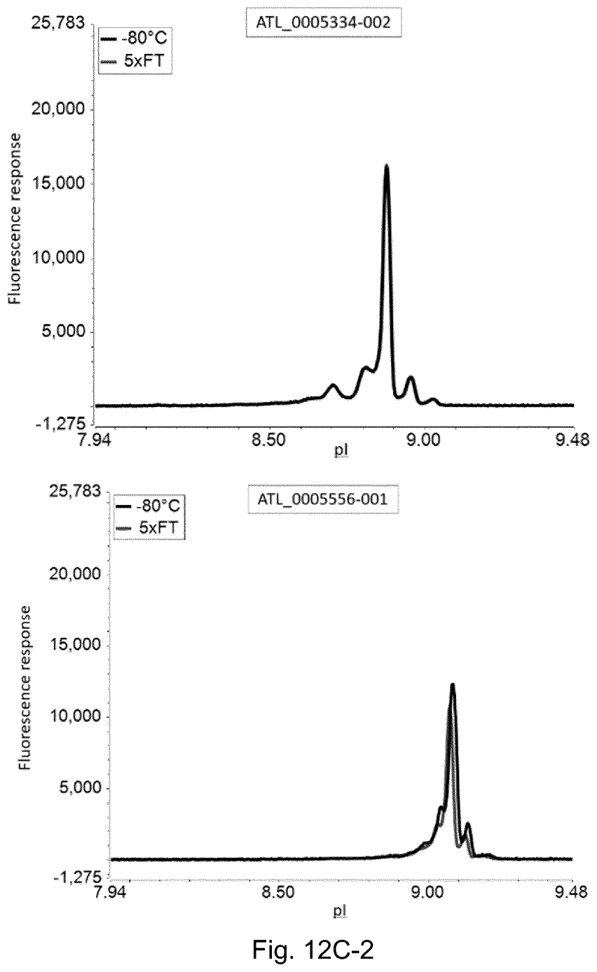

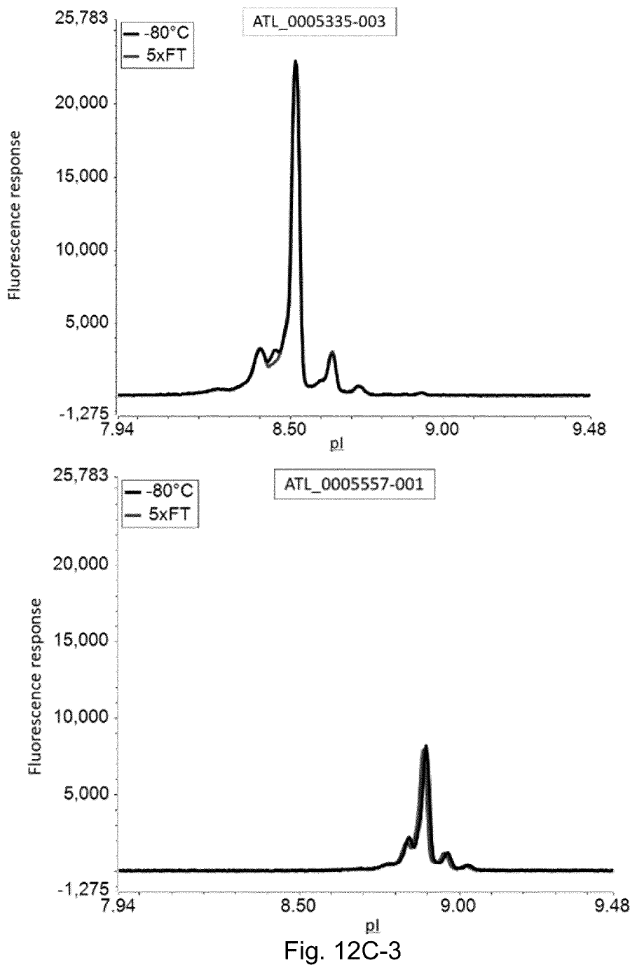

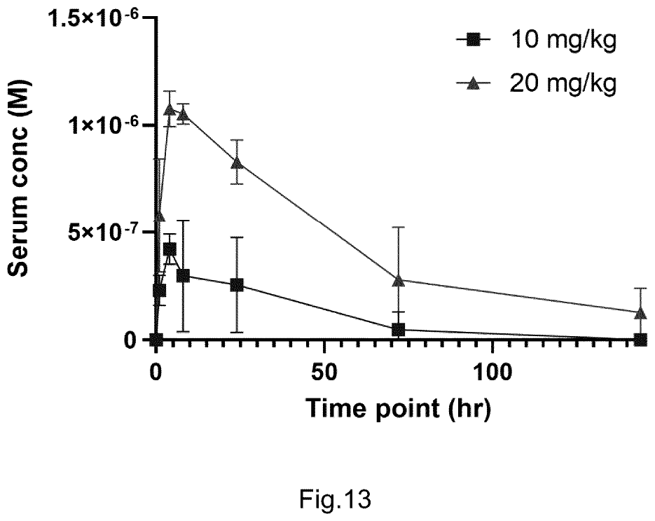

Capillary isolelectric focussing (cIEF) was used to assess charge heterogeneity of the antibodies. Prior to subjecting the antibodies to temperature stress or freeze thaw cycles, the isoelectric points (pI) of all six antibodies were determined using cIEF (see FIG. 12A and Table 6). The pI values were within the typical range of 7.5-9 for antibodies, appropriate for downstream processing.

TABLE 6
Main peak pl and main peak area (%) for the six indicated antibodies
shown in FIG. 12 prior to the stability study (T = 0).

| Antibody | Main Peak pl | Main Peak Area (%) |
|---|---|---|
| ATL_0005331 | 8.68 | 68.5 |
| ATL_0005334 | 8.87 | 61.9 |
| ATL_0005335 | 8.52 | 62.6 |
| ATL_0005555 | 8.93 | 62.9 |
| ATL_0005556 | 9.08 | 63.4 |
| ATL_0005557 | 8.89 | 63.0 |

An increase in acidic charge variants was observed for the highest temperature stress condition following incubation at 40° C. for three weeks but not at any of the other temperatures tested (FIG. 12B) or following 5× freeze thaw cycles (FIG. 12C). In conclusion the antibodies do not undergo a significant change in biophysical properties over an accelerated temperature stress study performed in an unoptimized formulation buffer at 5 mg/ml.

### Example 5—In Vivo PK Profile

To determine the serum PK of the lead antibodies, an in vivo PK experiment was performed with ATL_0005335 (parent antibody of ATL_0005567). Antibody levels were assessed via ELISA of serum samples collected 1, 4, 8, 24, 72 and 144 hours after administration. ATL_0005335 shows a linear PK and does not show an altered clearance compared with a non-binding IgG1 antibody in wild-type animals. This suggests that the antibody is not binding unexpectedly to molecules outside the CNS which would have implications for pharmacokinetics and potentially safety.

### Example 6—Optimisation of Lead Antibodies

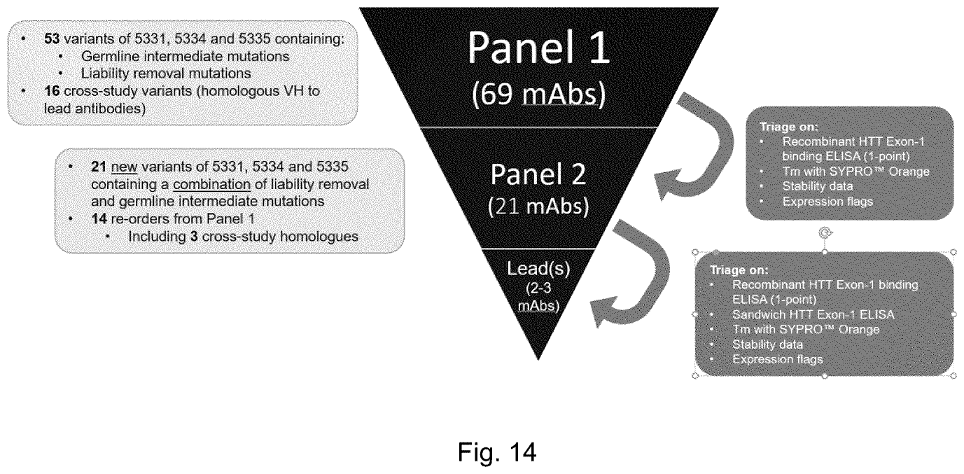

To identify the optimal candidate for preclinical testing and ultimately for testing in the clinic, antibodies were subjected to various rounds of triaging as shown in FIG. 14. In order to identify lead antibodies with optimal development characteristics, a first panel of antibodies was designed and consisted of variants of ATL_5331; 5334; and 5335 containing germline intermediate mutations and liability removal mutations. Reversing to germline framework sequences reduces the potential immunogenicity of the antibodies, whereas mutating sequence motifs to remove potential liabilities can help to minimise future manufacturing and pharmacokinetic issues caused by amino acid modifications.

Tables 7 and 8 show the VH and VL sequences for parent antibodies, their germline equivalents, and equivalents with additional development mutations.

TABLE 7
VH sequences of tested antibodies and prior art antibody ATL_5059.

| | Parent | VH | |
|---|---|---|---|
| Antibody | Antibody | Sequence | VH Residues |
| ATL_0005577 | ATL_0005331 | AC_0001291 | EVQLVESGGGLVKPGGSLRLSCAASGFTFNKAW |
| | | | MNWVRQAPGKGLEWVGRIKSGIDAGTTDYAAPV |
| | | | QGRFTISRDDSKNTVYLQMNSLNTEDTAVYYCIP |
| | | | PPYYYYYGLDVWGQGTTVTVSS (SEQ ID NO: 31) |
| ATL_0005891 | ATL_0005331 | AC_0001291 | EVQLVESGGGLVKPGGSLRLSCAASGFTFNKAW |
| | | | MNWVRQAPGKGLEWVGRIKSGIDAGTTDYAAPV |
| | | | QGRFTISRDDSKNTVYLQMNSLNTEDTAVYYCIP |
| | | | PPYYYYYGLDVWGQGTTVTVSS (SEQ ID NO: 31) |
| ATL_0005586 | ATL_0005334 | AC_0001291 | EVQLVESGGGLVKPGGSLRLSCAASGFTFNKAW |
| | | | MNWVRQAPGKGLEWVGRIKSGIDAGTTDYAAPV |
| | | | QGRFTISRDDSKNTVYLQMNSLNTEDTAVYYCIP |
| | | | PPYYYYYGLDVWGQGTTVTVSS (SEQ ID NO: 31) |
| ATL_0005890 | ATL_0005331 | AC_0001291 | EVQLVESGGGLVKPGGSLRLSCAASGFTFNKAW |
| | | | MNWVRQAPGKGLEWVGRIKSGIDAGTTDYAAPV |
| | | | QGRFTISRDDSKNTVYLQMNSLNTEDTAVYYCIP |
| | | | PPYYYYYGLDVWGQGTTVTVSS (SEQ ID NO: 31) |
| ATL_0005559 | ATL_0005331 | AC_0000737 | EVQLVESGGGLVKPGGSLRLSCAASGFTFNKAW |
| | | | MNWVRQAPGKGLEWVGRIKSGIDGGTTDYAAPV |
| | | | QGRFTISRDDSKNTVYLQMNSLNTEDTAVYYCIP |
| | | | PPYYYYYGLDVWGQGTTVTVSS (SEQ ID NO: 19) |
| ATL_0005572 | ATL_0005335 | AC_0000737 | EVQLVESGGGLVKPGGSLRLSCAASGFTFNKAW |
| | | | MNWVRQAPGKGLEWVGRIKSGIDGGTTDYAAPV |
| | | | QGRFTISRDDSKNTVYLQMNSLNTEDTAVYYCIP |
| | | | PPYYYYYGLDVWGQGTTVTVSS (SEQ ID NO: 19) |
| ATL_0005567 | ATL_0005335 | AC_0000737 | EVQLVESGGGLVKPGGSLRLSCAASGFTFNKAW |
| | | | MNWVRQAPGKGLEWVGRIKSGIDGGTTDYAAPV |
| | | | QGRFTISRDDSKNTVYLQMNSLNTEDTAVYYCIP |
| | | | PPYYYYYGLDVWGQGTTVTVSS (SEQ ID NO: 19) |
| ATL 0005335 | ATL 0005042 | AC_0000737 | EVQLVESGGGLVKPGGSLRLSCAASGFTFNKAW |
| | | | MNWVRQAPGKGLEWVGRIKSGIDGGTTDYAAPV |
| | | | QGRFTISRDDSKNTVYLQMNSLNTEDTAVYYCIP |
| | | | PPYYYYYGLDVWGQGTTVTVSS (SEQ ID NO: 19) |
| ATL_0005901 | ATL_0005334 | AC_0001760 | EVQLVESGGGLVKPGGSLRLSCAASGFTFNKAW |
| | | | MSWVRQAPGKGLEWVGRIKSGIDAGTTDYAAPV |
| | | | KGRFTISRDDSKATLYLQMNSLKTEDTAVYYCIPP |
| | | | PYYYYYGLDVWGQGTTVTVSS (SEQ ID NO: 12) |
| ATL_0005563 | ATL_0005331 | AC_0000737 | EVQLVESGGGLVKPGGSLRLSCAASGFTFNKAW |
| | | | MNWVRQAPGKGLEWVGRIKSGIDGGTTDYAAPV |
| | | | QGRFTISRDDSKNTVYLQMNSLNTEDTAVYYCIP |
| | | | PPYYYYYGLDVWGQGTTVTVSS (SEQ ID NO: 19) |
| ATL_0005896 | ATL_0005331 | AC_0001754 | EVQLVESGGGLVKPGGSLRLSCAASGFTFNKAW |
| | | | MSWVRQAPGKGLEWVGRIKSGIDAGTTDYAAPV |
| | | | KGRFTISRDDSKNTLYLQMNSLKTEDTAVYYCIPP |
| | | | PYYYYYGLDVWGQGTTVTVSS (SEQ ID NO: 7) |
| ATL_0005894 | ATL 0005331 | AC_0001754 | EVQLVESGGGLVKPGGSLRLSCAASGFTFNKAW |
| | | | MSWVRQAPGKGLEWVGRIKSGIDAGTTDYAAPV |
| | | | KGRFTISRDDSKNTLYLQMNSLKTEDTAVYYCIPP |
| | | | PYYYYYGLDVWGQGTTVTVSS (SEQ ID NO: 7) |
| ATL_0005895 | ATL_0005331 | AC_0001754 | EVQLVESGGGLVKPGGSLRLSCAASGFTFNKAW |
| | | | MSWVRQAPGKGLEWVGRIKSGIDAGTTDYAAPV |
| | | | KGRFTISRDDSKNTLYLQMNSLKTEDTAVYYCIPP |
| | | | PYYYYYGLDVWGQGTTVTVSS (SEQ ID NO: 7) |
| ATL_0005900 | ATL_0005334 | AC_0001269 | EVQLVESGGGLVKPGGSLRLSCAASGFTFNKAW |
| | | | MSWVRQAPGKGLEWVGRIKSGIDGGTTDYAAPV |
| | | | KGRFTISRDDSKNTLYLQMNSLKTEDTAVYYCIPP |
| | | | PYYYYYGLDVWGQGTTVTVSS (SEQ ID NO: 28) |
| ATL_0005331 | ATL 0005042 | AC_0000737 | EVQLVESGGGLVKPGGSLRLSCAASGFTFNKAW |
| | | | MNWVRQAPGKGLEWVGRIKSGIDGGTTDYAAPV |
| | | | QGRFTISRDDSKNTVYLQMNSLNTEDTAVYYCIP |
| | | | PPYYYYYGLDVWGQGTTVTVSS (SEQ ID NO: 19) |
| ATL_0005334 | ATL 0005042 | AC_0000737 | EVQLVESGGGLVKPGGSLRLSCAASGFTFNKAW |
| | | | MNWVRQAPGKGLEWVGRIKSGIDGGTTDYAAPV |
| | | | QGRFTISRDDSKNTVYLQMNSLNTEDTAVYYCIP |
| | | | PPYYYYYGLDVWGQGTTVTVSS (SEQ ID NO: 19) |
| ATL_0005555 | ATL_0005331 | AC_0001269 | EVQLVESGGGLVKPGGSLRLSCAASGFTFNKAW |
| | | | MSWVRQAPGKGLEWVGRIKSGIDGGTTDYAAPV |
| | | | KGRFTISRDDSKNTLYLQMNSLKTEDTAVYYCIPP |
| | | | PYYYYYGLDVWGQGTTVTVSS (SEQ ID NO: 28) |
| ATL_0005556 | ATL_0005334 | AC_0001269 | EVQLVESGGGLVKPGGSLRLSCAASGFTFNKAW |
| | | | MSWVRQAPGKGLEWVGRIKSGIDGGTTDYAAPV |
| | | | KGRFTISRDDSKNTLYLQMNSLKTEDTAVYYCIPP |
| | | | PYYYYYGLDVWGQGTTVTVSS (SEQ ID NO: 28) |
| ATL_0005557 | ATL_0005335 | AC_0001269 | EVQLVESGGGLVKPGGSLRLSCAASGFTFNKAW |
| | | | MSWVRQAPGKGLEWVGRIKSGIDGGTTDYAAPV |
| | | | KGRFTISRDDSKNTLYLQMNSLKTEDTAVYYCIPP |
| | | | PYYYYYGLDVWGQGTTVTVSS (SEQ ID NO: 28) |
| ATL_0005059 | NA | AC_0000754 | EVQLVESGGGLVKPGGSLTISCAASGFTFSNAW |
| | | | MNWVRQAPGKGLEWVGHIRTQAEGGTSDYAAP |
| | | | VKGRFTISRDDSKNTLYLQMNSLKTEDTAVYYCIP |
| | | | PPYYYYYGLDVWGQGTTVTVSS (SEQ ID NO: 33) |

TABLE 8
VL sequences of tested antibodies and prior art antibody ATL_5059

| | Parent | VL | |
|---|---|---|---|
| Antibody | Antibody | Sequence | VL Residues |
| ATL_0005577 | ATL_0005331 | AC_0001036 | QSALTQPRSVSGSPGQSITISCTGTSSDVGS |
| | | | YNLVSWFQQHPGKAPKLIIYEVNKRPSGVPD |
| | | | RFSGSKSGNTASLTVSGLQAEDEADYYCGS |
| | | | YAGTNNVFGTGTKLTVL (SEQ ID NO: 22) |
| ATL_0005891 | ATL_0005331 | AC_0001277 | QSALTQPRSVSGSPGQSVTISCTGTSSDVGS |
| | | | YNLVSWYQQHPGKAPKLIIYEVNKRPSGVPD |
| | | | RFSGSKSGNTASLTISGLQAEDEADYYCGSY |
| | | | AGTNNVFGTGTKVTVL (SEQ ID NO: 32) |
| ATL_0005586 | ATL_0005334 | AC_0001280 | QSALTQPASVSGSPGQSITISCTGTSSDVGG |
| | | | YKLVSWYQQHPGRAPKLMIYEVSKRPSGVS |
| | | | SRFSGSKSGSTASLTISGLQAEDEADYYCSS |
| | | | YAGSSVVFGGGTKLTVL (SEQ ID NO: 13) |
| ATL_0005890 | ATL_0005331 | AC_0001270 | QSALTQPRSVSGSPGQSVTISCTGTSSDVGS |
| | | | YNLVSWYQQHPGKAPKLMIYEVNKRPSGVP |
| | | | DRFSGSKSGNTASLTISGLQAEDEADYYCGS |
| | | | YAGTNNVFGTGTKVTVL (SEQ ID NO: 29) |
| ATL_0005559 | ATL_0005331 | AC_0001273 | QSALTQPRSVSGSPGQSITISCTGTSSDVGS |
| | | | YNLVSWFQQHPGKAPKLIIYEVNKRPSGVPD |
| | | | RFSGSKSGATASLTVSGLQAEDEADYYCGS |
| | | | YAGTNNVFGTGTKLTVL (SEQ ID NO: 42) |
| ATL_0005572 | ATL_0005335 | AC_0001286 | QSALTQPASVSGSPGQSITISCTGTSSDIGSY |
| | | | NLVSWYQQHPGKAPKPLIYEGSKRPSGVSN |
| | | | RFSGSKSGNTASLTISGLQAEDEADYYCSSY |
| | | | AGFNTLVFGGGTKLTVL (SEQ ID NO: 39) |
| ATL_0005567 | ATL_0005335 | AC_0001281 | QSALTQPASVSGSPGQSITISCTGTSSDIGSY |
| | | | NLVSWYQQHPGNAPKPLIYEGSKRPSGVSA |
| | | | RFSGSKSGNTASLTISGLQPEDEADYYCSSY |
| | | | AGFSTLVFGGGTKVTVL (SEQ ID NO: 20) |
| ATL_0005335 | ATL 0005042 | AC_0001040 | QSALTQPASVSGSPGQSITISCTGTSSDIGSY |
| | | | NLVSWYQQHPGNAPKPLIYEGSKRPSGVSA |
| | | | RFSGSKSGNTASLTISGLQPEDEADYYCSSY |
| | | | AGFNTLVFGGGTKVTVL (SEQ ID NO: 26) |
| ATL_0005901 | ATL_0005334 | AC_0001280 | QSALTQPASVSGSPGQSITISCTGTSSDVGG |
| | | | YKLVSWYQQHPGRAPKLMIYEVSKRPSGVS |
| | | | SRFSGSKSGSTASLTISGLQAEDEADYYCSS |
| | | | YAGSSVVFGGGTKLTVL (SEQ ID NO: 13) |
| ATL_0005563 | ATL_0005331 | AC_0001277 | QSALTQPRSVSGSPGQSVTISCTGTSSDVGS |
| | | | YNLVSWYQQHPGKAPKLIIYEVNKRPSGVPD |
| | | | RFSGSKSGNTASLTISGLQAEDEADYYCGSY |
| | | | AGTNNVFGTGTKVTVL (SEQ ID NO: 32) |
| ATL_0005896 | ATL_0005331 | AC_0001752 | QSALTQPRSVSGSPGQSVTISCTGTSSDVGS |
| | | | YNLVSWYQQHPGKAPKLMIYEVNKRPSGVP |
| | | | DRFSGSKSGATASLTISGLQAEDEADYYCGS |
| | | | YAGTNNVFGTGTKVTVL (SEQ ID NO: 40) |
| ATL_0005894 | ATL_0005331 | AC_0001755 | QSALTQPRSVSGSPGQSVTISCTGTSSDVGS |
| | | | YNLVSWYQQHPGKAPKLIIYEVNKRPSGVPD |
| | | | RFSGSKSGATASLTISGLQAEDEADYYCGSY |
| | | | AGTANVFGTGTKVTVL (SEQ ID NO: 41) |
| ATL_0005895 | ATL_0005331 | AC_0001756 | QSALTQPRSVSGSPGQSVTISCTGTSSDVGS |
| | | | YNLVSWYQQHPGKAPKLMIYEVNKRPSGVP |
| | | | DRFSGSKSGATASLTISGLQAEDEADYYCGS |
| | | | YAGTANVFGTGTKVTVL (SEQ ID NO: 8) |
| ATL_0005900 | ATL_0005334 | AC_0001280 | QSALTQPASVSGSPGQSITISCTGTSSDVGG |
| | | | YKLVSWYQQHPGRAPKLMIYEVSKRPSGVS |
| | | | SRFSGSKSGSTASLTISGLQAEDEADYYCSS |
| | | | YAGSSVVFGGGTKLTVL (SEQ ID NO: 13) |
| ATL_0005331 | ATL_0005042 | AC_0001036 | QSALTQPRSVSGSPGQSITISCTGTSSDVGS |
| | | | YNLVSWFQQHPGKAPKLIIYEVNKRPSGVPD |
| | | | RFSGSKSGNTASLTVSGLQAEDEADYYCGS |
| | | | YAGTNNVFGTGTKLTVL (SEQ ID NO: 22) |
| ATL_0005334 | ATL_0005042 | AC_0001039 | QSALTQPASVSGSPGQSITISCTGTSSDVGG |
| | | | YKLVSWYQQHPGRAPKLMIYEVSKRPSGVS |
| | | | SRFSGSKSGSTASLTISGLQAEDEADYYCCS |
| | | | YAGSSVVFGGGTKLTVL (SEQ ID NO: 24) |
| ATL_0005335 | ATL_0005042 | AC_0001040 | QSALTQPASVSGSPGQSITISCTGTSSDIGSY |
| | | | NLVSWYQQHPGNAPKPLIYEGSKRPSGVSA |
| | | | RFSGSKSGNTASLTISGLQPEDEADYYCSSY |
| | | | AGFNTLVFGGGTKVTVL (SEQ ID NO: 26) |
| ATL 0005555 | ATL_0005331 | AC_0001270 | QSALTQPRSVSGSPGQSVTISCTGTSSDVGS |
| | | | YNLVSWYQQHPGKAPKLMIYEVNKRPSGVP |
| | | | DRFSGSKSGNTASLTISGLQAEDEADYYCGS |
| | | | YAGTNNVFGTGTKVTVL (SEQ ID NO: 29) |
| ATL_0005556 | ATL 0005334 | AC_0001039 | QSALTQPASVSGSPGQSITISCTGTSSDVGG |
| | | | YKLVSWYQQHPGRAPKLMIYEVSKRPSGVS |
| | | | SRFSGSKSGSTASLTISGLQAEDEADYYCCS |
| | | | YAGSSVVFGGGTKLTVL (SEQ ID NO: 24) |
| ATL_0005557 | ATL_0005335 | AC_0001271 | QSALTQPASVSGSPGQSITISCTGTSSDIGSY |
| | | | NLVSWYQQHPGKAPKLMIYEGSKRPSGVSN |
| | | | RFSGSKSGNTASLTISGLQAEDEADYYCSSY |
| | | | AGFNTLVFGGGTKLTVL (SEQ ID NO: 30) |
| ATL_0005059 | NA | AC_0000755 | QSALTQPASVSGSPGQSITISCTGASSDVGT |
| | | | YDLVSWYQQHPGKAPKLIIYEVNKRPSGVSY |
| | | | RFSASKSANTASLTISGLQAEDEAEYYCCSY |
| | | | AGYSTVFGGGTKLTVL (SEQ ID NO: 34) |

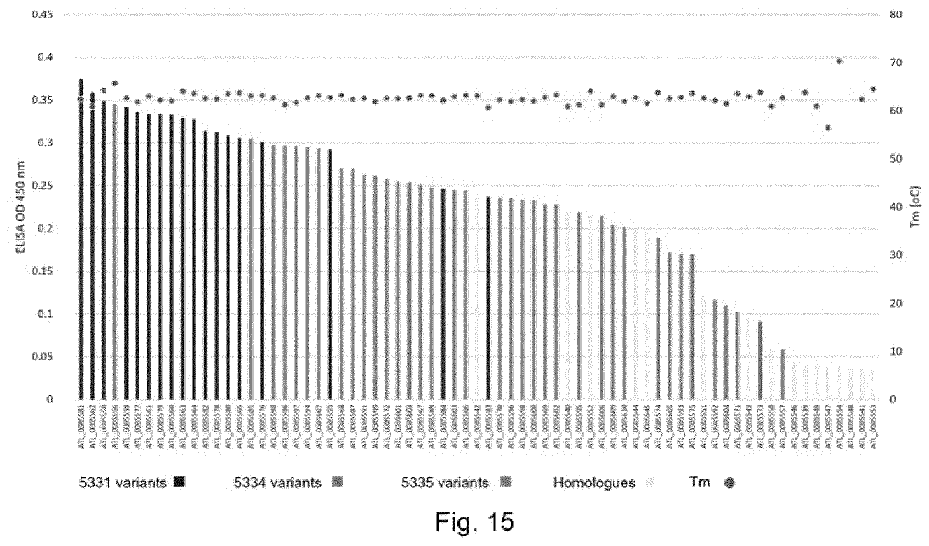

A total of 69 mAbs (monoclonal antibodies) were primarily triaged based on binding to HTT exon 1 in ELISA and thermostability analysis, determined by measuring the Tm with SYPRO™ Orange in a thermal shift assay (See FIG. 15). There were no differences in thermostability with a similar Tm for all variants (Tm1 (mean)=62.4±2.1° C. (% RSD)) (FIG. 15). Comparing the binding ELISA all 5331 variants were ranked highly, whereas 5334 variants had variable rankings and 5335 variants were lowly ranked.

A second variant panel was subsequently designed containing a combination of selected mutations and antibodies were then further triaged based on their binding to HTT exon 1 and thermostability to select lead antibodies.

Lead antibodies were primarily selected based on affinity to HTT (using 1-point binding ELISA) and binding potency, as determined by sandwich HTT ELISA (ranking on 48Q HTT Exo-1 EC50, E50<50 nM; then on EC50 (25Q:48Q) and 48Q), and, secondly, based on germline mutations and liability removing mutations (removing low yield mutations, stability flags, etc; for example, G54A removes an aspartate isomerisation motif).

Antibodies with lower EC50 values, as determined by HTT sandwich ELISA, have a greater binding potency, and therefore affinity, to HTT exon 1. First, antibodies were ranked based on their EC50 values for binding to 48Q HTT Exon-1. Antibodies with an EC50 lower than 50 nm were triaged and further ranked based in EC50 as well as 25Q:48Q ratio. The higher the ratio of 25Q:48Q binding, the higher the potency of the antibody for 48Q HTT over 25Q HTT (i.e. the lower the ec50 of 48Q v the ec50 of 25Q the better). Further factors that were taken into consideration for selection of the lead antibodies was the number of germline mutations and liability removing mutations, as these antibodies pose less of a safety hazard.

Finally, three lead antibodies were selected ATL_5901 (5334 variant, rank 1); ATL_5895 (5331 variant, rank 1); and ATL_5567 (5335 variant, rank 3).

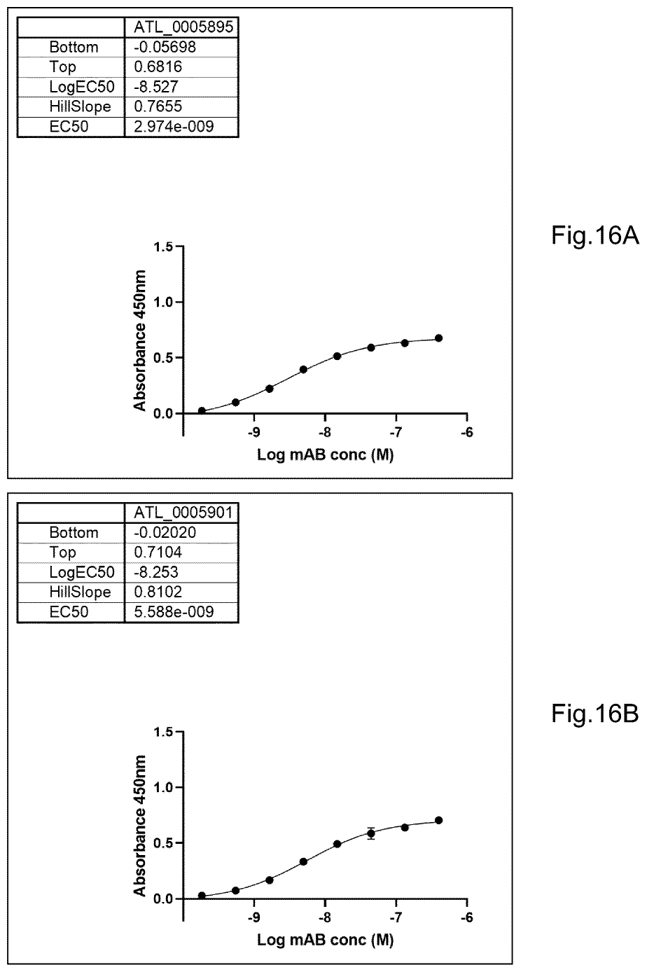

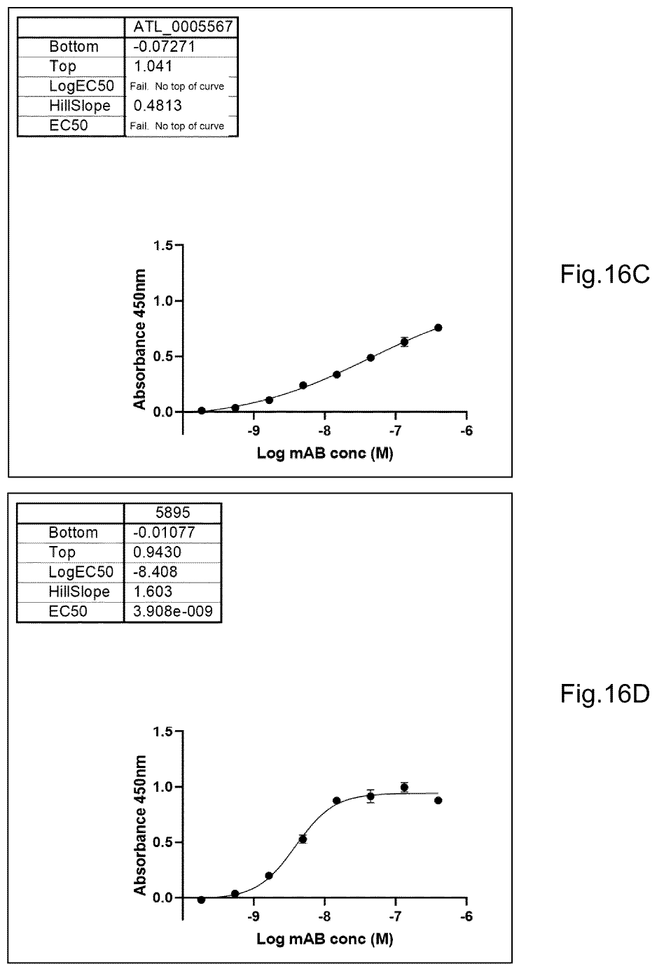

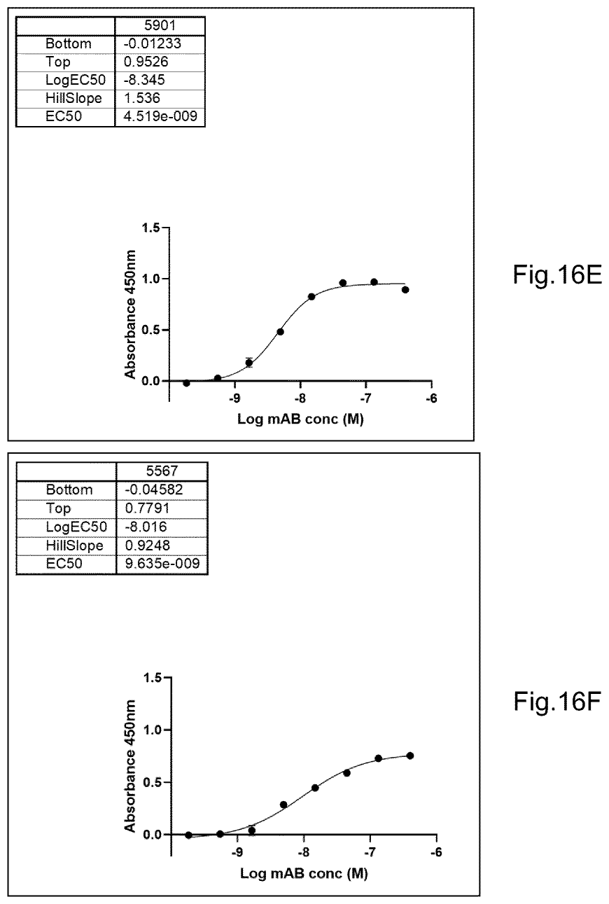

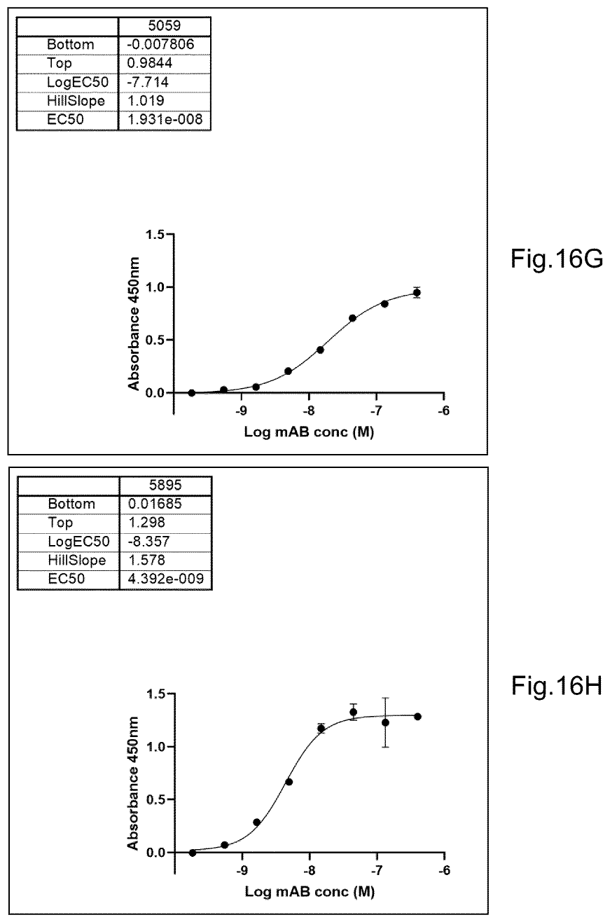

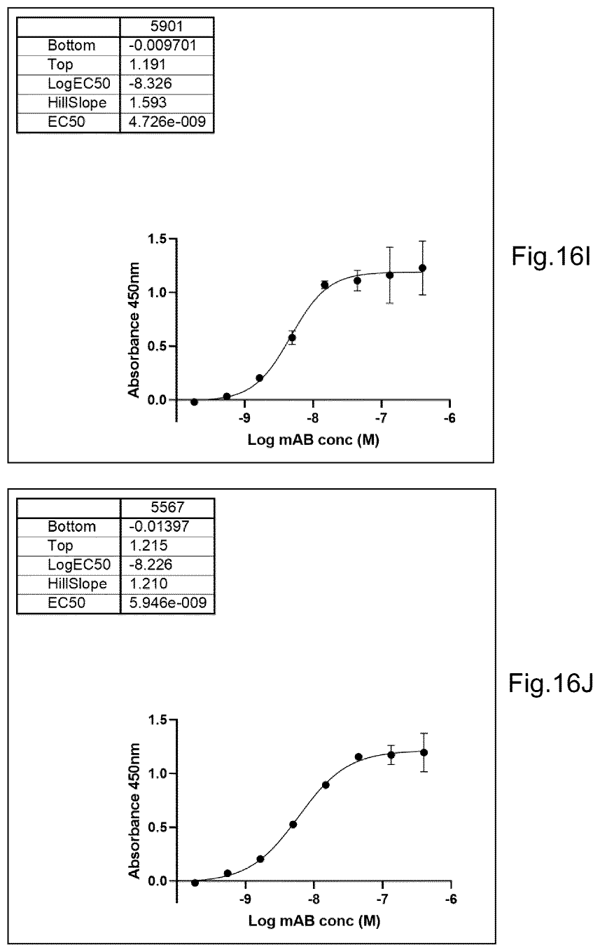

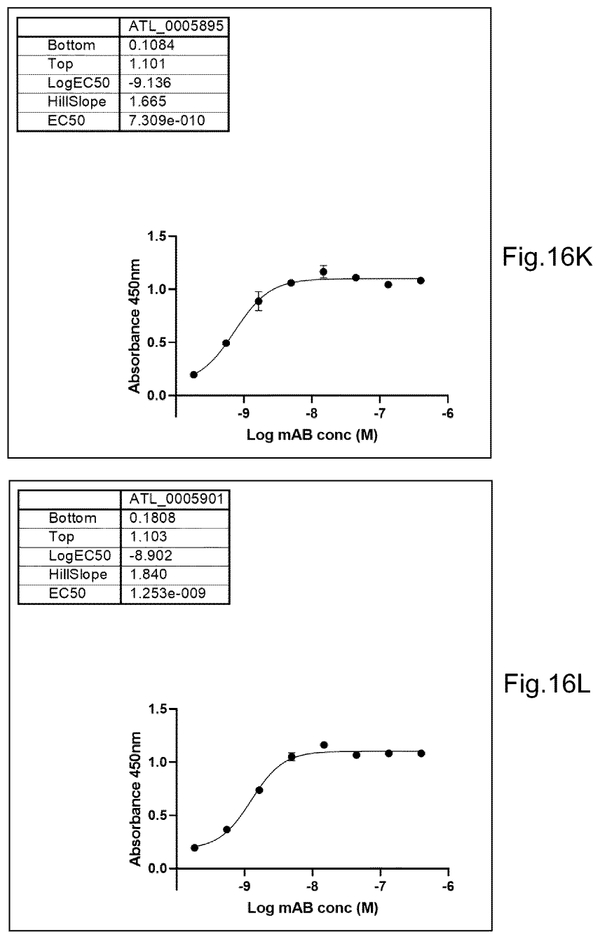

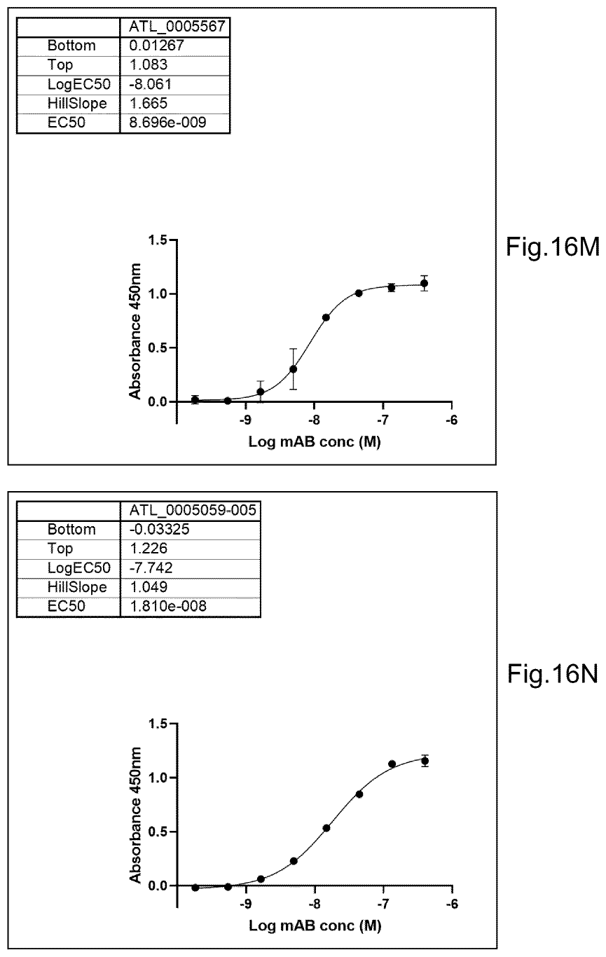

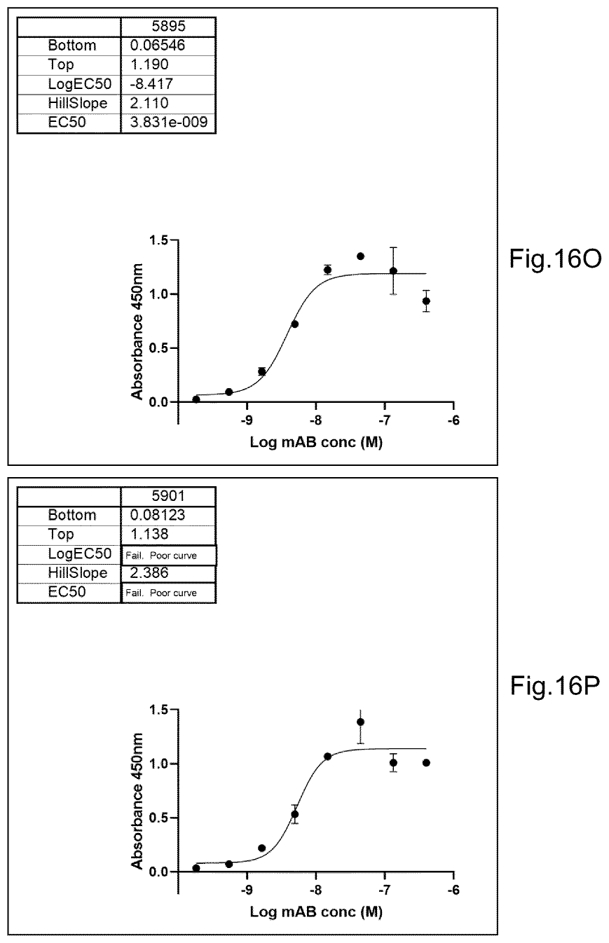

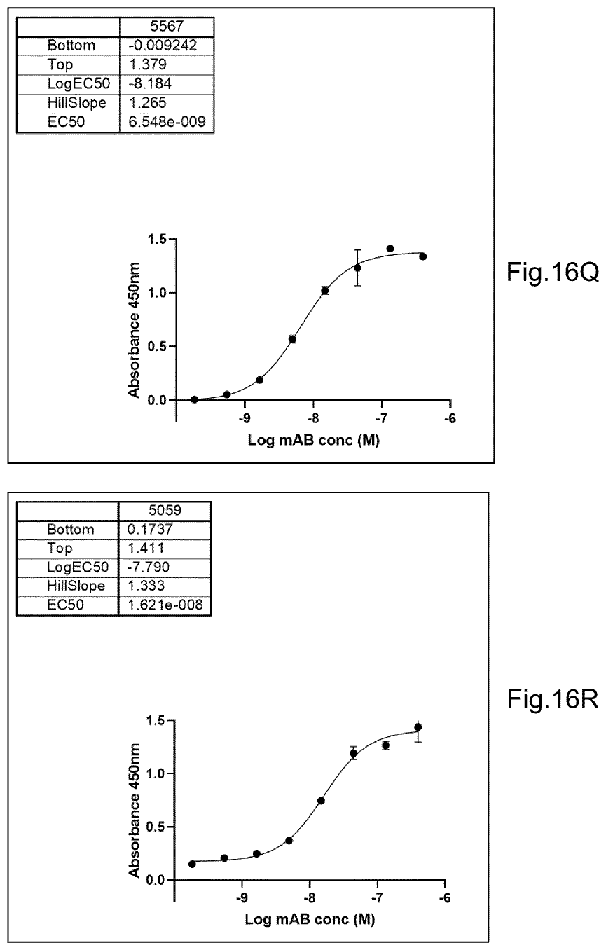

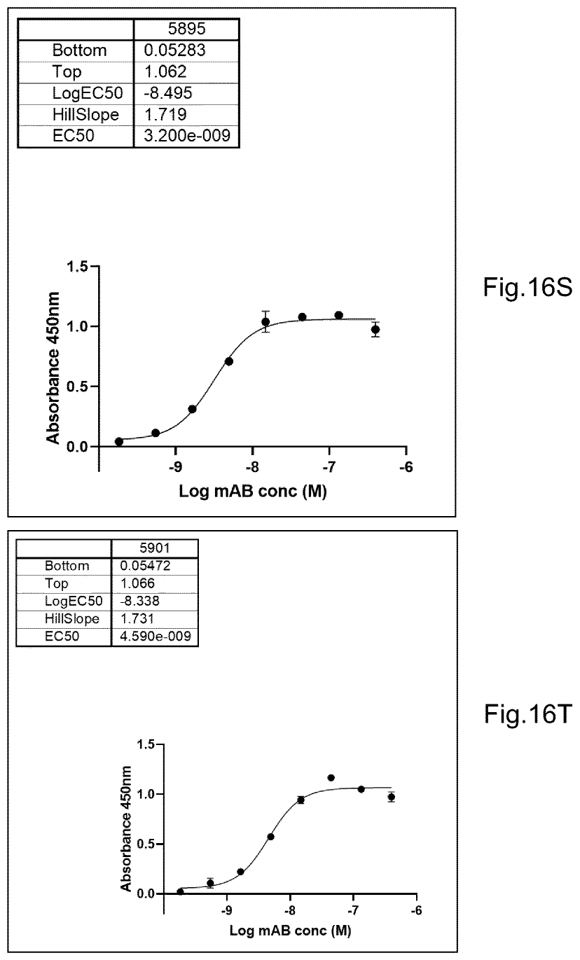

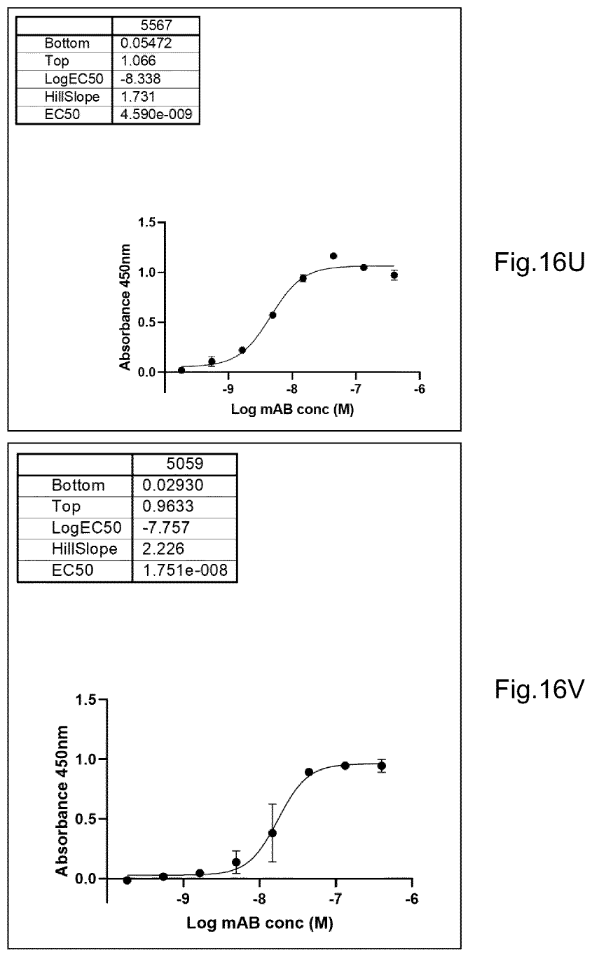

ATL_5901 and ATL_5895 had similarly low EC50 levels (see FIG. 16 and Tables 9-10) and contained developability and germline mutations. ATL_5567 was the best performing 5335-derived variant.

TABLE 9
EC50 for antibodies v HTT Exon 1 25Q (nM)

| | Biological | Biological | Biological | |
|---|---|---|---|---|
| | repeat 1 | repeat 2 | repeat 3 | Average |
| ATL_0005895 | 2.974 | 3.908 | 4.392 | 3.709533 |
| ATL_0005901 | 5.588 | 4.519 | 4.726 | 4.923389 |
| ATL_0005567 | Fail; No top | 9.635 | 5.946 | 7.568997 |
| | of curve | | | |
| ATL_0005059/ | Not | 19.31 | Not | |
| NI-302.8F1 | assessed | | assessed | 19.31 |

TABLE 10
EC50 for antibodies v HTT Exon 1 48Q (nM)

| | Biological | Biological | Biological | |
|---|---|---|---|---|
| | repeat 1 | repeat 2 | repeat 3 | Average |
| ATL_0005895 | 0.7309 | 3.831 | 3.2 | 2.077017 |
| ATL_0005901 | 1.253 | Fail. Poor | 4.59 | 2.398181 |
| | | curve | | |
| ATL 0005567 | 8.696 | 6.548 | 4.59 | 6.393622 |
| ATL_0005059/ | 18.1 | 16.21 | 17.51 | 17.25504 |
| NI-302.8F1 | | | | |

TABLE 11
Ratio of EC50 for 25Q:48Q HTT Exon 1

| | Ratio 25Q:48Q |
|---|---|
| ATL_0005895 | 1.785991 |
| ATL_0005901 | 2.052968 |
| ATL_0005567 | 1.183835 |
| ATL_0005059/NI-302.8F1 | 1.119093 |

All three lead antibodies showed consistently higher binding potency (up to 9-fold) for HTT Exon 1 25Q (Table 9); HTT Exon 1 48Q (Table 10), and a higher 25Q:48Q ratio (Table 11), compared with reference antibody NI-302.8F1 described in U.S. Pat. No. 11,401,325 B2.

These data therefore show that the three lead antibodies ATL_5901 and ATL_5895, and ATL_5667 have improved binding properties compared with prior art antibodies. The lead antibodies have also been optimised in terms of germline and sequence liability removal mutations to potentially decrease immunogenicity and increase shelf life, stability, and suitability for manufacture.

The suitability for manufacture of ATL_5895 was further validated by verifying that no aggregation was observed following incubation at 40° C. (vs −80° C.) for 4 weeks (by SEC-HPLC) as explained above, and that no aggregation was observed following 10 freeze-thaw cycles (by SEC-HPLC) as explained above. ATL_5895 also has a pI within appropriate range for downstream processing (i.e. 7.5E-9).

### Example 7—Binding to Disease Relevant HTT

Huntington's disease is caused by pathological expansion of a cytosine-adenine-guanine (CAG) triplet repeat in the huntingtin (HTT) gene that results in production of mutant HTT protein. As demonstrated in Example 6, ATL_5331; 5334; 5335; 5895; 5901; and 5567 are all able to bind 48Q HTT exon 1 (i.e. mutated HTT) in an ELISA, which indicates that the antibodies can bind pathological length forms of HTT. To determine if antibodies are capable of binding high molecular weight HTT (suggesting a more pathological aggregate like state of HTT), immunoprecipitation experiments were performed with ATL_0005335 (parent antibody of ATL_0005567).

To confirm that the antibodies of the invention are capable of binding to disease relevant HTT, binding of ATL 5335 to a U-2 OS cell line expressing 110 CAG repeats, resulting in a high molecular weight species HTT (HTT₁₁₀), akin to mutated HTT protein, was tested by Western blot after immunoprecipitation with ATL_5335. As shown in FIG. 17A, ATL_5335 is able to immunoprecipitate HTT with a molecular weight of >180 kDa (HTT₁₁₀).

R6/2 transgenic mice express the 5′ end of the human HTT gene, including exon 1 with approximately 120 CAG repeats, and display a neurological phenotype similar to the features of HD in humans, including the production of pathological HTT aggregates. FIG. 17B shows that ATL_0005895, ATL 0005901 and ATL_0005567 are able to immunoprecipitate high molecular weight HTT (in this case >250 kDa, which is the highest molecular weight protein marker used in this gel ladder) derived from brain homogenates from R6/2 mice. These data therefor show that the present antibodies are capable of binding aggregated HTT protein.

To confirm that ATL_0005895 is able to bind disease relevant HTT in the human brain, brain homogenate was prepared from superior temporal gyrus tissue from the postmortem brain of a Huntington's Disease patient. The material was incubated with beads coupled to an isotype control antibody or ATLX1095, followed by analysis by Western Blot. High Molecular weight HTT material was successfully immunoprecipitated by ATL_0005895 and not by the isotype control antibody (FIGS. 17C and 17D). In particular, FIG. 17C shows that ATL_0005895 is able to immunoprecipitate high molecular weight HTT from human brain homogenate derived from a huntingtin disease patient, as quantified in FIG. 17D. These data show that the present antibodies are capable of binding highly disease relevant aggregated HTT protein from human brain tissue.

### Example 8—Binding of Anti-HTT Antibodies to Seed Competent HTT Species

Self-propagating protein aggregates drive pathogenesis in many neurodegenerative diseases, including HD, which is characterised by the pathological aggregation of toxic mHTT species. It was therefore relevant to test the ability of the ATL_5895 and ATL_5901 to bind to these seed competent HTT species and inhibit pathological HTT aggregation.

To test the anti-seeding potential of anti-HTT antibodies ATL_5895 and ATL_5901, the aggregation rate of fluorescence resonance energy transfer (FRET)-tagged recombinant HTT was assessed via a FRET-based mHTT aggregate seeding (FRASE) assay as described in Ast, Anne et al. “mHTT Seeding Activity: A Marker of Disease Progression and Neurotoxicity in Models of Huntington's Disease.” Molecular cell vol. 71, 5 (2018): 675-688.

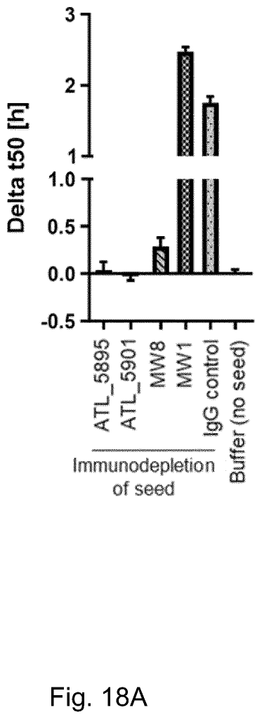

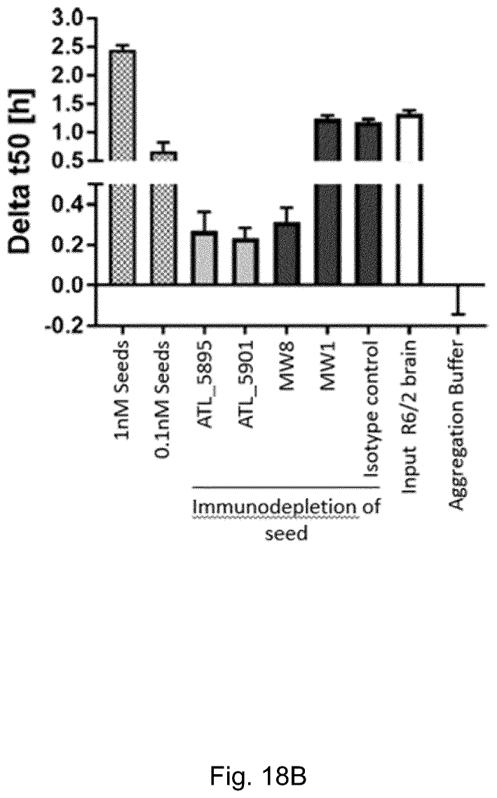

FIG. 18 shows the results of the FRASE assay using recombinant HTT seeds (FIG. 18A) and brain homogenates from R6/2 mice (FIG. 18B). Immunodepletion of pathological HTT, or “seed”, with antibodies ATL_0005895 and ATL_0005901 reduced in vitro aggregation rate of the seeding-competent HTT species. Notably, MW8 (Millipore; MABN2529), a mouse monoclonal IgG2A antibody to human HTT, binds to aggregated HTT and also achieves this effect to some extent, while MW1 (Millipore; MABN2427), which binds to the PolyQ region of HTT, does not achieve this (see FIGS. 18A and B).

Together, these results indicate that the antibodies described herein are capable of reducing the rate of self-propagation of toxic HTT species, suggesting these antibodies may be able slow down disease progression by reducing HTT seeding.

### Example 9—Phagocytosis Assay

As explained in examples 7 and 8 above, HD is characterised by the aggregation of mHTT species, which have the ability to self-propagate/seed thereby driving HD pathogenesis. An important immunological defence mechanism in the central nervous system is phagocytic clearance of neurotoxic proteins, such as these mHTT species, by microglia.

To investigate whether binding of anti-HTT antibody ATL_5895 to Exon 1 of HTT improves the phagocytic clearance of HTT by microglia, an in vitro assay using induced pluripotent stem cell (iPSC)-derived microglia was used to monitor phagocytic activity in culture (see “Materials and Methods” section above).

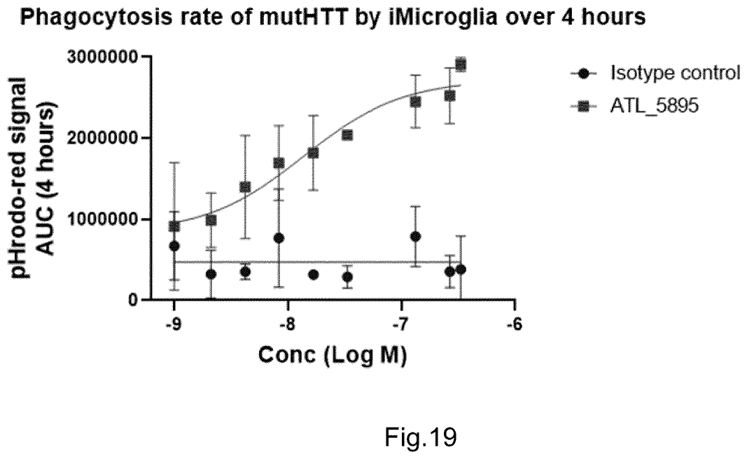

FIG. 19 shows the results of the assay with anti-HTT antibody ATL_5895 versus the isotype control antibody ATL5338, which binds fluorescein. FIG. 19 shows that binding of ATL_5895 increases the uptake of 48Q HTT-coated beads in iPSC microglia in a dose-dependent manner compared with beads treated with isotype control.

The results of this experiment indicate that the antibodies described herein increase phagocytotic clearance of toxic mHTT species.

### Example 10—In Vivo PK Study

The pharmacokinetic properties of a selection of anti-HTT antibodies were tested in vivo to assess circulating levels of antibody as well as CNS-penetrating ability.

Antibody levels were assessed via ELISA of serum samples collected at 1, 4, 8, 24, 72 and 144 hours and of CSF samples collected 4 and 144 hours post administration.

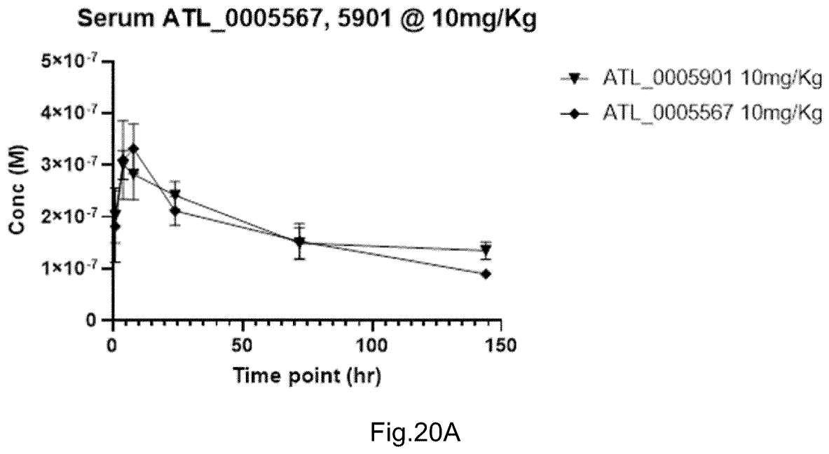

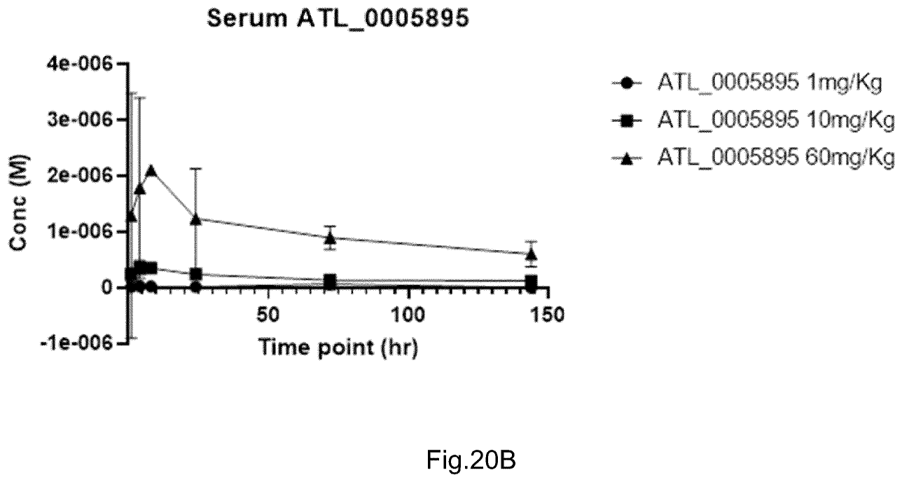

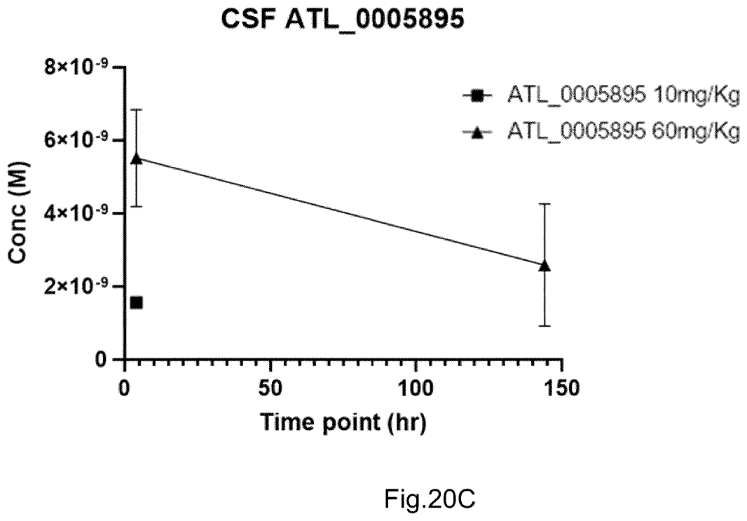

FIG. 20 show the results of the PK study. ATL_5901 and ATL_5567 exhibit a linear serum PK profile when administered at a concentration of 10 mg/kg (FIG. 20A). FIG. 20B shows the serum concentrations of ATL_5895 after treatment with 1, 10, or 60 mg/kg ATL_5895 and all of these concentrations result in a linear serum PK profile. Importantly, ATL_5895 also shows evidence of CNS penetration (FIG. 20C). Typical penetrance of IgG1 in CNS is 0.1-0.3%. This data indicates CNS exposure above EC50 of antibody is achievable.

A Proof-of-concept pharmacology study in the R6/1 mouse model is described in Example 15.

### Example 11—Affinity Maturation ATL 5895

As demonstrated in the above examples, ATL_5895 binds disease relevant HTT, inhibits seeding, and increases phagocytic clearance of pathological HTT. The inventors therefore investigated whether there was scope for affinity maturation of the ATL_5895 antibody in order to identify further high affinity HTT-binding antibodies.

Affinity maturation of ATL_5895 was performed by phage display, with phage libraries containing mutants in one or more of the following amino acids: YCIPPPYYYYYGLDV sequence (“extended CDR3H”), in the VH of ATL_5895; GSYAGTANV sequence (CDR3L), in the VL of ATL_5895. “Extended CDR” refers to a region including the CDR3H as well as positions outside the CDR3H, this instance four amino acids. In this case, 103 was one of the positions selected for “hard randomisation” (described under “phage display” in the “materials and Methods” section above). Residues close to but outside the CDRs were mutated in order to introduce more subtle changes into the antibody's function. Three rounds of phage selections were performed against human HTT Exon1 48Q or biotinylated GYSLPQPQPPPPPPPPPP peptide. The extended CDR3H and CDR3L region of the antibodies identified through affinity maturation by phage display are shown in Table 12. The full VH and VL sequences of these antibodies are provided in Table 1.

TABLE 12
Sequences of extended CDR3H and CDR3L of antibodies identified through phage
display. HCDR3 is located between positions 95 and 102 in Kabat numbering.
Extended CDR3H includes positions. Extended CDR3H shown above includes
positions 91-102.

| Antibody | Extended CDR3H | CDR3L |
|---|---|---|
| ATL_5895 | YCIP PPYYYYYGLDV (SEQ ID NO: 163) | GSYAGTANV (SEQ ID NO: 6) |
| ATL_6194 | YCVPPPYYYYYGLDV | VSYAGTANV |
| ATL_6195 | YCTPPPYYYYYGLDV | GSYAGTANV |
| ATL_6199 | WCSP PPYYYYYGLDV(SEQ ID NO: 164) | GSYAGTANV (SEQ ID NO: 6) |
| ATL_6200 | YCVP PPYYYYYGLDV (SEQ ID NO: 165) | GSYAGTANV (SEQ ID NO: 6) |
| ATL_6202 | YCSP PPFYYYYGLDV (SEQ ID NO: 166) | VSYGGTENV (SEQ ID NO: 162) |
| ATL_6203 | YCIP SPYYYYYGLDV (SEQ ID NO: 167) | VSYAGTANV (SEQ ID NO: 161) |
| ATL_6204 | YCIP PPYYYYYGLDV (SEQ ID NO: 163) | VVSFAGTANV (SEQ ID NO: 160) |
| ATL_6205 | WCVP PPYYYYYGLDV (SEQ ID NO: 168) | VSYAGTANV (SEQ ID NO: 161) |

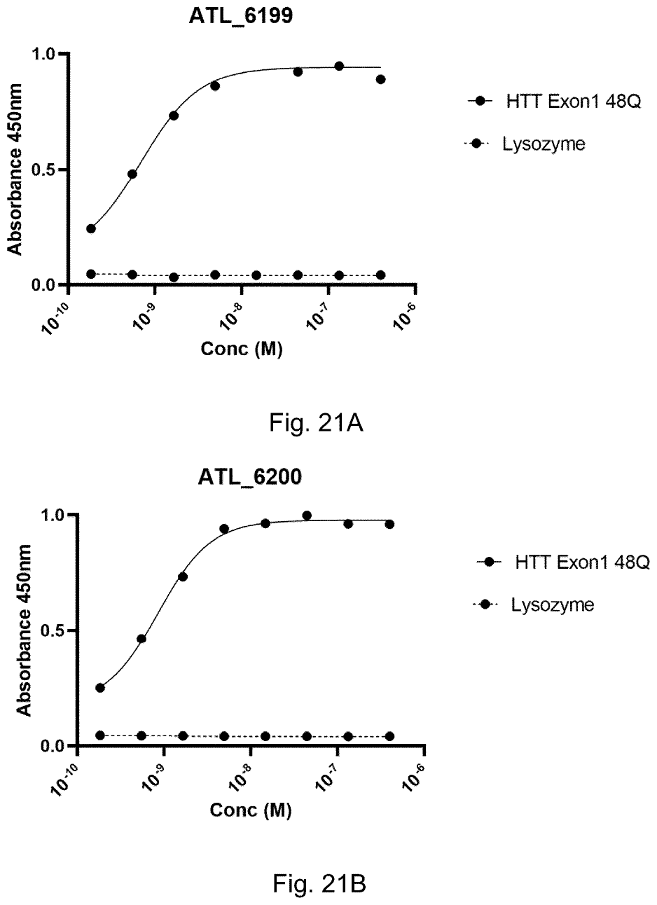

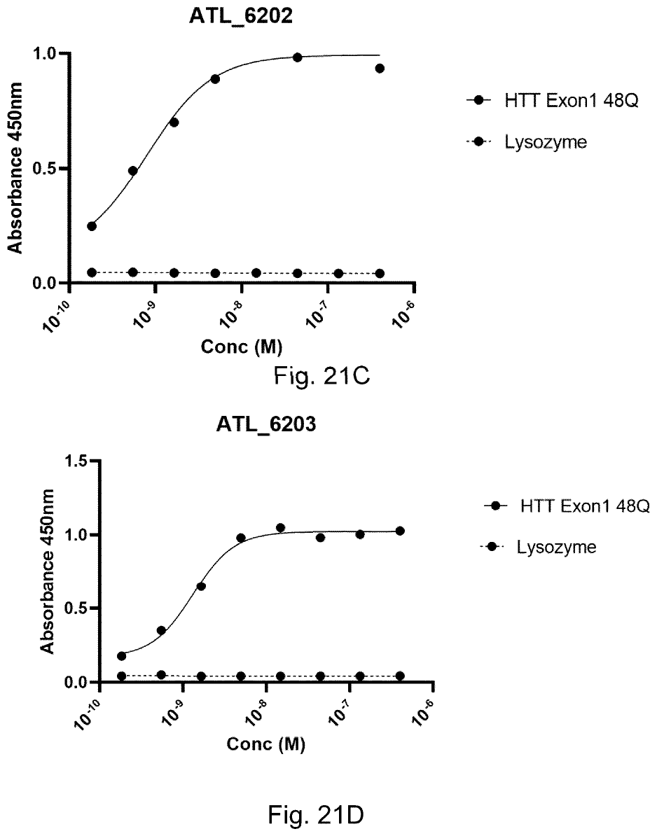

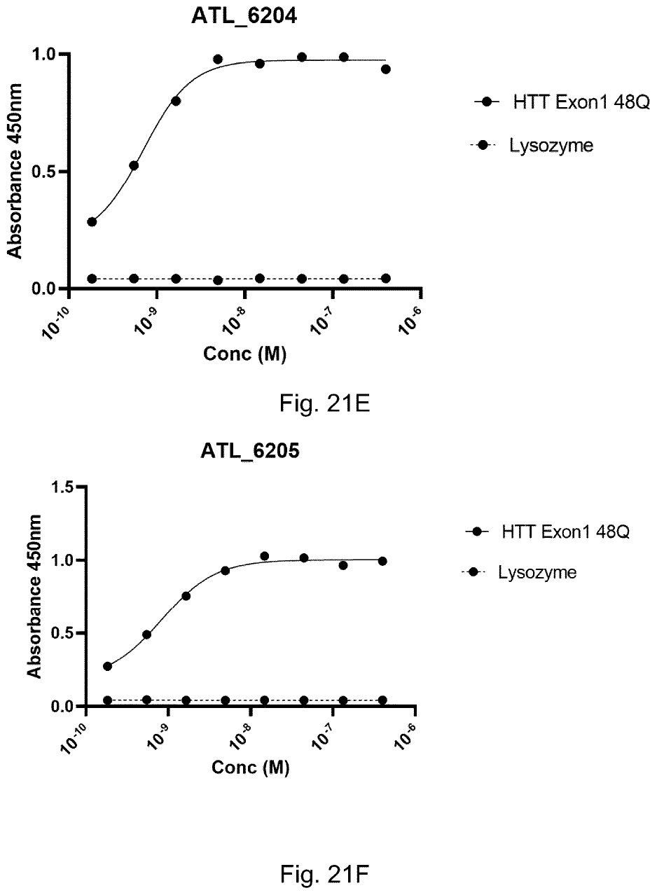

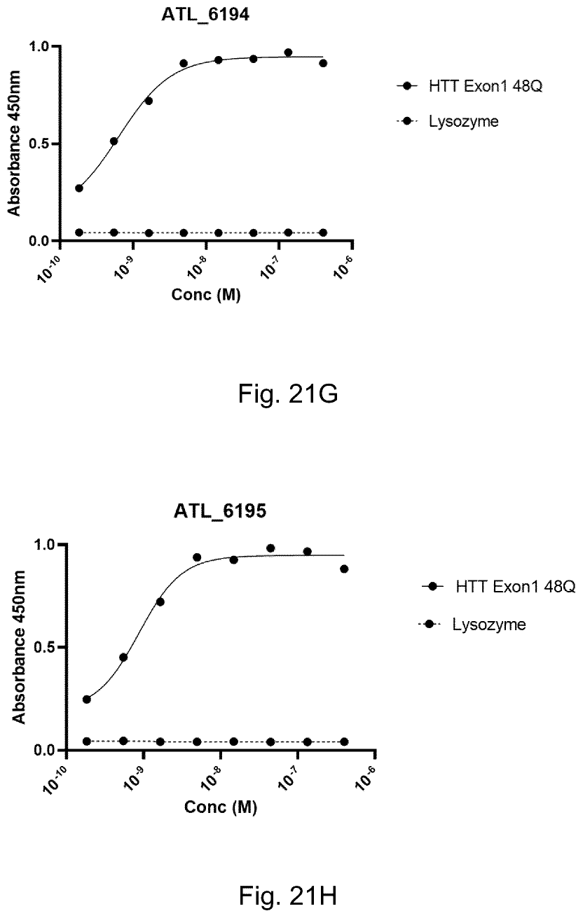

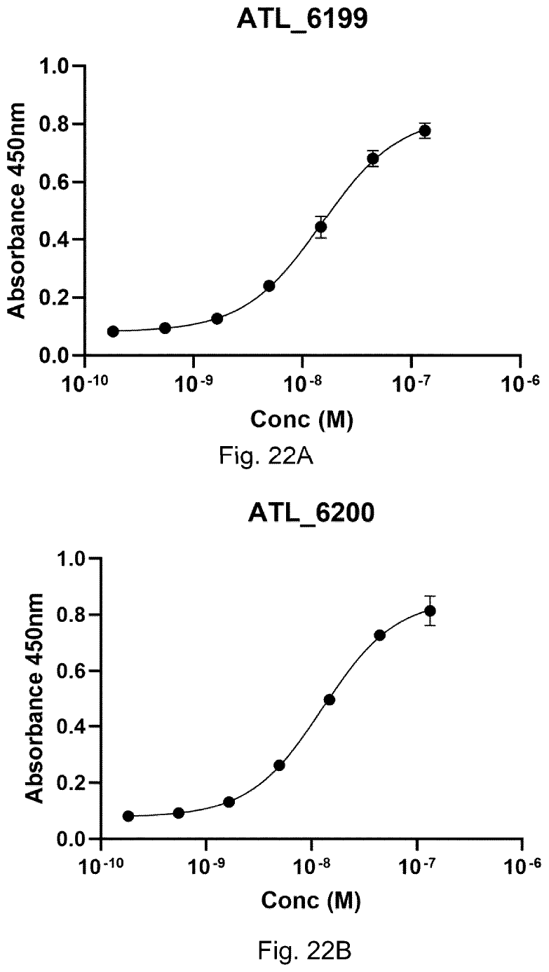

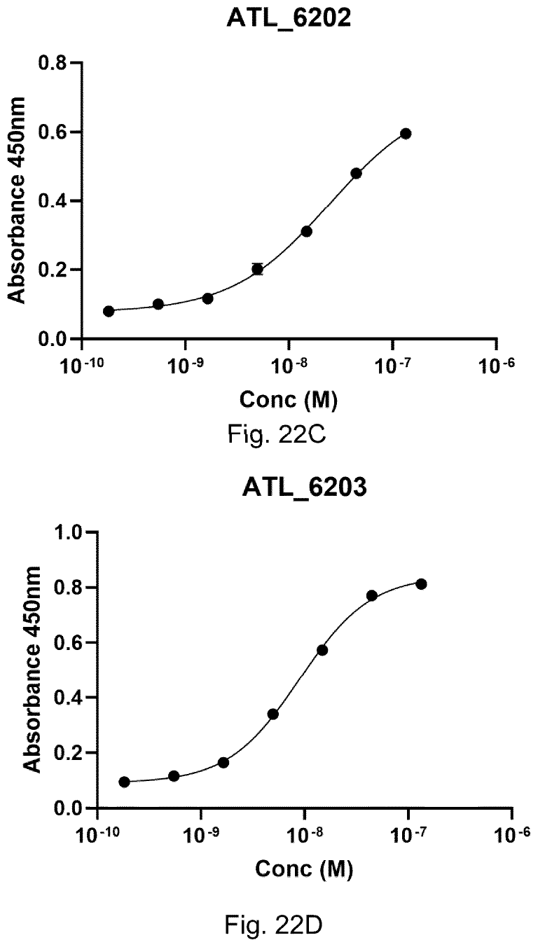

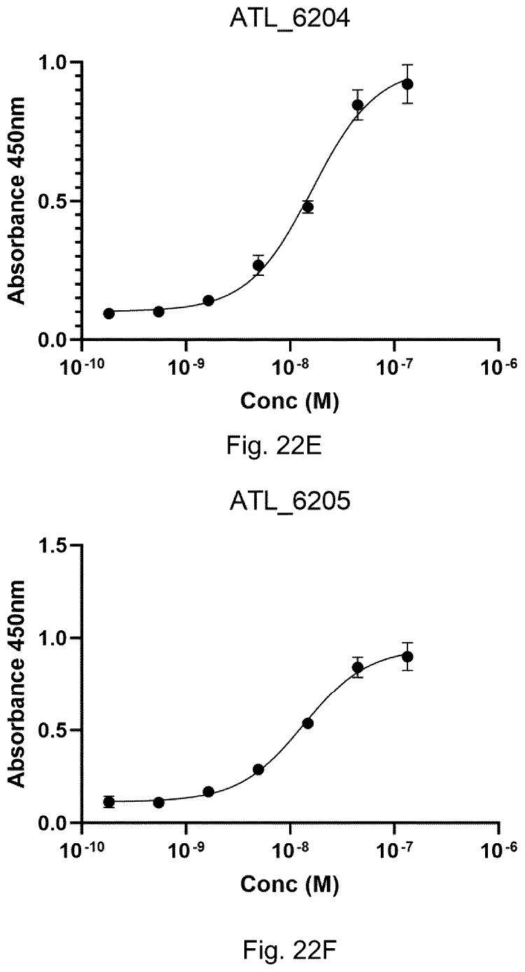

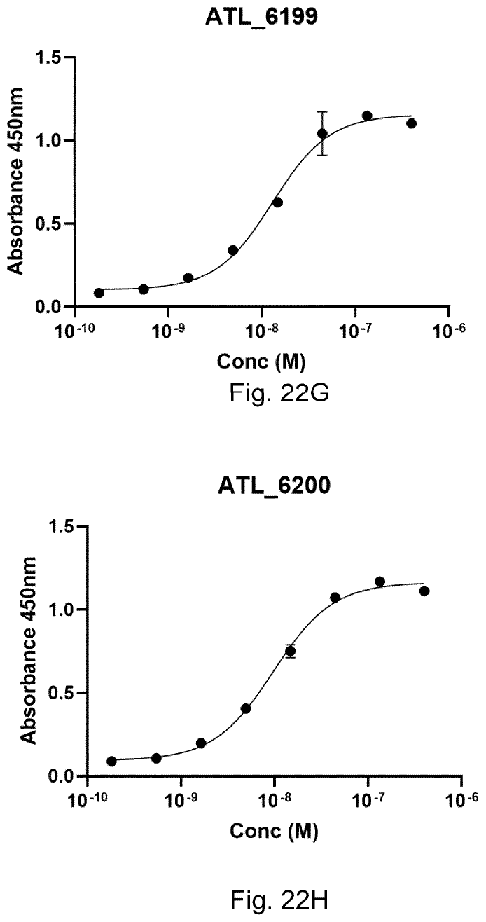

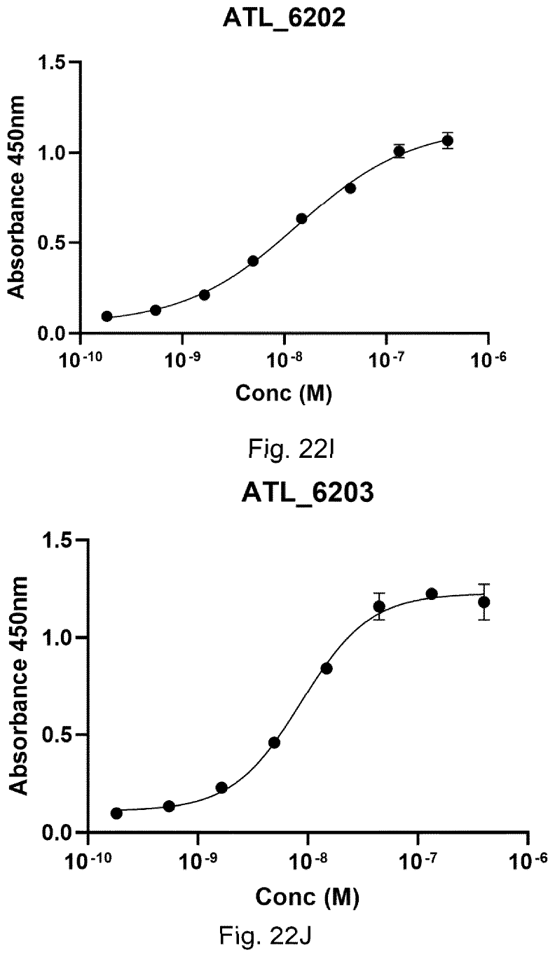

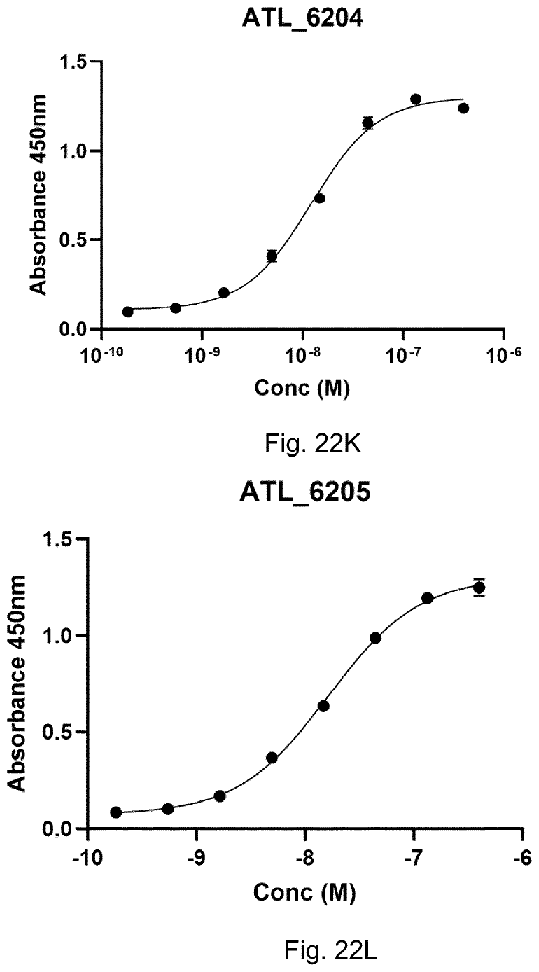

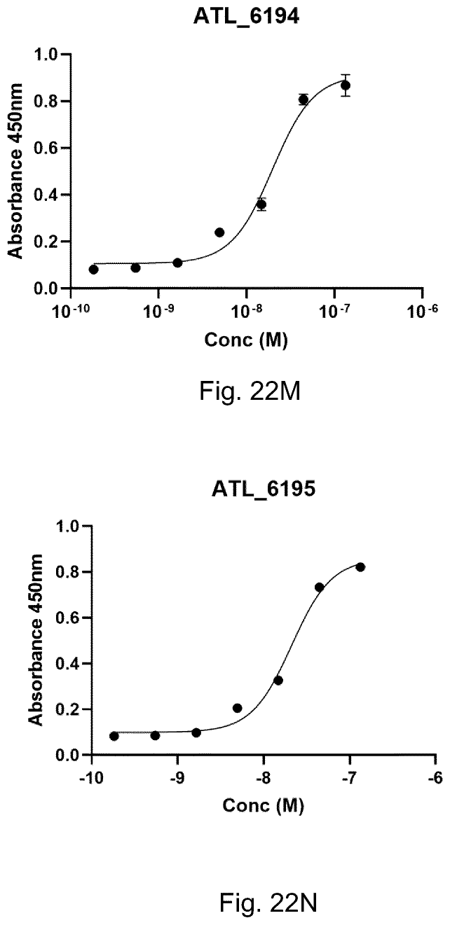

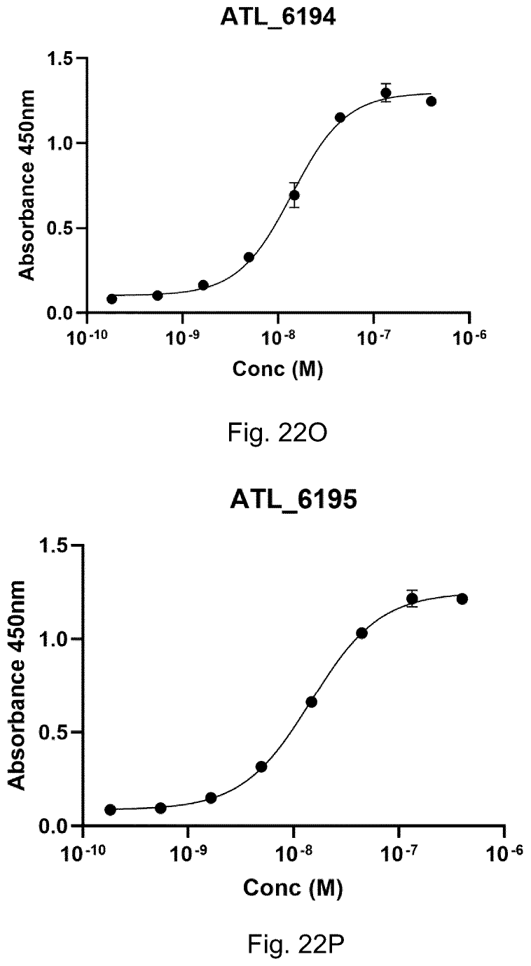

FIGS. 21 and 22 show the results of an indirect and sandwich ELISA, respectively, for binding of the newly identified antibodies to Exon 1 48Q HTT. Table 13 shows the EC50 values derived from the sandwich ELISA results in FIG. 22.

TABLE 13
EC50 values derived from sandwich ELISA.

| | HTT EC50 (M) | HTT EC50 (M) |
|---|---|---|
| | Experiment 1 (133 nM | Experiment 2 (400 nM |
| Antibody | starting concentration) | starting concentration) |
| ATL_6194 | 1.976e−8 | 1.40e−8 |
| ATL_6195 | 2.112e−8 | 1.47e−8 |
| ATL_6199 | 1.50e−8 | 1.31e−8 |
| ATL_6200 | 1.28e−8 | 9.75e−9 |
| ATL_6202 | Top of curve not reached | 1.35e−8 |
| ATL_6203 | 8.91e−9 | 8.72e−9 |
| ATL_6204 | 1.62e−8 | 1.22e−8 |
| ATL_6205 | 1.34e−8 | 1.64e−8 |
| ATL_5338 | No binding (negative | No binding (negative |
| | control) | control) |

The above data show that the additional antibodies identified through phage display are capable of binding to exon 1 48Q HTT with high affinity.

Antibodies ATL_6205, ATL_6202, ATL_6194, and ATL_6203 were used as a basis for further optimisation in Example 17. Antibody ATL_6195 was also selected for further investigation because homologue sequences were found in an HD resilient patient.

### Example 12—HD Patient Produces HTT-Binding Antibody

In order to identify further anti-HTT binding antibodies, the inventors studied the B cell repertoire of brain samples from patients diagnosed with HD via sequencing in order to identify antibodies homologous to the antibodies described herein. Brain samples were from the European Network of Brain Banking (ENBB). The identification of the anti-HTT binding antibodies in an HD patient further support the disease relevance of the antibodies of the present disclosure.

Two antibodies with homology to ATL_5895 were identified within the same ENBB patient, and they were paired with the VLs of ATL5895 and ATL5901 to generate four new antibodies. The two homologous antibodies identified from the ENBB patient showed differences in the FW regions compared with ATL5895 and ATL5901. Therefore, four further variants that only contained the HCDR3 of the two homologous antibodies, with the rest of the antibody being ATL5895 were also generated. Table 14 contains details on the VH and VL pairings, as well as CDR3 usage of the newly generated antibodies.

Table 16 shows the VH CDR sequences for each of these antibody variants, together with the VH CDR sequence of ATL5895 and ATL5901. The full VH and VL sequences of these antibodies are provided in Table 1.

TABLE 14
VH and VL pairings of new antibody variants

| Antibody | VH | VL |
|---|---|---|
| ATL6183 | ENBB homologous antibody VH1 CDR3; | ATL5895 VL |
| | ATL5895 VH | |
| ATL6184 | ENBB homologous antibody VH1 CDR3; | ATL5901 VL |
| | and ATL5901 VH | |
| ATL6185 | ENBB homologous antibody VH2 CDR3 | ATL5895 VL |
| | and ATL5895 VH | |
| ATL6186 | ENBB homologous antibody VH2 CDR3 | ATL5901VL |
| | and ATL5901 VH | |

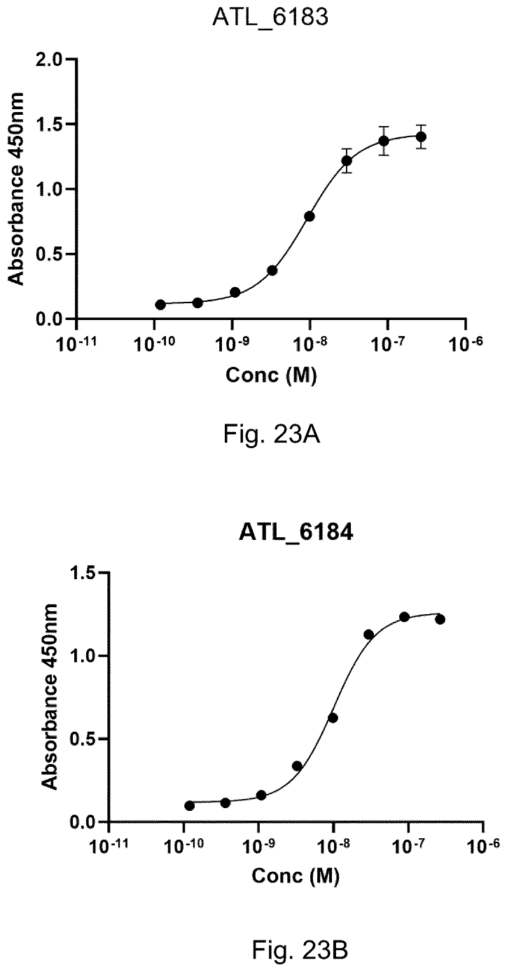

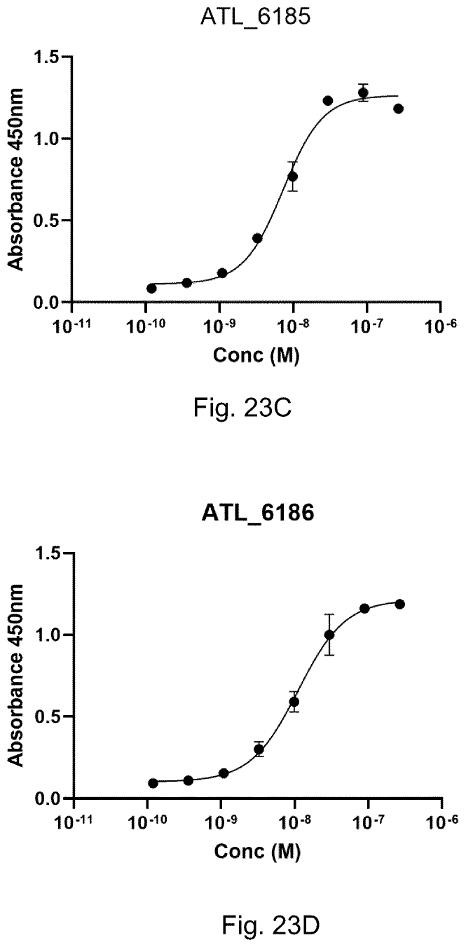

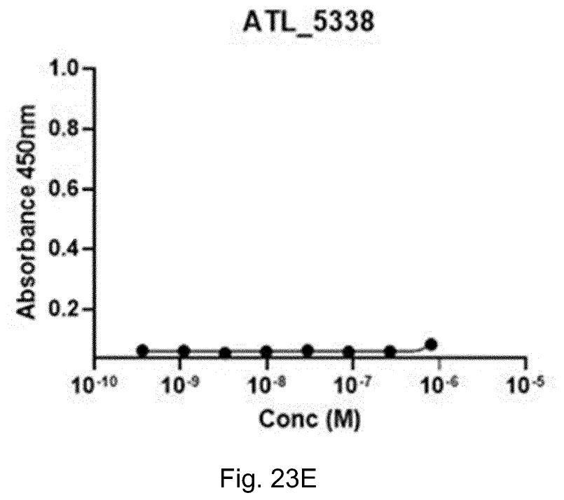

FIG. 23 shows the results of a sandwich ELISA, respectively, for binding of the newly identified antibodies to Exon 1 48Q HTT. All antibodies were shown to bind to HTT, whereas the control antibody ATL_5338 (isotype control, binds to fluorescein) did not bind HTT. Table 15 shows the EC50 values derived from the sandwich ELISA results shown in FIG. 23.

TABLE 15
EC50 values derived from sandwich
ELISA for indicated antibodies.

| | HTT EC50 | Antigen control EC50 |
|---|---|---|
| Antibody | (HTT sandwich ELISA) | (Lysozyme ELISA) |
| ATL_0005338 | Did not reach top of curve (neg | No binding |
| | control) | |
| ATL_0006183 | 9.20E−09 | No binding |
| ATL_0006184 | 1.01E−08 | No binding |
| ATL_0006185 | 7.31E−09 | No binding |
| ATL_0006186 | 1.11E−08 | No binding |

Together, these data confirm that the antibodies of the present invention are capable of binding disease relevant HTT.

TABLE 16
VH CDRs of patient derived antibodies.

| Antibody | VH CDR1 | VH CR2 | VH CDR3 |
|---|---|---|---|
| ATL6183 | KAWMS | RIKSGIDAGTTDYAAPVKG | PPYYYYGLDV |
| ATL6184 | KAWMS | RIKSGIDAGTTDYAAPVKG | PPYYYYGLDV |
| ATL6185 | KAWMS | RIKSGIDAGTTDYAAPVKG | PPYYYYGLNV |
| ATL6186 | KAWMS | RIKSGIDAGTTDYAAPVKG | PPYYYYGLNV |
| ATL5895 | KAWMS | RIKSGIDAGTTDYAAPVKG | PPYYYYYGLDV |
| ATL5901 | KAWMS | RIKSGIDAGTTDYAAPVKG | PPYYYYYGLDV |
| ATL5567 | KAWMN | RIKSGIDGGTTDYAAPVQG | PPYYYYYGLDV |

FIG. 24 shows the sequences of the FW regions and CDRs for the VH (FIG. 24A) and VL (FIG. 24B) of some antibodies of the present disclosure.

### Example 13—In Vitro Selectivity Assay

To investigate the selectivity of antibodies, ATL_5895 and ATL_5567 were first run in a library screen for binding against fixed HEK293 cells over-expressing 6105 individual full-length human plasma membrane proteins, secreted and cell surface-tethered human secreted proteins, as well as a further 400 human heterodimers to identify library interactions. This library screen was followed by a series of confirmatory screens in which all library interactions were re-expressed in fixed and live cells, and probed with each test antibody or control treatment, to determine which interactions were repeatable and specific to each test antibody (see “Materials and Methods” section above). This was performed on both fixed and live cells. HTT was spotted as an antigen in gelatin on to the screening slide as a positive control (as HTT is not part of this particular screening panel).

ATL_5895 and ATL_5567 both strongly bound to the HTT positive control (2 replicates per condition, 2 μg/mL antibody for ATL5895, 5 μg/mL antibody for ATL5567 and isotype control ATL5338). Another poly Q protein, CACNA1A, was present and was not detected by any of the antibodies. ATLX-1095 and ATL5567 showed no confirmed hits in this screening panel demonstrating their selectivity. Rituximab was used as a positive control for CD20 and was a hit for this antigen as expected.

This data shows that the ATL_5895 and ATL_5567 antibodies described herein specifically bind to HTT.

### Example 14—Live Animal PET/CT Scans to Assess Pharmacokinetics and Brain Penetration

To assess pharmacokinetics and brain penetration of ATL_5895, a PET labelled version of the antibody was prepared and live animal PET as well as gamma counting of post-mortem tissues was performed (see “Materials and Methods” section above).

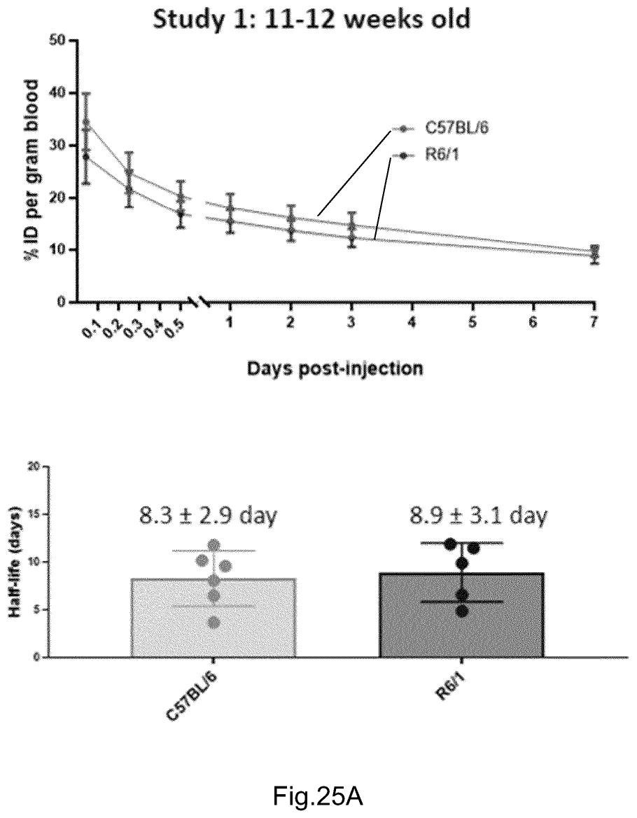

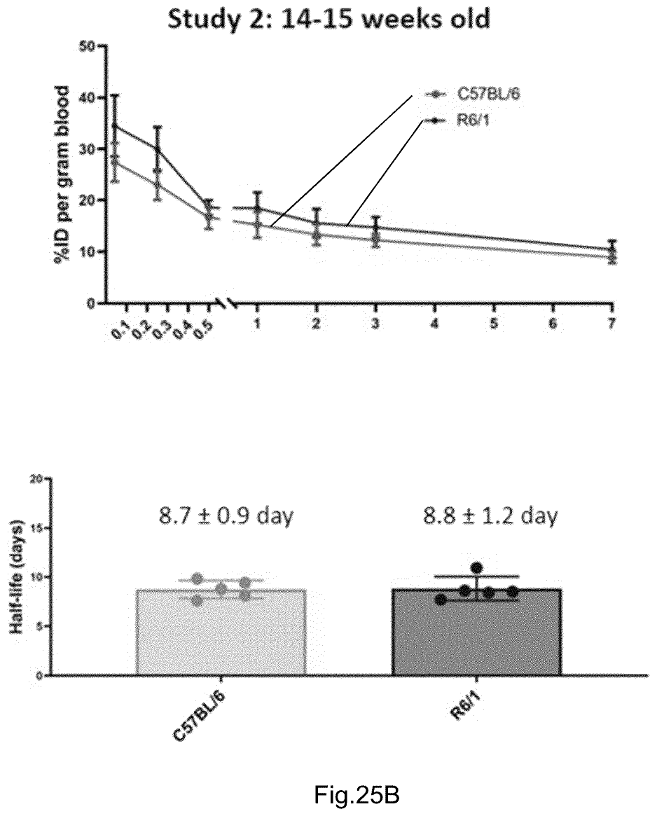

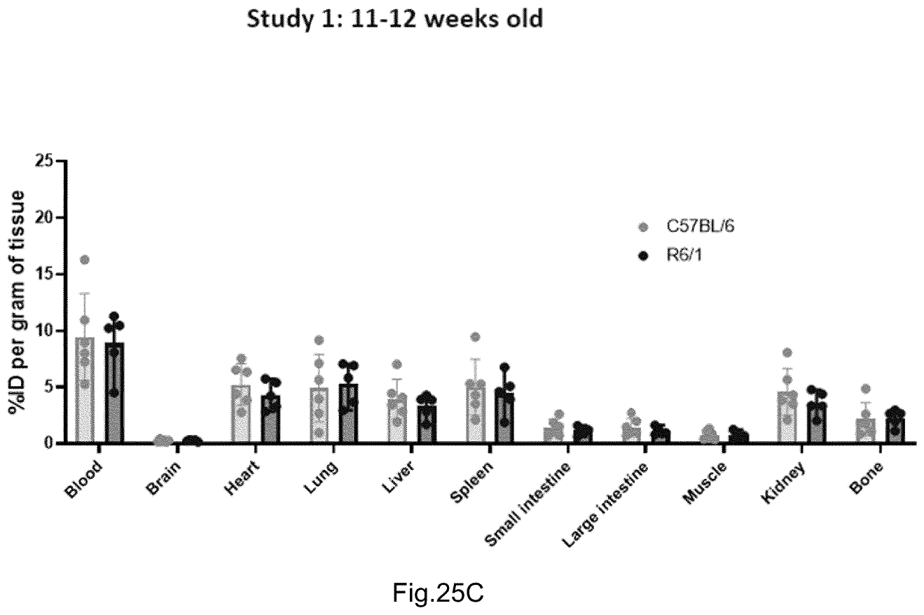

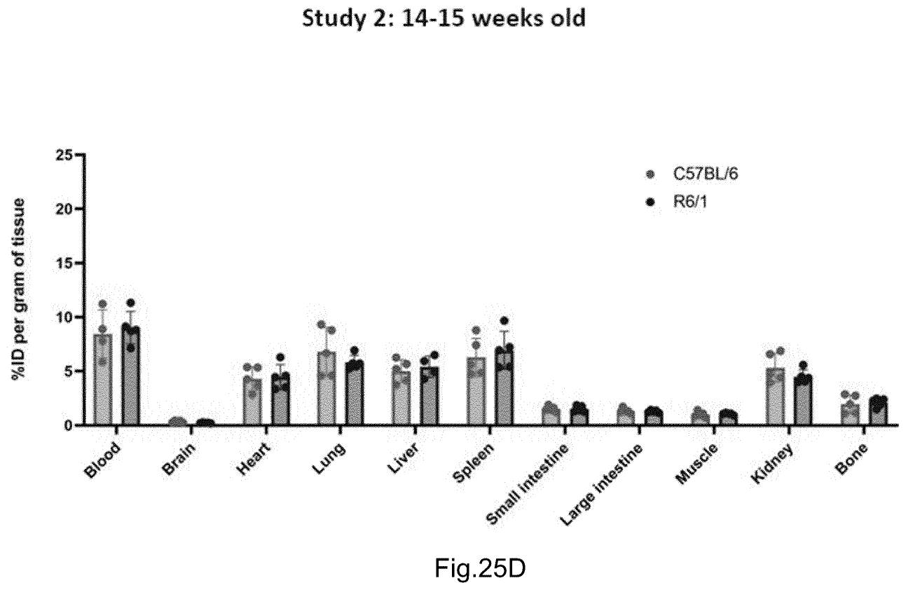

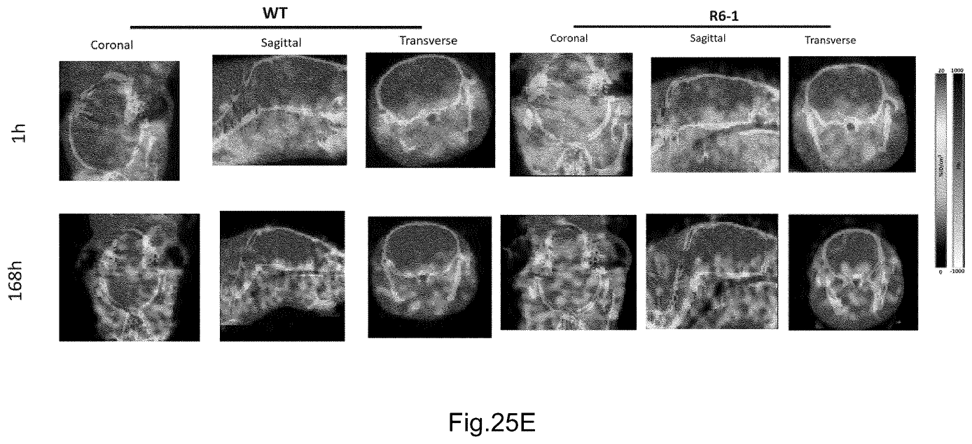

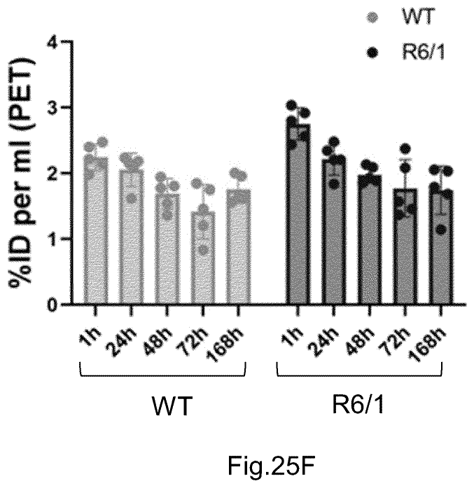

FIG. 25 shows the results of the live animal PET experiment and the gamma counting assay. Half-life was in the range of 8-9 days for the labelled antibody in both C57BL/6J and R6/1 mice in 11-12- and 14-15-week-old cohorts (FIGS. 25A and 25B). The mean brain: blood ratio was estimated to be 0.03±0.004 for both groups at 11-12 weeks and 0.04±0.01 for both groups at 14-15 weeks, as calculated by post-mortem gamma counting (FIGS. 25C and 25D). As demonstrated in Example 10, standard/classical PK experiments showed an exposure of ~5.5 nM in CSF (FIG. 20C), indicating that brain levels in this experiment may be underestimated. Live animal PET demonstrated a typical human IgG1 biodistribution of the labelled antibody, with the majority detected in the blood (FIGS. 25E and 25F)—although as explained above, the antibody was also detected in the brain and CSF.

### Example 15—In Vivo PD Assay

R6/1 is a transgenic mouse model of Huntington's disease which exhibits a progressive neurological phenotype that mimics many of the features of Huntington's Disease (Mangiarini et al; Cell; 1996) including an accumulation of aggregates over time (Hansson et al; EJN; 2001). These mice ubiquitously express a transgene comprising the 5′ end of mutated human huntingtin comprising approximately 1 kb of 5′ UTR sequences, exon 1 (carrying expanded CAG repeats with 115 to 150 CAG repeats) and the first 262 bp of intron 1. To assess the effect of ATL_5895 (ATLX_1095) on HTT aggregate load in R6/1 mice, mice were treated for up to 12 weeks (from age 5 weeks) with vehicle or ATL_5895. HTT aggregate load was assessed in the brain of these mice using an immunoassay (MSD). Soluble mutHTT (2B7; MW1+) was assessed in plasma with an increase observed, suggesting target engagement and clearance of HTT being driven by antibody-HTT complex in plasma.

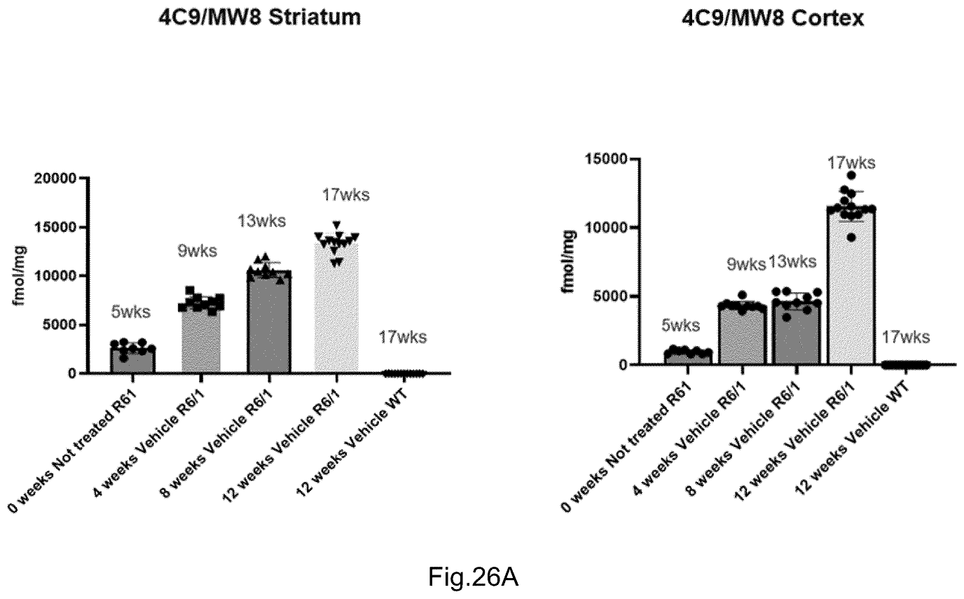

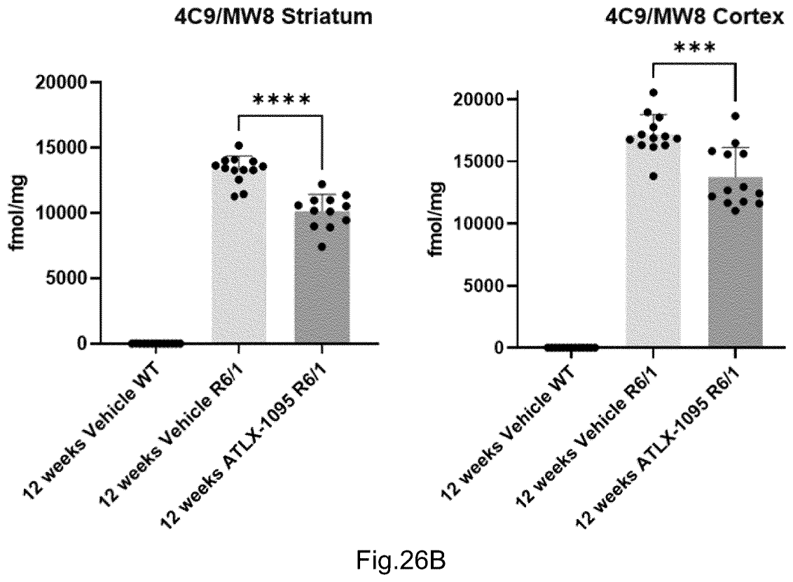

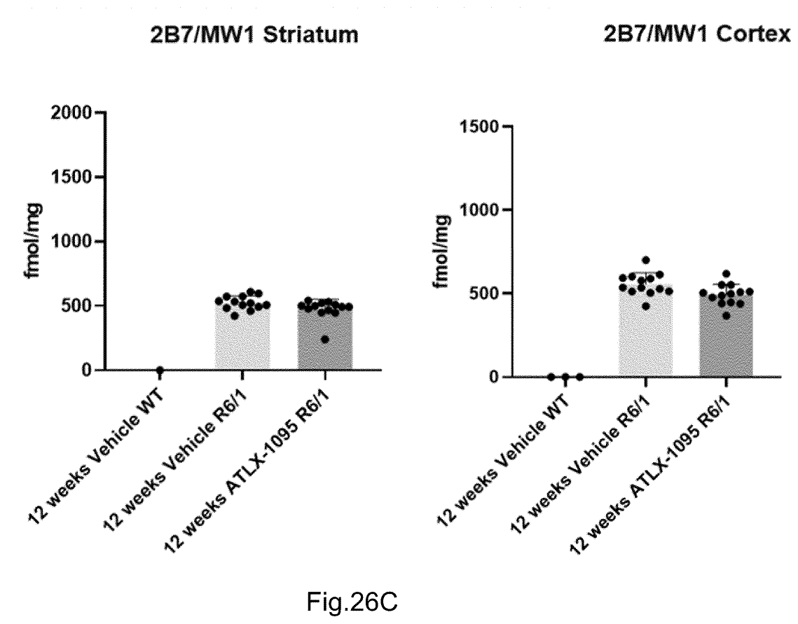

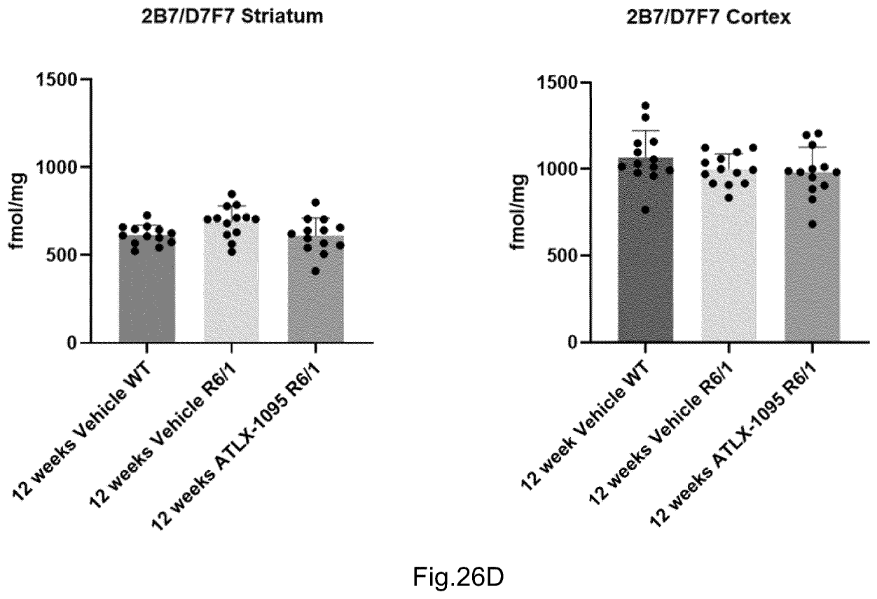

FIG. 26 shows the results of a meso scale discovery (MSD) assay to assess the effect of ATL_5895 (ATLX_1095) on HTT aggregate load in the striatum and cortex of R6/1 mice. As expected, HTT aggregates increased in the R6/1 mice over time in striatum and cortex (4C9/MW8+HTT; FIG. 26A) and concurrently there was a decrease in soluble mutant HTT over time as assessed by MSD assay (2B7/MW1). ATL_5895 treatment of R6/1 mice for 12 weeks resulted in a statistically significant decrease in HTT aggregates (as detected by MW8/4C9+ antibodies) in the striatum and cortex (FIG. 26B). There was no decrease in soluble HTT (as detected by 2B7/MW1 antibodies) or endogenous mouse HTT (as detected by 2B7/D7F7 antibodies) in the striatum and cortex (FIGS. 26C and 26D, respectively). Together, these results indicate that ATL_5895 (ATLX_1095) can selectively reduce HTT aggregates in the striatum and cortex of R6/1 mouse model of Huntington's disease without affecting endogenous HTT levels.

Plasma neurofilament light (NEFL) levels were assessed but no difference between WT and R6/1 mice was observed, thus there was not a phenotype for antibody to rescue.

### Example 16—Manufacturability

A stability assessment of the antibody ATLX-1095 (ATL_5895) was also performed, including a 4-week thermal stability study, 10× freeze-thaw cycle study, thermostability assessment (Tm and Tagg) and solubility study. The biophysical properties of the antibody was assessed following exposure to different stress conditions including (1) a 4 week thermal stability study in which the antibody was subjected to temperatures of −80° C., +4° C., +21° C. and +40° C. and (2) 10× freeze-thaw cycles. Stressed antibody samples were evaluated for purity, aggregation, degradation and changes in charge heterogeneity and binding using SEC-HPLC, CE-SDS, cIEF and ELISA compared to −80° C. control conditions.

The melting temperature (Tm) and aggregation temperature (Tagg) of unstressed antibody was assessed by DSF and SLS.

Antibody solubility was assessed by concentrating ATLX_1095 (ATL_5895) to 89.96 mg/mL using Tangential flow filtration (TFF). Samples were assessed for aggregation by SEC-HPLC analysis, after concentration and after 1 week at 21° C.

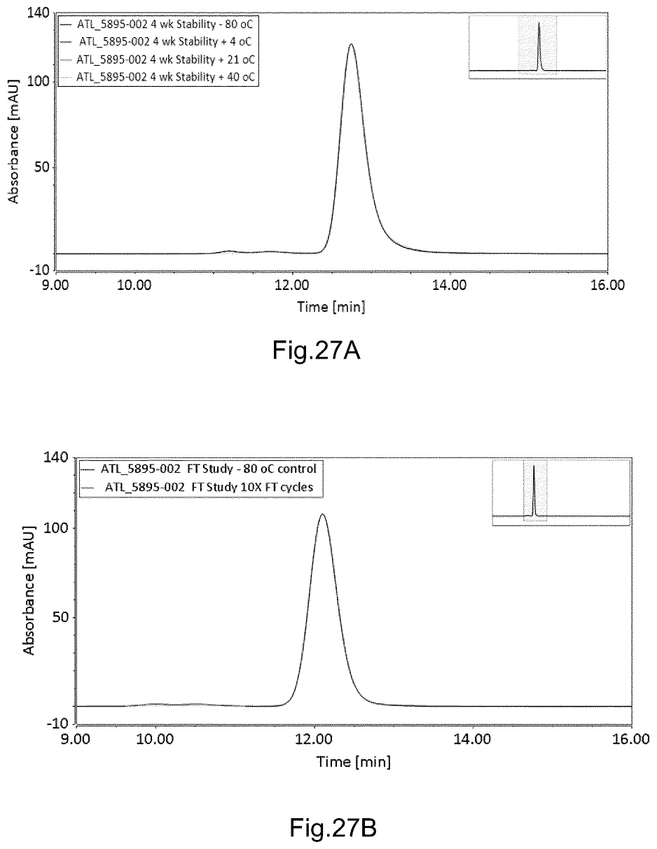

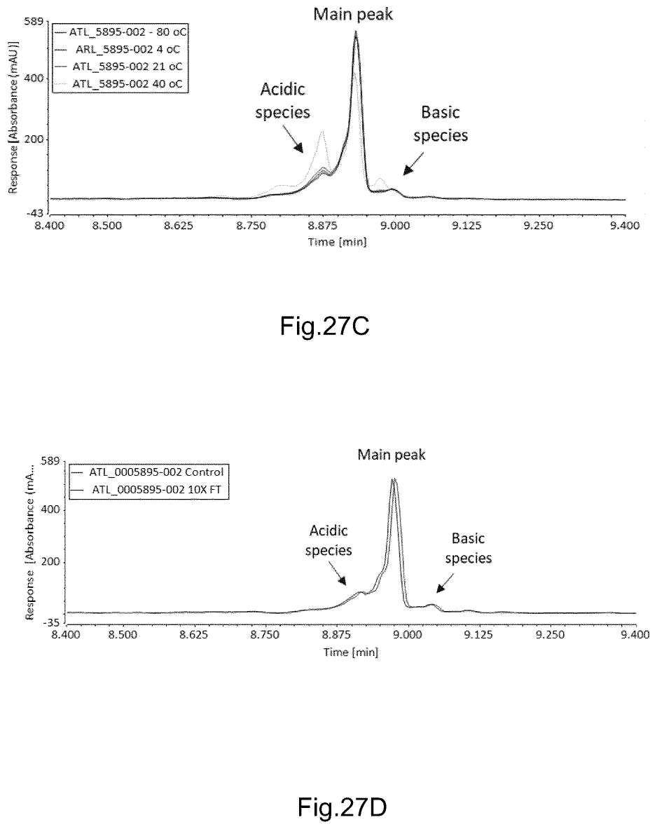

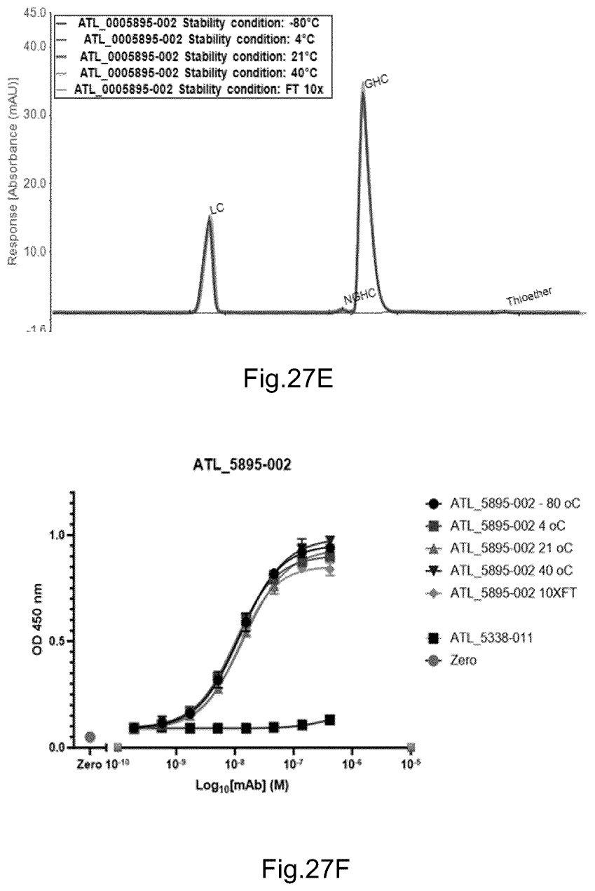

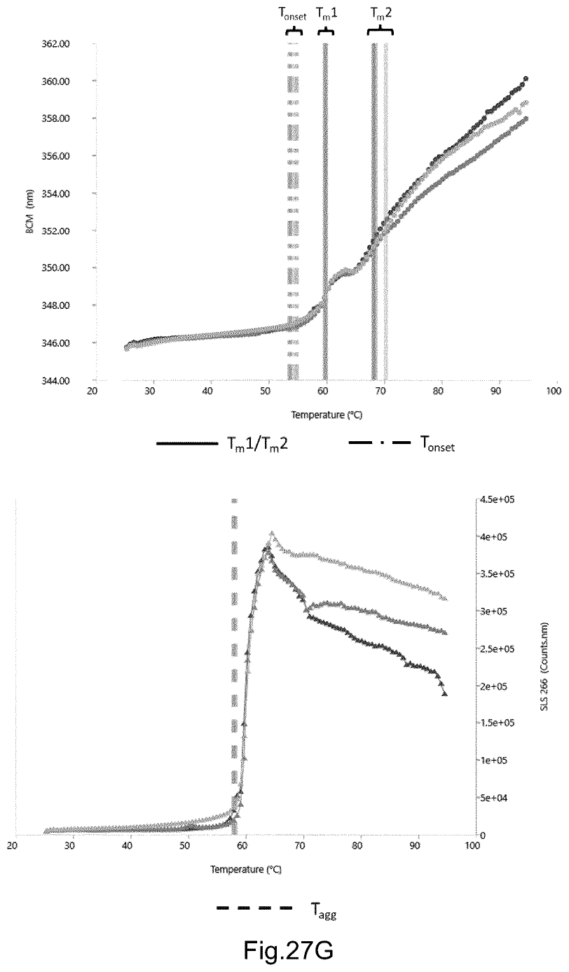

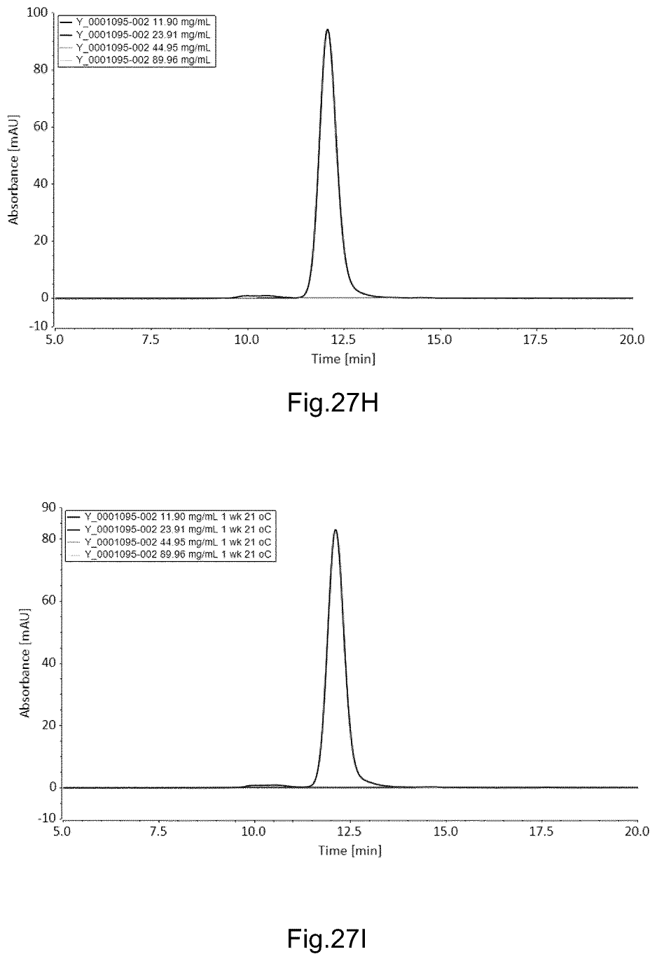

Protein aggregation during antibody storage must be kept to a minimum as it can cause immunogenic reactions. Size exclusion chromatography (SEC)-HPLC was used to evaluate the purity and aggregation of the antibody in the 4-week stability study as well as those subjected to 10× freeze thaw cycles. FIGS. 27A-B show SEC-HPLC chromatograms of ATL_5895 after 4 week thermal stability and 10× freeze-thaw, respectively. Both show no increase in soluble aggregate formation following incubation at −80° C., 4° C., room temperature (21° C.) and 40° C. for 4 weeks and after 10× freeze-thaw cycles respectively. The results in Table 17 shows that monomeric purity remained high (>95%).

TABLE 17
Summary of SEC-HPLC ATL_5895-002 4-week stability
study and 10x freeze-thaw study SEC-HPLC data showing
monomeric purity (%), high molecular weight (HMWS)
species (%) and low molecular weight (LMWS) species (%).

| Antibody | Condition | | % HMWS | % Monomer | % LMWS |
|---|---|---|---|---|---|
| ATL_5895 | −80° | C. | 2.41 | 97.59 | 0.00 |
| | +4° | C. | 2.12 | 97.88 | 0.00 |
| | +21° | C. | 2.13 | 97.87 | 0.00 |
| | +40° | C. | 1.47 | 97.54 | 1.00 |

| | | | |
|---|---|---|---|
| 10x FT cycles | 2.11 | 97.89 | 0.00 |

Capillary isoelectric focusing (cIEF) was used to assess charge heterogeneity of the antibody samples. The isoelectric point (pI) of unstressed ATL_5895 was determined using cIEF. The pI of ATL 5895 was 8.93, which is within the typical range of 7.5-9 for antibodies, appropriate for downstream processing (main peak area 71.84%). Changes in charge heterogeneity following temperature stress (−80° C., 4° C., room temperature (21° C.) and 40° C. for 4 weeks) and 10× freeze-thaw cycles were assessed by cIEF. An increase in acidic and basic charge variants and a decrease in main peak was observed for the highest temperature stress condition following incubation at 40° C. for 4-weeks but not at any of the other temperatures tested (FIG. 27C) or following 10× freeze thaw cycles (FIG. 27D). These results are also summarised in Table 18, which show stable % basic and acidic species after most treatments (main isoform % approx. 70%).

TABLE 18
Capillary isoelectric focussing results for ATL_5895
after temperature stress or 10x freeze thaw cycles.

| | | Acidic | Main | Basic |
|---|---|---|---|---|
| | | species | isoform | species |
| | | rel. area | rel. area | rel. area |
| Antibody | Condition | (%) | (%) | (%) |

| | | | | | |
|---|---|---|---|---|---|
| ATL_5895 | −80° | C. | 20.02 | 71.84 | 8.13 |
| | + 4° | C. | 19.73 | 71.34 | 8.93 |
| | + 21° | C. | 21.72 | 69.47 | 8.81 |
| | + 40° | C. | 43.34 | 45.42 | 11.24 |

| | | | |
|---|---|---|---|
| 10x FT cycles | 19.23 | 72.65 | 8.12 |

FIG. 27E and Table 19 show results of CE-SDS on samples of antibodies subjected to the indicated treatments (reduced prior to CE-SDS).

TABLE 19
Reduced CE-SDS light chain (LC), non-glycosylated
heavy chain (NGHC), heavy chain (HC) and thioether peak
area of ATL_5895 4-week thermal stability samples
and 10x freeze-thaw study samples.

| | | | LC | NGHC | HC | Thioether |
|---|---|---|---|---|---|---|
| | | | peak | peak | peak | peak |
| | | | area | area | area | area |
| Antibody | Condition | | (%) | (%) | (%) | (%) |
| ATL_5895 | −80° | C. | 25.98 | 0.81 | 72.86 | 0.35 |
| | + 4° | C. | 25.85 | 0.75 | 72.94 | 0.47 |
| | + 21° | C. | 25.82 | 0.74 | 72.74 | 0.49 |
| | + 40° | C. | 26.11 | 0.91 | 71.80 | 0.92 |

| | | | | |
|---|---|---|---|---|
| 10x FT cycles | 26.24 | 0.72 | 72.61 | 0.43 |

HTT Exon-1 Sandwich ELISA was performed to assess changes in binding of ATL_5895 to 48Q HTT Exon-1 following temperature stress {−80° C., 4° C., room temperature (21° C.) and 40° C. for 4 weeks} and 10× freeze-thaw cycles. FIG. 27F shows no significant changes in binding to 48Q HTT Exon-1 following temperature stress {−80° C., 4° C., room temperature (21° C.) and 40° C. for 4 weeks} and 10× freeze-thaw cycles compared to isotype control antibody (ATL_5338-011).

A protein thermal shift and light scattering assay was performed to determine the melting temperature (Tm1/Tm2) and aggregation temperature (Tagg/Tonset) of ATL_5895 (see “Materials and Methods” section above). FIG. 27G shows the results of the thermostability assay which are summarised in Table 20. These results demonstrate that ATL_5895 thermostability parameters are within typical range for an IgG1 antibody.

TABLE 20
Melting temperatures (Tm1/Tm2) and aggregation temperatures (Tagg) measured
for ATL_0005895 at 5 mg/mL in 20 mM Histidine-acetate, 150 mM NaCl pH 5.5.

| | | Replicate Number | | | | | |
|---|---|---|---|---|---|---|---|
| Sample | Parameter | 1 | 2 | 3 | Average (° C.) | % CV | SD |
| ATL_0005895 | Tm1 | 59.80 | 59.65 | 59.83 | 59.8 | 0.17 | 0.1 |
| | Tm2 | 68.08 | 68.42 | 70.23 | 68.9 | 1.68 | 1.16 |
| | Tonset | 53.58 | 54.78 | 54.15 | 54.2 | 1.11 | 0.6 |
| | Tagg | 57.83 | 58.21 | 57.67 | 57.9 | 0.48 | 0.28 |

To assess the solubility of ATL_5895 at different concentrations, a solubility assessment was performed (see “Materials and Methods” section above). No increase in aggregation was observed with ATL_5895 after concentration to a maximum of 89.96 mg/mL using tangential flow filtration (TFF) and following incubation at 21° C. for 1 week (FIG. 27H, FIG. 27I). Monomeric purity remained high (>97%) across all samples tested (Table 21, Table 22).

TABLE 21
Summary of initial (T0) ATL_0005895 solubility study SEC-HPLC
data showing monomeric purity (%), high molecular weight (HMWS)
species (%) and low molecular weight (LMWS) species (%).

| | Concen- | | | | |
|---|---|---|---|---|---|
| | tration | | HMWS | Monomer | LMWS |
| Sample | (mg/mL) | Condition | (%) | (%) | (%) |
| ATL_0005895 | 11.90 | T0 | 1.83 | 98.03 | 0.14 |
| ATL_0005895 | 23.91 | T0 | 1.88 | 97.98 | 0.14 |
| ATL_0005895 | 44.95 | T0 | 1.88 | 97.96 | 0.16 |
| ATL_0005895 | 89.96 | T0 | 1.89 | 97.96 | 0.15 |

TABLE 22
Summary of ATL_0005895 solubility study SEC-HPLC
data after 1 week at 21° C. showing monomeric
purity (%), high molecular weight (HMWS) species
(%) and low molecular weight (LMWS) species (%).

| | Concen- | | | | |
|---|---|---|---|---|---|
| | tration | | HMWS | Monomer | LMWS |
| Sample | (mg/mL) | Condition | (%) | (%) | (%) |
| ATL_0005895 | 11.90 | 1 week at | 1.97 | 97.83 | 0.19 |
| | | 21° C. | | | |
| ATL_0005895 | 23.91 | 1 week at | 2.07 | 97.72 | 0.21 |
| | | 21° C. | | | |
| ATL_0005895 | 44.95 | 1 week at | 2.22 | 97.53 | 0.25 |
| | | 21° C. | | | |
| ATL_0005895 | 89.96 | 1 week at | 2.41 | 97.36 | 0.23 |
| | | 21° C. | | | |

### Example 17—Affinity Matured HTT Antibody Combinations

Antibodies were generated using combinations of the mutations that were present in the affinity matured variants ATL_6205, ATL_6202, ATL_6194, and/or ATL_6203 (see Example 11 above).

To assess binding potency of the affinity matured antibodies to HTT exon 1 48Q protein, the antibodies were assessed in a sandwich ELISA format for binding to human HTT exon 1 48Q captured by the anti-polyQ specific antibody, clone MW1 (MerckMillipore; #MABN2427; see “Materials and Methods” section above). Lysozyme was used as a negative control antigen and no discernible binding was shown by any of the antibodies to this control. ATL5338 was used as a negative isotype control and showed no binding to HTT exon 1 48Q.

Table 23 shows the VH CDR sequences for each of the antibodies generated using combinations of the mutations that were present in the affinity matured variants, together with the VH CDR sequences of ATL5895. The full VH and VL sequences of these antibodies are provided in Table 1.

TABLE 23
VH CDRs of antibodies generated using combinations of the
mutations that were present in the affinity matured variants

| Antibody | VH CDR1 | VH CR2 | VH CDR3 |
|---|---|---|---|
| ATL 6374 | KAWMS | RIKSGIDAGTTDYAAPVKG | PPYYYYYGLDV |
| ATL 6375 | KAWMS | RIKSGIDAGTTDYAAPVKG | PPYYYYYGLDV |
| ATL 6376 | KAWMS | RIKSGIDAGTTDYAAPVKG | PPFYYYYGLDV |
| ATL 6377 | KAWMS | RIKSGIDAGTTDYAAPVKG | SPYYYYYGLDV |
| ATL 6378 | KAWMS | RIKSGIDAGTTDYAAPVKG | SPYYYYYGLDV |
| ATL_5895 | KAWMS | RIKSGIDAGTTDYAAPVKG | PPYYYYYGLDV |

Table 24 shows the EC50 values derived from the sandwich ELISA for binding of the newly generated antibodies to Exon 1 48Q HTT. All antibodies were shown to bind to HTT, whereas the control antibody ATL_5338 (isotype control, binds to fluorescein) did not bind HTT. EC50s calculated using a variable slope (four parameter) non-linear fit of the data. Each of the generated antibodies had similarly low EC50 levels. ATL_6375, ATL_6376 and ATL_6377 showed the greatest binding affinity of the antibodies tested. EC50 for ATL_5895 is shown in Table 10 (2.077017 E-09 M). EC50 for comparative antibody ATL_0005059/NI-302.8F1 is shown in Table 10 (1.725504 E-08 M).

TABLE 24
EC50 values derived from sandwich
ELISA for indicated antibodies.

| | HTT EC50 | Antigen control EC50 |
|---|---|---|
| Antibody | (HTT sandwich ELISA) | (Lysozyme ELISA) |
| ATL_6374 | 3.11E−08M | No binding |
| ATL_6375 | 2.01E−08M | No binding |
| ATL_6376 | 1.15E−08M | No binding |
| ATL_6377 | 2.38E−08M | No binding |
| ATL_6378 | 3.89E−08M | No binding |
| ATL_5338 | No binding (neg control) | No binding |

### Example 18—Murine HTT ELISA

To assess binding potency of antibodies to murine HTT protein, ATL5895, ATL6376 and ATL6377 were assessed in a direct ELISA format for binding to murine HTT and human lysozyme (as negative control antigen). ATL5338 was used as a negative isotype control.

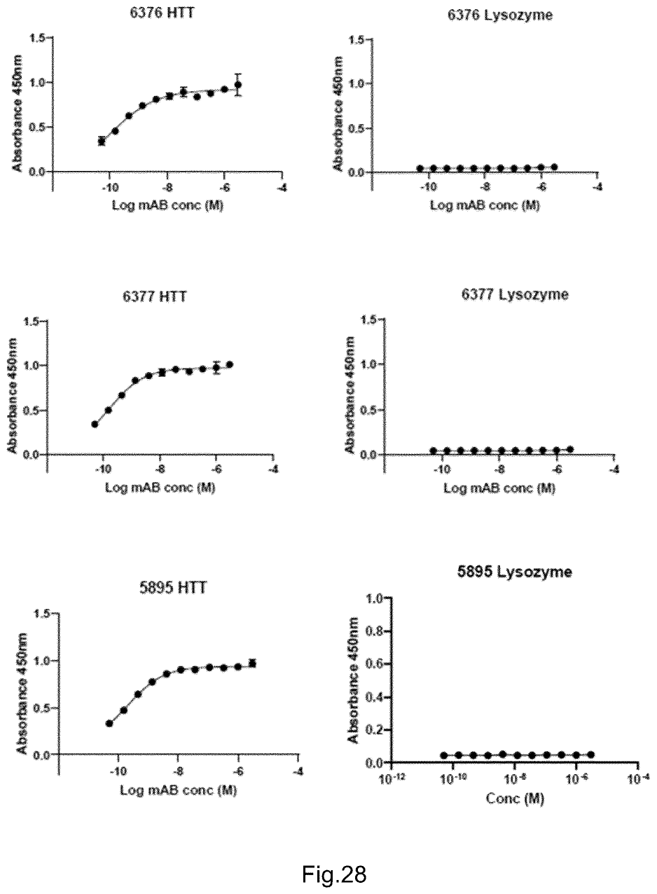

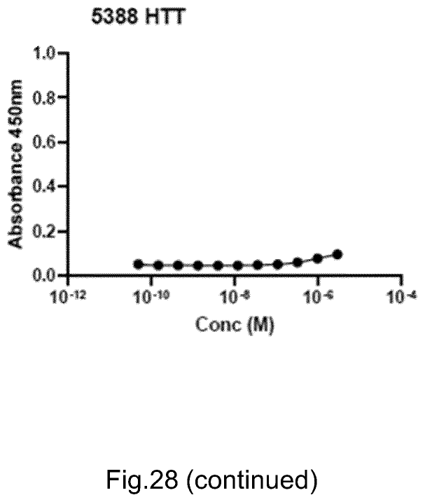

FIG. 28 shows the results of an indirect ELISA for binding of the antibodies to murine HTT. ATL5895, ATL6376 and ATL6377 all demonstrated binding to murine HTT, ATL5338 did not bind murine HTT (isotype control). Lysozyme showed no discernible binding to any of the antibodies assessed at concentrations less than 1 μM. These results indicate that ATL5895, ATL6376 and ATL6377 have cross species reactivity to murine HTT.

### Example 19—Binding of Affinity Matured Antibodies by Bio-Layer Interferometry

Binding interaction of ATL_5895 and affinity matured antibodies described in Examples 11 and 17 above was assessed by bio-layer interferometry (BLI) using an Octet instrument. HTT exon1 in the form of biotinylated peptide (SEQ ID NO: 174) or Mutant HTT exon1 48Q (SEQ ID NO: 44) was loaded onto either Streptavidin or GST biosensors (Sartorius, 18-5019 and 18-5096) respectively. Subsequently, the sensors were dipped into separate wells containing indicated concentrations of mAbs for 300s at 1000 rpm to measure the binding during this association phase. Binding responses are reported for the end of the association phase for all mAbs.

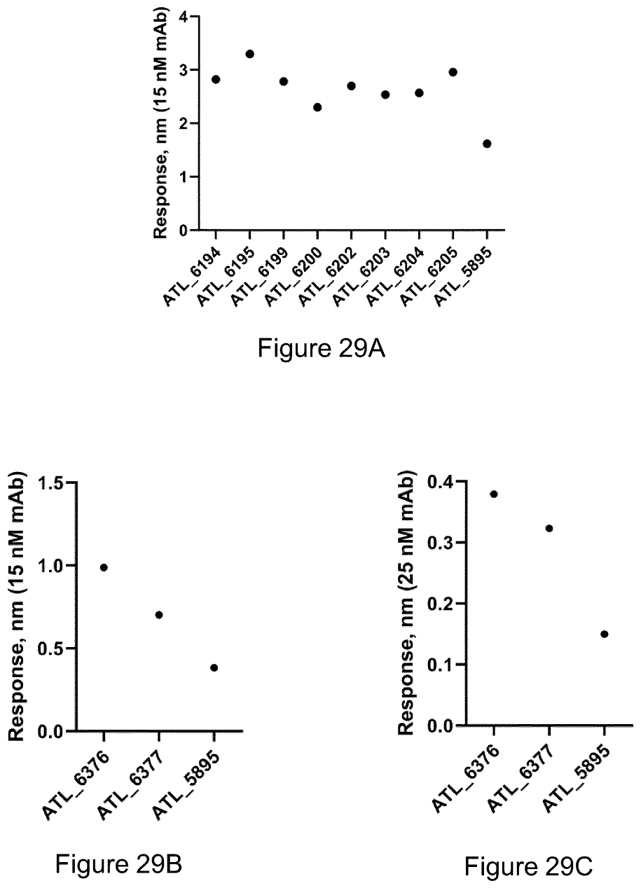

FIG. 29 shows the results of these experiments, indicating that affinity matured antibodies bind to HTT exon 1 and mutant HTT exon 1 and show increased propensity to bind in comparison to the parent ATL_5895.

1. An isolated antibody or antibody fragment thereof which specifically binds to Huntingtin (HTT) protein or a fragment thereof, the antibody comprising a heavy chain variable (VH) domain comprising CDRs HCDR1, HCDR2 and HCDR3, and a light chain variable (VL) domain comprising CDRs LCDR1, LCDR2 and LCDR3, wherein:
i. HCDR1 has amino acid sequence KAWMS (SEQ ID NO:1);
ii. HCDR2 has amino acid sequence RIKSGIDAGTTDYAAPVKG (SEQ ID NO:2);
iii. HCDR3 has amino acid sequence PPYYYYYGLDV (SEQ ID NO: 3), or a sequence comprising one or two substitutions compared with PPYYYYYGLDV (SEQ ID NO: 3), wherein the substitutions are at positions selected from: 95 and 97, wherein the substitutions are selected from: Y97F and P95S, wherein the position numbering is Kabat;
iv. LCDR1 has an amino acid sequence TGTSSDVGSYNLVS (SEQ ID NO:4);
v. LCDR2 has an amino acid sequence EVNKRPS (SEQ ID NO:5); and
vi. LCDR3 has an amino acid sequence GSYAGTANV (SEQ ID NO:6), or an amino acid sequence comprising one, two, three or four amino acid substitutions compared with GSYAGTANV (SEQ ID NO: 6), wherein the substitutions are selected from: A92G, A95E, and G89V, and Y91F, wherein the position numbering is Kabat.

2. The isolated antibody or fragment thereof according to claim 1, wherein:
the isolated antibody or fragment thereof has improved binding to mutated and/or aggregated HTT protein compared to non-mutated and/or non-aggregated HTT protein, wherein relative binding to mutated and/or aggregated and non-mutated and/or non-aggregated HTT is measured by determining the ratio of EC50 values for a HTT protein or fragment thereof comprising 25Q repeats in Exon 1 and a HTT protein or fragment thereof comprising 48Q repeats in Exon 1;
wherein the isolated antibody or fragment thereof has a ratio of EC50 for binding to a HTT protein or fragment thereof comprising 25Q repeats in Exon 1 and a HTT protein or fragment thereof comprising 48Q repeats in Exon 1 of at least 1.5, at least 1.6, at least 1.7, at least 1.8, at least 1.9 or at least 2, as measured by sandwich ELISA.

3. The isolated antibody or antibody fragment according to claim 1 or claim 2, wherein the antibody comprises:
(a) a heavy chain variable (VH) domain comprising CDRs HCDR1, HCD2 and HCDR3, wherein:
i. HCDR1 has amino acid sequence KAWMS (SEQ ID NO:1);
ii. HCDR2 has amino acid sequence RIKSGIDAGTTDYAAPVKG (SEQ ID NO:2);
iii. HCDR3 has amino acid sequence PPYYYYYGLDV (SEQ ID NO:3), and
a light chain variable (VL) domain selected from:
(1) a VL domain comprising CDRs LCDR1, LCDR2 and LCDR3, wherein:
i. LCDR1 has amino acid sequence TGTSSDVGSYNLVS (SEQ ID NO:4);
ii. LCDR2 has amino acid sequence EVNKRPS (SEQ ID NO:5);
iii. LCDR3 has amino acid sequence GSYAGTANV (SEQ ID NO:6);
(2) a VL domain comprising CDRs LCDR1, LCDR2 and LCDR3, wherein:
i. LCDR1 has amino acid sequence TGTSSDVGSYNLVS (SEQ ID NO:4);
ii. LCDR2 has amino acid sequence EVNKRPS (SEQ ID NO:5); and
iii. LCDR3 has amino acid sequence VSYGGTENV (SEQ ID NO:162);
(3) a VL domain comprising CDRs LCDR1, LCDR2 and LCDR3, wherein:
i. LCDR1 has amino acid sequence TGTSSDVGSYNLVS (SEQ ID NO:4);
ii. LCDR2 has amino acid sequence EVNKRPS (SEQ ID NO:5); and
iii. LCDR3 has amino acid sequence VSFAGTANV (SEQ ID NO: 160); or
(4) a VL domain comprising CDRs LCDR1, LCDR2 and LCDR3, wherein:
i. LCDR1 has amino acid sequence TGTSSDVGSYNLVS (SEQ ID NO:4);
ii. LCDR2 has amino acid sequence EVNKRPS (SEQ ID NO:5); and
iii. LCDR3 has amino acid sequence VSYAGTANV (SEQ ID NO: 161); or
(b) a heavy chain variable (VH) domain comprising CDRs HCDR1, HCD2 and HCDR3, wherein:
i. HCDR1 has amino acid sequence KAWMS (SEQ ID NO:1);
ii. HCDR2 has amino acid sequence RIKSGIDAGTTDYAAPVKG (SEQ ID NO:2);
iii. HCDR3 has amino acid sequence PPFYYYYGLDV (SEQ ID NO: 158); and
a light chain variable (VL) domain comprising CDRs LCDR1, LCDR2 and LCDR3, wherein:
i. LCDR1 has amino acid sequence TGTSSDVGSYNLVS (SEQ ID NO:4);
ii. LCDR2 has amino acid sequence EVNKRPS (SEQ ID NO:5); and
iii. LCDR3 comprising amino acid sequence VSYGGTENV (SEQ ID NO: 162); or
(c) a heavy chain variable (VH) domain comprising CDRs HCDR1, HCD2 and HCDR3, wherein:
i. HCDR1 has amino acid sequence KAWMS (SEQ ID NO:1);
ii. HCDR2 has amino acid sequence RIKSGIDAGTTDYAAPVKG (SEQ ID NO:2);
iii. HCDR3 has amino acid sequence SPYYYYYGLDV (SEQ ID NO: 157), and
a light chain variable (VL) domain selected from:
(1) a VL domain comprising CDRs LCDR1, LCDR2 and LCDR3, wherein:
i. LCDR1 has amino acid sequence TGTSSDVGSYNLVS (SEQ ID NO:4);
ii. LCDR2 has amino acid sequence EVNKRPS (SEQ ID NO:5);
iii. LCDR3 has amino acid sequence VSYAGTANV (SEQ ID NO: 161); or
(2) a VL domain comprising CDRs LCDR1, LCDR2 and LCDR3, wherein:
i. LCDR1 has amino acid sequence TGTSSDVGSYNLVS (SEQ ID NO:4);
ii. LCDR2 has amino acid sequence EVNKRPS (SEQ ID NO:5);
iii. LCDR3 has amino acid sequence VSYGGTENV (SEQ ID NO: 162).

4. The isolated antibody or fragment thereof according to any one of the preceding claims, wherein the VH domain is a human VH domain, and/or wherein the antibody or fragment thereof has a VH domain framework sequence selected from:
the framework sequence of ATL_0005895 VH:
(SEQ ID NO: 98)
EVQLVESGGGLVKPGGSLRLSCAASGFTFN
-[CDRH1]-
(SEQ ID NO: 99)
WVRQAPGKGLEWVG
-[CDRH2]-
(SEQ ID NO: 172)
RFTISRDDSKNTLYLQMNSLKTEDTAVYYCIP
-[CDRH3]-
(SEQ ID NO: 101)
WGQGTTVTVSS,
the framework sequence of ATL_0006376 VH:
(SEQ ID NO: 98)
EVQLVESGGGLVKPGGSLRLSCAASGFTFN
-[CDRH1]-
(SEQ ID NO: 99)
WVRQAPGKGLEWVG
-[CDRH2]-
(SEQ ID NO: 173)
RFTISRDDSKNTLYLQMNSLKTEDTAVYWCVP
-[CDRH3]-
(SEQ ID NO: 101)
WGQGTTVTVSS,
the framework sequence of ATL_0006199 VH:
(SEQ ID NO: 98)
EVQLVESGGGLVKPGGSLRLSCAASGFTFN
-[CDRH1]-
(SEQ ID NO: 99)
WVRQAPGKGLEWVG
-[CDRH2]-
(SEQ ID NO: 169)
RFTISRDDSKNTLYLQMNSLKTEDTAVYWCSP
-[CDRH3]-
(SEQ ID NO: 101)
WGQGTTVTVSS,
and
the framework sequence of ATL_0006200 VH:
(SEQ ID NO: 98)
EVQLVESGGGLVKPGGSLRLSCAASGFTFN
-[CDRH1]-
(SEQ ID NO: 99)
WVRQAPGKGLEWVG
-[CDRH2]-
(SEQ ID NO: 170)
RFTISRDDSKNTLYLQMNSLKTEDTAVYYCVP
-[CDRH3]-
(SEQ ID NO: 101)
WGQGTTVTVSS,
the framework sequence of ATL_0006202 VH:
(SEQ ID NO: 98)
EVQLVESGGGLVKPGGSLRLSCAASGFTFN
-[CDRH1]-
(SEQ ID NO: 99)
WVRQAPGKGLEWVG
-[CDRH2]-
(SEQ ID NO: 171)
RFTISRDDSKNTLYLQMNSLKTEDTAVYYCSP-
[CDRH3]-
(SEQ ID NO: 101)
WGQGTTVTVSS,
or
the framework sequence of ATL_006195 VH:
(SEQ ID NO: 98)
EVQLVESGGGLVKPGGSLRLSCAASGFTFN
-[CDRH1]-
(SEQ ID NO: 99)
WVRQAPGKGLEWVG
-[CDRH2]-
(SEQ ID NO: 180)
RFTISRDDSKNTLYLQMNSLKTEDTAVYYCTP
-[CDRH3]-
(SEQ ID NO: 101)
WGQGTTVTVSS.

5. The isolated antibody or fragment thereof according to any one of the preceding claims, wherein the VL domain is a human VL domain and/or wherein the antibody or fragment thereof has the framework sequence of ATL_0005895 VL: QSALTQPRSVSGSPGQSVTISC (SEQ ID NO: 131)
[CDRL1]-
(SEQ ID NO: 133)
WYQQHPGKAPKLMIY
[CDRL2]-
(SEQ ID NO: 138)
GVPDRFSGSKSGATASLTISGLQAEDEADYYC
[CDRL3]-
(SEQ ID NO: 139)
FGTGTKLTVL.

6. The isolated antibody or fragment thereof according to any one of the preceding claims, wherein HCDR1, HCDR2 and HCDR3 of the VH domain are within a germline framework and/or wherein LCDR1, LCDR2, LCDR3 of the VL domain are within a germline framework.

7. The isolated antibody or fragment thereof according to any one of the preceding claims, wherein:
the heavy chain variable domain comprises the amino acid sequence of any of ATL_5895 VH (SEQ ID NO: 7), ATL_6204 VH (SEQ ID NO: 7), ATL_6199 VH (SEQ ID NO: 144), ATL_6374 VH (SEQ ID NO: 148), ATL_6194 VH (SEQ ID NO:145), ATL_6375 VH (SEQ ID NO: 145), ATL_6200 VH (SEQ ID NO: 145), ATL_6202 VH (SEQ ID NO: 146), ATL_6203 VH (SEQ ID NO: 147), ATL_6205 VH (SEQ ID NO: 148), ATL_6376 VH (SEQ ID NO: 175), ATL_6377 VH (SEQ ID NO: 176), ATL_6195 VH (SEQ ID NO: 179), ATL_6378 VH (SEQ ID NO: 176); and
the light chain variable domain comprises the amino acid sequence of any of ATL_5895 VL (SEQ ID NO:8), ATL_6199 VL (SEQ ID NO: 8), ATL_6195 VL (SEQ ID NO: 8), ATL_6002 VL (SEQ ID NO: 8), ATL_6374 VL (SEQ ID NO:153), ATL_6375 (SEQ ID NO:153), ATL_6376 VL (SEQ ID NO: 153), ATL_6378 VL (SEQ ID NO: 153), ATL_6194 VL (SEQ ID NO: 154), ATL_6377 VL (SEQ ID NO: 154), ATL_6203 VL (SEQ ID NO: 154), ATL_6204 VL (SEQ ID NO: 155, ATL_6205 VL (SEQ ID NO: 156), ATL_6202 VL (SEQ ID NO: 159).

8. The isolated antibody or fragment thereof according to any one of the preceding claims, wherein:
The heavy chain variable domain comprises the amino acid sequence of any of ATL_5895 VH (SEQ ID NO: 7), ATL_6376 VH (SEQ ID NO: 175), ATL_6377 VH (SEQ ID NO: 176), or a sequence comprising at most 1, 2 or 3 mutations compared to any of these sequences; and
the light chain variable domain comprises the amino acid sequence of any of ATL_5895 VL (SEQ ID NO:8), ATL_6376 VL (SEQ ID NO: 153), ATL_6377 VL (SEQ ID NO: 154), or a sequence comprising at most 1, 2 or 3 mutations compared to any of these sequences.

9. The isolated antibody or fragment thereof according to any one of claims 1 to 8, wherein
a. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCIPPPYYYYYGLDVWGQGTTVTVSS (SEQ ID NO: 7) (ATL_5895 VH); and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCGSYAGTANVFGTGTKVTVL (SEQ ID NO:8) (ATL_5895 VL), or
b. the heavy chain variable domain comprises amino acid sequence EVQLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYWCVPPPYYYYYGLDVWGQGTTVTVSS (SEQ ID NO: 148) (ATL_6374 VH); and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCVSYGGTENVFGTGTKVTVL (SEQ ID NO:153) (ATL_6374 VL), or
c. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCVPPPYYYYYGLDVWGQGTTVTVSS (SEQ ID NO: 145) (ATL_6375 VH); and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCVSYGGTENVFGTGTKVTVL (SEQ ID NO:153) (ATL_6375 VL), or
d. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCVPPPFYYYYGLDVWGQGTTVTVSS (SEQ ID NO: 175) (ATL_6376 VH); and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCVSYGGTENVFGTGTKVTVL (SEQ ID NO:153) (ATL_6376 VL), or
e. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCVPSPYYYYYGLDVWGQGTTVTVSS (SEQ ID NO: 176) (ATL_6377 VH); and the light chain variable domain sequence comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCVSYAGTANVFGTGTKVTVL (SEQ ID NO: 154) (ATL_6377 VL), or
f. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCVPSPYYYYYGLDVWGQGTTVTVSS (SEQ ID NO: 176) (ATL_6378 VH); and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCVSYGGTENVFGTGTKVTVL (SEQ ID NO:153) (ATL_6378 VL), or
g. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYWCSPPPYYYYYGLDVWGQGTTVTVSS (SEQ ID NO: 144) (ATL_6199 VH) and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCGSYAGTANVFGTGTKVTVL (SEQ ID NO: 8) (ATL_6199 VL); or
h. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCVPPPYYYYYGLDVWGQGTTVTVSS (SEQ ID NO: 145) (ATL_6200 VH) and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCGSYAGTANVFGTGTKVTVL (SEQ ID NO: 8) (ATL_6002 VL); or
i. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCSPPPFYYYYGLDVWGQGTTVTVSS (SEQ ID NO: 146) (ATL_6202 VH) and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCVSYGGTENVFGTGTKVTVL (SEQ ID NO: 159) (ATL_6202 VL), or
j. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCIPSPYYYYYGLDVWGQGTTVTVSS (SEQ ID NO: 147) (ATL_6203 VH) and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCVSYAGTANVFGTGTKVTVL (SEQ ID NO: 154) (ATL_6203 VL), or
k. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCIPPPYYYYYGLDVWGQGTTVTVSS (SEQ ID NO: 7) (ATL_6204 VH) and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCVSFAGTANVFGTGTKVTVL (SEQ ID NO: 155) (ATL_6204 VL), or
l. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYWCVPPPYYYYYGLDVWGQGTTVTVSS (SEQ ID NO: 148) (ATL_6205 VH) and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCVSYAGTANVFGTGTKVTVL (SEQ ID NO: 156) (ATL_6205 VL), or
n. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCVPPPYYYYYGLDVWGQGTTVTVSS (SEQ ID NO: 145) (ATL_6194 VH) and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCVSYAGTANVFGTGTKVTVL (SEQ ID NO: 154) (ATL_6194 VL), or
o. the heavy chain variable domain comprises amino acid sequence EVOLVESGGGLVKPGGSLRLSCAASGFTFNKAWMSWVRQAPGKGLEWVGRIKSGIDAGTTDYAAP VKGRFTISRDDSKNTLYLQMNSLKTEDTAVYYCTPPPYYYYYGLDVWGQGTTVTVSS (SEQ ID NO: 179) (ATL_6195 VH) and the light chain variable domain comprises amino acid sequence QSALTQPRSVSGSPGQSVTISCTGTSSDVGSYNLVSWYQQHPGKAPKLMIYEVNKRPSGVPDRFS GSKSGATASLTISGLQAEDEADYYCGSYAGTANVFGTGTKVTVL (SEQ ID NO: 8) (ATL_6195 VL), or
p. the heavy chain variable domain comprises a variable domain comprising an amino acid sequence that has at least 95% sequence identity or that comprises at most 1, 2 or 3 substitutions compared to any one of the heavy chain variable domains in (a) to (n), and/or the light chain variable domain comprises an amino acid sequence that has at least 90%, at least 95% sequence identity, or that comprises at most 1, 2, 3, 4 or 5 substitutions compared to any one of the light chain variable domains in (a) to (n).

10. The isolated antibody or fragment thereof according to any one of the preceding claims, wherein:
the HTT protein is human or mouse HTT, and/or
the isolated antibody or fragment thereof binds to a region located within Exon 1 of HTT.

11. The isolated antibody or fragment thereof according to any one of the preceding claims, wherein:
the isolated antibody or fragment thereof binds to HTT or a fragment thereof comprising at least a portion of Exon 1, with a lower EC50 value compared with a reference antibody as measured by sandwich ELISA, optionally wherein the HTT has 25Q repeats or 48 repeats in Exon 1,
and/or
the isolated antibody or fragment thereof binds to HTT or a fragment thereof comprising at least a portion of Exon 1, with an EC50 value or at most 15 nM, at most 12 nM, at most 10 nM or at most 5 nM as measured using by sandwich ELISA, optionally wherein the HTT has 25Q repeats or 48 repeats in Exon 1 and/or wherein the HTT or HTT fragment comprises the sequence of SEQ ID NOs: 43, 44, 45 or 46 and/or wherein the sandwich ELISA is performed as described herein (Examples, Materials and Methods).

12. The isolated antibody or fragment thereof according to any preceding claim, wherein:
the isolated antibody or fragment thereof has a higher relative binding to mutated and/or aggregated and non-mutated and/or non-aggregated HTT compared to a reference antibody (such as e.g. ATL_0005059).

13. The isolated antibody or fragment thereof of claim 11 or claim 12, wherein the reference antibody comprises:
a. a heavy chain variable (VH) domain with the following CDRs:
i. HCDR1 with amino acid sequence NAWMN (SEQ ID NO: 35);
ii. HCDR2 with amino acid sequence HIRTQAEGGTSDYAAPVKG (SEQ ID NO: 36);
iii. HCDR3 with amino acid sequence PPYYYYYGLDV (SEQ ID NO: 3);
b. a light chain variable (VL) domain with the following CDRs:
i. LCDR1 with amino acid sequence TGASSDVGTYDLVS (SEQ ID NO: 37);
ii. LCDR2 with amino acid sequence EVNKRPS (SEQ ID NO:5); and
iii. LCDR3 with amino acid sequence CSYAGYSTV (SEQ ID NO: 38),
optionally wherein the reference antibody is NI-302.8F1 described in U.S. Pat. No. 11,401,325 B2.

14. The isolated antibody or fragment thereof according to any one of the preceding claims, wherein the isolated antibody or fragment thereof binds mutated and/or aggregated HTT protein as determined by measuring immunoprecipitation of mutant HTT with the isolated antibody or fragment thereof, optionally wherein mutant HTT is a HTT protein or fragment thereof, optionally a fragment comprising exon 1, that comprises more than 35 glutamine residues in its polyQ tract.

15. The isolated antibody or fragment thereof of any one of the preceding claims, wherein:
the isolated antibody or fragment thereof recognises an epitope in a region corresponding to Exon 1 of the HTT gene, and/or
the isolated antibody or fragment thereof recognises an epitope located in the polyP region of HTT, and/or wherein the isolated antibody or fragment thereof recognises an epitope comprising the amino acid sequence QQQQPPPPPPPPPPP (SEQ ID NO: 47) or PQPQPPPPPPPPPPP (SEQ ID NO: 48), and/or
the isolated antibody or fragment thereof is capable of crossing the blood-brain barrier, and/or
the isolated antibody or fragment thereof is a bispecific antibody further comprising a region that binds to the transferrin receptor.

16. The isolated antibody or fragment thereof of any one of the preceding claims, wherein the isolated antibody or fragment thereof increases phagocytosis of a HTT protein or fragment thereof comprising exon 1 comprising 48Q repeats by a cell, optionally wherein the cell is a microglia cell, optionally an iPSC derived human microglia cell, optionally wherein the isolated antibody or fragment thereof increases phagocytosis of a HTT protein or fragment thereof comprising exon 1 comprising 48Q repeats by a cell in a dose dependent manner and/or wherein the increased phagocytosis is measured by detecting phagocytosis of beads coated with the HTT protein or fragment coated with a pH-sensitive fluorescent dye and/or wherein increased phagocytosis is measured as described herein (Materials and Methods).

17. The isolated antibody or fragment thereof of any one of the preceding claims, wherein the isolated antibody or fragment thereof reduces the rate of aggregation of a HTT protein or fragment thereof comprising exon 1 comprising 48Q repeats in a cell free assay, optionally wherein the reduced rate of aggregation is measured using a FRASE assay and/or wherein the reduced rate of aggregation is measured as described herein (Materials and Methods) and/or wherein immunodepletion of a solution comprising the HTT protein or fragment thereof with the isolated antibody or fragment thereof results in a Delta t50 for aggregation of the HTT protein or fragment thereof of at most 0.1, at most 0.2, or at most 0.3, and/or wherein the aggregation of the HTT protein or fragment thereof is measured in the present of HTT fibrils and/or brain homogenate from one or more R6/2 mice.

18. The isolated antibody or fragment thereof according to any one of the preceding claims, wherein the isolated antibody or fragment thereof reduces aggregation of mutant HTT, wherein the aggregation of the mutant HTT is measured as the presence and/or concentration of HTT aggregates in the brain of a transgenic mouse model of Huntington's disease, optionally a R6/1 mouse, optionally wherein treatment of said mice with the isolated antibody or fragment thereof for 12 weeks or more results in a statistically significant decrease in the concentration of HTT aggregates in the striatum and/or cortex.

19. The isolated antibody or fragment thereof according to any preceding claim, wherein the antibody or fragment binds to mutant HTT in vivo.

20. The isolated antibody or fragment thereof according to claim 18 or claim 19, wherein the mutant HTT is a HTT protein or fragment thereof, optionally a fragment comprising exon 1, that comprises more than 35 glutamine residues, or between 115 and 150 glutamine residues in its polyQ tract.

21. The isolated antibody or fragment thereof according to any one of claims 18 to 20, wherein the isolated antibody or fragment thereof does not reduce the level of non-mutated and/or non-aggregated HTT in the brain of a transgenic mouse model of Huntington's disease treated with the isolated antibody or fragment thereof, wherein the level of the non-mutated and/or non-aggregated HTT is measured as the concentration of soluble and/or non-mutated HTT said mouse, optionally wherein the mouse is a R6/1 mouse.

22. The isolated antibody or fragment thereof according to any preceding claim, wherein the antibody or fragment thereof maintains a percentage monomer above 95% or above 97% after incubation at −80° C., 4° C., 21° C., 40° C. for 4 weeks and/or after 10× freeze-thaw cycles; and/or wherein the antibody or fragment thereof binds to a HTT protein comprising Exon 1 of HTT and 48 glutamine residues in its polyQ tract following incubation at −80° C., 4° C., 21° C. and 40° C. for 4 weeks and/or 10× freeze-thaw cycles, as assessed by ELISA, optionally wherein said binding is not significantly different from the binding of said antibody to said HTT protein prior to incubation and/or 10× freeze-thaw cycles.

23. A DNA molecule or set of DNA molecules encoding an antibody or antibody fragment thereof according to any one of the previous claims.

24. A vector or set of vectors encoding the DNA molecule or molecules according to claim 23.

25. A host cell comprising the vector or set of vectors according to claim 23.

26. A method of treating a disease or disorder in a subject, comprising administering to the subject an effective amount of an isolated antibody or antibody fragment thereof according to any one claims 1 to 22, optionally wherein the treatment prevents and/or reduces seeding and/or aggregation of mutant HTT in the subject.

27. The method according to claim 26, wherein the treatment prevents and/or reduces aggregation of mutant HTT in the subject without reducing the level of non-mutated and/or non-aggregated HTT in the subject.

28. The method according to claim 26 or claim 27, wherein the mutant HTT is a HTT protein or fragment thereof, optionally a fragment comprising exon 1, that comprises more than 35 glutamine residues or 115-150 glutamine residues in its polyQ tract.

29. Use of isolated antibody or antibody fragment thereof according to any one of claims 1 to 22 in the manufacture of a medicament for the treatment of a disorder or disease.

30. A composition comprising an isolated antibody or antibody fragment thereof according to any one of claims 1 to 22, optionally wherein the composition comprises a pharmaceutically acceptable excipient, vehicle or carrier, and/or wherein the composition is for use in the treatment of a disease or disorder.

31. The method, use, or composition for use according to any one of claims 26 to 30, wherein the disease or disorder is a neurodegenerative disease or disorder, optionally wherein the neurodegenerative disorder is Huntington's disease, Alzheimer's disease, or frontotemporal dementia.


SEQUENCE LISTING (186 sequences)

SEQ ID NO: 1 - protein, 5 aa, synthetic construct
KAWMS

SEQ ID NO: 2 - protein, 19 aa, synthetic construct
RIKSGIDAGTTDYAAPVKG

SEQ ID NO: 3 - protein, 11 aa, synthetic construct
PPYYYYYGLDV

SEQ ID NO: 4 - protein, 14 aa, synthetic construct
TGTSSDVGSYNLVS

SEQ ID NO: 5 - protein, 7 aa, synthetic construct
EVNKRPS

SEQ ID NO: 6 - protein, 9 aa, synthetic construct
GSYAGTANV

SEQ ID NO: 7 - protein, 122 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYYCIPPPYYYYYGLDVWGQGTTVTVSS

SEQ ID NO: 8 - protein, 109 aa, synthetic construct
QSALTQPRSVSGSPGQSVTISCTGTSSDVGSYNLVSWYQQHPGKAPKLMIYEVNKRPSGVPDRFSGSKSGATASLTISGLQAEDEADYYCGSYAGTANVFGTGTKVTVL

SEQ ID NO: 9 - protein, 14 aa, synthetic construct
TGTSSDVGGYKLVS

SEQ ID NO: 10 - protein, 7 aa, synthetic construct
EVSKRPS

SEQ ID NO: 11 - protein, 9 aa, synthetic construct
SSYAGSSVV

SEQ ID NO: 12 - protein, 122 aa, synthetic construct
EVQLVESGGGLVKPGGSLRLSCAASGFTFNKAWMSWVRQAPGKGLEWVGRIKSGIDAGTTDYAAPVKGRFTISRDDSKATLYLQMNSLKTEDTAVYYCIPPPYYYYYGLDVWGQGTTVTVSS

SEQ ID NO: 13 - protein, 109 aa, synthetic construct
QSALTQPASVSGSPGQSITISCTGTSSDVGGYKLVSWYQQHPGRAPKLMIYEVSKRPSGVSSRFSGSKSGSTASLTISGLQAEDEADYYCSSYAGSSVVFGGGTKLTVL

SEQ ID NO: 14 - protein, 5 aa, synthetic construct
KAWMN

SEQ ID NO: 15 - protein, 19 aa, synthetic construct
RIKSGIDGGTTDYAAPVQG

SEQ ID NO: 16 - protein, 14 aa, synthetic construct
TGTSSDIGSYNLVS

SEQ ID NO: 17 - protein, 7 aa, synthetic construct
EGSKRPS

SEQ ID NO: 18 - protein, 10 aa, synthetic construct
SSYAGFSTLV

SEQ ID NO: 19 - protein, 122 aa, synthetic construct
EVQLVESGGGLVKPGGSLRLSCAASGFTFNKAWMNWVRQAPGKGLEWVGRIKSGIDGGTTDYAAPVQGRFTISRDDSKNTVYLQMNSLNTEDTAVYYCIPPPYYYYYGLDVWGQGTTVTVSS

SEQ ID NO: 20 - protein, 110 aa, synthetic construct
QSALTQPASVSGSPGQSITISCTGTSSDIGSYNLVSWYQQHPGNAPKPLIYEGSKRPSGVSARFSGSKSGNTASLTISGLQPEDEADYYCSSYAGFSTLVFGGGTKVTVL

SEQ ID NO: 21 - protein, 9 aa, synthetic construct
GSYAGTNNV

SEQ ID NO: 22 - protein, 109 aa, synthetic construct
QSALTQPRSVSGSPGQSITISCTGTSSDVGSYNLVSWFQQHPGKAPKLIIYEVNKRPSGVPDRFSGSKSGNTASLTVSGLQAEDEADYYCGSYAGTNNVFGTGTKLTVL

SEQ ID NO: 23 - protein, 9 aa, synthetic construct
CSYAGSSVV

SEQ ID NO: 24 - protein, 109 aa, synthetic construct
QSALTQPASVSGSPGQSITISCTGTSSDVGGYKLVSWYQQHPGRAPKLMIYEVSKRPSGVSSRFSGSKSGSTASLTISGLQAEDEADYYCCSYAGSSVVFGGGTKLTVL

SEQ ID NO: 25 - protein, 10 aa, synthetic construct
SSYAGFNTLV

SEQ ID NO: 26 - protein, 110 aa, synthetic construct
QSALTQPASVSGSPGQSITISCTGTSSDIGSYNLVSWYQQHPGNAPKPLIYEGSKRPSGVSARFSGSKSGNTASLTISGLQPEDEADYYCSSYAGFNTLVFGGGTKVTVL

SEQ ID NO: 27 - protein, 19 aa, synthetic construct
RIKSGIDGGTTDYAAPVKG

SEQ ID NO: 28 - protein, 122 aa, synthetic construct
EVQLVESGGGLVKPGGSLRLSCAASGFTFNKAWMSWVRQAPGKGLEWVGRIKSGIDGGTTDYAAPVKGRFTISRDDSKNTLYLQMNSLKTEDTAVYYCIPPPYYYYYGLDVWGQGTTVTVSS

SEQ ID NO: 29 - protein, 109 aa, synthetic construct
QSALTQPRSVSGSPGQSVTISCTGTSSDVGSYNLVSWYQQHPGKAPKLMIYEVNKRPSGVPDRFSGSKSGNTASLTISGLQAEDEADYYCGSYAGTNNVFGTGTKVTVL

SEQ ID NO: 30 - protein, 110 aa, synthetic construct
QSALTQPASVSGSPGQSITISCTGTSSDIGSYNLVSWYQQHPGKAPKLMIYEGSKRPSGVSNRFSGSKSGNTASLTISGLQAEDEADYYCSSYAGFNTLVFGGGTKLTVL

SEQ ID NO: 31 - protein, 122 aa, synthetic construct
EVQLVESGGGLVKPGGSLRLSCAASGFTFNKAWMNWVRQAPGKGLEWVGRIKSGIDAGTTDYAAPVQGRFTISRDDSKNTVYLQMNSLNTEDTAVYYCIPPPYYYYYGLDVWGQGTTVTVSS

SEQ ID NO: 32 - protein, 109 aa, synthetic construct
QSALTQPRSVSGSPGQSVTISCTGTSSDVGSYNLVSWYQQHPGKAPKLIIYEVNKRPSGVPDRFSGSKSGNTASLTISGLQAEDEADYYCGSYAGTNNVFGTGTKVTVL

SEQ ID NO: 33 - protein, 122 aa, synthetic construct
EVQLVESGGGLVKPGGSLTISCAASGFTFSNAWMNWVRQAPGKGLEWVGHIRTQAEGGTSDYAAPVKGRFTISRDDSKNTLYLQMNSLKTEDTAVYYCIPPPYYYYYGLDVWGQGTTVTVSS

SEQ ID NO: 34 - protein, 109 aa, synthetic construct
QSALTQPASVSGSPGQSITISCTGASSDVGTYDLVSWYQQHPGKAPKLIIYEVNKRPSGVSYRFSASKSANTASLTISGLQAEDEAEYYCCSYAGYSTVFGGGTKLTVL

SEQ ID NO: 35 - protein, 5 aa, synthetic construct
NAWMN

SEQ ID NO: 36 - protein, 19 aa, synthetic construct
HIRTQAEGGTSDYAAPVKG

SEQ ID NO: 37 - protein, 14 aa, synthetic construct
TGASSDVGTYDLVS

SEQ ID NO: 38 - protein, 9 aa, synthetic construct
CSYAGYSTV

SEQ ID NO: 39 - protein, 110 aa, synthetic construct
QSALTQPASVSGSPGQSITISCTGTSSDIGSYNLVSWYQQHPGKAPKPLIYEGSKRPSGVSNRFSGSKSGNTASLTISGLQAEDEADYYCSSYAGFNTLVFGGGTKLTVL

SEQ ID NO: 40 - protein, 109 aa, synthetic construct
QSALTQPRSVSGSPGQSVTISCTGTSSDVGSYNLVSWYQQHPGKAPKLMIYEVNKRPSGVPDRFSGSKSGATASLTISGLQAEDEADYYCGSYAGTNNVFGTGTKVTVL

SEQ ID NO: 41 - protein, 109 aa, synthetic construct
QSALTQPRSVSGSPGQSVTISCTGTSSDVGSYNLVSWYQQHPGKAPKLIIYEVNKRPSGVPDRFSGSKSGATASLTISGLQAEDEADYYCGSYAGTANVFGTGTKVTVL

SEQ ID NO: 42 - protein, 109 aa, synthetic construct
QSALTQPRSVSGSPGQSITISCTGTSSDVGSYNLVSWFQQHPGKAPKLIIYEVNKRPSGVPDRFSGSKSGATASLTVSGLQAEDEADYYCGSYAGTNNVFGTGTKLTVL

SEQ ID NO: 43 - protein, 343 aa, Homo sapiens
MKHHHHHHNTSSNSMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPTSGSGGGGGWMSENLYFQGAMATLEKLMKAFESLKSFQQQQQQQQQQQQQQQQQQQQQQQQQPPPPPPPPPPPQLPQPPPQAQPLLPQPQPPPPPPPPPPGPAVAEEPLHRP

SEQ ID NO: 44 - protein, 366 aa, Homo sapiens
MKHHHHHHNTSSNSMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPTSGSGGGGGWMSENLYFQGAMATLEKLMKAFESLKSFQQQQQQQQQQQQQQQQQQQQQQQQQQQQQQQQQQQQQQQQQQQQQQQQPPPPPPPPPPPQLPQPPPQAQPLLPQPQPPPPPPPPPPGPAVAEEPLHRP

SEQ ID NO: 45 - protein, 124 aa, synthetic construct
MGSSHHHHHHSQDPNSGGGGGWMSENLYFQGAMATLEKLMKAFESLKSFQQQQQQQQQQQQQQQQQQQQQQQQQPPPPPPPPPPPQLPQPPPQAQPLLPQPQPPPPPPPPPPGPAVAEEPLHRP

SEQ ID NO: 46 - protein, 147 aa, synthetic construct
MGSSHHHHHHSQDPNSGGGGGWMSENLYFQGAMATLEKLMKAFESLKSFQQQQQQQQQQQQQQQQQQQQQQQQQQQQQQQQQQQQQQQQQQQQQQQQPPPPPPPPPPPQLPQPPPQAQPLLPQPQPPPPPPPPPPGPAVAEEPLHRP

SEQ ID NO: 47 - protein, 15 aa, synthetic construct
QQQQPPPPPPPPPPP

SEQ ID NO: 48 - protein, 16 aa, synthetic construct
LPQPQPPPPPPPPPPP

SEQ ID NO: 49 - protein, 82 aa, Homo sapiens
MATLEKLMKAFESLKSFQQQQQQQQQQQQQQQQPPPPPPPPPPPQLPQPPPQAQPLLPQPQPPPPPPPPPPGPAVAEEPLHR

SEQ ID NO: 50 - protein, 89 aa, Macaca fascicularis
MATLEKLMKAFESLKSFQQQQQQQQQQQPPPPPPPPPPPPQLPQPPQAQPMLPQPQPPPPPPRHHPARLWLRSRCTDQRKNFQLPRKTV

SEQ ID NO: 51 - protein, 93 aa, Macaca fascicularis
MATLEKLMKAFESLKSFQQQQQQQQQQQQPPPPPPPPPPPPQLPQPPQAQPMLPQPQPPPPPPPPPPGPAVAEEPLHRPKKELSATKKDRVNH

SEQ ID NO: 52 - protein, 67 aa, Mus musculus
MATLEKLMKAFESLKSFQQQQQQQPPPQAPPPPPPPPPPQPPQPPPQGQPPPPPPPLPGPAEEPLHR

SEQ ID NO: 53 - protein, 122 aa, synthetic construct
EVQLVESGGGLVKPGGSLRLSCAASGFTFSNAWMSWVRQAPGKGLEWVGRIKSKTDSGTTDYAAPVRGRFTISRDDSKNTLFLQMNSLKTEDTAVYYCVPAPYYYYYGLDVWGQGTTVTVSS

SEQ ID NO: 54 - protein, 15 aa, synthetic construct
QQQQQQQQPPPPPPP

SEQ ID NO: 55 - protein, 15 aa, synthetic construct
QQQQQQQPPPPPPPP

SEQ ID NO: 56 - protein, 15 aa, synthetic construct
QQQQQQPPPPPPPPP

SEQ ID NO: 57 - protein, 15 aa, synthetic construct
QQQQQPPPPPPPPPP

SEQ ID NO: 58 - protein, 15 aa, synthetic construct
QQQPPPPPPPPPPPQ

SEQ ID NO: 59 - protein, 15 aa, synthetic construct
QQPPPPPPPPPPPQL

SEQ ID NO: 60 - protein, 15 aa, synthetic construct
PQAQPLLPQPQPPPP

SEQ ID NO: 61 - protein, 15 aa, synthetic construct
QAQPLLPQPQPPPPP

SEQ ID NO: 62 - protein, 15 aa, synthetic construct
AQPLLPQPQPPPPPP

SEQ ID NO: 63 - protein, 15 aa, synthetic construct
QPLLPQPQPPPPPPP

SEQ ID NO: 64 - protein, 15 aa, synthetic construct
PLLPQPQPPPPPPPP

SEQ ID NO: 65 - protein, 15 aa, synthetic construct
LLPQPQPPPPPPPPP

SEQ ID NO: 66 - protein, 15 aa, synthetic construct
LPQPQPPPPPPPPPP

SEQ ID NO: 67 - protein, 15 aa, synthetic construct
PQPQPPPPPPPPPPG

SEQ ID NO: 68 - protein, 15 aa, synthetic construct
QPQPPPPPPPPPPGP

SEQ ID NO: 69 - protein, 15 aa, synthetic construct
PQPPPPPPPPPPGPA

SEQ ID NO: 70 - protein, 15 aa, synthetic construct
QPPPPPPPPPPGPAV

SEQ ID NO: 71 - protein, 15 aa, synthetic construct
QQQPPPPPPPPPPPP

SEQ ID NO: 72 - protein, 15 aa, synthetic construct
PQAQPMLPQPQPPPP

SEQ ID NO: 73 - protein, 15 aa, synthetic construct
QAQPMLPQPQPPPPP

SEQ ID NO: 74 - protein, 15 aa, synthetic construct
AQPMLPQPQPPPPPP

SEQ ID NO: 75 - protein, 15 aa, synthetic construct
QPMLPQPQPPPPPPR

SEQ ID NO: 76 - protein, 15 aa, synthetic construct
QQQQQQQQQQPPPPP

SEQ ID NO: 77 - protein, 15 aa, synthetic construct
QQQQQQQQQPPPPPP

SEQ ID NO: 78 - protein, 15 aa, synthetic construct
QQPPPPPPPPPPPPQ

SEQ ID NO: 79 - protein, 15 aa, synthetic construct
QPPPPPPPPPPPPQL

SEQ ID NO: 80 - protein, 15 aa, synthetic construct
PPPPPPPPPPPPQLP

SEQ ID NO: 81 - protein, 15 aa, synthetic construct
QPMLPQPQPPPPPPP

SEQ ID NO: 82 - protein, 15 aa, synthetic construct
PMLPQPQPPPPPPPP

SEQ ID NO: 83 - protein, 15 aa, synthetic construct
MLPQPQPPPPPPPPP

SEQ ID NO: 84 - protein, 15 aa, synthetic construct
LPQPQPPPPPPPPPP

SEQ ID NO: 85 - protein, 15 aa, synthetic construct
QQQQQPPPQAPPPPP

SEQ ID NO: 86 - protein, 15 aa, synthetic construct
QQQQPPPQAPPPPPP

SEQ ID NO: 87 - protein, 15 aa, synthetic construct
QQQPPPQAPPPPPPP

SEQ ID NO: 88 - protein, 15 aa, synthetic construct
QQPPPQAPPPPPPPP

SEQ ID NO: 89 - protein, 15 aa, synthetic construct
QPPPQAPPPPPPPPP

SEQ ID NO: 90 - protein, 15 aa, synthetic construct
PPPQAPPPPPPPPPP

SEQ ID NO: 91 - protein, 15 aa, synthetic construct
PPQAPPPPPPPPPPQ

SEQ ID NO: 92 - protein, 15 aa, synthetic construct
PQAPPPPPPPPPPQP

SEQ ID NO: 93 - protein, 15 aa, synthetic construct
QAPPPPPPPPPPQPP

SEQ ID NO: 94 - protein, 15 aa, synthetic construct
PPQPPPQGQPPPPPP

SEQ ID NO: 95 - protein, 15 aa, synthetic construct
PQPPPQGQPPPPPPP

SEQ ID NO: 96 - protein, 15 aa, synthetic construct
QPPPQGQPPPPPPPL

SEQ ID NO: 97 - protein, 15 aa, synthetic construct
PPPQGQPPPPPPPLP

SEQ ID NO: 98 - protein, 30 aa, synthetic construct
EVQLVESGGGLVKPGGSLRLSCAASGFTFN

SEQ ID NO: 99 - protein, 14 aa, synthetic construct
WVRQAPGKGLEWVG

SEQ ID NO: 100 - protein, 32 aa, synthetic construct
RFTISRDDSKNTVYLQMNSLNTEDTAVYYCIP

SEQ ID NO: 101 - protein, 11 aa, synthetic construct
WGQGTTVTVSS

SEQ ID NO: 102 - protein, 22 aa, synthetic construct
QSALTQPRSVSGSPGQSITISC

SEQ ID NO: 103 - protein, 15 aa, synthetic construct
WFQQHPGKAPKLIIY

SEQ ID NO: 104 - protein, 32 aa, synthetic construct
GVPDRFSGSKSGNTASLTVSGLQAEDEADYYC

SEQ ID NO: 105 - protein, 10 aa, synthetic construct
FGTGTKLTVL

SEQ ID NO: 106 - protein, 22 aa, synthetic construct
QSALTQPASVSGSPGQSITISC

SEQ ID NO: 107 - protein, 15 aa, synthetic construct
WYQQHPGRAPKLMIY

SEQ ID NO: 108 - protein, 32 aa, synthetic construct
GVSSRFSGSKSGSTASLTISGLQAEDEADYYC

SEQ ID NO: 109 - protein, 10 aa, synthetic construct
FGGGTKLTVL

SEQ ID NO: 110 - protein, 15 aa, synthetic construct
WYQQHPGNAPKPLIY

SEQ ID NO: 111 - protein, 32 aa, synthetic construct
GVSARFSGSKSGNTASLTISGLQPEDEADYYC

SEQ ID NO: 112 - protein, 10 aa, synthetic construct
FGGGTKVTVL

SEQ ID NO: 113 - protein, 15 aa, synthetic construct
  VARIANT 1: note = X can be P or Q
  VARIANT 3: note = X can be P or Q
XQXQPPPPPPPPPPP

SEQ ID NO: 114 - protein, 122 aa, synthetic construct
EVQLVESGGGLVKPGGSLRLSCAASGFTFSNAWMNWVRQAPGKGLEWVGRIKSKTDSGTTDYAAPVRGRFTISRDDSKNTLYLQMNSLKSEDTAVYYCVPPPYYYYYGLDVWGQGTTVTVSS

SEQ ID NO: 115 - protein, 122 aa, synthetic construct
EVQLVESGGGLVKPGGSLRLSCAASGFTFSNAWMSWVRQAPGKGLEWVGRIKSKTDSGTTHYAAPVRGRFTISRDDSKNTLFMQMNSLKTEQTAFYYCVPAPYYYYCGLDVWGQGTTVTVSS

SEQ ID NO: 116 - protein, 122 aa, synthetic construct; identical to SEQ ID NO: 19

SEQ ID NO: 117 - protein, 122 aa, synthetic construct
EVQLVESGGGLVKPGGSLRLSCAASGFTFNKAWMSWVRQAPGKGLEWIGRIKPTTDGGTADYAAPVMGRFTISRDDSKNTLYLQMNSLKTEDTAVYYCVPPPYYYYNGLDVWGQGTTVTVSS

SEQ ID NO: 118 - protein, 120 aa, synthetic construct
EVQLVESGGGLVKPGGSLTISCAASGFTFSNAWMNWVRQAPGKGLEWVGHIRTQAEGGTSAAPVKGRFTISRDDSKNTLYLQMNSLKTEDTAVYYCIPPPYYYYYGLDVWGQGTTVTVSS

SEQ ID NO: 119 - protein, 22 aa, synthetic construct
MATLEKLMKAFESLKSFQQQQQ

SEQ ID NO: 120 - protein, 15 aa, synthetic construct
MATLEKLMKAFESLK

SEQ ID NO: 121 - protein, 15 aa, synthetic construct
ATLEKLMKAFESLKS

SEQ ID NO: 122 - protein, 15 aa, synthetic construct
TLEKLMKAFESLKSF

SEQ ID NO: 123 - protein, 15 aa, synthetic construct
LEKLMKAFESLKSFQ

SEQ ID NO: 124 - protein, 15 aa, synthetic construct
EKLMKAFESLKSFQQ

SEQ ID NO: 125 - protein, 15 aa, synthetic construct
KLMKAFESLKSFQQQ

SEQ ID NO: 126 - protein, 15 aa, synthetic construct
LMKAFESLKSFQQQQ

SEQ ID NO: 127 - protein, 15 aa, synthetic construct
MKAFESLKSFQQQQQ

SEQ ID NO: 128 - protein, 19 aa, synthetic construct
RIKSGIDAGTTDYAAPVQG

SEQ ID NO: 129 - protein, 32 aa, synthetic construct
RFTISRDDSKATLYLQMNSLKTEDTAVYYCIP

SEQ ID NO: 130 - protein, 32 aa, synthetic construct
RFTISRDDSKNTLYLQMNSLKTEDTAVYYCIP

SEQ ID NO: 131 - protein, 22 aa, synthetic construct
QSALTQPRSVSGSPGQSVTISC

SEQ ID NO: 132 - protein, 15 aa, synthetic construct
WYQQHPGKAPKLIIY

SEQ ID NO: 133 - protein, 15 aa, synthetic construct
WYQQHPGKAPKLMIY

SEQ ID NO: 134 - protein, 15 aa, synthetic construct
WYQQHPGKAPKPLIY

SEQ ID NO: 135 - protein, 32 aa, synthetic construct
GVPDRFSGSKSGNTASLTISGLQAEDEADYYC

SEQ ID NO: 136 - protein, 32 aa, synthetic construct
GVPDRFSGSKSGATASLTVSGLQAEDEADYYC

SEQ ID NO: 137 - protein, 32 aa, synthetic construct
GVSNRFSGSKSGNTASLTISGLQAEDEADYYC

SEQ ID NO: 138 - protein, 32 aa, synthetic construct
GVPDRFSGSKSGATASLTISGLQAEDEADYYC

SEQ ID NO: 139 - protein, 10 aa, synthetic construct
FGTGTKVTVL

SEQ ID NO: 140 - protein, 109 aa, synthetic construct
QSALTQPASVSGSPGQSITISCTGTTSDVGSYNLVSWYQQHPGKAPKLMIYEVTKRPSGISDRFSGSKSGNTASLTLSGLQPEDEAVYHCCSYAGGSVVFGGGTKLTVL

SEQ ID NO: 141 - protein, 109 aa, synthetic construct
QSALTQPASVSGSPGQSITISCTGTSSDVGSYNLASWYQQHPGKAPKLMIYEVSKRPSGVSNRFSGSKSGNTASLTISGLQAEDEADYYCCSYAGSSTVFGGGTKLTVL

SEQ ID NO: 142 - protein, 10 aa, synthetic construct
YPYDVPDYAG

SEQ ID NO: 143 - protein, 14 aa, synthetic construct
KEVPALTAVETGAT

SEQ ID NO: 144 - protein, 122 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYWCSPPPYYYYYGLDVWGQGTTVTVSS

SEQ ID NO: 145 - protein, 122 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYYCVPPPYYYYYGLDVWGQGTTVTVSS

SEQ ID NO: 146 - protein, 122 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYYCSPPPFYYYYGLDVWGQGTTVTVSS

SEQ ID NO: 147 - protein, 122 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYYCIPSPYYYYYGLDVWGQGTTVTVSS

SEQ ID NO: 148 - protein, 122 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYWCVPPPYYYYYGLDVWGQGTTVTVSS

SEQ ID NO: 149 - protein, 121 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYYCTPPPYYYYGLDVWGQGTTVTVSS

SEQ ID NO: 150 - protein, 121 aa, synthetic construct
EVQLVESGGGLVKPGGSLRLSCAASGFTFNKAWMSWVRQAPGKGLEWVGRIKSGIDAGTTDYAAPVKGRFTISRDDSKATLYLQMNSLKTEDTAVYYCTPPPYYYYGLDVWGQGTTVTVSS

SEQ ID NO: 151 - protein, 121 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYYCTPPPYYYYGLNVWGQGTTVTVSS

SEQ ID NO: 152 - protein, 121 aa, synthetic construct
EVQLVESGGGLVKPGGSLRLSCAASGFTFNKAWMSWVRQAPGKGLEWVGRIKSGIDAGTTDYAAPVKGRFTISRDDSKATLYLQMNSLKTEDTAVYYCTPPPYYYYGLNVWGQGTTVTVSS

SEQ ID NO: 153 - protein, 109 aa, synthetic construct
QSALTQPRSVSGSPGQSVTISCTGTSSDVGSYNLVSWYQQHPGKAPKLMIYEVNKRPSGVPDRFSGSKSGATASLTISGLQAEDEADYYCVSYGGTENVFGTGTKVTVL

SEQ ID NO: 154 - protein, 109 aa, synthetic construct
QSALTQPRSVSGSPGQSVTISCTGTSSDVGSYNLVSWYQQHPGKAPKLMIYEVNKRPSGVPDRFSGSKSGATASLTISGLQAEDEADYYCVSYAGTANVFGTGTKVTVL

SEQ ID NO: 155 - protein, 109 aa, synthetic construct
QSALTQPRSVSGSPGQSVTISCTGTSSDVGSYNLVSWYQQHPGKAPKLMIYEVNKRPSGVPDRFSGSKSGATASLTISGLQAEDEADYYCVSFAGTANVFGTGTKVTVL

SEQ ID NO: 156 - protein, 109 aa, synthetic construct; identical to SEQ ID NO: 154

SEQ ID NO: 157 - protein, 11 aa, synthetic construct
SPYYYYYGLDV

SEQ ID NO: 158 - protein, 11 aa, synthetic construct
PPFYYYYGLDV

SEQ ID NO: 159 - protein, 10 aa, synthetic construct
PPYYYYGLNV

SEQ ID NO: 160 - protein, 9 aa, synthetic construct
VSFAGTANV

SEQ ID NO: 161 - protein, 9 aa, synthetic construct
VSYAGTANV

SEQ ID NO: 162 - protein, 9 aa, synthetic construct
VSYGGTENV

SEQ ID NO: 163 - protein, 15 aa, synthetic construct
YCIPPPYYYYYGLDV

SEQ ID NO: 164 - protein, 15 aa, synthetic construct
WCSPPPYYYYYGLDV

SEQ ID NO: 165 - protein, 15 aa, synthetic construct
YCVPPPYYYYYGLDV

SEQ ID NO: 166 - protein, 15 aa, synthetic construct
YCSPPPFYYYYGLDV

SEQ ID NO: 167 - protein, 15 aa, synthetic construct
YCIPSPYYYYYGLDV

SEQ ID NO: 168 - protein, 15 aa, synthetic construct
WCVPPPYYYYYGLDV

SEQ ID NO: 169 - protein, 32 aa, synthetic construct
RFTISRDDSKNTLYLQMNSLKTEDTAVYWCSP

SEQ ID NO: 170 - protein, 32 aa, synthetic construct
RFTISRDDSKNTLYLQMNSLKTEDTAVYYCVP

SEQ ID NO: 171 - protein, 32 aa, synthetic construct
RFTISRDDSKNTLYLQMNSLKTEDTAVYYCSP

SEQ ID NO: 172 - protein, 32 aa, synthetic construct; identical to SEQ ID NO: 130

SEQ ID NO: 173 - protein, 32 aa, synthetic construct
RFTISRDDSKNTLYLQMNSLKTEDTAVYWCVP

SEQ ID NO: 174 - protein, 18 aa, synthetic construct
GYSLPQPQPPPPPPPPPP

SEQ ID NO: 175 - protein, 122 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYYCVPPPFYYYYGLDVWGQGTTVTVSS

SEQ ID NO: 176 - protein, 122 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYYCVPSPYYYYYGLDVWGQGTTVTVSS

SEQ ID NO: 177 - protein, 99 aa, synthetic construct
MGSSHHHHHHSQDPNSGGGGGWMSENLYFQGAMATLEKLMKAFESLKSFQQQQQQQPPPQAPPPPPPPPPQPPQPPPQGQPPPPPPPLPGPAEEPLHRP

SEQ ID NO: 178 - protein, 32 aa, synthetic construct; identical to SEQ ID NO: 173

SEQ ID NO: 179 - protein, 122 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYYCTPPPYYYYYGLDVWGQGTTVTVSS

SEQ ID NO: 180 - protein, 32 aa, synthetic construct
RFTISRDDSKNTLYLQMNSLKTEDTAVYYCTP

SEQ ID NO: 181 - protein, 122 aa, synthetic construct; identical to SEQ ID NO: 53

SEQ ID NO: 182 - protein, 109 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYYCVPPPYYYYYGL

SEQ ID NO: 183 - protein, 100 aa, synthetic construct
QSALTQPRSVSGSPGQSVTISCTGTSSDVGSYNLVSWYQQHPGKAPKLMIYEVNKRPSGVPDRFSGSKSGATASLTISGLQAEDEADYYCVSYAGTANVF

SEQ ID NO: 184 - protein, 109 aa, synthetic construct
EVQLVESGGGLVKPGGSLRLSCAASGFTFNKAWMSWVRQAPGKGLEWVGRIKSGIDAGTTDYAAPVKGRFTISRDDSKNTLYLQMNSLKTEDTAVYYCTPPPYYYYYGL

SEQ ID NO: 185 - protein, 100 aa, synthetic construct
QSALTQPRSVSGSPGQSVTISCTGTSSDVGSYNLVSWYQQHPGKAPKLMIYEVNKRPSGVPDRFSGSKSGATASLTISGLQAEDEADYYCGSYAGTANVF

SEQ ID NO: 186 - protein, 15 aa, synthetic construct
YCTPPPYYYYYGLDV